(19) United States
(12) Patent Application Publication
(10) Pub. No.: US 2024/0044871 A1
(43) Pub. Date: Feb. 8, 2024

(54) CHIMERIC RECEPTOR POLYPEPTIDE AND METHODS OF ACTIVATION THEREOF

(71) Applicant: REFUGE BIOTECHNOLOGIES, INC., Menlo Park, CA (US)

(72) Inventors: Francesco M. MARINCOLA, Menlo Park, CA (US); Rona HARARI-STEINFELD, Sunnyvale, CA (US); Alper KEARNEY, Belmont, CA (US); Dharmeshkumar PATEL, Fremont, CA (US)

(21) Appl. No.: 18/096,908

(22) Filed: Jan. 13, 2023

Related U.S. Application Data
(63) Continuation of application No. 17/833,004, filed on Jun. 6, 2022, now abandoned.
Application No. 17/833,004 is a continuation of application No. PCT/US20/64363, filed on Dec. 10, 2020.
(60) Provisional application No. 62/946,339, filed on Dec. 10, 2019.

(51) Int. Cl. G01N 33/50

(52) U.S. Cl. CPC G01N 33/5047; G01N 33/5023; G01N 2333/55; G01N 2333/525; G01N 2333/57

(57) ABSTRACT

Described herein are methods for assessing activity of engineered cells, such as engineered immune cells by using non-cellular substrates. Also described herein are methods for determining suitability of the engineered cells for treatment (e.g., via administration of the engineered cells) of a subject in need thereof.

### CROSS REFERENCE

This application is a continuation of U.S. application Ser. No. 17/833,004, filed Jun. 6, 2022, which is a continuation of International Patent Application No. PCT/US20/64363, filed Dec. 10, 2020, which claims the benefit of U.S. Provisional Application No. 62/946,339, filed on Dec. 10, 2019, each of which is incorporated herein by reference in its entirety.

### BACKGROUND

The use of immune cells for targeting and killing pathogens is one of the cornerstones of modern medicine. Modifications made to the targeting moieties of the immune cells further expand the use of immune cells to any target that can be recognized and bound by the modified targeting moieties. For example, immune cells can be modified to recognize and bind to a chosen target (e.g., an antigen), where binding to the target triggers immune cell activation signaling cascades. Accordingly, the modified immune cells have been extensive examined and implemented in development of therapies to treat a wide variety of diseases.

However, manufacturing and assuring consistent activities of the modified immune cells present an ongoing challenge. The modified immune cells are often produced in batches, which lead to inconsistent cellular characteristics and activities. Defective modified immune cells can lead to off-target effects, low activity, reduced cellular proliferation, exhaustion, etc. Administering such defective modified immune cells can also cause adverse events ranging from ineffective treatment to massive inflammatory response which has led to death of patients.

### SUMMARY

In view of the foregoing, there exists a considerable need for methods to assay engineered cells (e.g., modified immune cells) prior to these cells being administered to subjects in need thereof or used in any other applications.

In one aspect, the present disclosure describes a method of assessing a plurality of cells for use in a cell therapy for a subject in need thereof, comprising: providing the plurality of cells capable of expressing one or more chimeric receptors comprising a ligand binding domain specific for an antigen; assessing an activity of a first sub-population of the plurality of cells upon treatment with a non-cellular substrate comprising the antigen; and assessing a suitability of the plurality of cells for the cell therapy based upon a threshold of the activity, wherein the threshold of the activity comprises one or more members selected from the group consisting of: (i) at least about 50% viability over at least about 24 hours; (ii) at least about 10% increase in a percentage of the plurality of cells expressing a cell activation marker over at least about 24 hours, as compared to control cells; (iii) at least about 5% increase in a percentage of the plurality of cells expressing the one or more chimeric receptors over 48 hours, as compared to control cells; (iv) at least about 5% increase in a percentage of the plurality of cells expressing an immune checkpoint inhibitor over at least about 24 hours, as compared to control cells; (v) at least about 10% increase in expression of a cytokine over at least about 24 hours, as compared to control cells; (vi) at least about 20% increase in a percentage of the plurality of cells in a proliferative state over at least about 48 hours, as compared to control cells; or (vii) at least about 10% change in a percentage of the plurality of cells expressing a cell exhaustion marker over at least about 24 hours, as compared to control cells.

In some embodiments, the threshold of the activity comprises two, three, four, five, or all members of (i)-(vi). In some embodiments, the threshold of the activity comprises two of (i)-(vi). In some embodiments, the threshold of the activity comprises three of (i)-(vi). In some embodiments, the threshold of the activity comprises four of (i)-(vi). In some embodiments, the threshold of the activity comprises five of (i)-(vi). In some embodiments, the threshold of the activity comprises all of (i)-(vi).

In some embodiments, the threshold of the activity comprises (i) at least about 50%, 60%, 70%, 80%, or 90% viability over at least about 24 hours.

In some embodiments, the threshold of the activity comprises (ii) at least about 10%, 20%, 40%, or 60% increase in the percentage of the plurality of cells expressing the cell activation marker over at least about 24 hours, as compared to control cells.

In some embodiments, the threshold of the activity comprises (iii) at least about 5%, 10%, 15%, or 20% increase in the percentage of the plurality of cells expressing the one or more chimeric receptors over 48 hours, as compared to control cells.

In some embodiments of any of the methods disclosed herein, for example in paragraph [0009], the plurality of cells is capable of expressing an additional heterologous polypeptide, and wherein the threshold of the activity comprises at least about 5%, 10%, 15%, or 20% increase in the percentage of the plurality of cells expressing both (1) the one or more chimeric receptors and (2) the additional heterologous polypeptide over 48 hours, as compared to control cells.

In some embodiments of any of the methods disclosed herein, for example in paragraphs [0009]-[0010], the additional heterologous polypeptide is an actuator moiety capable of regulating expression of a target gene in the plurality of cells. In some embodiments, the target gene is an endogenous gene. In some embodiments, the target gene is a heterologous gene.

In some embodiments of any of the methods disclosed herein, for example in paragraphs [0009]-[0011], the actuator moiety comprises a Cas endonuclease or a modification thereof.

In one embodiment of the method described herein, the threshold of the activity comprises (iv) at least about 5%, 10%, 20%, 40%, or 60% increase in the percentage of the plurality of cells expressing the immune checkpoint inhibitor over at least about 24 hours, as compared to control cells.

In some embodiments, the threshold of the activity comprises (v) at least about 10%, 20%, 50%, 100%, 200%, 500%, or 1000% increase in expression of the cytokine over at least about 24 hours, as compared to control cells.

In one embodiment, the threshold of the activity comprises (vi) at least about 20%, 40%, 60%, 80%, or 100% increase in the percentage of the plurality of cells in the proliferative state over at least about 48 hours, as compared to control cells.

In one embodiment, the threshold of the activity comprises (vii) at least about 10%, 20%, 30%, 40%, or 50% change in the percentage of the plurality of cells expressing the cell exhaustion marker over at least about 24 hours, as compared to control cells.

In some embodiments of any of the methods disclosed herein, for example in paragraph [0009], the change is an increase in the percentage of the plurality of cells.

In one embodiment of the methods disclosed herein, for example in paragraph [0016], the change is a decrease in the percentage of the plurality of cells.

In one aspect, the present disclosure describes a method of assessing activity of a plurality cells expressing one or more chimeric receptors comprising a ligand binding domain specific for an antigen, comprising: assessing an activity of a first sub-population of the plurality of cells upon treatment with a first non-cellular substrate comprising the antigen at a first surface density; assessing the activity of a second sub-population of the plurality of cells upon treatment with a second non-cellular substrate comprising the antigen at a second surface density that is lower than the first surface density.

In one embodiment of the methods disclosed herein, for example in paragraph [0019], the first non-cellular substrate and the second non-cellular substrate are the same.

In one embodiment of the methods disclosed herein, for example in paragraph [0019], the first non-cellular substrate and the second non-cellular substrate are different.

In some embodiments of any of the methods disclosed herein, for example in paragraphs [0019]-[0021], the first surface density is at least 2-fold greater than the second surface density.

In some embodiments of any of the methods disclosed herein, for example in paragraphs [0019]-[0022], the method further comprises comparing the activity of the first sub-population and the activity of the second sub-population.

In some embodiments of any of the methods disclosed herein, for example in paragraph [0023], the method further comprises assessing a suitability of the plurality of cells for use in a cell therapy for a subject in need thereof, based at least in part on the comparing.

In some embodiments of any of the methods disclosed herein, for example in paragraph [0024], the method further comprises determining that the plurality of cells is suitable for the use when the activity of the first sub-population and the activity of the second sub-population are about the same.

In some embodiments of any of the methods disclosed herein, for example in paragraph [0024], the method further comprises determining that the plurality of cells is suitable for the use when the activity of the first sub-population is greater than the activity of the second sub-population by at least 10%.

In one aspect, the present disclosure describes a method of assessing activity of a plurality of cells expressing one or more chimeric receptors comprising a ligand binding domain specific for an antigen, comprising: assessing an activity of a first sub-population of the plurality of cells upon treatment with a non-cellular substrate comprising a first antigen and a second antigen that are different, wherein a cell of the first sub-population expresses a first chimeric receptor comprising a first ligand binding domain specific for the first antigen.

In some embodiments of any of the methods disclosed herein, for example in paragraph [0027], the first chimeric receptor comprises a second ligand binding domain specific for the second antigen.

In some embodiments of any of the methods disclosed herein, for example in paragraph [0027], the cell expresses a second chimeric receptor comprising a second ligand binding domain specific for the second antigen.

In some embodiments of any of the methods disclosed herein, for example in paragraph [0027], the cell expresses an endogenous receptor configured to bind the second antigen.

In some embodiments of any of the methods disclosed herein, for example in paragraphs [0027]-[0030], the second antigen is an immune checkpoint inhibitor.

In some embodiments of any of the methods disclosed herein, for example in paragraphs [0027]-[0031], the method further comprising assessing a suitability of the plurality of cells for use in a cell therapy for a subject in need thereof, based at least in part on the assessing.

In some embodiments of any one of the method disclosed herein, the activity comprises one or more members selected from the group consisting of viability, expression of a cell activation marker, expression of one or more chimeric receptors, expression of an immune checkpoint inhibitor, expression of a cytokine, proliferation, expression of a cell exhaustion marker, chemotaxis, and metabolism.

In some embodiments of any one of the method disclosed herein, the cytokine comprises one or more members selected from the group consisting of IFN gamma, TNF alpha, IL-2, IL-4, and IL-10.

In some embodiments of any one of the method disclosed herein, the cytokine comprises one or more members selected from the group consisting of IFN gamma, TNF alpha, and IL-2.

In some embodiments of any one of the method disclosed herein, the immune checkpoint inhibitor comprises one or more members selected from the group consisting of PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GALS, adenosine, and TGF.

In some embodiments of any one of the method disclosed herein, the cell activation marker comprises one or more members selected from the group consisting of CD69, CD25, and HLA-DR.

In some embodiments of any one of the method disclosed herein, the cell exhaustion marker comprise one or more members selected from the group consisting of PD1, Tim3, Lag3, IL-2, TNF alpha, and IFN gamma.

In some embodiments of any one of the method disclosed herein, the antigen is not CD3 or CD28.

In some embodiments of any one of the method disclosed herein, the antigen is not CD3 and CD28.

In some embodiments of any one of the method disclosed herein, the non-cellular substrate is selected from the group consisting of a plate, a membrane, a matrix, a chip, and a plurality of beads.

In some embodiments of any one of the method disclosed herein, the antigen is coupled to the non-cellular substrate.

In some embodiments of any one of the method disclosed herein, the one or more chimeric receptors comprise a chimeric antigen receptor (CAR) or a modified T cell receptor (TCR).

In some embodiments of any one of the method disclosed herein, the plurality of cells comprises an immune cell.

In some embodiments of any one of the method disclosed herein, the immune cell is selected from the group consisting of a myeloid cell, a T cell such as alpha beta cytotoxic T cell, a gamma delta T cell, a regulatory T cell, a natural killer T cell, a B cell, and a natural killer cell.

In some embodiments of any of the methods disclosed herein, for example in paragraph [0045], the immune cell is T cell.

In some embodiments of any of the methods disclosed herein, for example in paragraph [0045], the immune cell is NK cell.

In some embodiments of any one of the method disclosed herein, the antigen, the first antigen, or the second antigen is selected from the group consisting of 707-AP, a biotinylated molecule, a-Actinin-4, abl-bcr alb-b3 (b2a2), abl-bcr alb-b4 (b3a2), adipophilin, AFP, AIM-2, Annexin II, ART-4, BAGE, b-Catenin, bcr-abl, bcr-abl p190 (e1a2), bcr-abl p210 (b2a2), bcr-abl p210 (b3a2), BING-4, CAG-3, CAIX, CAMEL, Caspase-8, CD171, CD19, CD20, CD22, CD23, CD24, CD30, CD33, CD38, CD44v7/8, CDC27, CDK-4, CEA, CLCA2, Cyp-B, DAM-10, DAM-6, DEK-CAN, EGFRvIII, EGP-2, EGP-40, ELF2, Ep-CAM, EphA2, EphA3, erb-B2, erb-B3, erb-B4, ES-ESO-1a, ETV6/AML, FBP, fetal acetylcholine receptor, FGF-5, FN, G250, GAGE-1, GAGE-2, GAGE-3, GAGE-4, GAGE-5, GAGE-6, GAGE-7B, GAGE-8, GD2, GD3, GnT-V, Gp100, gp75, Her-2, HLA-A*0201-R170I, HMW-MAA, HSP70-2 M, HST-2 (FGF6), HST-2/neu, hTERT, iCE, IL-11Rα, IL-13Rα2, KDR, KIAA0205, K-RAS, L1-cell adhesion molecule, LAGE-1, LDLR/FUT, Lewis Y, MAGE-1, MAGE-10, MAGE-12, MAGE-2, MAGE-3, MAGE-4, MAGE-6, MAGE-A1, MAGE-A2, MAGE-A3, MAGE-A6, MAGE-B1, MAGE-B2, Malic enzyme, Mammaglobin-A, MART-1/Melan-A, MART-2, MC1R, M-CSF, mesothelin, MUC1, MUC16, MUC2, MUM-1, MUM-2, MUM-3, Myosin, NA88-A, Neo-PAP, NKG2D, NPM/ALK, N-RAS, NY-ESO-1, OA1, OGT, oncofetal antigen (h5T4), OS-9, P polypeptide, P15, P53, PRAME, PSA, PSCA, PSMA, PTPRK, RAGE, ROR1, RU1, RU2, SART-1, SART-2, SART-3, SOX10, SSX-2, Survivin, Survivin-2B, SYT/SSX, TAG-72, TEL/AML1, TGFaRII, TGFbRII, TP1, TRAG-3, TRG, TRP-1, TRP-2, TRP-2/INT2, TRP-2-6b, Tyrosinase, VEGF-R2, WT1, α-folate receptor, κ-light chain, a tumor associated antigen, and a neoantigen.

In some embodiments of any one of the method disclosed herein, the antigen, the first antigen, or the second antigen comprises HER2 or PD-L1.

In some embodiments of any one of the method disclosed herein, the method is performed with a number of the non-cellular substrate (S) and a number of the plurality of cells (C) at a ratio of about 10:1 to about 1:200 (S:C).

In some embodiments of any of the methods disclosed herein, for example in paragraph [0050], the ratio is about 10:1 to about 1:50.

In some embodiments of any of the methods disclosed herein, for example in paragraph [0051], the ratio is about 10:1 to about 1:10.

In some embodiments of any one of the method disclosed herein, the method is performed in absence of a cell expressing the antigen, the first antigen, or the second antigen.

In some embodiments of any one of the method disclosed herein, the method is performed in vitro or ex vivo.

In some embodiments of any one of the method disclosed herein, the plurality of cells are mammalian cells.

In some embodiments of any one of the method disclosed herein, the plurality of cells are human cells.

In some embodiments of any one of the method disclosed herein, for example in paragraphs [0004]-[0056], the plurality of cells are autologous to the subject.

In some embodiments of any one of the method disclosed herein, for example in paragraphs [0004]-[0056], the plurality of cells are allogeneic to the subject.

In one aspect, the present disclosure provides a method of assessing a plurality of cells for use in a cell therapy for a subject in need thereof, comprising: providing the plurality of cells expressing one or more chimeric receptors comprising a ligand binding domain specific for an antigen; assessing activity of a first sub-population of the plurality of cells upon treatment with a non-cellular substrate comprising the antigen; and assessing a suitability of the plurality of cells for the cell therapy based upon a threshold of the activity, wherein the threshold is one or more members selected from the group consisting of: (i) a cytokine secretion exceeding at least 200 picogram from 100,000 plated cells over 24 hours, (ii) at least 2-fold increase in expression of a mRNA encoding a cytokine over 24 hours, (iii) at least 2-fold increase in cell number over 48 hours, and (iv) at least 50% cytotoxicity against a plurality of target cells over 72 hours.

In some embodiments, the method further comprises administering a second sub-population of the plurality of cells to the subject for the cell therapy when the first sub-population meets the threshold.

In another aspect, the present disclosure provides a method of assessing activity of a plurality cells expressing one or more chimeric receptors comprising a ligand binding domain specific for an antigen, comprising: assessing a first activity of a first sub-population of the plurality of cells upon treatment with a first non-cellular substrate comprising the antigen at a first surface density; assessing a second activity of a second sub-population of the plurality of cells upon treatment with a second non-cellular substrate comprising the antigen at a second surface density that is different than the first surface density. In some embodiments, the first and second non-cellular substrates are the same. In some embodiments, the first and second non-cellular substrates are different. In some embodiments, the first surface density is at least 2-fold greater than the second surface density.

In another aspect, the present disclosure provides a method of assessing activity of a plurality of cells expressing one or more chimeric receptors comprising a ligand binding domain specific for an antigen, comprising: assessing activity of a first sub-population of the plurality of cells upon treatment with a non-cellular substrate comprising a first antigen and a second antigen, wherein a cell of the first sub-population expresses a first chimeric receptor comprising a first ligand binding domain specific for the first antigen. In some embodiments, the first chimeric receptor comprises a second ligand binding domain specific for the second antigen. In some embodiments, the cell expresses a second chimeric receptor comprising a second ligand binding domain specific for the second antigen. In some embodiments, the cell expresses an endogenous receptor configured to bind the second antigen. In some embodiments, the second antigen is an immune checkpoint inhibitor. In some embodiments, the first antigen and the second antigen are presented on the non-cellular substrate at the same surface density. In some embodiments, the first antigen and the second antigen are presented on the non-cellular substrate at different surface densities.

In some embodiments of any one of the methods provided herein, the activity comprises cytokine secretion, gene expression, cell proliferation, cytotoxicity against a target cell, cell death, chemotaxis, cellular metabolism, and/or cell exhaustion.

In some embodiments of any one of the methods provided herein, the cytotoxicity is against a tumor cell or a cancer cell.

In some embodiments of any one of the methods provided herein, the cytokine comprises Interferon gamma, IL-1, IL-2, IL-6, TNF-α, IL-10, and/or IL-1RA.

In some embodiments of any one of the methods provided herein, the immune checkpoint inhibitor is selected from the group consisting of: PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MEW class I, MEW class II, GALS, adenosine, TGF (e.g., TGFbeta), variations thereof, and combinations thereof.

In some embodiments of any one of the methods provided herein, the antigen is not CD3 and/or CD28.

In some embodiments of any one of the methods provided herein, the non-cellular substrate is a plate, a membrane, a matrix, a chip, or a plurality of beads.

In some embodiments of any one of the methods provided herein, the antigen is coupled to the non-cellular substrate.

In some embodiments of any one of the methods provided herein, the one or more chimeric receptors comprise a chimeric antigen receptor (CAR) and/or a modified T cell receptor (TCR).

In some embodiments of any one of the methods provided herein, the plurality of cells comprises an immune cell.

In some embodiments of any one of the methods provided herein, the immune cell comprises a myeloid cell, a T cell such as alpha beta cytotoxic T cell, a gamma delta T cell, a regulatory T cell, a natural killer T cell, a B cell, and/or a natural killer cell.

In some embodiments of any one of the methods provided herein, the antigen is selected from the group consisting of 707-AP, a biotinylated molecule, a-Actinin-4, abl-bcr alb-b3 (b2a2), abl-bcr alb-b4 (b3a2), adipophilin, AFP, AIM-2, Annexin II, ART-4, BAGE, b-Catenin, bcr-abl, bcr-abl p190 (e1a2), bcr-abl p210 (b2a2), bcr-abl p210 (b3a2), BING-4, CAG-3, CAIX, CAMEL, Caspase-8, CD171, CD19, CD20, CD22, CD23, CD24, CD30, CD33, CD38, CD44v7/8, CDC27, CDK-4, CEA, CLCA2, Cyp-B, DAM-10, DAM-6, DEK-CAN, EGFRvIII, EGP-2, EGP-40, ELF2, Ep-CAM, EphA2, EphA3, erb-B2, erb-B3, erb-B4, ES-ESO-1a, ETV6/AML, FBP, fetal acetylcholine receptor, FGF-5, FN, G250, GAGE-1, GAGE-2, GAGE-3, GAGE-4, GAGE-5, GAGE-6, GAGE-7B, GAGE-8, GD2, GD3, GnT-V, Gp100, gp75, Her-2, HLA-A*0201-R170I, HMW-MAA, HSP70-2 M, HST-2 (FGF6), HST-2/neu, hTERT, iCE, IL-11Rα, IL-13Rα2, KDR, KIAA0205, K-RAS, L1-cell adhesion molecule, LAGE-1, LDLR/FUT, Lewis Y, MAGE-1, MAGE-10, MAGE-12, MAGE-2, MAGE-3, MAGE-4, MAGE-6, MAGE-A1, MAGE-A2, MAGE-A3, MAGE-A6, MAGE-B1, MAGE-B2, Malic enzyme, Mammaglobin-A, MART-1/Melan-A, MART-2, MC1R, M-CSF, mesothelin, MUC1, MUC16, MUC2, MUM-1, MUM-2, MUM-3, Myosin, NA88-A, Neo-PAP, NKG2D, NPM/ALK, N-RAS, NY-ESO-1, OA1, OGT, oncofetal antigen (h5T4), OS-9, P polypeptide, P15, P53, PRAME, PSA, PSCA, PSMA, PTPRK, RAGE, ROR1, RU1, RU2, SART-1, SART-2, SART-3, SOX10, SSX-2, Survivin, Survivin-2B, SYT/SSX, TAG-72, TEL/AML1, TGFaRII, TGFbRII, TP1, TRAG-3, TRG, TRP-1, TRP-2, TRP-2/INT2, TRP-2-6b, Tyrosinase, VEGF-R2, WT1, α-folate receptor, κ-light chain, a tumor associated antigen, and a neoantigen.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets fourth illustrative embodiments, in which the principles of the invention are utilized, and the accompanying drawings of which:

FIG. 1A-FIG. 1B show functional titration of HER2-CAR-T cells using HER2 conjugated beads. HER2-CAR-T cells were co-cultured with HER2-beads for 3 days at different bead to T cell ratios. Flow cytometry was used to measure CD69 (FIG. 1A) and PD-1 (FIG. 1B) expressions. Beads with different HER2 densities were used (0.1% and 0.5% represent % of total volume during the conjugation process), BSA beads were use as negative control. Skov3 cells were used as HER2+ control.

FIG. 2 shows cytokine secretion by HER2-CAR-T after stimulation with HER2 conjugated beads. Conventional HER2-CAR cells were cocultured with HER2-beads at 1:1 cell:bead ratio. BSA beads were used as negative control. Supernatants were collected at the indicated time points and cytokine secretion was measured for those time points.

FIG. 3A-FIG. 3E show a schematic flow diagram of an assay set up for assessing effect of antigen coated beads on CAR T cell potentiation. Engineered HER-2 CAR-T cells were collected by centrifugation, counted and plated in 96-well plate at 50K cells/well in T-cell growth media without cytokines (RPMI, 10% human serum, 1% Glutamax, 1% Pen/Strep mix). HER2-conjugated beads with different densities (low-mid-high, BSA as negative control, HER2 positive tumor cells as positive control, see FIG. 1A) were washed and added to the cells at different bead: cell ratios (5:1-1:125). Cells are supernatants were harvested at different time points (6 hrs-96 hrs).

FIG. 4A-FIG. 4D show data from antigen quantification on beads. Antigen binding capacity (ABC) and antigen density were determined on HER2 and PDL1+ beads, cells expressing HER2 and/or PDL1, as well as negative controls. FIG. 4A, HER2 antigen binding capacity (ABC vs. mean fluorescence intensity (MFI)). FIG. 4B, HER2 antigen density on beads. FIG. 4C, PD-L1 antigen binding capacity and FIG. 4D, antigen density (D). SKOV3, 231 and Fadu are cell lines. 231 PDL1 and Fadu PDL1 are PDL1 positive cells. Fadu-Luc are luciferase positive PDL 1.

FIG. 5 shows data indicating viability measurements of engineered HER-2 CAR-T cells cocultured with HER2 bead at indicated bead: cell ratio at 24 and 72 hours (h). BSA coated beads serve as control. FaDU-PDL1 cells was used as positive control. Top left graph, using low antigen density beads; Top right graph, using mid antigen density beads; Middle left graph, using high antigen density beads; Middle right graph, using positive control cells. Lower graph shows summary data of low, mid and high antigen density beads and positive control cells at 72 hours at the indicated bead: cell ratio. Data sets: 1a-d, RB340 72 h; 2a-d, NT 72 h; 3a-d, RB340 24 h; 4a-d, NT 24 h. RB-340—engineered HER2 cells, HER2-CAR-PD1sgRNA+dCAS9-KRAB): NT-non-transduced control. A “RB-340” engineered cell is a T cell engineered to express (i) anti-HER2 CAR that is fused to a protease and (ii) an adaptor of the CAR that is fused to an actuator (e.g., a dCas9-repressor or dCas9-activator) via a peptide substrate of the protease. A “NT” cell is a control T cell that is not engineered.

FIG. 6 shows data indicating CD69 expression in antigen coated bead stimulated engineered HER-2 CAR-T cells (RB-340) and non-transduced control cells (NT) at 24 and 72 h. Top left graph, using low antigen density beads; Top right graph, using mid antigen density beads; Middle left graph, using high antigen density beads; Middle right graph, using positive control cells. Lower graph shows summary data of low, mid and high antigen density beads and positive control cells at 72 hours at the indicated bead: cell ratio. Data sets: 1a-d, RB340 72 h; 2a-d, NT 72h; 3a-d, RB340 24 h; 4a-d, NT 24h.

FIG. 7 shows data in RB-340 cells are engineered with 2 major elements—HER2-CAR (along with a sgRNA) and dCAS9-KRAB (dCK) (RB-340 cells). Expression of CAR and dCK are monitored in the presence of the beads at concentrations indicated, at low, medium and high antigen density. Data sets: 1a-d, RB340 72 h; 2a-d, NT 72h; 3a-d, RB340 24 h; 4a-d, NT 24h.

FIG. 8 shows data indicating PD-1 expression in antigen coated bead stimulated engineered HER-2 CAR-T cells (RB-340) and non-transduced control cells (NT) at 24 and 72 h. Top left graph, using low antigen density beads; Top right graph, using mid antigen density beads; Middle left graph, using high antigen density beads; Middle right graph, using positive control cells. Lower graph shows summary data of low, mid and high antigen density beads and positive control cells at 72 hours at the indicated bead: cell ratio. Data sets: 1a-d, RB340 72h; 2a-d, NT 72h; 3a-d, RB340 24h; 4a-d, NT 24h.

FIG. 9 shows IL2 secretion by antigen coated bead stimulated engineered HER-2 CAR-T cells (RB-340) and non-transduced control cells (NT) at 24 and 72h. Top left graph, using low antigen density beads; Top right graph, using mid antigen density beads; bottom left graph, using high antigen density beads; bottom right graph, using positive control cells. Data sets: 1a-d, RB340 24h; 2a-d, NT 24h; 3a-d, RB340 72h; 4a-d, NT 72h.

FIG. 10 shows TNF secretion by antigen coated bead stimulated engineered HER-2 CAR-T cells (CAR, without dCas9 or sgRNA) and non-transduced control cells (NT) at 24 and 72h. Top left graph, using low antigen density beads; Top right graph, using mid antigen density beads; bottom left graph, using high antigen density beads; bottom right graph, using positive control cells. Data sets: 1a-d, RB340 24h; 2a-d, NT 24h; 3a-d, RB340 72h; 4a-d, NT 72h.

FIG. 11 shows IFN gamma (IFNG) secretion by antigen coated bead stimulated engineered HER-2 CAR-T cells (CAR, without dCas9 or sgRNA) and non-transduced (NT) control cells at 24 and 72h. Top left graph, using low antigen density beads; Top right graph, using mid antigen density beads; bottom left graph, using high antigen density beads; bottom right graph, using positive control cells. Data sets: 1a-d, RB340 24h; 2a-d, NT 24h; 3a-d, RB340 72h; 4a-d, NT 72h.

FIG. 12 shows data indicating inflammatory cytokine release in an experiment using conventional HER2-CAR-T cells (“d16 Cony”. without dCas9 or sgRNA) that were incubated with HER-2-high beads or control beads (coated with BSA only). Controls also include HER2 beads or BSA beads only, in absence of any cells.

FIG. 13A shows data indicating a percentage of PD1 positive cells in an experiment comparing conventional HER2-CAR-T cells (“d16 Cony”. without dCas9 or sgRNA), treated with either HER2 beads or control BSA beads. FIG. 13B shows data indicating a percentage of PD1 positive cells from an experiment comparing RB-340—HER2-CAR-anti PD1 sgRNA+dCas9-KRAB transcription repressor and control cells (HER2-CAR-Ctrl sgRNA that does not bind PD1 gene+dCas9-KRAB transcription repressor). Cells were incubated with HER-2 conjugated beads or control (BSA) beads. PD-1 expression level was measured by flow cytometry at various time point.

FIG. 14 shows RNA expression data for dCAS9 and cytokines upon bead stimulation. RB-340—HER2-CAR-PD1sgRNA+dCAS9-KRAB and non-transduced control cells (NT) were incubated with HER2-high beads or control (BSA) beads. RB-340-1 CART are experimental samples stimulated for 0, 6, 14, 24, 48 hrs with HER2-hi or BSA beads (1:1) stimulation and RNA was extracted at various time points and qPCR was performed to assess IFNγ and dCAS9-KRAB mRNA expression. LdCK, Q8 transduced T cells from the same donor at day 8 were used as a positive control.

FIG. 15A-FIG. 15D show proliferation data of CAR T cells upon bead stimulation. Conventional HER2-CAR-T cells were incubated with either HER2 beads or FaDu-PDL1 cells (HER2+ tumor cells). Celltrace Violet fluorescent (CTV) dye was added to the culture to follow cell proliferation.

FIG. 16A-FIG. 16C show data from experiment investigating T cell exhaustion. CAR T cells were stimulated with HER2+ tumor cells for 1-2 weeks followed by stimulation with HER2 or BSA beads. Flow cytometry was used to measure exhaustion marker Tim3 as well as intracellular cytokine production of TNFα and IFNγ.

FIG. 17 shows data from experimental using multiple antigen coated beads. Beads were conjugated to both HER2 (antigen) and PD-L1 (co-signal). HER2-PDL1 beads with 3 PDL densities were used (low-mid-high) and BSA beads were used as control. Beads were incubated with conventional HER2-CAR T cells for 24 or 72 hours. Flowcytometry was used to evaluate CD69 and PD1 expression. Datasets: 1, conventional CAR T cells 24h; 2, conventional CAR T cells 72h; 3, NT 24h; 4, NT 72h.

FIG. 18 shows ELISA data for cytokine release in experiment using multiple antigen conjugated beads as described in FIG. 17.

FIG. 19 shows antigen HER2 PDL1 bead parameters, antigen binding capacity and antigen density.

FIGS. 20A-20D illustrates a system comprising a gene modulating polypeptide (GMP) comprising an actuator moiety that is recruited to a transmembrane chimeric receptor polypeptide upon receptor modification (e.g., ligand binding and phosphorylation at an intracellular region), the actuator moiety is cleaved and released. Upon release, the actuator moiety enters the nucleus to regulate the expression and/or activity of a target gene or edit a nucleic acid sequence. 201, receptor binding moiety of GMP; 202a, actuator moiety; 202b, cleavage recognition site; 203, phosphorylation of intracellular region of the receptor; 205, antigen interacting extracellular domain of the receptor; 206, intracellular cleavage moiety of the receptor.

FIGS. 20E-20H show an analogous system to that in FIGS. 20A-20D, wherein receptor modification comprises a conformational change.

FIGS. 21A-21D schematically demonstrates release of an actuator moiety from the GMP. The system comprises a second adaptor polypeptide comprising a cleavage moiety is also recruited to the modified receptor. 301, receptor binding moiety of GMP; 302a, actuator moiety; 302b, cleavage recognition site; 303, phosphorylation of the intracellular region of the receptor; 305, antigen interacting extracellular domain of the receptor; 306, cleavage moiety on a second adaptor polypeptide, 307.

FIGS. 21E-21H show an analogous system to that in FIGS. 21A-21D, wherein receptor modification comprises a conformational change.

FIG. 22 illustrates an illustrative system comprising a transmembrane receptor useful for regulating expression of at least one target gene.

### DETAILED DESCRIPTION

The practice of some methods disclosed herein employ, unless otherwise indicated, conventional techniques of immunology, biochemistry, chemistry, molecular biology, microbiology, cell biology, genomics and recombinant DNA, which are within the skill of the art. See for example Sambrook and Green, Molecular Cloning: A Laboratory Manual, 4th Edition (2012); the series Current Protocols in Molecular Biology (F. M. Ausubel, et al. eds.); the series Methods In Enzymology (Academic Press, Inc.), PCR 2: A Practical Approach (M. J. MacPherson, B. D. Hames and G. R. Taylor eds. (1995)), Harlow and Lane, eds. (1988) Antibodies, A Laboratory Manual, and Culture of Animal Cells: A Manual of Basic Technique and Specialized Applications, 6th Edition (R.I. Freshney, ed. (2010)).

As used in the specification and claims, the singular forms “a,” “an,” and “the” include plural references unless the context clearly dictates otherwise. For example, the term “a chimeric transmembrane receptor polypeptide” includes a plurality of chimeric transmembrane receptor polypeptides.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

As used herein, a “cell” can generally refer to a biological cell. A cell can be the basic structural, functional and/or biological unit of a living organism. A cell can originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant (e.g. cells from plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, ferns, clubmosses, hornworts, liverworts, mosses), an algal cell, (e.g., Botryococcus braunii, Chlamydomonas reinhardtii, Nannochloropsis gaditana, Chlorella pyrenoidosa, Sargassum patens C. Agardh, and the like), seaweeds (e.g. kelp), a fungal cell (e.g., a yeast cell, a cell from a mushroom), an animal cell, a cell from an invertebrate animal (e.g. fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), and etcetera. Sometimes a cell is not originating from a natural organism (e.g. a cell can be a synthetically made, sometimes termed an artificial cell).

The term “hematopoietic stem cells,”, “hematopoietic progenitor cells,” or “hematopoietic precursor cells,” as used interchangeably herein, generally refers to cells which are committed to a hematopoietic lineage but are capable of further hematopoietic differentiation (e.g., into T cells) and include, multipotent hematopoietic stem cells (hematoblasts), myeloid progenitors, megakaryocyte progenitors, erythrocyte progenitors, and lymphoid progenitors. Hematopoietic stem cells (HSCs) are multipotent stem cells that give rise to all the blood cell types including myeloid (monocytes and macrophages, neutrophils, basophils, eosinophils, erythrocytes, megakaryocytes/platelets, dendritic cells), and lymphoid lineages (T cells, B cells, NK cells).

The term “immune cell” or “lymphocyte” generally refers to a differentiated hematopoietic cell. Non-limiting examples of an immune cell can include a T cell, an NK cell, a monocyte, an innate lymphocyte, a tumor-infiltrating lymphocyte, a macrophage, a granulocyte, etc.

The term “chimeric antigen receptor” or alternatively a “CAR” may be used herein to generally refer to a recombinant polypeptide construct comprising at least an extracellular antigen binding domain, a transmembrane domain, and a cytoplasmic signaling domain (also referred to herein as “an intracellular or intrinsic signaling domain”) comprising a functional signaling domain derived from a stimulatory molecule. In some cases, the stimulatory molecule may be the zeta chain associated with the T cell receptor complex. In some cases, the intracellular signaling domain further comprises one or more functional signaling domains derived from at least one costimulatory molecule. In some cases, the costimulatory molecule may comprise 4-1BB (i.e., CD137), CD27, and/or CD28. In one aspect, the CAR comprises an optional leader sequence at the amino-terminus (N-ter) of the CAR fusion protein. In one aspect, the CAR further comprises a leader sequence at the N-terminus of the extracellular antigen recognition domain, wherein the leader sequence is optionally cleaved from the antigen recognition domain (e.g., a scFv) during cellular processing and localization of the CAR to the cellular membrane. In some cases, the CAR may further comprise a GMP, as described in the present disclosure.

The CAR, as used herein, may be a first-, second-, third-, or fourth-generation CAR system, a functional variant thereof, or any combination thereof. First-generation CARs (e.g., CD19R or CD19CAR) include an antigen binding domain with specificity for a particular antigen (e.g., an antibody or antigen-binding fragment thereof such as an scFv, a Fab fragment, a VHH domain, or a VH domain of a heavy-chain only antibody), a transmembrane domain derived from an adaptive immune receptor (e.g., the transmembrane domain from the CD28 receptor), and a signaling domain derived from an adaptive immune receptor (e.g., one or more (e.g., three) ITAM domains derived from the intracellular region of the CD3 receptor or FcεRIγ). Second-generation CARs modify the first-generation CAR by addition of a co-stimulatory domain to the intracellular signaling domain portion of the CAR (e.g., derived from co-stimulatory receptors that act alongside T-cell receptors such as CD28, CD137/4-1BB, and CD134/OX40), which abrogates the need for administration of a co-factor (e.g., IL-2) alongside a first-generation CAR. Third-generation CARs add multiple co-stimulatory domains to the intracellular signaling domain portion of the CAR (e.g., CD3-CD28-OX40, or CD3-CD28-41BB). Fourth-generation CARs modify second- or third-generation CARs by the addition of an activating cytokine (e.g., IL-12, IL-23, or IL-27) to the intracellular signaling portion of the CAR (e.g., between one or more of the costimulatory domains and the CD3t ITAM domain) or under the control of a CAR-induced promoter (e.g., the NFAT/IL-2 minimal promoter).

The term “antigen,” as used herein, refers to a molecule or a fragment thereof capable of being bound by a selective binding agent. As an example, an antigen can be a ligand that can be bound by a selective binding agent such as a receptor. As another example, an antigen can be an antigenic molecule that can be bound by a selective binding agent such as an immunological protein (e.g., an antibody). An antigen can also refer to a molecule or fragment thereof capable of being used in an animal to produce antibodies capable of binding to that antigen.

The term “antibody,” as used herein, refers to a proteinaceous binding molecule with immunoglobulin-like functions. The term antibody includes antibodies (e.g., monoclonal and polyclonal antibodies), as well as derivatives, variants, and fragments thereof. Antibodies include, but are not limited to, immunoglobulins (Ig's) of different classes (i.e. IgA, IgG, IgM, IgD and IgE) and subclasses (such as IgG1, IgG2, etc.). A derivative, variant or fragment thereof can refer to a functional derivative or fragment which retains the binding specificity (e.g., complete and/or partial) of the corresponding antibody. Antigen-binding fragments include Fab, Fab′, F(ab′)₂, variable fragment (Fv), single chain variable fragment (scFv), minibodies, diabodies, and single-domain antibodies (“sdAb” or “nanobodies” or “camelids”). The term antibody includes antibodies and antigen-binding fragments of antibodies that have been optimized, engineered or chemically conjugated. Examples of antibodies that have been optimized include affinity-matured antibodies. Examples of antibodies that have been engineered include Fc optimized antibodies (e.g., antibodies optimized in the fragment crystallizable region) and multispecific antibodies (e.g., bispecific antibodies).

The terms “Fc receptor” or “FcR,” as used herein, generally refers to a receptor, or any derivative, variant or fragment thereof, that can bind to the Fc region of an antibody. In certain embodiments, the FcR is one which binds an IgG antibody (a gamma receptor, Fcgamma R) and includes receptors of the Fcgamma RI (CD64), Fcgamma RH (CD32), and Fcgamma RIII (CD16) subclasses, including allelic variants and alternatively spliced forms of these receptors. Fcgamma MI receptors include Fcgamma RITA (an “activating receptor”) and Fcgamma RIIB (an “inhibiting receptor”), which have similar amino acid sequences that differ primarily in the cytoplasmic domains thereof. The term “FcR” also includes the neonatal receptor, FcRn, which is responsible for the transfer of maternal IgGs to the fetus.

The term “expression” refers to one or more processes by which a polynucleotide is transcribed from a DNA template (such as into an mRNA or other RNA transcript) and/or the process by which a transcribed mRNA is subsequently translated into peptides, polypeptides, or proteins. Transcripts and encoded polypeptides can be collectively referred to as “gene product.” If the polynucleotide is derived from genomic DNA, expression can include splicing of the mRNA in a eukaryotic cell. “Up-regulated,” with reference to expression, generally refers to an increased expression level of a polynucleotide (e.g., RNA such as mRNA) and/or polypeptide sequence relative to its expression level in a wild-type state while “down-regulated” generally refers to a decreased expression level of a polynucleotide (e.g., RNA such as mRNA) and/or polypeptide sequence relative to its expression in a wild-type state. The term “nucleotide,” as used herein, generally refers to a base-sugar-phosphate combination. A nucleotide can comprise a synthetic nucleotide. A nucleotide can comprise a synthetic nucleotide analog. Nucleotides can be monomeric units of a nucleic acid sequence (e.g. deoxyribonucleic acid (DNA) and ribonucleic acid (RNA)). The term nucleotide can include ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof. Such derivatives can include, for example, [αS]dATP, 7-deaza-dGTP and 7-deaza-dATP, and nucleotide derivatives that confer nuclease resistance on the nucleic acid molecule containing them. The term nucleotide as used herein can refer to dideoxyribonucleoside triphosphates (ddNTPs) and their derivatives. Illustrative examples of dideoxyribonucleoside triphosphates can include, but are not limited to, ddATP, ddCTP, ddGTP, ddITP, and ddTTP. A nucleotide may be unlabeled or detectably labeled by well-known techniques. Labeling can also be carried out with quantum dots. Detectable labels can include, for example, radioactive isotopes, fluorescent labels, chemiluminescent labels, bioluminescent labels and enzyme labels. Fluorescent labels of nucleotides may include but are not limited fluorescein, 5-carboxyfluorescein (FAM), 2′7′-dimethoxy-4′5-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′dimethylaminophenylazo) benzoic acid (DABCYL), Cascade Blue, Oregon Green, Texas Red, Cyanine and 5-(2′-aminoethyl)aminonaphthalene-1-sulfonic acid (EDANS). Specific examples of fluorescently labeled nucleotides can include [R6G]dUTP, [TAMRA]dUTP, [R110]dCTP, [R6G] dCTP, [TAMRA] dCTP, [JOE] ddATP, [R6G] ddATP, [FAM] ddCTP, [R110]ddCTP, [TAMRA]ddGTP, [ROX]ddTTP, [dR6G]ddATP, [dR110]ddCTP, [dTAMRA]ddGTP, and [dROX]ddTTP available from Perkin Elmer, Foster City, Calif. FluoroLink DeoxyNucleotides, FluoroLink Cy3-dCTP, FluoroLink Cy5-dCTP, FluoroLink Fluor X-dCTP, FluoroLink Cy3-dUTP, and FluoroLink Cy5-dUTP available from Amersham, Arlington Heights, Ill.; Fluorescein-15-dATP, Fluorescein-12-dUTP, Tetramethyl-rodamine-6-dUTP, IR770-9-dATP, Fluorescein-12-ddUTP, Fluorescein-12-UTP, and Fluorescein-15-2′-dATP available from Boehringer Mannheim, Indianapolis, Ind.; and Chromosome Labeled Nucleotides, BODIPY-FL-14-UTP, BODIPY-FL-4-UTP, BODIPY-TMR-14-UTP, BODIPY-TMR-14-dUTP, BODIPY-TR-14-UTP, BODIPY-TR-14-dUTP, Cascade Blue-7-UTP, Cascade Blue-7-dUTP, fluorescein-12-UTP, fluorescein-12-dUTP, Oregon Green 488-5-dUTP, Rhodamine Green-5-UTP, Rhodamine Green-5-dUTP, tetramethylrhodamine-6-UTP, tetramethylrhodamine-6-dUTP, Texas Red-5-UTP, Texas Red-5-dUTP, and Texas Red-12-dUTP available from Molecular Probes, Eugene, Oreg. Nucleotides can also be labeled or marked by chemical modification. A chemically-modified single nucleotide can be biotin-dNTP. Some non-limiting examples of biotinylated dNTPs can include, biotin-dATP (e.g., bio-N6-ddATP, biotin-14-dATP), biotin-dCTP (e.g., biotin-11-dCTP, biotin-14-dCTP), and biotin-dUTP (e.g. biotin-11-dUTP, biotin-16-dUTP, biotin-20-dUTP).

The term “polynucleotide,” “oligonucleotide,” or “nucleic acid,” as used interchangeably herein, generally refers to a polymeric form of nucleotides of any length, either deoxyribonucleotides or ribonucleotides, or analogs thereof, either in single-, double-, or multi-stranded form. A polynucleotide can be exogenous or endogenous to a cell. A polynucleotide can exist in a cell-free environment. A polynucleotide can be a gene or fragment thereof. A polynucleotide can be DNA. A polynucleotide can be RNA. A polynucleotide can have any three dimensional structure, and can perform any function, known or unknown. A polynucleotide can comprise one or more analogs (e.g. altered backbone, sugar, or nucleobase). If present, modifications to the nucleotide structure can be imparted before or after assembly of the polymer. Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g. rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudourdine, dihydrouridine, queuosine, and wyosine. Non-limiting examples of polynucleotides include coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, cell-free polynucleotides including cell-free DNA (cfDNA) and cell-free RNA (cfRNA), nucleic acid probes, and primers. The sequence of nucleotides can be interrupted by non-nucleotide components.

The term “gene,” as used herein, refers to a nucleic acid (e.g., DNA such as genomic DNA and cDNA) and its corresponding nucleotide sequence that is involved in encoding an RNA transcript. The term as used herein with reference to genomic DNA includes intervening, non-coding regions as well as regulatory regions and can include 5′ and 3′ ends. In some uses, the term encompasses the transcribed sequences, including 5′ and 3′ untranslated regions (5′-UTR and 3′-UTR), exons and introns. In some genes, the transcribed region will contain “open reading frames” that encode polypeptides. In some uses of the term, a “gene” comprises only the coding sequences (e.g., an “open reading frame” or “coding region”) necessary for encoding a polypeptide. In some cases, genes do not encode a polypeptide, for example, ribosomal RNA genes (rRNA) and transfer RNA (tRNA) genes. In some cases, the term “gene” includes not only the transcribed sequences, but in addition, also includes non-transcribed regions including upstream and downstream regulatory regions, enhancers and promoters. A gene can refer to an “endogenous gene” or a native gene in its natural location in the genome of an organism. A gene can refer to an “exogenous gene” or a non-native gene. A non-native gene can refer to a gene not normally found in the host organism but which is introduced into the host organism by gene transfer. A non-native gene can also refer to a gene not in its natural location in the genome of an organism. A non-native gene can also refer to a naturally occurring nucleic acid or polypeptide sequence that comprises mutations, insertions and/or deletions (e.g., non-native sequence).

The terms “transfection” or “transfected” refer to introduction of a nucleic acid into a cell by non-viral or viral-based methods. The nucleic acid molecules may be gene sequences encoding complete proteins or functional portions thereof.

The term “expression” refers to one or more processes by which a polynucleotide is transcribed from a DNA template (such as into an mRNA or other RNA transcript) and/or the process by which a transcribed mRNA is subsequently translated into peptides, polypeptides, or proteins. Transcripts and encoded polypeptides can be collectively referred to as “gene product.” If the polynucleotide is derived from genomic DNA, expression can include splicing of the mRNA in a eukaryotic cell. “Up-regulated,” with reference to expression, generally refers to an increased expression level of a polynucleotide (e.g., RNA such as mRNA) and/or polypeptide sequence relative to its expression level in a wild-type state while “down-regulated” generally refers to a decreased expression level of a polynucleotide (e.g., RNA such as mRNA) and/or polypeptide sequence relative to its expression in a wild-type state. Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell.

The term “expression cassette,” “expression construct,” or “expression vector” refers to a nucleic acid that includes a nucleotide sequence such as a coding sequence and a template sequence, and sequences necessary for expression of the coding sequence. The expression cassette can be viral or non-viral. For instance, an expression cassette includes a nucleic acid construct, which when introduced into a host cell, results in transcription and/or translation of a RNA or polypeptide, respectively. Antisense constructs or sense constructs that are not or cannot be translated are expressly included by this definition. One of skill will recognize that the inserted polynucleotide sequence need not be identical, but may be only substantially similar to a sequence of the gene from which it was derived.

A “plasmid,” as used herein, generally refers to a non-viral expression vector, e.g., a nucleic acid molecule that encodes for genes and/or regulatory elements necessary for the expression of genes. A “viral vector,” as used herein, generally refers to a viral-derived nucleic acid that is capable of transporting another nucleic acid into a cell. A viral vector is capable of directing expression of a protein or proteins encoded by one or more genes carried by the vector when it is present in the appropriate environment. Examples for viral vectors include, but are not limited to retroviral, adenoviral, lentiviral and adeno-associated viral vectors.

The term “promoter,” as used herein, refers to a polynucleotide sequence capable of driving transcription of a coding sequence in a cell. Thus, promoters used in the polynucleotide constructs of the disclosure include cis-acting transcriptional control elements and regulatory sequences that are involved in regulating or modulating the timing and/or rate of transcription of a gene. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) gene transcription. A “constitutive promoter” is one that is capable of initiating transcription in nearly all tissue types, whereas a “tissue-specific promoter” initiates transcription only in one or a few particular tissue types. An “inducible promoter” is one that initiates transcription only under particular environmental conditions, developmental conditions, or drug or chemical conditions.

The terms “complement,” “complements,” “complementary,” and “complementarity,” as used herein, generally refer to a sequence that is fully complementary to and hybridizable to the given sequence. In some cases, a sequence hybridized with a given nucleic acid is referred to as the “complement” or “reverse-complement” of the given molecule if its sequence of bases over a given region is capable of complementarily binding those of its binding partner, such that, for example, A-T, A-U, G-C, and G-U base pairs are formed. In general, a first sequence that is hybridizable to a second sequence is specifically or selectively hybridizable to the second sequence, such that hybridization to the second sequence or set of second sequences is preferred (e.g. thermodynamically more stable under a given set of conditions, such as stringent conditions commonly used in the art) to hybridization with non-target sequences during a hybridization reaction. Typically, hybridizable sequences share a degree of sequence complementarity over all or a portion of their respective lengths, such as between 25%-100% complementarity, including at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, and 100% sequence complementarity. Sequence identity, such as for the purpose of assessing percent complementarity, can be measured by any suitable alignment algorithm, including but not limited to the Needleman-Wunsch algorithm (see e.g. the EMBOSS Needle aligner available at www.ebi.ac.uk/Tools/psa/emboss needle/nucleotide.html, optionally with default settings), the BLAST algorithm (see e.g. the BLAST alignment tool available at blast.ncbi.nlm.nih.gov/Blast.cgi, optionally with default settings), or the Smith-Waterman algorithm (see e.g. the EMBOSS Water aligner available at www.ebi.ac.uk/Tools/psa/emboss water/nucleotide.html, optionally with default settings). Optimal alignment can be assessed using any suitable parameters of a chosen algorithm, including default parameters.

Complementarity can be perfect or substantial/sufficient. Perfect complementarity between two nucleic acids can mean that the two nucleic acids can form a duplex in which every base in the duplex is bonded to a complementary base by Watson-Crick pairing. Substantial or sufficient complementary can mean that a sequence in one strand is not completely and/or perfectly complementary to a sequence in an opposing strand, but that sufficient bonding occurs between bases on the two strands to form a stable hybrid complex in set of hybridization conditions (e.g., salt concentration and temperature). Such conditions can be predicted by using the sequences and standard mathematical calculations to predict the Tm of hybridized strands, or by empirical determination of Tm by using routine methods.

The terms “actuator moiety,” “actuator domain,” and “gene modulating domain,” as used herein, refers to a moiety which can regulate expression or activity of a gene and/or edit a nucleic acid sequence, whether exogenous or endogenous. An actuator moiety can regulate expression of a gene at the transcription level and/or the translation level. An actuator moiety can regulate gene expression at the transcription level, for example, by regulating the production of mRNA from DNA, such as chromosomal DNA or cDNA. In some embodiments, an actuator moiety recruits at least one transcription factor that binds to a specific DNA sequence, thereby controlling the rate of transcription of genetic information from DNA to mRNA. An actuator moiety can itself bind to DNA and regulate transcription by physical obstruction, for example preventing proteins such as RNA polymerase and other associated proteins from assembling on a DNA template. An actuator moiety can regulate expression of a gene at the translation level, for example, by regulating the production of protein from mRNA template. In some embodiments, an actuator moiety regulates gene expression by affecting the stability of an mRNA transcript. In some embodiments, an actuator moiety regulates expression of a gene by editing a nucleic acid sequence (e.g., a region of a genome). In some embodiments, an actuator moiety regulates expression of a gene by editing an mRNA template. Editing a nucleic acid sequence can, in some cases, alter the underlying template for gene expression.

The term “targeting sequence,” as used herein, refers to a nucleotide sequence and the corresponding amino acid sequence which encodes a targeting polypeptide which mediates the localization (or retention) of a protein to a sub-cellular location, e.g., plasma membrane or membrane of a given organelle, nucleus, cytosol, mitochondria, endoplasmic reticulum (ER), Golgi, chloroplast, apoplast, peroxisome or other organelle. For example, a targeting sequence can direct a protein (e.g., a receptor polypeptide or an adaptor polypeptide) to a nucleus utilizing a nuclear localization signal (NLS); outside of a nucleus of a cell, for example to the cytoplasm, utilizing a nuclear export signal (NES); mitochondria utilizing a mitochondrial targeting signal; the endoplasmic reticulum (ER) utilizing an ER-retention signal; a peroxisome utilizing a peroxisomal targeting signal; plasma membrane utilizing a membrane localization signal; or combinations thereof.

As used herein, “fusion” can refer to a protein and/or nucleic acid comprising one or more non-native sequences (e.g., moieties). A fusion can comprise one or more of the same non-native sequences. A fusion can comprise one or more of different non-native sequences. A fusion can be a chimera. A fusion can comprise a nucleic acid affinity tag. A fusion can comprise a barcode. A fusion can comprise a peptide affinity tag. A fusion can provide for subcellular localization of the site-directed polypeptide (e.g., a nuclear localization signal (NLS) for targeting to the nucleus, a mitochondrial localization signal for targeting to the mitochondria, a chloroplast localization signal for targeting to a chloroplast, an endoplasmic reticulum (ER) retention signal, and the like). A fusion can provide a non-native sequence (e.g., affinity tag) that can be used to track or purify. A fusion can be a small molecule such as biotin or a dye such as Alexa fluor dyes, Cyanine3 dye, Cyanine5 dye.

A fusion can refer to any protein with a functional effect. For example, a fusion protein can comprise methyltransferase activity, demethylase activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, remodelling activity, protease activity, oxidoreductase activity, transferase activity, hydrolase activity, lyase activity, isomerase activity, synthase activity, synthetase activity, or demyristoylation activity. An effector protein can modify a genomic locus. A fusion protein can be a fusion in a Cas protein. An fusion protein can be a non-native sequence in a Cas protein.

As used herein, “non-native” can refer to a nucleic acid or polypeptide sequence that is not found in a native nucleic acid or protein. Non-native can refer to affinity tags. Non-native can refer to fusions. Non-native can refer to a naturally occurring nucleic acid or polypeptide sequence that comprises mutations, insertions and/or deletions. A non-native sequence may exhibit and/or encode for an activity (e.g., enzymatic activity, methyltransferase activity, acetyltransferase activity, kinase activity, ubiquitinating activity, etc.) that can also be exhibited by the nucleic acid and/or polypeptide sequence to which the non-native sequence is fused. A non-native nucleic acid or polypeptide sequence may be linked to a naturally-occurring nucleic acid or polypeptide sequence (or a variant thereof) by genetic engineering to generate a chimeric nucleic acid and/or polypeptide sequence encoding a chimeric nucleic acid and/or polypeptide.

The term “regulating” with reference to expression or activity, as used herein, refers to altering the level of expression or activity. Regulation can occur at the transcription level and/or translation level.

The terms “peptide,” “polypeptide,” and “protein” are used interchangeably herein to refer to a polymer of at least two amino acid residues joined by peptide bond(s). This term does not connote a specific length of polymer, nor is it intended to imply or distinguish whether the peptide is produced using recombinant techniques, chemical or enzymatic synthesis, or is naturally occurring. The terms apply to naturally occurring amino acid polymers as well as amino acid polymers comprising at least one modified amino acid. In some cases, the polymer can be interrupted by non-amino acids. The terms include amino acid chains of any length, including full length proteins, and proteins with or without secondary and/or tertiary structure (e.g., domains). The terms also encompass an amino acid polymer that has been modified, for example, by disulfide bond formation, glycosylation, lipidation, acetylation, phosphorylation, oxidation, and any other manipulation such as conjugation with a labeling component. The terms “amino acid” and “amino acids,” as used herein, generally refer to natural and non-natural amino acids, including, but not limited to, modified amino acids and amino acid analogues. Modified amino acids can include natural amino acids and non-natural amino acids, which have been chemically modified to include a group or a chemical moiety not naturally present on the amino acid. Amino acid analogues can refer to amino acid derivatives. The term “amino acid” includes both D-amino acids and L-amino acids.

The terms “derivative,” “variant,” and “fragment,” when used herein with reference to a polypeptide, refers to a polypeptide related to a wild type polypeptide, for example either by amino acid sequence, structure (e.g., secondary and/or tertiary), activity (e.g., enzymatic activity) and/or function. Derivatives, variants and fragments of a polypeptide can comprise one or more amino acid variations (e.g., mutations, insertions, and deletions), truncations, modifications, or combinations thereof compared to a wild type polypeptide.

The terms “subject,” “individual,” and “patient” are used interchangeably herein to refer to a vertebrate, preferably a mammal such as a human. Mammals include, but are not limited to, murines, simians, humans, farm animals, sport animals, and pets. Tissues, cells and their progeny of a biological entity obtained in vivo or cultured in vitro are also encompassed.

The terms “treatment” and “treating,” as used herein, refer to an approach for obtaining beneficial or desired results including but not limited to a therapeutic benefit and/or a prophylactic benefit. For example, a treatment can comprise administering a system or cell population disclosed herein. By therapeutic benefit is meant any therapeutically relevant improvement in or effect on one or more diseases, conditions, or symptoms under treatment. For prophylactic benefit, a composition can be administered to a subject at risk of developing a particular disease, condition, or symptom, or to a subject reporting one or more of the physiological symptoms of a disease, even though the disease, condition, or symptom may not have yet been manifested.

The term “effective amount” or “therapeutically effective amount” refers to the quantity of a composition, for example a composition comprising immune cells such as lymphocytes (e.g., T lymphocytes and/or NK cells) comprising a system of the present disclosure, that is sufficient to result in a desired activity upon administration to a subject in need thereof. Within the context of the present disclosure, the term “therapeutically effective” refers to that quantity of a composition that is sufficient to delay the manifestation, arrest the progression, relieve or alleviate at least one symptom of a disorder treated by the methods of the present disclosure.

I. Introduction

Immune cells (e.g., T cells, NK cells) can be engineered to exhibit a specific affinity to one or more specific antigens (e.g., cancer or tumor antigens) for adoptive immunotherapy for treatment of cancers (e.g., solid tumors, lymphoma, etc.). In some cases, the immune cells can be engineered to express heterologous receptors (e.g., chimeric antigen receptors or “CAR”) capable of binding to one or more specific antigens, thereby targeting cancer cells in a subject in need thereof.

In some cases, prior to administration of the engineered immune cells to the subject, the engineered immune cells can be cultured in the presence of a population of target cells (e.g., cancer or tumor cells expressing the one or more specific antigens) to test (or assess) one or more activities of the engineered immune cells (e.g., for example, viability, proliferation, and/or cytotoxicity against a target cell (e.g., a cancer or tumor cell). The testing can be used to determine whether or not the engineered immune cells are suitable for use in cell therapy for the subject. However, such testing based on the population of target cells can vary from batch to batch, or over-time within a single batch (e.g., due to change in cellular activity of the population of target cells during the testing, such as proliferation or cell death of the population of target cells), and can be unreliable and sub-optimal.

Thus, there remains a significant unmet need for an alternative cell-free method for testing one or more activities of the engineered immune cells to determine whether or not the engineered immune cells are suitable for use in cell therapy for the subject.

II. Methods and Systems

In one aspect, the present disclosure provides a method of assessing plurality of cells for use in cell therapies. The method comprises providing the plurality of cells expressing one or more chimeric receptors comprising a ligand binding domain specific for an antigen. The method further comprises assessing activities of a first sub-population of the plurality of cells upon treatment with non-cellular substrates comprising the antigen. The method further comprises assessing a suitability of the plurality of cells for cell therapy based on thresholds of the activity of the first sub-population of the plurality of the cells treated with the non-cellular substrates comprising the antigen.

In one aspect, the present disclosure describes a method of assessing a plurality of cells for use in a cell therapy for a subject in need thereof, comprising: providing the plurality of cells capable of expressing one or more chimeric receptors comprising a ligand binding domain specific for an antigen; assessing an activity of a first sub-population of the plurality of cells upon treatment with a non-cellular substrate comprising the antigen; and assessing a suitability of the plurality of cells for the cell therapy based upon a threshold of the activity.

In some embodiments, the thresholds can be based on comparisons between the first sub-population of plurality of cells and control cells. In some embodiments, the thresholds can be based on comparisons between the activities of the first sub-population of the plurality of cells before treated with the antigen and the activities of the controls cells before treated with the antigen. In some embodiments, the thresholds can be based on comparisons between the activities of the first sub-population of the plurality of cells after treated with the antigen and the activities of the controls cells after treated with the antigen. In some embodiments, the thresholds can be based on comparisons between the activities of the first sub-population of the plurality of cells before treated with the antigen and the activities of the first sub-population of the plurality of cells after treated with the antigen. In some embodiments, the thresholds can be determined based on a single time point or across multiple time points.

In one embodiment, the threshold of the activity comprises that the cells exhibit at least about 50% viability over at least about 24 hours. In some embodiments, the threshold comprises at least about 60% viability, at least about 70% viability, at least about 80% viability, at least about 90% viability or at least about 95% viability or at least about 98% viability of cells over at least 24 hours. In some embodiments, the threshold comprises at least about 50%, at least about 60% viability, at least about 70% viability, at least about 80% viability, at least about 90% viability or at least about 95% viability or at least about 98% viability of cells over at least 36 hours. In some embodiments, the threshold comprises at least about 50%, at least about 60% viability, at least about 70% viability, at least about 80% viability, at least about 90% viability or at least about 95% viability or at least about 98% viability of cells over at least 48 hours. In some embodiments, the threshold comprises at least about 50%, at least about 60% viability, at least about 70% viability, at least about 80% viability, at least about 90% viability or at least about 95% viability or at least about 98% viability of cells over at least 72 hours. In some embodiments, the threshold comprises at least about 50% over at least 72 hours.

In one embodiment, the threshold of the activity comprises that the cells exhibit at least about 10% increase in a percentage of the plurality of cells expressing a cell activation marker over at least about 24 hours, as compared to control cells. In one embodiment, the threshold of the activity comprises, at least about 20% increase in a percentage of the plurality of cells expressing a cell activation marker over at least about 24 hours, as compared to control cells. In one embodiment, the threshold of the activity comprises, at least about 30% increase in a percentage of the plurality of cells expressing a cell activation marker over at least about 24 hours, as compared to control cells. In one embodiment, the threshold of the activity comprises, at least about 40% increase in a percentage of the plurality of cells expressing a cell activation marker over at least about 24 hours, as compared to control cells. In one embodiment, the threshold of the activity comprises, at least about 50% increase in a percentage of the plurality of cells expressing a cell activation marker over at least about 24 hours, as compared to control cells. In one embodiment, the threshold of the activity comprises, at least about 60% increase in a percentage of the plurality of cells expressing a cell activation marker over at least about 24 hours, as compared to control cells. In one embodiment, the threshold of the activity comprises, at least about 70% increase in a percentage of the plurality of cells expressing a cell activation marker over at least about 24 hours, as compared to control cells. In one embodiment, the threshold of the activity comprises, at least about 80% increase in a percentage of the plurality of cells expressing a cell activation marker over at least about 24 hours, as compared to control cells. In one embodiment, the threshold of the activity comprises, at least about 90% increase in a percentage of the plurality of cells expressing a cell activation marker over at least about 24 hours, as compared to control cells. In one embodiment, the threshold of the activity comprises, at least about 95% increase in a percentage of the plurality of cells expressing a cell activation marker over at least about 24 hours, as compared to control cells. In one embodiment, the threshold of the activity comprises, at least about 100% increase in a percentage of the plurality of cells expressing a cell activation marker over at least about 24 hours, as compared to control cells.

In some embodiments a T cell activation marker may comprise an increase in one or more of CD69 (early activation marker), CD25 (late activation marker), HLA-DR (late-stage activation marker), CD3, CD4, CD44, CD94, CD183 (CXCR3), CD195 (CCR5), CD193 (CCR3), CD184 (CXCR4), CD196 (CCR6), CD161, CD185 (CXCR5), CD197 (CCR7), CCR10, AhR, CD127, cytokines, such as IL-2, IFN-γ, TNF-α, IL-4, IL-5, IL-6, IL-13, IL-9, IL-10, IL-13, IL-22, IL-10, IL-12, IL-21, IL-35 and TGFb1, depending on the T cell type and activation profile, or intracellular markers, such as transcription factors, e.g., T-bet, STAT1, STAT4, GATA3, STAT6, RORγt, STAT3, IRF4, GATA3, STAT6, PU.1, BNC2, FOXO4, STAT3, Bc16, FoxP3, STAT5, Smad2 among others.

In some embodiments, the T cell activation marker may include, but not limited to cytotoxicity markers, such as CD44, LAP-1 (CD107a), CD178, Granzyme B or Perforin. In some embodiments, an activation marker may include one or more of, but not limited to, CD25 (IL2RA) CD44, CD69, CD95 (FasR), CD134 (OX40), CD137 (4-1BB), KLRG1, or proliferation marker Ki-67.

In one embodiment, the threshold of the activity comprises that the cells exhibit at least about 10% increase in a percentage of the plurality of cells expressing a cell activation marker over at least about 48 hours, as compared to control cells. In one embodiment, the threshold of the activity comprises, at least about 20% increase in a percentage of the plurality of cells expressing a cell activation marker over at least about 48 hours, as compared to control cells. In one embodiment, the threshold of the activity comprises, at least about 30% increase in a percentage of the plurality of cells expressing a cell activation marker over at least about 48 hours, as compared to control cells. In one embodiment, the threshold of the activity comprises, at least about 40% increase in a percentage of the plurality of cells expressing a cell activation marker over at least about 48 hours, as compared to control cells. In one embodiment, the threshold of the activity comprises, at least about 50% increase in a percentage of the plurality of cells expressing a cell activation marker over at least about 48 hours, as compared to control cells. In one embodiment, the threshold of the activity comprises, at least about 60% increase in a percentage of the plurality of cells expressing a cell activation marker over at least about 48 hours, as compared to control cells. In one embodiment, the threshold of the activity comprises, at least about 70% increase in a percentage of the plurality of cells expressing a cell activation marker over at least about 48 hours, as compared to control cells. In one embodiment, the threshold of the activity comprises, at least about 80% increase in a percentage of the plurality of cells expressing a cell activation marker over at least about 48 hours, as compared to control cells. In one embodiment, the threshold of the activity comprises, at least about 90% increase in a percentage of the plurality of cells expressing a cell activation marker over at least about 48 hours, as compared to control cells. In one embodiment, the threshold of the activity comprises, at least about 95% increase in a percentage of the plurality of cells expressing a cell activation marker over at least about 48 hours, as compared to control cells. In one embodiment, the threshold of the activity comprises, at least about 100% increase in a percentage of the plurality of cells expressing a cell activation marker over at least about 48 hours, as compared to control cells.

In one embodiment, the threshold of the activity comprises that the cells exhibit at least about 10% increase in a percentage of the plurality of cells expressing a cell activation marker over at least about 72 hours, as compared to control cells. In one embodiment, the threshold of the activity comprises, at least about 20% increase in a percentage of the plurality of cells expressing a cell activation marker over at least about 72 hours, as compared to control cells. In one embodiment, the threshold of the activity comprises, at least about 30% increase in a percentage of the plurality of cells expressing a cell activation marker over at least about 72 hours, as compared to control cells. In one embodiment, the threshold of the activity comprises, at least about 40% increase in a percentage of the plurality of cells expressing a cell activation marker over at least about 72 hours, as compared to control cells. In one embodiment, the threshold of the activity comprises, at least about 50% increase in a percentage of the plurality of cells expressing a cell activation marker over at least about 72 hours, as compared to control cells. In one embodiment, the threshold of the activity comprises, at least about 60% increase in a percentage of the plurality of cells expressing a cell activation marker over at least about 72 hours, as compared to control cells. In one embodiment, the threshold of the activity comprises, at least about 70% increase in a percentage of the plurality of cells expressing a cell activation marker over at least about 72 hours, as compared to control cells. In one embodiment, the threshold of the activity comprises, at least about 80% increase in a percentage of the plurality of cells expressing a cell activation marker over at least about 72 hours, as compared to control cells. In one embodiment, the threshold of the activity comprises, at least about 90% increase in a percentage of the plurality of cells expressing a cell activation marker over at least about 72 hours, as compared to control cells. In one embodiment, the threshold of the activity comprises, at least about 95% increase in a percentage of the plurality of cells expressing a cell activation marker over at least about 72 hours, as compared to control cells. In one embodiment, the threshold of the activity comprises, at least about 100% increase in a percentage of the plurality of cells expressing a cell activation marker over at least about 72 hours, as compared to control cells.

In one embodiment, the threshold of the activity comprises that the cells exhibit at least about 5% increase in a percentage of the plurality of cells expressing the one or more chimeric receptors over 48 hours, as compared to control cells. In one embodiment, the threshold of the activity comprises that the cells exhibit at least about 7.5% increase in a percentage of the plurality of cells expressing the one or more chimeric receptors over 48 hours, as compared to control cells. In one embodiment, the threshold of the activity comprises that the cells exhibit at least about 10% increase in a percentage of the plurality of cells expressing the one or more chimeric receptors over 48 hours, as compared to control cells. In one embodiment, the threshold of the activity comprises that the cells exhibit at least about 12.5% increase in a percentage of the plurality of cells expressing the one or more chimeric receptors or the actuator moiety over 48 hours, as compared to control cells. In one embodiment, the threshold of the activity comprises that the cells exhibit at least about 15% increase in a percentage of the plurality of cells expressing the one or more chimeric receptors or the actuator moiety over 48 hours, as compared to control cells. In one embodiment, the threshold of the activity comprises that the cells exhibit at least about 17.5% increase in a percentage of the plurality of cells expressing the one or more chimeric receptors or the actuator moiety over 48 hours, as compared to control cells. In one embodiment, the threshold of the activity comprises that the cells exhibit at least about 20% increase in a percentage of the plurality of cells expressing the one or more chimeric receptors or the actuator moiety over 48 hours, as compared to control cells. In one embodiment, the threshold of the activity comprises that the cells exhibit at least about 22.5% increase in a percentage of the plurality of cells expressing the one or more chimeric receptors or the actuator moiety over 48 hours, as compared to control cells. In one embodiment, the threshold of the activity comprises that the cells exhibit at least about 25% increase in a percentage of the plurality of cells expressing the one or more chimeric receptors or the actuator moiety over 48 hours, as compared to control cells.

In one embodiment, the threshold of the activity comprises that the cells exhibit at least about 5% increase in a percentage of the plurality of cells expressing an immune checkpoint inhibitor over at least about 24 hours, as compared to control cells. In one embodiment, the threshold of the activity comprises that the cells exhibit at least about 10% increase in a percentage of the plurality of cells expressing an immune checkpoint inhibitor over at least about 24 hours, as compared to control cells. In one embodiment, the threshold of the activity comprises that the cells exhibit at least about 20% increase in a percentage of the plurality of cells expressing an immune checkpoint inhibitor over at least about 24 hours, as compared to control cells. In one embodiment, the threshold of the activity comprises that the cells exhibit at least about 30% increase in a percentage of the plurality of cells expressing an immune checkpoint inhibitor over at least about 24 hours, as compared to control cells. In one embodiment, the threshold of the activity comprises that the cells exhibit at least about 40% increase in a percentage of the plurality of cells expressing an immune checkpoint inhibitor over at least about 24 hours, as compared to control cells. In one embodiment, the threshold of the activity comprises that the cells exhibit at least about 50% increase in a percentage of the plurality of cells expressing an immune checkpoint inhibitor over at least about 24 hours, as compared to control cells. In one embodiment, the threshold of the activity comprises that the cells exhibit at least about 60% increase in a percentage of the plurality of cells expressing an immune checkpoint inhibitor over at least about 24 hours, as compared to control cells. In one embodiment, the threshold of the activity comprises that the cells exhibit at least about 70% increase in a percentage of the plurality of cells expressing an immune checkpoint inhibitor over at least about 24 hours, as compared to control cells. In one embodiment, the threshold of the activity comprises that the cells exhibit at least about 80% increase in a percentage of the plurality of cells expressing an immune checkpoint inhibitor over at least about 24 hours, as compared to control cells.

In one embodiment, the threshold of the activity comprises that the cells exhibit at least about 5% increase in a percentage of the plurality of cells expressing an immune checkpoint inhibitor over at least about 48 hours, as compared to control cells. In one embodiment, the threshold of the activity comprises that the cells exhibit at least about 10% increase in a percentage of the plurality of cells expressing an immune checkpoint inhibitor over at least about 48 hours, as compared to control cells. In one embodiment, the threshold of the activity comprises that the cells exhibit at least about 20% increase in a percentage of the plurality of cells expressing an immune checkpoint inhibitor over at least about 48 hours, as compared to control cells. In one embodiment, the threshold of the activity comprises that the cells exhibit at least about 30% increase in a percentage of the plurality of cells expressing an immune checkpoint inhibitor over at least about 48 hours, as compared to control cells. In one embodiment, the threshold of the activity comprises that the cells exhibit at least about 40% increase in a percentage of the plurality of cells expressing an immune checkpoint inhibitor over at least about 48 hours, as compared to control cells. In one embodiment, the threshold of the activity comprises that the cells exhibit at least about 50% increase in a percentage of the plurality of cells expressing an immune checkpoint inhibitor over at least about 48 hours, as compared to control cells. In one embodiment, the threshold of the activity comprises that the cells exhibit at least about 60% increase in a percentage of the plurality of cells expressing an immune checkpoint inhibitor over at least about 48 hours, as compared to control cells. In one embodiment, the threshold of the activity comprises that the cells exhibit at least about 70% increase in a percentage of the plurality of cells expressing an immune checkpoint inhibitor over at least about 48 hours, as compared to control cells. In one embodiment, the threshold of the activity comprises that the cells exhibit at least about 80% increase in a percentage of the plurality of cells expressing an immune checkpoint inhibitor over at least about 48 hours, as compared to control cells.

In one embodiment, the threshold of the activity comprises that the cells exhibit at least about 5% increase in a percentage of the plurality of cells expressing an immune checkpoint inhibitor over at least about 72 hours, as compared to control cells. In one embodiment, the threshold of the activity comprises that the cells exhibit at least about 10% increase in a percentage of the plurality of cells expressing an immune checkpoint inhibitor over at least about 72 hours, as compared to control cells. In one embodiment, the threshold of the activity comprises that the cells exhibit at least about 20% increase in a percentage of the plurality of cells expressing an immune checkpoint inhibitor over at least about 72 hours, as compared to control cells. In one embodiment, the threshold of the activity comprises that the cells exhibit at least about 30% increase in a percentage of the plurality of cells expressing an immune checkpoint inhibitor over at least about 72 hours, as compared to control cells. In one embodiment, the threshold of the activity comprises that the cells exhibit at least about 40% increase in a percentage of the plurality of cells expressing an immune checkpoint inhibitor over at least about 72 hours, as compared to control cells. In one embodiment, the threshold of the activity comprises that the cells exhibit at least about 50% increase in a percentage of the plurality of cells expressing an immune checkpoint inhibitor over at least about 72 hours, as compared to control cells. In one embodiment, the threshold of the activity comprises that the cells exhibit at least about 60% increase in a percentage of the plurality of cells expressing an immune checkpoint inhibitor over at least about 72 hours, as compared to control cells. In one embodiment, the threshold of the activity comprises that the cells exhibit at least about 70% increase in a percentage of the plurality of cells expressing an immune checkpoint inhibitor over at least about 72 hours, as compared to control cells. In one embodiment, the threshold of the activity comprises that the cells exhibit at least about 80% increase in a percentage of the plurality of cells expressing an immune checkpoint inhibitor over at least about 72 hours, as compared to control cells.

In one embodiment, the threshold of the activity comprises that the cells exhibit at least about 10% increase in expression of a cytokine over at least about 24 hours, as compared to control cells. In one embodiment, the threshold of the activity comprises that the cells exhibit at least about 20% increase in expression of a cytokine over at least about 24 hours, as compared to control cells. In one embodiment, the threshold of the activity comprises that the cells exhibit at least about 30% increase in expression of a cytokine over at least about 24 hours, as compared to control cells. In one embodiment, the threshold of the activity comprises that the cells exhibit at least about 40% increase in expression of a cytokine over at least about 24 hours, as compared to control cells. In one embodiment, the threshold of the activity comprises that the cells exhibit at least about 50% increase in expression of a cytokine over at least about 24 hours, as compared to control cells. In one embodiment, the threshold of the activity comprises that the cells exhibit at least about 60% increase in expression of a cytokine over at least about 24 hours, as compared to control cells. In one embodiment, the threshold of the activity comprises that the cells exhibit at least about 70% increase in expression of a cytokine over at least about 24 hours, as compared to control cells. In one embodiment, the threshold of the activity comprises that the cells exhibit at least about 80% increase in expression of a cytokine over at least about 24 hours, as compared to control cells. In one embodiment, the threshold of the activity comprises that the cells exhibit at least about 90% increase in expression of a cytokine over at least about 24 hours, as compared to control cells. In one embodiment, the threshold of the activity comprises that the cells exhibit at least about 100% increase in expression of a cytokine over at least about 24 hours, as compared to control cells. In one embodiment, the threshold of the activity comprises that the cells exhibit at least about 120% increase in expression of a cytokine over at least about 24 hours, as compared to control cells. In one embodiment, the threshold of the activity comprises that the cells exhibit at least about 140% increase in expression of a cytokine over at least about 24 hours, as compared to control cells. In one embodiment, the threshold of the activity comprises that the cells exhibit at least about 160% increase in expression of a cytokine over at least about 24 hours, as compared to control cells. In one embodiment, the threshold of the activity comprises that the cells exhibit at least about 180% increase in expression of a cytokine over at least about 24 hours, as compared to control cells. In one embodiment, the threshold of the activity comprises that the cells exhibit at least about 200% increase in expression of a cytokine over at least about 24 hours, as compared to control cells. In one embodiment, the threshold of the activity comprises that the cells exhibit at least about 220% increase in expression of a cytokine over at least about 24 hours, as compared to control cells. In one embodiment, the threshold of the activity comprises that the cells exhibit at least about 240% increase in expression of a cytokine over at least about 24 hours, as compared to control cells. In one embodiment, the threshold of the activity comprises that the cells exhibit at least about 250% increase in expression of a cytokine over at least about 24 hours, as compared to control cells. In one embodiment, the threshold of the activity comprises that the cells exhibit at least about 300% increase in expression of a cytokine over at least about 24 hours, as compared to control cells. In one embodiment, the threshold of the activity comprises that the cells exhibit at least about 350% increase in expression of a cytokine over at least about 24 hours, as compared to control cells. In one embodiment, the threshold of the activity comprises that the cells exhibit at least about 400% increase in expression of a cytokine over at least about 24 hours, as compared to control cells. In one embodiment, the threshold of the activity comprises that the cells exhibit at least about 450% increase in expression of a cytokine over at least about 24 hours, as compared to control cells. In one embodiment, the threshold of the activity comprises that the cells exhibit at least about 500% increase in expression of a cytokine over at least about 24 hours, as compared to control cells. In one embodiment, the threshold of the activity comprises that the cells exhibit at least about 550% increase in expression of a cytokine over at least about 24 hours, as compared to control cells. In one embodiment, the threshold of the activity comprises that the cells exhibit at least about 600% increase in expression of a cytokine over at least about 24 hours, as compared to control cells. In one embodiment, the threshold of the activity comprises that the cells exhibit at least about 650% increase in expression of a cytokine over at least about 24 hours, as compared to control cells. In one embodiment, the threshold of the activity comprises that the cells exhibit at least about 700% increase in expression of a cytokine over at least about 24 hours, as compared to control cells. In one embodiment, the threshold of the activity comprises that the cells exhibit at least about 750% increase in expression of a cytokine over at least about 24 hours, as compared to control cells. In one embodiment, the threshold of the activity comprises that the cells exhibit at least about 800% increase in expression of a cytokine over at least about 24 hours, as compared to control cells. In one embodiment, the threshold of the activity comprises that the cells exhibit at least about 850% increase in expression of a cytokine over at least about 24 hours, as compared to control cells. In one embodiment, the threshold of the activity comprises that the cells exhibit at least about 900% increase in expression of a cytokine over at least about 24 hours, as compared to control cells. In one embodiment, the threshold of the activity comprises that the cells exhibit at least about 950% increase in expression of a cytokine over at least about 24 hours, as compared to control cells. In one embodiment, the threshold of the activity comprises that the cells exhibit at least about 1000% increase in expression of a cytokine over at least about 24 hours, as compared to control cells. In one embodiment, the threshold of the activity comprises that the cells exhibit about 1500%, about 2000%, about 2500%, or about 3000% increase in expression of a cytokine over at least about 24 hours, as compared to control cells. In some embodiments, the threshold of the activity comprises that the cells exhibit at least about 100%, at least about 500%, at least about 1000%, about 2000% or about 3000% increase in expression of a cytokine over at least about 48 hours, as compared to control cells. In some embodiments, the threshold of the activity comprises that the cells exhibit at least about 100%, at least about 500%, at least about 1000%, about 2000% or about 3000% increase in expression of a cytokine over at least about 72 hours, as compared to control cells. In some embodiments, the cytokine may be TNFa. In some embodiments, the cytokine may be IFNg. In some embodiments, the cytokine may be IL-2. In some embodiments, the cytokine may be IL-4. In some embodiments, the cytokine may be IL-6. In some embodiments, the cytokine may be IL-10. In some embodiments, the cytokine may be TGF-b.

In some embodiments the threshold activity may comprise a decrease in one or more markers, such as a cytokine or a chemokine or an anti-inflammatory marker or an inhibitor of immune response. In some embodiments, for example, the threshold activity may be a decrease in IL-10 or TGF beta release. In some embodiments, the threshold activity may be a decrease in IL-35. In some embodiments, the threshold activity may be a decrease in IL2RA. In some embodiments, the threshold activity may be a decrease in CTLA4.

In one embodiment, the threshold of the activity comprises that the cells exhibit at least about 20% increase in a percentage of the plurality of cells in a proliferative state over at least about 48 hours, as compared to control cells. In one embodiment, the threshold of the activity comprises that the cells exhibit at least about 30% increase in a percentage of the plurality of cells in a proliferative state over at least about 48 hours, as compared to control cells. In one embodiment, the threshold of the activity comprises that the cells exhibit at least about 40% increase in a percentage of the plurality of cells in a proliferative state over at least about 48 hours, as compared to control cells. In one embodiment, the threshold of the activity comprises that the cells exhibit at least about 50% increase in a percentage of the plurality of cells in a proliferative state over at least about 48 hours, as compared to control cells. In one embodiment, the threshold of the activity comprises that the cells exhibit at least about 60% increase in a percentage of the plurality of cells in a proliferative state over at least about 48 hours, as compared to control cells. In one embodiment, the threshold of the activity comprises that the cells exhibit at least about 70% increase in a percentage of the plurality of cells in a proliferative state over at least about 48 hours, as compared to control cells. In one embodiment, the threshold of the activity comprises that the cells exhibit at least about 80% increase in a percentage of the plurality of cells in a proliferative state over at least about 48 hours, as compared to control cells. In one embodiment, the threshold of the activity comprises that the cells exhibit at least about 90% increase in a percentage of the plurality of cells in a proliferative state over at least about 48 hours, as compared to control cells. In one embodiment, the threshold of the activity comprises that the cells exhibit at least about 100% increase in a percentage of the plurality of cells in a proliferative state over at least about 48 hours, as compared to control cells. In one embodiment, the threshold of the activity comprises that the cells exhibit at least about 200% increase in a percentage of the plurality of cells in a proliferative state over at least about 48 hours, as compared to control cells. In one embodiment, the threshold of the activity comprises that the cells exhibit at least about 250% increase in a percentage of the plurality of cells in a proliferative state over at least about 48 hours, as compared to control cells. In one embodiment, the threshold of the activity comprises that the cells exhibit at least about 300% increase in a percentage of the plurality of cells in a proliferative state over at least about 48 hours, as compared to control cells.

In one embodiment, the threshold of the activity comprises that the cells exhibit at least about 20% increase in a percentage of the plurality of cells in a proliferative state over at least about 72 hours, as compared to control cells. In one embodiment, the threshold of the activity comprises that the cells exhibit at least about 30% increase in a percentage of the plurality of cells in a proliferative state over at least about 72 hours, as compared to control cells. In one embodiment, the threshold of the activity comprises that the cells exhibit at least about 40% increase in a percentage of the plurality of cells in a proliferative state over at least about 72 hours, as compared to control cells. In one embodiment, the threshold of the activity comprises that the cells exhibit at least about 50% increase in a percentage of the plurality of cells in a proliferative state over at least about 72 hours, as compared to control cells. In one embodiment, the threshold of the activity comprises that the cells exhibit at least about 60% increase in a percentage of the plurality of cells in a proliferative state over at least about 72 hours, as compared to control cells. In one embodiment, the threshold of the activity comprises that the cells exhibit at least about 70% increase in a percentage of the plurality of cells in a proliferative state over at least about 72 hours, as compared to control cells. In one embodiment, the threshold of the activity comprises that the cells exhibit at least about 80% increase in a percentage of the plurality of cells in a proliferative state over at least about 72 hours, as compared to control cells. In one embodiment, the threshold of the activity comprises that the cells exhibit at least about 90% increase in a percentage of the plurality of cells in a proliferative state over at least about 72 hours, as compared to control cells. In one embodiment, the threshold of the activity comprises that the cells exhibit at least about 100% increase in a percentage of the plurality of cells in a proliferative state over at least about 72 hours, as compared to control cells. In one embodiment, the threshold of the activity comprises that the cells exhibit at least about 200% increase in a percentage of the plurality of cells in a proliferative state over at least about 72 hours, as compared to control cells. In one embodiment, the threshold of the activity comprises that the cells exhibit at least about 250% increase in a percentage of the plurality of cells in a proliferative state over at least about 72 hours, as compared to control cells. In one embodiment, the threshold of the activity comprises that the cells exhibit at least about 300% increase in a percentage of the plurality of cells in a proliferative state over at least about 72 hours, as compared to control cells.

In one embodiment, the threshold of the activity comprises that the cells exhibit at least about 20% increase in a percentage of the plurality of cells in a proliferative state over at least about 96 hours, as compared to control cells. In one embodiment, the threshold of the activity comprises that the cells exhibit at least about 30% increase in a percentage of the plurality of cells in a proliferative state over at least about 96 hours, as compared to control cells. In one embodiment, the threshold of the activity comprises that the cells exhibit at least about 40% increase in a percentage of the plurality of cells in a proliferative state over at least about 96 hours, as compared to control cells. In one embodiment, the threshold of the activity comprises that the cells exhibit at least about 50% increase in a percentage of the plurality of cells in a proliferative state over at least about 96 hours, as compared to control cells. In one embodiment, the threshold of the activity comprises that the cells exhibit at least about 60% increase in a percentage of the plurality of cells in a proliferative state over at least about 96 hours, as compared to control cells. In one embodiment, the threshold of the activity comprises that the cells exhibit at least about 70% increase in a percentage of the plurality of cells in a proliferative state over at least about 96 hours, as compared to control cells. In one embodiment, the threshold of the activity comprises that the cells exhibit at least about 80% increase in a percentage of the plurality of cells in a proliferative state over at least about 96 hours, as compared to control cells. In one embodiment, the threshold of the activity comprises that the cells exhibit at least about 90% increase in a percentage of the plurality of cells in a proliferative state over at least about 96 hours, as compared to control cells. In one embodiment, the threshold of the activity comprises that the cells exhibit at least about 100% increase in a percentage of the plurality of cells in a proliferative state over at least about 96 hours, as compared to control cells. In one embodiment, the threshold of the activity comprises that the cells exhibit at least about 200% increase in a percentage of the plurality of cells in a proliferative state over at least about 96 hours, as compared to control cells. In one embodiment, the threshold of the activity comprises that the cells exhibit at least about 250% increase in a percentage of the plurality of cells in a proliferative state over at least about 96 hours, as compared to control cells. In one embodiment, the threshold of the activity comprises that the cells exhibit at least about 300% increase in a percentage of the plurality of cells in a proliferative state over at least about 96 hours, as compared to control cells.

In one embodiment, the threshold of the activity comprises that the cells exhibit at least about 10% change in a percentage of the plurality of cells expressing a cell exhaustion marker over at least about 24 hours, as compared to control cells. In one embodiment, the threshold of the activity comprises that the cells exhibit at least about 20% change in a percentage of the plurality of cells expressing a cell exhaustion marker over at least about 24 hours, as compared to control cells. In one embodiment, the threshold of the activity comprises that the cells exhibit at least about 30% change in a percentage of the plurality of cells expressing a cell exhaustion marker over at least about 24 hours, as compared to control cells. In one embodiment, the threshold of the activity comprises that the cells exhibit at least about 40% change in a percentage of the plurality of cells expressing a cell exhaustion marker over at least about 24 hours, as compared to control cells. In one embodiment, the threshold of the activity comprises that the cells exhibit at least about 50% change in a percentage of the plurality of cells expressing a cell exhaustion marker over at least about 24 hours, as compared to control cells.

In one embodiment, the threshold of the activity comprises that the cells exhibit at least about 60% change in a percentage of the plurality of cells expressing a cell exhaustion marker over at least about 24 hours, as compared to control cells. In one embodiment, the threshold of the activity comprises that the cells exhibit at least about 70% change in a percentage of the plurality of cells expressing a cell exhaustion marker over at least about 24 hours, as compared to control cells. In one embodiment, the threshold of the activity comprises that the cells exhibit at least about 80% change in a percentage of the plurality of cells expressing a cell exhaustion marker over at least about 24 hours, as compared to control cells. In one embodiment, the threshold of the activity comprises that the cells exhibit at least about 90% change in a percentage of the plurality of cells expressing a cell exhaustion marker over at least about 24 hours, as compared to control cells. In one embodiment, the threshold of the activity comprises that the cells exhibit at least about 100% change in a percentage of the plurality of cells expressing a cell exhaustion marker over at least about 24 hours, as compared to control cells. In one embodiment, the threshold of the activity comprises that the cells exhibit at least about 200%, about 300%, about 500% change in a percentage of the plurality of cells expressing a cell exhaustion marker over at least about 24 hours, as compared to control cells. Exemplary exhaustion markers may include TIM3, LAG, TNFa, PD-1 or encompasses any other marker known to one of skill in the art.

In some embodiments, the threshold may include but not limited to, a function or a change that is induced by the CAR or the actuator moiety. In some embodiments, the cell may, for example, express actuator moiety operatively coupled to the CAR, wherein actuator moiety is capable of reducing expression of a target gene (e.g., an endogenous gene, such as an immune checkpoint inhibitor gene). In such embodiment, the threshold may include at least about 5%, at least about 10%, at least about 15%, at least about 20%, at least about 25%, at least about 50%, or nearly about 100%, decrease in a percentage of the plurality of cells expressing the target gene over at least about 24 hours, as compared to control cells. Alternatively, wherein actuator moiety is capable of reducing expression of a target gene (e.g., an endogenous gene, the threshold may include at least about 5% increase in a percentage of the plurality of cells expressing the target gene over at least about 24 hours, as compared to control cells). In some embodiments, an exhaustion marker may include one or more of, but not limited to, CD96 (TACTILE), CD152 (CTLA-4), CD160 (NK1), CD223 (LAG-3), CD244 (2B4), CD272 (BTLA), CD279 (PD1), CD366 (TIM-3), EOMES, ICOS, TIGIT, and VISTA.

In some embodiments, the plurality of cells can be a variety of immune cells, including any cell that is involved in an immune response. In some embodiments, immune cells comprise granulocytes such as basophils, eosinophils, and neutrophils; mast cells; monocytes which can develop into macrophages; antigen-presenting cells such as dendritic cells; and lymphocytes such as natural killer cells (NK cells), B cells, and T cells. In some embodiments, an immune cell is an immune effector cell. An immune effector cell refers to an immune cell that can perform a specific function in response to a stimulus. In some embodiments, an immune cell is an immune effector cell which can induce cell death. In some embodiments, the immune cell is a lymphocyte. In some embodiments, the lymphocyte is a NK cell. In some embodiments the lymphocyte is a T cell. In some embodiments, the T cell is an activated T cell. T cells include both naive and memory cells (e.g. central memory or Tcm, effector memory or TEM and effector memory RA or T_{EMRA}), effector cells (e.g. cytotoxic T cells or CTLs or Tc cells), helper cells (e.g. Th1, Th2, Th3, Th9, Th7, TFH), regulatory cells (e.g. Treg, and Trl cells), natural killer T cells (NKT cells), tumor infiltrating lymphocytes (TILs), lymphocyte-activated killer cells (LAKs), αβ T cells, γδ T cells, and similar unique classes of the T cell lineage. T cells can be divided into two broad categories: CD8+ T cells and CD4+ T cells, based on which protein is present on the cell's surface. T cells expressing a subject system can carry out multiple functions, including killing infected cells and activating or recruiting other immune cells. CD8+ T cells are referred to as cytotoxic T cells or cytotoxic T lymphocytes (CTLs). CTLs expressing a subject system can be involved in recognizing and removing virus-infected cells and cancer cells. CTLs have specialized compartments, or granules, containing cytotoxins that cause apoptosis, e.g., programmed cell death. CD4+ T cells can be subdivided into four sub-sets—Th1, Th2, Th17, and Treg, with “Th” referring to “T helper cell,” although additional sub-sets may exist. Th1 cells can coordinate immune responses against intracellular microbes, especially bacteria. They can produce and secrete molecules that alert and activate other immune cells, like bacteria-ingesting macrophages. Th2 cells are involved in coordinating immune responses against extracellular pathogens, like helminths (parasitic worms), by alerting B cells, granulocytes, and mast cells. Th17 cells can produce interleukin 17 (IL-17), a signaling molecule that activates immune and non-immune cells. Th17 cells are important for recruiting neutrophils.

In some embodiments, the non-cellular substrate is an inert, solid substrate that may offer a ligand for the ligand binding domain of the chimeric receptor at any possible contact surface of the receptor to the non-cellular substrate. The ligand may be an antigen. In some embodiments, the non-cellular substrate method may be a bead, that is conjugated with an antigen that said chimeric receptor comprised in the cell may specifically bind to. In some embodiments, the bead may be a spherical bead that is roughly the size of a cell, such as antigen presenting cell. The antigen density on the substrate may vary, and different sets of beads in which each set comprises a certain antigen density on its surface may be used for an assay to determine functional efficacies of the cells by a method described in the disclosure. In some embodiments, at least two, at least three, at least four or at least five antigen densities may be selected per assay. In some embodiments, the bead to cell ration may vary and one or more sets of bead: cell ratio may be included per assay. In some embodiments the method described herein may comprise a bead: cell ratio 50:1 to 1:500. In some embodiments the bead: cell ratio may be 5:1, 4:1, 3:1, 2:1 or 1:1. In some embodiments the bead: cell ratio may be 1:1, 1:2, 1:3, 1:4, 1:5, 1:6, 1:7, 1:8, 1:9, 1:10, 1:12, 1:15, 1:20, 1:25, 1:30, 1:35, 1:40, 1:45, 1:50, 1:60, 1:70, 1:80, 1:90, 1:100, or 1:125 or more. In some embodiments, the bead: cell ratio may include at least specifically 5:1, 1:1, 1:5 in an assay.

In some embodiments, control cells may be cells not treated with the non-cellular substrate comprising the antigen, e.g., cells prior to such treatment or cells treated with non-cellular substrates that do not comprise the antigen.

In some instances, the thresholds comprising cytokine secretion can be measured by amount of cytokine secreted by a defined number of cells. The amount of cytokine can be measured with any methods such as mass spectrometry, western blotting, ELISA, etc. In some cases, cytokine can be secreted into varying volume of liquid media. As such, measuring the secreted cytokine can include controlling for both the number of cells secreting the cytokine and the volume of liquid media where the cytokine is present. For example, an amount of cytokine secreted by 10,000 cells can be obtained from a 1 milliliter liquid media or from a 10 milliliter liquid media with an ELISA kit. In such case, the amount of cytokine secreted measured from the ELISA kit can be calculated to be the amount of cytokine secreted per cell, the amount of cytokine secreted per milliliter of liquid media, or the amount of cytokine secreted per cell per milliliter of liquid media. Any additional dilution steps can be considered for the calculation. One skilled in the art will know of modifications of the abovementioned exemplary method or any additional methods of calculating the amount of cytokine secreted (e.g., over a defined period of time).

In some embodiments, the thresholds comprise cytokine secretion. In some cases, the threshold may be defined by a cytokine secretion by a defined number of cells, wherein the cytokine secretion exceeds at least about 10 picograms (0.01 nanograms) to about 10 micrograms (10000 nanograms). In some cases, the threshold may be defined by a cytokine secretion exceeding at least about 0.01 nanograms to about 10,000 nanograms. In some cases, the threshold may be defined by a cytokine secretion exceeding at least at least about 0.01 nanograms. In some cases, the threshold may be defined by a cytokine secretion exceeding at least at most about 10,000 nanograms. In some cases, the threshold may be defined by a cytokine secretion exceeding at least about 0.01 nanograms to about 0.1 nanograms, about 0.01 nanograms to about 0.5 nanograms, about 0.01 nanograms to about 1 nanogram, about 0.01 nanograms to about 5 nanograms, about 0.01 nanograms to about 10 nanograms, about 0.01 nanograms to about 50 nanograms, about 0.01 nanograms to about 100 nanograms, about 0.01 nanograms to about 500 nanograms, about 0.01 nanograms to about 1,000 nanograms, about 0.01 nanograms to about 5,000 nanograms, about 0.01 nanograms to about 10,000 nanograms, about 0.1 nanograms to about 0.5 nanograms, about 0.1 nanograms to about 1 nanogram, about 0.1 nanograms to about 5 nanograms, about 0.1 nanograms to about 10 nanograms, about 0.1 nanograms to about 50 nanograms, about 0.1 nanograms to about 100 nanograms, about 0.1 nanograms to about 500 nanograms, about 0.1 nanograms to about 1,000 nanograms, about 0.1 nanograms to about 5,000 nanograms, about 0.1 nanograms to about 10,000 nanograms, about 0.5 nanograms to about 1 nanogram, about 0.5 nanograms to about 5 nanograms, about 0.5 nanograms to about 10 nanograms, about 0.5 nanograms to about 50 nanograms, about 0.5 nanograms to about 100 nanograms, about 0.5 nanograms to about 500 nanograms, about 0.5 nanograms to about 1,000 nanograms, about 0.5 nanograms to about 5,000 nanograms, about 0.5 nanograms to about 10,000 nanograms, about 1 nanogram to about 5 nanograms, about 1 nanogram to about 10 nanograms, about 1 nanogram to about 50 nanograms, about 1 nanogram to about 100 nanograms, about 1 nanogram to about 500 nanograms, about 1 nanogram to about 1,000 nanograms, about 1 nanogram to about 5,000 nanograms, about 1 nanogram to about 10,000 nanograms, about 5 nanograms to about 10 nanograms, about 5 nanograms to about 50 nanograms, about 5 nanograms to about 100 nanograms, about 5 nanograms to about 500 nanograms, about 5 nanograms to about 1,000 nanograms, about 5 nanograms to about 5,000 nanograms, about 5 nanograms to about 10,000 nanograms, about 10 nanograms to about 50 nanograms, about 10 nanograms to about 100 nanograms, about 10 nanograms to about 500 nanograms, about 10 nanograms to about 1,000 nanograms, about 10 nanograms to about 5,000 nanograms, about 10 nanograms to about 10,000 nanograms, about 50 nanograms to about 100 nanograms, about 50 nanograms to about 500 nanograms, about 50 nanograms to about 1,000 nanograms, about 50 nanograms to about 5,000 nanograms, about 50 nanograms to about 10,000 nanograms, about 100 nanograms to about 500 nanograms, about 100 nanograms to about 1,000 nanograms, about 100 nanograms to about 5,000 nanograms, about 100 nanograms to about 10,000 nanograms, about 500 nanograms to about 1,000 nanograms, about 500 nanograms to about 5,000 nanograms, about 500 nanograms to about 10,000 nanograms, about 1,000 nanograms to about 5,000 nanograms, about 1,000 nanograms to about 10,000 nanograms, or about 5,000 nanograms to about 10,000 nanograms. In some cases, the threshold may be defined by a cytokine secretion exceeding at least about 0.01 nanograms, about 0.1 nanograms, about 0.5 nanograms, about 1 nanogram, about 5 nanograms, about 10 nanograms, about 50 nanograms, about 100 nanograms, about 500 nanograms, about 1,000 nanograms, about 5,000 nanograms, or about 10,000 nanograms.

In some embodiments, the thresholds comprise cytokine secretion. In some cases, the threshold may be defined by a cytokine secretion by a defined number of cells, wherein the cytokine secretion exceeds at most about 10 picograms (0.01 nanograms) to about 10 micrograms (10000 nanograms). In some cases, the threshold may be defined by a cytokine secretion exceeding at most about 0.01 nanograms to about 10,000 nanograms. In some cases, the threshold may be defined by a cytokine secretion exceeding at most at least about 0.01 nanograms. In some cases, the threshold may be defined by a cytokine secretion exceeding at most at most about 10,000 nanograms. In some cases, the threshold may be defined by a cytokine secretion exceeding at most about 0.01 nanograms to about 0.1 nanograms, about 0.01 nanograms to about 0.5 nanograms, about 0.01 nanograms to about 1 nanogram, about 0.01 nanograms to about 5 nanograms, about 0.01 nanograms to about 10 nanograms, about 0.01 nanograms to about 50 nanograms, about 0.01 nanograms to about 100 nanograms, about 0.01 nanograms to about 500 nanograms, about 0.01 nanograms to about 1,000 nanograms, about 0.01 nanograms to about 5,000 nanograms, about 0.01 nanograms to about 10,000 nanograms, about 0.1 nanograms to about 0.5 nanograms, about 0.1 nanograms to about 1 nanogram, about 0.1 nanograms to about 5 nanograms, about 0.1 nanograms to about 10 nanograms, about 0.1 nanograms to about 50 nanograms, about 0.1 nanograms to about 100 nanograms, about 0.1 nanograms to about 500 nanograms, about 0.1 nanograms to about 1,000 nanograms, about 0.1 nanograms to about 5,000 nanograms, about 0.1 nanograms to about 10,000 nanograms, about 0.5 nanograms to about 1 nanogram, about 0.5 nanograms to about 5 nanograms, about 0.5 nanograms to about 10 nanograms, about 0.5 nanograms to about 50 nanograms, about 0.5 nanograms to about 100 nanograms, about 0.5 nanograms to about 500 nanograms, about 0.5 nanograms to about 1,000 nanograms, about 0.5 nanograms to about 5,000 nanograms, about 0.5 nanograms to about 10,000 nanograms, about 1 nanogram to about 5 nanograms, about 1 nanogram to about 10 nanograms, about 1 nanogram to about 50 nanograms, about 1 nanogram to about 100 nanograms, about 1 nanogram to about 500 nanograms, about 1 nanogram to about 1,000 nanograms, about 1 nanogram to about 5,000 nanograms, about 1 nanogram to about 10,000 nanograms, about 5 nanograms to about 10 nanograms, about 5 nanograms to about 50 nanograms, about 5 nanograms to about 100 nanograms, about 5 nanograms to about 500 nanograms, about 5 nanograms to about 1,000 nanograms, about 5 nanograms to about 5,000 nanograms, about 5 nanograms to about 10,000 nanograms, about 10 nanograms to about 50 nanograms, about 10 nanograms to about 100 nanograms, about 10 nanograms to about 500 nanograms, about 10 nanograms to about 1,000 nanograms, about 10 nanograms to about 5,000 nanograms, about 10 nanograms to about 10,000 nanograms, about 50 nanograms to about 100 nanograms, about 50 nanograms to about 500 nanograms, about 50 nanograms to about 1,000 nanograms, about 50 nanograms to about 5,000 nanograms, about 50 nanograms to about 10,000 nanograms, about 100 nanograms to about 500 nanograms, about 100 nanograms to about 1,000 nanograms, about 100 nanograms to about 5,000 nanograms, about 100 nanograms to about 10,000 nanograms, about 500 nanograms to about 1,000 nanograms, about 500 nanograms to about 5,000 nanograms, about 500 nanograms to about 10,000 nanograms, about 1,000 nanograms to about 5,000 nanograms, about 1,000 nanograms to about 10,000 nanograms, or about 5,000 nanograms to about nanograms. In some cases, the threshold may be defined by a cytokine secretion exceeding at most about 0.01 nanograms, about 0.1 nanograms, about 0.5 nanograms, about 1 nanogram, about 5 nanograms, about 10 nanograms, about 50 nanograms, about 100 nanograms, about 500 nanograms, about 1,000 nanograms, about 5,000 nanograms, or about 10,000 nanograms.

In some embodiments, the thresholds may be defined by a cytokine secretion by a defined number of cells, wherein the cytokine secretion exceeds at least about 10 picograms, 20 picograms, 50 picograms, 100 picograms, 200 picograms, 500 picograms, 1 nanogram, 2 nanograms, 5 nanograms, nanograms, 20 nanograms, 50 nanograms, 100 nanograms, 200 nanograms, 500 nanograms, 1 microgram, 2 micrograms, 5 micrograms, 10 micrograms, or more.

In some embodiments, the thresholds may be defined by a cytokine secretion by a defined number of cells, wherein the cytokine secretion exceeds at most about 10 micrograms, 5 micrograms, 1 microgram, 500 nanograms, 100 nanograms, 50 nanograms, 10 nanograms, 5 nanograms, 1 nanogram, 500 picograms, 100 picograms, 50 picograms, 10 picograms, or less.

In implementing any one of the methods provided in the present disclosure, or in any one of the systems provided in the present disclosure, a defined number of cells can be about 1000 cells, 2000 cells, 5000 cells, 10,000 cells, 20,000 cells, 50,000 cells, 100,000 cells, 200,000 cells, 500,000 cells, 1,000,000 cells, 2,000,000 cells, 5,000,000 cells, 10,000,000 cells, 20,000,000 cells, 50,000,000 cells, 100,000,000 cells, 200,000,000 cells, 500,000,000 cells, 1,000,000,000 cells, or more. In some cases, the defined number of cells can be at least 1000 cells, at least 2000 cells, at least 5000 cells, at least cells, at least 20,000 cells, at least 50,000 cells, at least 100,000 cells, at least 200,000 cells, at least 500,000 cells, at least 1,000,000 cells, at least 2,000,000 cells, at least 5,000,000 cells, at least cells, at least 20,000,000 cells, at least 50,000,000 cells, at least 100,000,000 cells, at least 200,000,000 cells, at least 500,000,000 cells, at least 1,000,000,000 cells, or more. In some cases, the defined number of cells can be at most 1,000,000,000 cells, at most 500,000,000 cells, at most 100,000,000 cells, at most 50,000,000 cells, at most 10,000,000 cells, at most 5,000,000 cells, at most 1,000,000 cells, at most 500,000 cells, at most 100,000 cells, at most 50,000 cells, at most 10,000 cells, at most 5,000 cells, at most 1,000 cells, or less.

In some cases, the thresholds may be defined by a cytokine secretion exceeding at least about picogram to about 10 microgram secreted by about 1000 cells, 2000 cells, 5000 cells, 10,000 cells, cells, 50,000 cells, 100,000 cells, 200,000 cells, 500,000 cells, 1,000,000 cells, 2,000,000 cells, cells, 10,000,000 cells, 20,000,000 cells, 50,000,000 cells, 100,000,000 cells, 200,000,000 cells, 500,000,000 cells, or 1,000,000,000 cells. In some cases, the thresholds may be defined by a cytokine secretion exceeding at least about 10 picograms to about 10 micrograms secreted by at least 1000 cells, at least 2000 cells, at least 5000 cells, at least 10,000 cells, at least 20,000 cells, at least cells, at least 100,000 cells, at least 200,000 cells, at least 500,000 cells, at least 1,000,000 cells, at least 2,000,000 cells, at least 5,000,000 cells, at least 10,000,000 cells, at least 20,000,000 cells, at least 50,000,000 cells, at least 100,000,000 cells, at least 200,000,000 cells, at least 500,000,000 cells, or at least 1,000,000,000 cells, or more. In some cases, the thresholds may be defined by a cytokine secretion exceeding at least about 10 picogram to about 10 microgram secreted by at most 1,000,000,000 cells, at most 500,000,000 cells, at most 100,000,000 cells, at most cells, at most 10,000,000 cells, at most 5,000,000 cells, at most 1,000,000 cells, at most 500,000 cells, at most 100,000 cells, at most 50,000 cells, at most 10,000 cells, at most 5,000 cells, at most 1,000 cells, or less.

In some cases, the thresholds of cytokine secretion may be determined by the amount of cytokine secreted by a defined number of cells over a period of time. In some cases, the thresholds of cytokine secretion may be determined by the amount of cytokine secreted by a defined number of cells over a period of at least about 1 minute, 2 minutes, 5 minutes, 10 minutes, 20 minutes, 30 minutes, 1 hour, 2 hours, 3 hours, 4 hours, 5 hours, 6 hours, 7 hours, 8 hours, 9 hours, 10 hours, 12 hours, 24 hours, 36 hours, 48 hours, 72 hours, or more. In some cases, the thresholds of cytokine secretion may be determined by the amount of cytokine secreted by a defined number of cells over a period of at most about 72 hours, 48 hours, 36 hours, 24 hours, 12 hours, 10 hours, 9 hours, 8 hours, 7 hours, 6 hours, 5 hours, 4 hours, 3 hours, 2 hours, 1 hour, 30 minutes, 20 minutes, 10 minutes, 5 minutes, 2 minutes, 1 minute, or less.

In some embodiments, the thresholds of cytokine secretion may be defined by a cytokine secretion exceeding at least about 10 picograms to about 10 micrograms or at most about 10 picograms to about 10 micrograms secreted by about 1000 cells, 2000 cells, 5000 cells, 10,000 cells, cells, 50,000 cells, 100,000 cells, 200,000 cells, 500,000 cells, 1,000,000 cells, 2,000,000 cells, cells, 10,000,000 cells, 20,000,000 cells, 50,000,000 cells, 100,000,000 cells, 200,000,000 cells, 500,000,000 cells, or 1,000,000,000 cells over a period of 1 minute, 2 minutes, 5 minutes, 10 minutes, 20 minutes, 30 minutes, 1 hour, 2 hours, 5 hours, 10 hours, 12 hours, 24 hours, 36 hours, 48 hours, 72 hours, or longer. In some embodiments, the thresholds of cytokine secretion may be defined by a cytokine secretion exceeding at least about 10 picograms to about 10 micrograms, about 10 picograms to about 10 micrograms, or at most about 10 picograms to about 10 micrograms secreted by at least 1000 cells, at least 2000 cells, at least 5000 cells, at least 10,000 cells, at least 20,000 cells, at least 50,000 cells, at least 100,000 cells, at least 200,000 cells, at least 500,000 cells, at least 1,000,000 cells, at least 2,000,000 cells, at least 5,000,000 cells, at least 10,000,000 cells, at least 20,000,000 cells, at least 50,000,000 cells, at least 100,000,000 cells, at least 200,000,000 cells, at least 500,000,000 cells, at least 1,000,000,000 cells, or more over a period of about 1 minute, 2 minutes, 5 minutes, 10 minutes, 20 minutes, 30 minutes, 1 hour, 2 hours, 5 hours, 10 hours, 12 hours, 24 hours, 36 hours, 48 hours, 72 hours, or longer. In some embodiments, the thresholds of cytokine secretion may be defined by a cytokine secretion exceeding at least about 10 picograms to about 10 micrograms, about 10 picograms to about 10 micrograms, or at most about 10 picograms to about 10 micrograms secreted by at most 1,000,000,000 cells, at most 500,000,000 cells, at most 100,000,000 cells, at most cells, at most 10,000,000 cells, at most 5,000,000 cells, at most 1,000,000 cells, at most 500,000 cells, at most 100,000 cells, at most 50,000 cells, at most 10,000 cells, at most 5,000 cells, at most 1,000 cells, or less over a period of about 1 minute, 2 minutes, 5 minutes, 10 minutes, 20 minutes, 30 minutes, 1 hour, 2 hours, 5 hours, 10 hours, 12 hours, 24 hours, 36 hours, 48 hours, 72 hours, or longer.

In implementing any one of the methods provided in the present disclosure, or in any one of the systems provided in the present disclosure, a cytokine secreted by the first and second sub-population of the plurality of cells can be a pro-inflammatory cytokine or anti-inflammatory cytokine. Pro-inflammatory cytokines refer to cytokines involved in inducing or amplifying an inflammatory reaction. Pro-inflammatory cytokines can work with various cells of the immune system, such as neutrophils and leukocytes, to generate an immune response. Certain cytokines can function as anti-inflammatory cytokines. Anti-inflammatory cytokines refer to cytokines involved in the reduction of an inflammatory reaction. Anti-inflammatory cytokines, in some cases, can regulate a pro-inflammatory cytokine response. Some cytokines can function as both pro- and anti-inflammatory cytokines.

In some embodiments, the expression of a cytokine having pro-inflammatory functions can be up-regulated in an immune cell. Up-regulating the expression of a cytokine having pro-inflammatory functions can be useful, for example, to stimulate an immune response against a target cell in immunotherapy. However, excessive amounts of pro-inflammatory cytokines can, in some cases, cause detrimental effects, such as chronic systemic elevations in the body. In some embodiments, the expression of a cytokine having pro-inflammatory functions is down-regulated. Such down-regulation can decrease and/or minimize detrimental effects.

Examples of cytokines that are regulatable by systems and compositions of the present disclosure include, but are not limited to lymphokines, monokines, and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-alpha; mullerian-inhibiting substance; mouse gonadotropin-associated peptide; inhibin; activin; vascular endothelial growth factor; integrin; thrombopoietin (TPO); nerve growth factors such as NGF-alpha; platelet-growth factor; transforming growth factors (TGFs) such as TGF-alpha, TGF-beta, TGF-beta1, TGF-beta2, and TGF-beta3; insulin-like growth factor-I and —II; erythropoietin (EPO); Flt-3L; stem cell factor (SCF); osteoinductive factors; interferons (IFNs) such as IFN-α, IFN-β, IFN-γ; colony stimulating factors (CSFs) such as macrophage-CSF (M-CSF); granulocyte-macrophage-CSF (GM-CSF); granulocyte-CSF (G-CSF); macrophage stimulating factor (MSP); interleukins (ILs) such as IL-1, IL-1a, IL-1b, IL-1RA, IL-18, IL-2, IL-3, IL-4, IL-5, IL-6, IL-7, IL-8, IL-9, IL-10, IL-11, IL-12, IL-13, IL-14, IL-15, IL-16, IL-17, IL-20; a tumor necrosis factor such as CD154, LT-beta, TNF-alpha, TNF-beta, 4-1BBL, APRIL, CD70, CD153, GITRL, LIGHT, OX40L, TALL-1, TRAIL, TWEAK, TRANCE; and other polypeptide factors including LIF, oncostatin M (OSM) and kit ligand (KL). Cytokine receptors refer to the receptor proteins which bind cytokines. Cytokine receptors may be both membrane-bound and soluble.

In some embodiments, the cytokines secreted by the first and second sub-population of the plurality of cells can be IL18, IL18BP, ILIA, IL1B, IL1F10, IL1F3/IL1RA, IL1F5, IL1F6, IL1F7, IL1F8, IL1RL2, IL1F9, IL33, BAFF/BLyS/TNFSF138, 4-1BBL, CD153/CD30L/TNFSF8, CD40LG, CD70, Fas Ligand/FASLG/CD95L/CD178, EDA-A1, TNFSF14/LIGHT/CD258, TNFA, LTA/TNFB/TNFSF1, LTB/TNFC, CD70/CD27L/TNFSF7, TNFSF10/TRAIL/APO-2L(CD253), RANKL/OPGL/TNFSF11(CD254), TNFSF12, TNF-alpha/TNFA, TNFSF13, TL1A/TNFSF15, OX-40L/TNFSF4/CD252, CD40L/CD154/TNFSF5, IFNA1, IFNA10, IFNA13, IFNA14, IFNA2, IFNA4, IFNA7, IFNB1, IFNE, IFNG, IFNZ, IFNA8, IFNA5/IFNaG, IFN(D/IFNω1, CLCF1, CNTF, IL11, IL31, IL6, Leptin, LIF, OSM, CCL1/TCA3, CCL11, CCL12/MCP-5, CCL13/MCP-4, CCL14, CCL15, CCL16, CCL17/TARC, CCL18, CCL19, CCL2/MCP-1, CCL20, CCL21, CCL22/MDC, CCL23, CCL24, CCL25, CCL26, CCL27, CCL28, CCL3, CCL3L3, CCL4, CCL4L1/LAG-1, CCL5, CCL6, CCL7, CCL8, CCL9, CX3CL1, CXCL1, CXCL10, CXCL11, CXCL12, CXCL13, CXCL14, CXCL15, CXCL16, CXCL17, CXCL2/MIP-2, CXCL3, CXCL4, CXCL5, CXCL6, CXCL7/Ppbp, CXCL9, IL8/CXCL8, XCL1, XCL2, Cerberus (protein), CD131, CD132, FMS-like tyrosine kinase 3 ligand, Foxp3, GATA-3, FAM19A1, FAM19A2, FAM19A3, FAM19A4, and FAM19A5.

Cytokine production can be evaluated using a variety of methods. Cytokine production can be evaluated by assaying cell culture media (e.g., in vitro production) in which the modified immune cells are grown or sera (e.g., in vivo production) obtained from a subject having the modified immune cells for the presence of one or more cytokines. Cytokine levels can be quantified in various suitable units, including concentration, using any suitable assay. In some embodiments, cytokine protein is detected. In some embodiments, mRNA transcripts of cytokines are detected. Examples of cytokine assays include enzyme-linked immunosorbent assays (ELISA), immunoblot, immunofluorescence assays, radioimmunoassays, antibody arrays which allow various cytokines in a sample to be detected in parallel, bead-based arrays, quantitative PCR, microarray, etc. Other suitable methods may include proteomics approaches (2-D gels, MS analysis etc).

In some embodiments, the thresholds comprise an increase of expression of mRNA encoding a cytokine. In some cases, the increase of cytokine mRNA expression can be increased by at least 0.1 fold to about 1000 fold. In some cases, the increase of cytokine mRNA expression can be increased by at least 0.1 fold to about 20 fold, at least about 0.1 fold to about 50 fold, at least about 0.1 fold to about 100 fold, at least about 0.1 fold to about 200 fold, at least about 0.1 fold to about 500 fold, at least about 0.1 fold to about 1,000 fold, at least about 2 fold to about 50 fold, at least about 2 fold to about 100 fold, at least about 2 fold to about 200 fold, at least about 2 fold to about 500 fold, at least about 2 fold to about 1,000 fold, at least about 10 fold to about 100 fold, at least about 10 fold to about 200 fold, at least about 10 fold to about 500 fold, at least about 10 fold to about 1,000 fold, at least about fold to about 200 fold, at least about 50 fold to about 500 fold, at least about 50 fold to about 1,000 fold, at least about 100 fold to about 500 fold, at least about 100 fold to about 1,000 fold, or at least about 500 fold to about 1,000 fold.

In some embodiments, the thresholds comprise an increase of expression of mRNA encoding a cytokine. In some cases, the increase of cytokine mRNA expression can be increased by at most 0.1 fold to about 1000 fold. In some cases, the increase of cytokine mRNA expression can be increased by at most 0.1 fold to about 20 fold, at most about 0.1 fold to about 50 fold, at most about 0.1 fold to about 100 fold, at most about 0.1 fold to about 200 fold, at most about 0.1 fold to about 500 fold, at most about 0.1 fold to about 1,000 fold, at most about 2 fold to about 50 fold, at most about 2 fold to about 100 fold, at most about 2 fold to about 200 fold, at most about 2 fold to about 500 fold, at most about 2 fold to about 1,000 fold, at most about 10 fold to about 100 fold, at most about 10 fold to about 200 fold, at most about 10 fold to about 500 fold, at most about 10 fold to about 1,000 fold, at most about 50 fold to about 200 fold, at most about 50 fold to about 500 fold, at most about 50 fold to about 1,000 fold, at most about 100 fold to about 500 fold, at most about 100 fold to about 1,000 fold, or at most about 500 fold to about 1,000 fold.

In some embodiments, the thresholds comprise an increase of expression of mRNA encoding a cytokine. In some cases, the increase of cytokine mRNA expression can be increased by at least about fold, 0.2 fold, 1 fold, 2 fold, 5 fold, 10 fold, 50 fold, 100 fold, 500 fold, 1,000 fold, 5,000 fold, fold, 50,000 fold, 100,000 fold, or more. In some cases, the increase of cytokine mRNA expression can be increased by at most about 100,000 fold, 50,000 fold, 10,000 fold, 5,000 fold, 1,000 fold, 500 fold, 200 fold, 100 fold, 50 fold, 20 fold, 10 fold, 5 fold, 2 fold, 1 fold, 0.5 fold, 0.2 fold, 0.1 fold, or less.

In some cases, the thresholds of expression of mRNA encoding a cytokine may be determined by an increase of expression of mRNA encoding a cytokine as measured from mRNA isolated from a defined number of cells. In some cases, the defined number of cells can be about 1000 cells, 2000 cells, 5000 cells, 10,000 cells, 20,000 cells, 50,000 cells, 100,000 cells, 200,000 cells, 500,000 cells, 1,000,000 cells, 2,000,000 cells, 5,000,000 cells, 10,000,000 cells, 20,000,000 cells, 50,000,000 cells, 100,000,000 cells, 200,000,000 cells, 500,000,000 cells, 1,000,000,000 cells, or more. In some cases, the defined number of cells can be at least 1000 cells, at least 2000 cells, at least 5000 cells, at least cells, at least 20,000 cells, at least 50,000 cells, at least 100,000 cells, at least 200,000 cells, at least 500,000 cells, at least 1,000,000 cells, at least 2,000,000 cells, at least 5,000,000 cells, at least cells, at least 20,000,000 cells, at least 50,000,000 cells, at least 100,000,000 cells, at least 200,000,000 cells, at least 500,000,000 cells, at least 1,000,000,000 cells, or more. In some cases, the defined number of cells can be at most 1,000,000,000 cells, at most 500,000,000 cells, at most 100,000,000 cells, at most 50,000,000 cells, at most 10,000,000 cells, at most 5,000,000 cells, at most 1,000,000 cells, at most 500,000 cells, at most 100,000 cells, at most 50,000 cells, at most 10,000 cells, at most 5,000 cells, at most 1,000 cells, or less.

In some cases, the thresholds may be defined by expression of mRNA encoding a cytokine increased by at least about 0.1 fold to about 1000 fold, about 0.1 fold to about 1000 fold, or at most about 0.1 fold to about 1000 fold as measured from mRNA isolated from about 1000 cells, 2000 cells, 5000 cells, 10,000 cells, 20,000 cells, 50,000 cells, 100,000 cells, 200,000 cells, 500,000 cells, 1,000,000 cells, 2,000,000 cells, 5,000,000 cells, 10,000,000 cells, 20,000,000 cells, 50,000,000 cells, 100,000,000 cells, 200,000,000 cells, 500,000,000 cells, or 1,000,000,000 cells. In some cases, the thresholds may be defined by expression of mRNA encoding a cytokine increased by at least about 0.1 fold to about 1000 fold, about 0.1 fold to about 1000 fold, or at most about 0.1 fold to about 1000 fold as measured from mRNA isolated from at least 1000 cells, at least 2000 cells, at least 5000 cells, at least 10,000 cells, at least 20,000 cells, at least 50,000 cells, at least 100,000 cells, at least 200,000 cells, at least 500,000 cells, at least 1,000,000 cells, at least 2,000,000 cells, at least 5,000,000 cells, at least 10,000,000 cells, at least 20,000,000 cells, at least 50,000,000 cells, at least 100,000,000 cells, at least 200,000,000 cells, at least 500,000,000 cells, at least 1,000,000,000 cells, or more. In some cases, the thresholds may be defined by expression of mRNA encoding a cytokine increased by at least about fold to about 1000 fold, about 0.1 fold to about 1000 fold, or at most about 0.1 fold to about 1000 fold as measured from mRNA isolated from at most 1,000,000,000 cells, at most 500,000,000 cells, at most 100,000,000 cells, at most 50,000,000 cells, at most 10,000,000 cells, at most 5,000,000 cells, at most 1,000,000 cells, at most 500,000 cells, at most 100,000 cells, at most 50,000 cells, at most 10,000 cells, at most 5,000 cells, at most 1,000 cells, or less. In some cases, the thresholds of expression of mRNA encoding a cytokine may be determined by the increase of expression of mRNA encoding a cytokine as measured from mRNA isolated from a defined number of cells over a period of time. In some cases, the thresholds of expression of mRNA encoding a cytokine may be measured from mRNA isolated from a defined number of cells over a period of about 1 minute, 2 minutes, 5 minutes, minutes, 20 minutes, 30 minutes, 1 hour, 2 hours, 5 hours, 10 hours, 12 hours, 24 hours, 36 hours, 48 hours, 72 hours, or longer. In some embodiments, the thresholds of expression of mRNA encoding a cytokine may be determined by the increase by at least about 0.1 fold to about 1000 fold, about 0.1 fold to about 1000 fold, or at most about 0.1 fold to about 1000 fold as measured from mRNA isolated from 1000 cells, 2000 cells, 5000 cells, 10,000 cells, 20,000 cells, 50,000 cells, 100,000 cells, 200,000 cells, 500,000 cells, 1,000,000 cells, 2,000,000 cells, 5,000,000 cells, 10,000,000 cells, 20,000,000 cells, 50,000,000 cells, 100,000,000 cells, 200,000,000 cells, 500,000,000 cells, or 1,000,000,000 cells over a period of about 1 minute, 2 minutes, 5 minutes, 10 minutes, 20 minutes, 30 minutes, 1 hour, 2 hours, 5 hours, 10 hours, 12 hours, 24 hours, 36 hours, 48 hours, 72 hours, or longer. In some embodiments, the thresholds of expression of mRNA encoding a cytokine may be determined by the increase by at least about 0.1 fold to about 1000 fold, about 0.1 fold to about 1000 fold, or at most about 0.1 fold to about 1000 fold as measured from mRNA isolated from at least 1000 cells, at least 2000 cells, at least 5000 cells, at least 10,000 cells, at least 20,000 cells, at least 50,000 cells, at least 100,000 cells, at least 200,000 cells, at least 500,000 cells, at least 1,000,000 cells, at least 2,000,000 cells, at least 5,000,000 cells, at least 10,000,000 cells, at least 20,000,000 cells, at least 50,000,000 cells, at least 100,000,000 cells, at least 200,000,000 cells, at least 500,000,000 cells, at least 1,000,000,000 cells, or more over a period of 1 minute, 2 minutes, 5 minutes, 10 minutes, 20 minutes, minutes, 1 hour, 2 hours, 5 hours, 10 hours, 12 hours, 24 hours, 36 hours, 48 hours, 72 hours, or longer. In some embodiments, the thresholds of expression of mRNA encoding a cytokine may be determined by the increase by at least about 0.1 fold to about 1000 fold, about 0.1 fold to about 1000 fold, or at most about 0.1 fold to about 1000 fold as measured from mRNA isolated from at most 1,000,000,000 cells, at most 500,000,000 cells, at most 100,000,000 cells, at most 50,000,000 cells, at most 10,000,000 cells, at most 5,000,000 cells, at most 1,000,000 cells, at most 500,000 cells, at most 100,000 cells, at most 50,000 cells, at most 10,000 cells, at most 5,000 cells, at most 1,000 cells, or less over a period of 1 minute, 2 minutes, 5 minutes, 10 minutes, 20 minutes, 30 minutes, 1 hour, 2 hours, 5 hours, 10 hours, 12 hours, 24 hours, 36 hours, 48 hours, 72 hours, or longer.

In implementing any one of the methods provided in the present disclosure, or in any one of the systems provided in the present disclosure, an mRNA of interest may encode a cytokine as described in the present disclosure.

In some embodiments, the thresholds comprise an increased rate of cellular proliferation of the first sub-population of the plurality of cells. In some cases, the increased rate of cellular proliferation of the first sub-population of the plurality of cells can be increased by at least 0.1 fold to about 1000 fold. In some cases, the increased rate of cellular proliferation of the first sub-population of the plurality of cells can be increased by at least 0.1 fold to about 20 fold, at least about 0.1 fold to about fold, at least about 0.1 fold to about 100 fold, at least about 0.1 fold to about 200 fold, at least about fold to about 500 fold, at least about 0.1 fold to about 1,000 fold, at least about 2 fold to about 50 fold, at least about 2 fold to about 100 fold, at least about 2 fold to about 200 fold, at least about 2 fold to about 500 fold, at least about 2 fold to about 1,000 fold, at least about 10 fold to about 100 fold, at least about 10 fold to about 200 fold, at least about 10 fold to about 500 fold, at least about 10 fold to about 1,000 fold, at least about 50 fold to about 200 fold, at least about 50 fold to about 500 fold, at least about 50 fold to about 1,000 fold, at least about 100 fold to about 500 fold, at least about 100 fold to about 1,000 fold, or at least about 500 fold to about 1,000 fold.

In some embodiments, the thresholds comprise an increased rate of cellular proliferation of the first sub-population of the plurality of cells. In some cases, the increased rate of cellular proliferation of the plurality of cells can be increased by at most 0.1 fold to about 1000 fold. In some cases, the increased rate of cellular proliferation of the plurality of cells can be increased by at most 0.1 fold to about 20 fold, at most about 0.1 fold to about 50 fold, at most about 0.1 fold to about 100 fold, at most about 0.1 fold to about 200 fold, at most about 0.1 fold to about 500 fold, at most about 0.1 fold to about 1,000 fold, at most about 2 fold to about 50 fold, at most about 2 fold to about 100 fold, at most about 2 fold to about 200 fold, at most about 2 fold to about 500 fold, at most about 2 fold to about 1,000 fold, at most about 10 fold to about 100 fold, at most about 10 fold to about 200 fold, at most about 10 fold to about 500 fold, at most about 10 fold to about 1,000 fold, at most about 50 fold to about 200 fold, at most about 50 fold to about 500 fold, at most about 50 fold to about 1,000 fold, at most about 100 fold to about 500 fold, at most about 100 fold to about 1,000 fold, or at most about 500 fold to about 1,000 fold.

In some embodiments, the thresholds comprise an increased rate of cellular proliferation of the plurality of cells. In some cases, the increased rate of cellular proliferation of the first sub-population of the plurality of cells can be increased by at 0.1 fold to about 1000 fold. In some cases, the increased rate of cellular proliferation of the plurality of cells can be increased by at 0.1 fold to about 20 fold, at about 0.1 fold to about 50 fold, at about 0.1 fold to about 100 fold, at about 0.1 fold to about 200 fold, at about 0.1 fold to about 500 fold, at about 0.1 fold to about 1,000 fold, at about 2 fold to about 50 fold, at about 2 fold to about 100 fold, at about 2 fold to about 200 fold, at about 2 fold to about 500 fold, at about 2 fold to about 1,000 fold, at about 10 fold to about 100 fold, at about 10 fold to about 200 fold, at about 10 fold to about 500 fold, at about 10 fold to about 1,000 fold, at about 50 fold to about 200 fold, at about 50 fold to about 500 fold, at about 50 fold to about 1,000 fold, at about 100 fold to about 500 fold, at about 100 fold to about 1,000 fold, or at about 500 fold to about 1,000 fold.

In some embodiments, the thresholds comprise an increased rate of cellular proliferation of the first sub-population of the plurality of cells. In some cases, the increased rate of cellular proliferation of the plurality of cells can be increased by at least about 0.1 fold, 1 fold, 2 fold, 5 fold, 10 fold, 50 fold, 100 fold, 500 fold, 1000 fold, or more. In some cases, the increased rate of cellular proliferation of the plurality of cells can be increased by at most about 1000 fold, 500 fold, 100 fold, 50 fold, 10 fold, 5 fold, 2 fold, 1 fold, 0.1 fold, or less.

In some cases, the thresholds may be defined by cellular proliferation of the plurality of cells increased by at least about 0.1 fold to about 1000 fold, about 0.1 fold to about 1000 fold, or at most about 0.1 fold to about 1000 fold as measured from 1000 cells, 2000 cells, 5000 cells, 10,000 cells, cells, 50,000 cells, 100,000 cells, 200,000 cells, 500,000 cells, 1,000,000 cells, 2,000,000 cells, cells, 10,000,000 cells, 20,000,000 cells, 50,000,000 cells, 100,000,000 cells, 200,000,000 cells, 500,000,000 cells, or 1,000,000,000 cells. In some cases, the thresholds may be defined by cellular proliferation of the first sub-population of the plurality of cells increased by at least about 0.1 fold to about 1000 fold, about 0.1 fold to about 1000 fold, or at most about 0.1 fold to about 1000 fold as measured from at least 1000 cells, at least 2000 cells, at least 5000 cells, at least 10,000 cells, at least 20,000 cells, at least 50,000 cells, at least 100,000 cells, at least 200,000 cells, at least 500,000 cells, at least 1,000,000 cells, at least 2,000,000 cells, at least 5,000,000 cells, at least 10,000,000 cells, at least 20,000,000 cells, at least 50,000,000 cells, at least 100,000,000 cells, at least 200,000,000 cells, at least 500,000,000 cells, at least 1,000,000,000 cells, or more. In some cases, the thresholds may be defined by cellular proliferation of the plurality of cells increased by at least about 0.1 fold to about 1000 fold, about 0.1 fold to about 1000 fold, or at most about 0.1 fold to about 1000 fold as measured from at most 1,000,000,000 cells, at most 500,000,000 cells, at most 100,000,000 cells, at most 50,000,000 cells, at most 10,000,000 cells, at most 5,000,000 cells, at most 1,000,000 cells, at most 500,000 cells, at most 100,000 cells, at most 50,000 cells, at most 10,000 cells, at most 5,000 cells, at most 1,000 cells, or less.

In some cases, the thresholds of cellular proliferation of the first sub-population of the plurality of cells may be determined by the increased rate of cellular proliferation of the first sub-population of the plurality of cells as measured from a defined number of cells over a period of time. In some cases, the thresholds of cellular proliferation of the first sub-population of the plurality of cells may be measured from a defined number of cells over a period of 1 minute, 2 minutes, 5 minutes, 10 minutes, minutes, 30 minutes, 1 hour, 2 hours, 5 hours, 10 hours, 12 hours, 24 hours, 36 hours, 48 hours, 72 hours, or longer. In some embodiments, the thresholds of increased rate of cellular proliferation of the plurality of cells may be by at least about 0.1 fold to about 1000 fold, about 0.1 fold to about 1000 fold, or at most about 0.1 fold to about 1000 fold as measured from 1000 cells, 2000 cells, 5000 cells, cells, 20,000 cells, 50,000 cells, 100,000 cells, 200,000 cells, 500,000 cells, 1,000,000 cells, 2,000,000 cells, 5,000,000 cells, 10,000,000 cells, 20,000,000 cells, 50,000,000 cells, 100,000,000 cells, 200,000,000 cells, 500,000,000 cells, or 1,000,000,000 cells over a period of 1 minute, 2 minutes, 5 minutes, 10 minutes, 20 minutes, 30 minutes, 1 hour, 2 hours, 5 hours, 10 hours, 12 hours, 24 hours, 36 hours, 48 hours, 72 hours, or longer. In some embodiments, the thresholds of increased rate of cellular proliferation of the first sub-population of the plurality of cells may be by at least about fold to about 1000 fold, about 0.1 fold to about 1000 fold, or at most about 0.1 fold to about 1000 fold as measured from at least 1000 cells, at least 2000 cells, at least 5000 cells, at least 10,000 cells, at least 20,000 cells, at least 50,000 cells, at least 100,000 cells, at least 200,000 cells, at least 500,000 cells, at least 1,000,000 cells, at least 2,000,000 cells, at least 5,000,000 cells, at least 10,000,000 cells, at least 20,000,000 cells, at least 50,000,000 cells, at least 100,000,000 cells, at least 200,000,000 cells, at least 500,000,000 cells, at least 1,000,000,000 cells, or more over a period of 1 minute, 2 minutes, 5 minutes, 10 minutes, 20 minutes, 30 minutes, 1 hour, 2 hours, 5 hours, 10 hours, 12 hours, 24 hours, 36 hours, 48 hours, 72 hours, or longer. In some embodiments, the thresholds of increased rate of cellular proliferation of the first sub-population of the plurality of cells may be determined by at least about 0.1 fold to about 1000 fold, about 0.1 fold to about 1000 fold, or at most about 0.1 fold to about 1000 fold as measured from at most 1,000,000,000 cells, at most 500,000,000 cells, at most 100,000,000 cells, at most 50,000,000 cells, at most 10,000,000 cells, at most 5,000,000 cells, at most 1,000,000 cells, at most 500,000 cells, at most 100,000 cells, at most 50,000 cells, at most 10,000 cells, at most 5,000 cells, at most 1,000 cells, or less over a period of 1 minute, 2 minutes, 5 minutes, minutes, 20 minutes, 30 minutes, 1 hour, 2 hours, 5 hours, 10 hours, 12 hours, 24 hours, 36 hours, 48 hours, 72 hours, or longer.

In some embodiments, the thresholds comprise an increased percentage of cytotoxicity against a plurality of target cells, said cytotoxicity is exerted from the activity of the first sub-population of the plurality of cells. In some cases, cytotoxicity can include loss of cell number of the target cells, cellular activity, apoptosis, necrosis, injury, changes in cell morphology, nuclear condensation, DNA damage, stress response, senescence, or any other abnormal cellular functions or responses.

In some cases, the increased percentage of cytotoxicity against the plurality of target cells can be increased by at least 1% to about 1,000%. In some cases, the increased percentage of cytotoxicity against the plurality of target cells can be increased by at least 1% to about 20%, at least about 1% to about 50%, at least about 1% to about 100%, at least about 1% to about 200%, at least about 1% to about 500%, at least about 1% to about 1,000%, at least about 2% to about 50%, at least about 2% to about 100%, at least about 2% to about 200%, at least about 2% to about 500%, at least about 2% to about 1,000%, at least about 10% to about 100%, at least about 10% to about 200%, at least about 10% to about 500%, at least about 10% to about 1,000%, at least about 50% to about 200%, at least about 50% to about 500%, at least about 50% to about 1,000%, at least about 100% to about 500%, at least about 100% to about 1,000%, or at least about 500% to about 1,000%.

In some cases, the increased percentage of cytotoxicity against the plurality of target cells can be increased by at most 1% to about 1,000%. In some cases, the increased percentage of cytotoxicity against the plurality of target cells can be increased by at most 1% to about 20%, at most about 1% to about 50%, at most about 1% to about 100%, at most about 1% to about 200%, at most about 1% to about 500%, at most about 1% to about 1,000%, at most about 2% to about 50%, at most about 2% to about 100%, at most about 2% to about 200%, at most about 2% to about 500%, at most about 2% to about 1,000%, at most about 10% to about 100%, at most about 10% to about 200%, at most about 10% to about 500%, at most about 10% to about 1,000%, at most about 50% to about 200%, at most about 50% to about 500%, at most about 50% to about 1,000%, at most about 100% to about 500%, at most about 100% to about 1,000%, or at most about 500% to about 1,000%.

In some cases, the increased percentage of cytotoxicity against the plurality of target cells can be increased by at about 1% to about 1,000%. In some cases, the increased percentage of cytotoxicity against the plurality of target cells can be increased by at about 1% to about 20%, at about 1% to about 50%, at about 1% to about 100%, at about 1% to about 200%, at about 1% to about 500%, at about 1% to about 1,000%, at about 2% to about 50%, at about 2% to about 100%, at about 2% to about 200%, at about 2% to about 500%, at about 2% to about 1,000%, at about 10% to about 100%, at about 10% to about 200%, at about 10% to about 500%, at about 10% to about 1,000%, at about 50% to about 200%, at about 50% to about 500%, at about 50% to about 1,000%, at about 100% to about 500%, at about 100% to about 1,000%, or at about 500% to about 1,000%.

In some cases, the increased percentage of cytotoxicity against the plurality of target cells can be increased by at least about 1%, 2%, 5%, 10%, 20%, 50%, 100%, 200%, 500%, 1,000%, or more. In some cases, the increased percentage of cytotoxicity against the plurality of target cells can be increased by at most about 1,000%, 500%, 200%, 100%, 50%, 10%, 5%, 2%, 1%, or less.

In some cases, the thresholds of increased percentage of cytotoxicity against the plurality of target cells can be measured from a defined number of cells (e.g., a defined number of the first sub-population of cells), as provided in the present disclosure.

In some cases, the thresholds may be defined by an increased percentage of cytotoxicity of the plurality of target cells by at least about 1% to about 1,000%, about 1% to about 1,000%, or at most about 1% to about 1,000% as measured from cytotoxicity exerted by first sub-population cells numbering from 1000 cells, 2000 cells, 5000 cells, 10,000 cells, 20,000 cells, 50,000 cells, 100,000 cells, 200,000 cells, 500,000 cells, 1,000,000 cells, 2,000,000 cells, 5,000,000 cells, 10,000,000 cells, cells, 50,000,000 cells, 100,000,000 cells, 200,000,000 cells, 500,000,000 cells, or 1,000,000,000 cells. In some cases, the thresholds may be defined by increased percentage of cytotoxicity of the plurality of target cells by at least about 1% to about 1,000%, about 1% to about 1,000%, or at most about 1% to about 1,000% as measured from cytotoxicity exerted by first sub-population of cells numbering from at least 1000 cells, at least 2000 cells, at least 5000 cells, at least cells, at least 20,000 cells, at least 50,000 cells, at least 100,000 cells, at least 200,000 cells, at least 500,000 cells, at least 1,000,000 cells, at least 2,000,000 cells, at least 5,000,000 cells, at least cells, at least 20,000,000 cells, at least 50,000,000 cells, at least 100,000,000 cells, at least 200,000,000 cells, at least 500,000,000 cells, at least 1,000,000,000 cells, or more. In some cases, the thresholds may be defined by an increased cytotoxicity of the plurality of target cells by at least about 1% to about 1,000%, about 1% to about 1,000%, or at most about 1% to about 1,000% as measured from cytotoxicity exerted by first sub-population of cells numbering from at most 1,000,000,000 cells, at most 500,000,000 cells, at most 100,000,000 cells, at most 50,000,000 cells, at most 10,000,000 cells, at most 5,000,000 cells, at most 1,000,000 cells, at most 500,000 cells, at most 100,000 cells, at most 50,000 cells, at most 10,000 cells, at most 5,000 cells, at most 1,000 cells, or less.

In some cases, the thresholds of increased cytotoxicity of the plurality of target cells may be determined by the increased percentage of cytotoxicity against the plurality of target cells exerted by a defined number of first sub-population of cells over a period of time. In some cases, the thresholds of increased cytotoxicity may be measured from a defined number of first sub-population of cells over a period of 1 minute, 2 minutes, 5 minutes, 10 minutes, 20 minutes, 30 minutes, 1 hour, 2 hours, 5 hours, 10 hours, 12 hours, 24 hours, 36 hours, 2 days, 5 days, 10 days, or longer. In some embodiments, the thresholds of increased cytotoxicity of the plurality of target cells may be by at least about 1% to about 1,000%, about 1% to about 1,000%, or at most about 1% to about 1,000% as measured from cytotoxicity exerted by first sub-population of cells numbering from 1000 cells, 2000 cells, 5000 cells, 10,000 cells, 20,000 cells, 50,000 cells, 100,000 cells, 200,000 cells, 500,000 cells, 1,000,000 cells, 2,000,000 cells, 5,000,000 cells, 10,000,000 cells, 20,000,000 cells, 50,000,000 cells, 100,000,000 cells, 200,000,000 cells, 500,000,000 cells, or 1,000,000,000 cells over a period of 1 minute, 2 minutes, 5 minutes, 10 minutes, 20 minutes, 30 minutes, 1 hour, 2 hours, 5 hours, 10 hours, 12 hours, 24 hours, 36 hours, 2 days, 5 days, 10 days, or longer. In some embodiments, the thresholds of increased cytotoxicity of the plurality of target cells may be by at least about 1% to about 1,000%, about 1% to about 1,000%, or at most about 1% to about 1,000% as measured from cytotoxicity exerted by first sub-population of cells numbering from at least 1000 cells, at least 2000 cells, at least 5000 cells, at least 10,000 cells, at least 20,000 cells, at least 50,000 cells, at least 100,000 cells, at least 200,000 cells, at least 500,000 cells, at least 1,000,000 cells, at least 2,000,000 cells, at least cells, at least 10,000,000 cells, at least 20,000,000 cells, at least 50,000,000 cells, at least 100,000,000 cells, at least 200,000,000 cells, at least 500,000,000 cells, at least 1,000,000,000 cells, or more over a period of 1 minute, 2 minutes, 5 minutes, 10 minutes, 20 minutes, 30 minutes, 1 hour, 2 hours, 5 hours, 10 hours, 12 hours, 24 hours, 36 hours, 2 days, 5 days, 10 days, or longer. In some embodiments, the thresholds of increased cytotoxicity of the plurality of target cells may be determined by at least about 1% to about 1,000%, about 1% to about 1,000%, or at most about 1% to about 1,000% as measured from cytotoxicity exerted by first sub-population of cells numbering from at most 1,000,000,000 cells, at most 500,000,000 cells, at most 100,000,000 cells, at most 50,000,000 cells, at most 10,000,000 cells, at most 5,000,000 cells, at most 1,000,000 cells, at most 500,000 cells, at most 100,000 cells, at most 50,000 cells, at most 10,000 cells, at most 5,000 cells, at most 1,000 cells, or less over a period of 1 minute, 2 minutes, 5 minutes, 10 minutes, 20 minutes, 30 minutes, 1 hour, 2 hours, 5 hours, 10 hours, 12 hours, 24 hours, 36 hours, 2 days, 5 days, 10 days, or longer.

In some embodiments, the thresholds comprise an increased rate of cell migration of the first sub-population cells as determined by chemotaxis assay. In some cases, the increased rate cell migration of the first sub-population can be induced by a chemotaxis attractant. In some cases, the increased rate cell migration of the first sub-population can be induced by a chemotaxis repellent. In some instances, the rate of cell migration of the first sub-population can be increased by at least 0.1 fold to about 1000 fold. In some cases, the rate of cell migration of the first sub-population can be increased by at least 0.1 fold to about 20 fold, at least about 0.1 fold to about 50 fold, at least about 0.1 fold to about 100 fold, at least about 0.1 fold to about 200 fold, at least about 0.1 fold to about 500 fold, at least about 0.1 fold to about 1,000 fold, at least about 2 fold to about 50 fold, at least about 2 fold to about 100 fold, at least about 2 fold to about 200 fold, at least about 2 fold to about 500 fold, at least about 2 fold to about 1,000 fold, at least about 10 fold to about 100 fold, at least about 10 fold to about 200 fold, at least about 10 fold to about 500 fold, at least about 10 fold to about 1,000 fold, at least about 50 fold to about 200 fold, at least about 50 fold to about 500 fold, at least about 50 fold to about 1,000 fold, at least about 100 fold to about 500 fold, at least about 100 fold to about 1,000 fold, or at least about 500 fold to about 1,000 fold.

In some embodiments, the thresholds comprise an increase of rate of cell migration of the first sub-population cells as determined by chemotaxis assay. In some cases, the rate of cell migration of the first sub-population can be increased by at most 0.1 fold to about 1000 fold. In some cases, the rate of cell migration of the first sub-population can be increased by at most 0.1 fold to about 20 fold, at most about 0.1 fold to about 50 fold, at most about 0.1 fold to about 100 fold, at most about 0.1 fold to about 200 fold, at most about 0.1 fold to about 500 fold, at most about 0.1 fold to about 1,000 fold, at most about 2 fold to about 50 fold, at most about 2 fold to about 100 fold, at most about 2 fold to about 200 fold, at most about 2 fold to about 500 fold, at most about 2 fold to about 1,000 fold, at most about 10 fold to about 100 fold, at most about 10 fold to about 200 fold, at most about 10 fold to about 500 fold, at most about 10 fold to about 1,000 fold, at most about 50 fold to about 200 fold, at most about 50 fold to about 500 fold, at most about 50 fold to about 1,000 fold, at most about 100 fold to about 500 fold, at most about 100 fold to about 1,000 fold, or at most about 500 fold to about 1,000 fold.

In some embodiments, the thresholds comprise an increase of rate of cell migration of the first sub-population cells as determined by chemotaxis assay. In some cases, the rate of cell migration of the first sub-population can be increased by 0.1 fold to about 1000 fold. In some cases, the rate of cell migration of the first sub-population can be increased by 0.1 fold to about 20 fold, about 0.1 fold to about 50 fold, about 0.1 fold to about 100 fold, about 0.1 fold to about 200 fold, about 0.1 fold to about 500 fold, about 0.1 fold to about 1,000 fold, about 2 fold to about 50 fold, about 2 fold to about 100 fold, about 2 fold to about 200 fold, about 2 fold to about 500 fold, about 2 fold to about 1,000 fold, about 10 fold to about 100 fold, about 10 fold to about 200 fold, about 10 fold to about 500 fold, about 10 fold to about 1,000 fold, about 50 fold to about 200 fold, about 50 fold to about 500 fold, about 50 fold to about 1,000 fold, about 100 fold to about 500 fold, about 100 fold to about 1,000 fold, or about 500 fold to about 1,000 fold.

In some embodiments, the thresholds comprise an increase of rate of cell migration of the first sub-population cells as determined by chemotaxis assay. In some cases, the rate of cell migration of the first sub-population can be increased by at least about 0.1 fold, 0.2 fold, 0.5 fold, 1 fold, 2 fold, 5 fold, 10 fold, 20 fold, 50 fold, 100 fold, 200 fold, 500 fold, 1000 fold, or more. In some cases, the rate of cell migration of the first sub-population can be increased by at most about 1000 fold, 500 fold, 200 fold, 100 fold, 50 fold, 20 fold, 10 fold, 5 fold, 2 fold, 1 fold, 0.5 fold, 0.2 fold, 0.1 fold, or less. In some cases, the rate of cell migration of the first sub-population can be increased by about 0.1 fold, 0.2 fold, 0.5 fold, 1 fold, 2 fold, 5 fold, 10 fold, 20 fold, 50 fold, 100 fold, 200 fold, 500 fold, 1000 fold, or more.

In some cases, the thresholds of rate of cell migration of the first sub-population cells as determined by chemotaxis assay may be determined by an increased rate of cell migration of the first sub-population cells as determined by chemotaxis assay as determined from a defined number of cells, as provided herein.

In some cases, the thresholds may be defined by rate of cell migration of the first sub-population cells as determined by chemotaxis assay. In some embodiments, the rate of cell migration of the first sub-population cells is increased by at least about 0.1 fold to about 1000 fold, about 0.1 fold to about 1000 fold, or at most about 0.1 fold to about 1000 fold as measured from 1000 cells, 2000 cells, 5000 cells, 10,000 cells, 20,000 cells, 50,000 cells, 100,000 cells, 200,000 cells, 500,000 cells, 1,000,000 cells, 2,000,000 cells, 5,000,000 cells, 10,000,000 cells, 20,000,000 cells, 50,000,000 cells, 100,000,000 cells, 200,000,000 cells, 500,000,000 cells, or 1,000,000,000 cells. In some cases, the rate of cell migration of the first sub-population cells is increased by at least about 0.1 fold to about 1000 fold, about 0.1 fold to about 1000 fold, or at most about 0.1 fold to about 1000 fold as measured from at least 1000 cells, at least 2000 cells, at least 5000 cells, at least 10,000 cells, at least 20,000 cells, at least 50,000 cells, at least 100,000 cells, at least 200,000 cells, at least 500,000 cells, at least 1,000,000 cells, at least 2,000,000 cells, at least 5,000,000 cells, at least 10,000,000 cells, at least cells, at least 50,000,000 cells, at least 100,000,000 cells, at least 200,000,000 cells, at least 500,000,000 cells, at least 1,000,000,000 cells, or more. In some cases, the rate of cell migration of the first sub-population cells may be increased by at least about 0.1 fold to about 1000 fold, about fold to about 1000 fold, or at most about 0.1 fold to about 1000 fold as measured from at most 1,000,000,000 cells, at most 500,000,000 cells, at most 100,000,000 cells, at most 50,000,000 cells, at most 10,000,000 cells, at most 5,000,000 cells, at most 1,000,000 cells, at most 500,000 cells, at most 100,000 cells, at most 50,000 cells, at most 10,000 cells, at most 5,000 cells, at most 1,000 cells, or less.

In some cases, the thresholds of rate of cell migration of the first sub-population cells as determined by chemotaxis assay may be determined by the increase of rate of cell migration of the first sub-population cells as determined by chemotaxis assay from a defined number of cells over a period of time. In some cases, the thresholds of rate of cell migration of the first sub-population cells may be measured from a defined number of cells over a period of 1 minute, 2 minutes, 5 minutes, 10 minutes, 20 minutes, 30 minutes, 1 hour, 2 hours, 5 hours, 10 hours, 12 hours, 24 hours, 36 hours, 48 hours, 72 hours, or longer. In some embodiments, the thresholds of rate of cell migration of the first sub-population cells as determined by chemotaxis assay may be determined by the increase by at least about 0.1 fold to about 1000 fold, about 0.1 fold to about 1000 fold, or at most about 0.1 fold to about 1000 fold as measured from 1000 cells, 2000 cells, 5000 cells, 10,000 cells, 20,000 cells, 50,000 cells, 100,000 cells, 200,000 cells, 500,000 cells, 1,000,000 cells, 2,000,000 cells, 5,000,000 cells, cells, 20,000,000 cells, 50,000,000 cells, 100,000,000 cells, 200,000,000 cells, 500,000,000 cells, or 1,000,000,000 cells over a period of 1 minute, 2 minutes, 5 minutes, 10 minutes, minutes, 30 minutes, 1 hour, 2 hours, 5 hours, 10 hours, 12 hours, 24 hours, 36 hours, 48 hours, 72 hours, or longer. In some embodiments, the thresholds of rate of cell migration of the first sub-population cells as determined by chemotaxis assay may be determined by the increase by at least about 0.1 fold to about 1000 fold, about 0.1 fold to about 1000 fold, or at most about 0.1 fold to about 1000 fold as measured from at least 1000 cells, at least 2000 cells, at least 5000 cells, at least 10,000 cells, at least 20,000 cells, at least 50,000 cells, at least 100,000 cells, at least 200,000 cells, at least 500,000 cells, at least 1,000,000 cells, at least 2,000,000 cells, at least 5,000,000 cells, at least cells, at least 20,000,000 cells, at least 50,000,000 cells, at least 100,000,000 cells, at least 200,000,000 cells, at least 500,000,000 cells, at least 1,000,000,000 cells, or more over a period of 1 minute, 2 minutes, 5 minutes, 10 minutes, 20 minutes, 30 minutes, 1 hour, 2 hours, 5 hours, 10 hours, 12 hours, 24 hours, 36 hours, 48 hours, 72 hours, or longer. In some embodiments, the thresholds of rate of cell migration of the first sub-population cells may be increased by at least about 0.1 fold to about 1000 fold, about 0.1 fold to about 1000 fold, or at most about 0.1 fold to about 1000 fold as measured from at most 1,000,000,000 cells, at most 500,000,000 cells, at most 100,000,000 cells, at most 50,000,000 cells, at most 10,000,000 cells, at most 5,000,000 cells, at most 1,000,000 cells, at most 500,000 cells, at most 100,000 cells, at most 50,000 cells, at most 10,000 cells, at most 5,000 cells, at most 1,000 cells, or less cells over a period of 1 minute, 2 minutes, 5 minutes, 10 minutes, 20 minutes, 30 minutes, 1 hour, 2 hours, 5 hours, 10 hours, 12 hours, 24 hours, 36 hours, 48 hours, 72 hours, or longer.

In some embodiments, the thresholds comprise exhaustion of the first sub-population of the plurality of cells. In some instances, exhaustion can comprise loss of cellular activity, decreased cytokine production, reduced or absence of responsiveness to antigen stimulation, decreased cellular metabolism, apoptosis, necrosis, injury, changes in cell morphology, nuclear condensation, DNA damage, stress response, senescence, or any other abnormal cellular functions.

In some embodiments, the thresholds comprise an increased exhaustion of the first sub-population of the plurality of cells. In some cases, the increased exhaustion of the first sub-population of the plurality of cells can be increased by at least 1% to about 1,000%. In some cases, the increased exhaustion of the first sub-population of the plurality of cells can be increased by at least 1% to about 20%, at least about 1% to about 50%, at least about 1% to about 100%, at least about 1% to about 200%, at least about 1% to about 500%, at least about 1% to about 1,000%, at least about 2% to about 50%, at least about 2% to about 100%, at least about 2% to about 200%, at least about 2% to about 500%, at least about 2% to about 1,000%, at least about 10% to about 100%, at least about 10% to about 200%, at least about 10% to about 500%, at least about 10% to about 1,000%, at least about 50% to about 200%, at least about 50% to about 500%, at least about 50% to about 1,000%, at least about 100% to about 500%, at least about 100% to about 1,000%, or at least about 500% to about 1,000%.

In some cases, the increased exhaustion of the first sub-population of the plurality of cells can be increased by at most 1% to about 1,000%. In some cases, the increased exhaustion of the first sub-population of the plurality of cells can be increased by at most 1% to about 20%, at most about 1% to about 50%, at most about 1% to about 100%, at most about 1% to about 200%, at most about 1% to about 500%, at most about 1% to about 1,000%, at most about 2% to about 50%, at most about 2% to about 100%, at most about 2% to about 200%, at most about 2% to about 500%, at most about 2% to about 1,000%, at most about 10% to about 100%, at most about 10% to about 200%, at most about 10% to about 500%, at most about 10% to about 1,000%, at most about 50% to about 200%, at most about 50% to about 500%, at most about 50% to about 1,000%, at most about 100% to about 500%, at most about 100% to about 1,000%, or at most about 500% to about 1,000%.

In some cases, the increased exhaustion of the first sub-population of the plurality of cells can be increased by 1% to about 1,000%. In some cases, the increased exhaustion of the first sub-population of the plurality of cells can be increased by 1% to about 20%, about 1% to about 50%, about 1% to about 100%, about 1% to about 200%, about 1% to about 500%, about 1% to about 1,000%, about 2% to about 50%, about 2% to about 100%, about 2% to about 200%, about 2% to about 500%, about 2% to about 1,000%, about 10% to about 100%, about 10% to about 200%, about 10% to about 500%, about 10% to about 1,000%, about 50% to about 200%, about 50% to about 500%, about 50% to about 1,000%, about 100% to about 500%, about 100% to about 1,000%, or about 500% to about 1,000%.

In some cases, the increased exhaustion of the first sub-population of the plurality of cells can be by at least about 1%, 2%, 5%, 10%, 20%, 50%, 100%, 200%, 500%, 1,000%, or more. In some cases, the increased exhaustion of the first sub-population of the plurality of cells can be by at most about 1,000%, 500%, 200%, 100%, 50%, 10%, 5%, 2%, 1%, or less. In some cases, the increased exhaustion of the first sub-population of the plurality of cells can be by about 1%, 2%, 5%, 10%, 20%, 50%, 100%, 200%, 500%, 1,000%, or more.

In some cases, the thresholds of the increased exhaustion of the first sub-population of the plurality of cells can be measured from a defined number of cells, as provided herein.

In some cases, the thresholds of the increased exhaustion of the first sub-population of the plurality of cells can be increased by at least about 1% to about 1,000%, about 1% to about 1,000%, or at most about 1% to about 1,000% as measured from 1000 cells, 2000 cells, 5000 cells, 10,000 cells, cells, 50,000 cells, 100,000 cells, 200,000 cells, 500,000 cells, 1,000,000 cells, 2,000,000 cells, cells, 10,000,000 cells, 20,000,000 cells, 50,000,000 cells, 100,000,000 cells, 200,000,000 cells, 500,000,000 cells, or 1,000,000,000 cells. In some cases, the thresholds of the increased exhaustion of the first sub-population of the plurality of cells can be increased by at least about 1% to about 1,000%, about 1% to about 1,000%, or at most about 1% to about 1,000% as measured from at least 1000 cells, at least 2000 cells, at least 5000 cells, at least 10,000 cells, at least 20,000 cells, at least 50,000 cells, at least 100,000 cells, at least 200,000 cells, at least 500,000 cells, at least 1,000,000 cells, at least 2,000,000 cells, at least 5,000,000 cells, at least 10,000,000 cells, at least 20,000,000 cells, at least 50,000,000 cells, at least 100,000,000 cells, at least 200,000,000 cells, at least 500,000,000 cells, at least 1,000,000,000 cells, or more. In some cases, the thresholds of the increased exhaustion of the first sub-population of the plurality of cells can be increased by at least about 1% to about 1,000%, about 1% to about 1,000%, or at most about 1% to about 1,000% as measured from at most 1,000,000,000 cells, at most 500,000,000 cells, at most 100,000,000 cells, at most 50,000,000 cells, at most 10,000,000 cells, at most 5,000,000 cells, at most 1,000,000 cells, at most 500,000 cells, at most 100,000 cells, at most 50,000 cells, at most 10,000 cells, at most 5,000 cells, at most 1,000 cells, or less.

In some cases, the thresholds of the increased exhaustion of the first sub-population of the plurality of cells can be measured from a defined number of cells over a period of time. In some cases, the thresholds of the increased exhaustion of the first sub-population of the plurality of cells can be measured from a defined number of cells over a period of 1 minute, 2 minutes, 5 minutes, 10 minutes, minutes, 30 minutes, 1 hour, 2 hours, 5 hours, 10 hours, 12 hours, 24 hours, 36 hours, 2 days, 5 days, 10 days, or longer. In some embodiments, the thresholds of increased exhaustion of the first sub-population of the plurality of cells may be by at least about 1% to about 1,000%, about 1% to about 1,000%, or at most about 1% to about 1,000% as measured from 1000 cells, 2000 cells, 5000 cells, cells, 20,000 cells, 50,000 cells, 100,000 cells, 200,000 cells, 500,000 cells, 1,000,000 cells, 2,000,000 cells, 5,000,000 cells, 10,000,000 cells, 20,000,000 cells, 50,000,000 cells, 100,000,000 cells, 200,000,000 cells, 500,000,000 cells, or 1,000,000,000 cells over a period of 1 minute, 2 minutes, 5 minutes, 10 minutes, 20 minutes, 30 minutes, 1 hour, 2 hours, 5 hours, 10 hours, 12 hours, 24 hours, 36 hours, 2 days, 5 days, 10 days, or longer. In some embodiments, the thresholds of increased exhaustion of the first sub-population of the plurality of cells may be by at least about 1% to about 1,000%, about 1% to about 1,000%, or at most about 1% to about 1,000% as measured from at least 1000 cells, at least 2000 cells, at least 5000 cells, at least 10,000 cells, at least 20,000 cells, at least 50,000 cells, at least 100,000 cells, at least 200,000 cells, at least 500,000 cells, at least 1,000,000 cells, at least 2,000,000 cells, at least 5,000,000 cells, at least 10,000,000 cells, at least 20,000,000 cells, at least 50,000,000 cells, at least 100,000,000 cells, at least 200,000,000 cells, at least 500,000,000 cells, at least 1,000,000,000 cells, or more over a period of 1 minute, 2 minutes, 5 minutes, 10 minutes, 20 minutes, 30 minutes, 1 hour, 2 hours, 5 hours, 10 hours, 12 hours, 24 hours, 36 hours, 2 days, 5 days, 10 days, or longer. In some embodiments, the thresholds of increased exhaustion of the first sub-population of the plurality of cells may be determined by at least about 1% to about 1,000%, about 1% to about 1,000%, or at most about 1% to about 1,000% as measured from at most 1,000,000,000 cells, at most 500,000,000 cells, at most 100,000,000 cells, at most 50,000,000 cells, at most 10,000,000 cells, at most 5,000,000 cells, at most 1,000,000 cells, at most 500,000 cells, at most 100,000 cells, at most 50,000 cells, at most 10,000 cells, at most 5,000 cells, at most 1,000 cells, or less over a period of 1 minute, 2 minutes, 5 minutes, 10 minutes, 20 minutes, 30 minutes, 1 hour, 2 hours, 5 hours, 10 hours, 12 hours, 24 hours, 36 hours, 2 days, 5 days, 10 days, or longer.

In some embodiments, the thresholds comprise changes in cellular metabolism of the first sub-population cells. In some embodiments, the thresholds comprise changes in increasing cellular metabolism of the first sub-population cells. In some embodiments, the thresholds comprise changes in decreasing cellular metabolism of the first sub-population cells. In some cases, the changes in cellular metabolism of the first sub-population can comprise changes in ATP production, glucose uptake, glycolysis, cellular respiration, mitochondrial membrane potential, fatty acid oxidation, oxidative phosphorylation, steroids or cholesterol synthesis, cytokine production, expression of cellular metabolic genes or pathways, or a combination thereof. In some instances, changes in cellular metabolism of the first sub-population can be increased or decreased by at least 0.1 fold to about 1000 fold. In some cases, the rate of cell migration of the first sub-population can be increased or decreased by at least 0.1 fold to about 20 fold, at least about 0.1 fold to about 50 fold, at least about 0.1 fold to about 100 fold, at least about 0.1 fold to about 200 fold, at least about 0.1 fold to about 500 fold, at least about 0.1 fold to about 1,000 fold, at least about 2 fold to about 50 fold, at least about 2 fold to about 100 fold, at least about 2 fold to about 200 fold, at least about 2 fold to about 500 fold, at least about 2 fold to about 1,000 fold, at least about 10 fold to about 100 fold, at least about 10 fold to about 200 fold, at least about 10 fold to about 500 fold, at least about 10 fold to about 1,000 fold, at least about 50 fold to about 200 fold, at least about 50 fold to about 500 fold, at least about 50 fold to about 1,000 fold, at least about 100 fold to about 500 fold, at least about 100 fold to about 1,000 fold, or at least about 500 fold to about 1,000 fold.

In some embodiments, the thresholds comprise changes in cellular metabolism of the first sub-population cells. In some cases, changes in cellular metabolism of the first sub-population can be increased by at most 0.1 fold to about 1000 fold. In some cases, the rate of cell migration of the first sub-population can be increased or decreased by at most 0.1 fold to about 20 fold, at most about 0.1 fold to about 50 fold, at most about 0.1 fold to about 100 fold, at most about 0.1 fold to about 200 fold, at most about 0.1 fold to about 500 fold, at most about 0.1 fold to about 1,000 fold, at most about 2 fold to about 50 fold, at most about 2 fold to about 100 fold, at most about 2 fold to about 200 fold, at most about 2 fold to about 500 fold, at most about 2 fold to about 1,000 fold, at most about 10 fold to about 100 fold, at most about 10 fold to about 200 fold, at most about 10 fold to about 500 fold, at most about 10 fold to about 1,000 fold, at most about 50 fold to about 200 fold, at most about 50 fold to about 500 fold, at most about 50 fold to about 1,000 fold, at most about 100 fold to about 500 fold, at most about 100 fold to about 1,000 fold, or at most about 500 fold to about 1,000 fold.

In some embodiments, the thresholds comprise changes in cellular metabolism of the first sub-population cells as determined. In some cases, the changes in cellular metabolism of the first sub-population can be increased or decreased by about 0.1 fold to about 1000 fold. In some cases, the changes in cellular metabolism of the first sub-population can be increased or decreased by about 0.1 fold to about 20 fold, about 0.1 fold to about 50 fold, about 0.1 fold to about 100 fold, about 0.1 fold to about 200 fold, about 0.1 fold to about 500 fold, about 0.1 fold to about 1,000 fold, about 2 fold to about 50 fold, about 2 fold to about 100 fold, about 2 fold to about 200 fold, about 2 fold to about 500 fold, about 2 fold to about 1,000 fold, about 10 fold to about 100 fold, about 10 fold to about 200 fold, about 10 fold to about 500 fold, about 10 fold to about 1,000 fold, about 50 fold to about 200 fold, about 50 fold to about 500 fold, about 50 fold to about 1,000 fold, about 100 fold to about 500 fold, about 100 fold to about 1,000 fold, or about 500 fold to about 1,000 fold.

In some embodiments, the thresholds comprise changes in cellular metabolism of the first sub-population cells. In some cases, the changes in cellular metabolism of the first sub-population can be increased or decreased by at least about 0.1 fold, 1 fold, 2 fold, 5 fold, 10 fold, 50 fold, 100 fold, 500 fold, 1000 fold, or more. In some cases, the changes in cellular metabolism of the first sub-population can be increased or decreased by at most about 1000 fold, 500 fold, 100 fold, 50 fold, 10 fold, 5 fold, 2 fold, 1 fold, 0.1 fold, or less.

In some cases, the thresholds of changes in cellular metabolism of the first sub-population cells may be determined from a defined number of cells, as provided herein.

In some embodiments, the changes in cellular metabolism of the first sub-population cells is increased or decreased by at least about 0.1 fold to about 1000 fold, about 0.1 fold to about 1000 fold, or at most about 0.1 fold to about 1000 fold as measured from 1000 cells, 2000 cells, 5000 cells, cells, 20,000 cells, 50,000 cells, 100,000 cells, 200,000 cells, 500,000 cells, 1,000,000 cells, 2,000,000 cells, 5,000,000 cells, 10,000,000 cells, 20,000,000 cells, 50,000,000 cells, 100,000,000 cells, 200,000,000 cells, 500,000,000 cells, or 1,000,000,000 cells. In some cases, the changes in cellular metabolism of the first sub-population cells is increased or decreased by at least about 0.1 fold to about 1000 fold, about 0.1 fold to about 1000 fold, or at most about 0.1 fold to about 1000 fold as measured from at least 1000 cells, at least 2000 cells, at least 5000 cells, at least 10,000 cells, at least cells, at least 50,000 cells, at least 100,000 cells, at least 200,000 cells, at least 500,000 cells, at least 1,000,000 cells, at least 2,000,000 cells, at least 5,000,000 cells, at least 10,000,000 cells, at least cells, at least 50,000,000 cells, at least 100,000,000 cells, at least 200,000,000 cells, at least 500,000,000 cells, at least 1,000,000,000 cells, or more. In some cases, the changes in cellular metabolism of the first sub-population cells may be increased or decreased by at least about 0.1 fold to about 1000 fold, about 0.1 fold to about 1000 fold, or at most about 0.1 fold to about 1000 fold as measured from at most 1,000,000,000 cells, at most 500,000,000 cells, at most 100,000,000 cells, at most 50,000,000 cells, at most 10,000,000 cells, at most 5,000,000 cells, at most 1,000,000 cells, at most 500,000 cells, at most 100,000 cells, at most 50,000 cells, at most 10,000 cells, at most 5,000 cells, at most 1,000 cells, or less.

In some cases, the thresholds of changes in cellular metabolism of the first sub-population cells as determined by chemotaxis assay may be determined from a defined number of cells over a period of time. In some cases, the thresholds of changes in cellular metabolism of the first sub-population cells may be measured from a defined number of cells over a period of 1 minute, 2 minutes, 5 minutes, 10 minutes, 20 minutes, 30 minutes, 1 hour, 2 hours, 5 hours, 10 hours, 12 hours, 24 hours, 36 hours, 48 hours, 72 hours, or longer. In some embodiments, the thresholds of changes in cellular metabolism of the first sub-population cells may be increased or decreased by at least about 0.1 fold to about 1000 fold, about 0.1 fold to about 1000 fold, or at most about 0.1 fold to about 1000 fold as measured from 1000 cells, 2000 cells, 5000 cells, 10,000 cells, 20,000 cells, 50,000 cells, 100,000 cells, 200,000 cells, 500,000 cells, 1,000,000 cells, 2,000,000 cells, 5,000,000 cells, 10,000,000 cells, 20,000,000 cells, cells, 100,000,000 cells, 200,000,000 cells, 500,000,000 cells, or 1,000,000,000 cells over a period of 1 minute, 2 minutes, 5 minutes, 10 minutes, 20 minutes, 30 minutes, 1 hour, 2 hours, 5 hours, 10 hours, 12 hours, 24 hours, 36 hours, 48 hours, 72 hours, or longer. In some embodiments, the thresholds of changes in cellular metabolism of the first sub-population cells may be increased or decreased by at least about 0.1 fold to about 1000 fold, about 0.1 fold to about 1000 fold, or at most about 0.1 fold to about 1000 fold as measured from at least 1000 cells, at least 2000 cells, at least 5000 cells, at least 10,000 cells, at least 20,000 cells, at least 50,000 cells, at least 100,000 cells, at least 200,000 cells, at least 500,000 cells, at least 1,000,000 cells, at least 2,000,000 cells, at least 5,000,000 cells, at least 10,000,000 cells, at least 20,000,000 cells, at least 50,000,000 cells, at least 100,000,000 cells, at least 200,000,000 cells, at least 500,000,000 cells, at least 1,000,000,000 cells, or more over a period of 1 minute, 2 minutes, 5 minutes, 10 minutes, 20 minutes, 30 minutes, 1 hour, 2 hours, 5 hours, 10 hours, 12 hours, 24 hours, 36 hours, 48 hours, 72 hours, or longer. In some embodiments, the thresholds of changes in cellular metabolism of the first sub-population cells may be increased or decreased by at least about 0.1 fold to about 1000 fold, about 0.1 fold to about 1000 fold, or at most about 0.1 fold to about 1000 fold as measured from at most 1,000,000,000 cells, at most 500,000,000 cells, at most 100,000,000 cells, at most 50,000,000 cells, at most 10,000,000 cells, at most 5,000,000 cells, at most 1,000,000 cells, at most 500,000 cells, at most 100,000 cells, at most 50,000 cells, at most 10,000 cells, at most 5,000 cells, at most 1,000 cells, or less over a period of 1 minute, 2 minutes, minutes, 10 minutes, 20 minutes, 30 minutes, 1 hour, 2 hours, 5 hours, 10 hours, 12 hours, 24 hours, 36 hours, 48 hours, 72 hours, or longer.

In some embodiments, when the first sub-population of the plurality of cells meets the thresholds a second sub-population of the plurality of cells can be administered to subjects as therapies. In some embodiments, the second sub-population of the plurality of cells administered to the subjects can be treated with antigen prior to the administration. In some embodiments, the second sub-population of the plurality of cells administered to the subjects without first being treated with antigen prior the administration. In some embodiments, the second sub-population of the plurality of cells can be expanded prior to the administration. In some embodiments, the second sub-population of the plurality of cells can be administered to the subjects without undergoing cellular expansion. In some embodiments, the second sub-population of the plurality of cells can be administered intrathecally, intraocularly, intravitreally, retinally, intravenously, intramuscularly, intraventricularly, intracerebrally, intracerebellarly, intracerebroventricularly, intraperenchymally, subcutaneously, or a combination thereof. In some embodiments, the second sub-population of the plurality of cells can be formulated into a pharmaceutical composition.

In some embodiments, the first and second sub-population of the plurality of cells can be immune cell, myeloid cell, a T cell such as alpha beta cytotoxic T cell, a gamma delta T cell, a regulatory T cell, a natural killer T cell, a B cell, a natural killer cell, macrophages, mast cells, endothelial cells, fibroblasts, or various stromal cells.

In some embodiments, the antigen that stimulates the first and second sub-population of the plurality of cells can be 707-AP, a biotinylated molecule, a-Actinin-4, abl-bcr alb-b3 (b2a2), abl-bcr alb-b4 (b3a2), adipophilin, AFP, AIM-2, Annexin II, ART-4, BAGE, b-Catenin, bcr-abl, bcr-abl p190 (e1a2), bcr-abl p210 (b2a2), bcr-abl p210 (b3a2), BING-4, CAG-3, CAIX, CAMEL, Caspase-8, CD171, CD19, CD20, CD22, CD23, CD24, CD30, CD33, CD38, CD44v7/8, CDC27, CDK-4, CEA, CLCA2, Cyp-B, DAM-10, DAM-6, DEK-CAN, EGFRvIII, EGP-2, EGP-40, ELF2, Ep-CAM, EphA2, EphA3, erb-B2, erb-B3, erb-B4, ES-ESO-1a, ETV6/AML, FBP, fetal acetylcholine receptor, FGF-5, FN, G250, GAGE-1, GAGE-2, GAGE-3, GAGE-4, GAGE-5, GAGE-6, GAGE-7B, GAGE-8, GD2, GD3, GnT-V, Gp100, gp75, Her-2, HLA-A*0201-R170I, HMW-MAA, HSP70-2 M, HST-2 (FGF6), HST-2/neu, hTERT, iCE, IL-11Rα, IL-13Rα2, KDR, KIAA0205, K-RAS, L1-cell adhesion molecule, LAGE-1, LDLR/FUT, Lewis Y, MAGE-1, MAGE-10, MAGE-12, MAGE-2, MAGE-3, MAGE-4, MAGE-6, MAGE-A1, MAGE-A2, MAGE-A3, MAGE-A6, MAGE-B1, MAGE-B2, Malic enzyme, Mammaglobin-A, MART-1/Melan-A, MART-2, MC1R, M-CSF, mesothelin, MUC1, MUC16, MUC2, MUM-1, MUM-2, MUM-3, Myosin, NA88-A, Neo-PAP, NKG2D, NPM/ALK, N-RAS, NY-ESO-1, OA1, OGT, oncofetal antigen (h5T4), OS-9, P polypeptide, P15, P53, PRAME, PSA, PSCA, PSMA, PTPRK, RAGE, ROR1, RU1, RU2, SART-1, SART-2, SART-3, SOX10, SSX-2, Survivin, Survivin-2B, SYT/SSX, TAG-72, TEL/AML1, TGFaRII, TGFbRII, TP1, TRAG-3, TRG, TRP-1, TRP-2, TRP-2/INT2, TRP-2-6b, Tyrosinase, VEGF-R2, WT1, α-folate receptor, κ-light chain, a tumor associated antigen, a neoantigen, or any combination or variation thereof. In some embodiments, the antigen is not CD3.

In some embodiments, the antigen is not CD28.

In some embodiments, the antigen is conjugated to a non-cellular substrate. Non-limiting examples of materials for non-cellular substrates include polymers, glass, plastics, metal, or any other synthetic material. In some embodiments the non-cellular substrate can be a plate, a membrane, a matrix, a chip, a plurality of beads, or any surface for the conjugation of the antigen.

In an aspect, the present disclosure provides a method of assessing activity of plurality of cells by treating the cells expressing one or more chimeric receptors comprising a ligand binding domain specific for an antigen at least one density. The method further comprises assessing a first activity of a first sub-population of the plurality of cells upon treatment with a first non-cellular substrate comprising the antigen at a first surface density. The method further comprises assessing a second activity of a second sub-population of the plurality of cells upon treatment with a second non-cellular substrate comprising the antigen at a second surface density that is different than the first surface density.

In some embodiments, the first and second activity can be any one of the threshold measurements. In some embodiments, the first activity is greater than the second activity by at least fold, 0.5 fold, 1 fold, 2 fold, 5 fold, 10 fold, 50 fold, 100 fold, 500 fold, 1,000 fold, 5,000 fold, fold, or more. In some embodiments, the first activity is greater than the second activity by about 0.1 fold, 0.5 fold, 1 fold, 2 fold, 5 fold, 10 fold, 50 fold, 100 fold, 500 fold, 1,000 fold, 5,000 fold, or 10,000 fold. In some embodiments, the first activity is greater than the second activity by at most 10,000 fold, 5,000 fold, 1,000 fold, 500 fold, 100 fold, 50 fold, 10 fold, 5 fold, 2 fold, 1 fold, 0.5 fold, 0.1 fold, or less. In some embodiments, the second activity is greater than the first activity by at least 0.1 fold, 0.5 fold, 2 fold, 5 fold, 10 fold, 50 fold, 100 fold, 500 fold, 1,000 fold, 5,000 fold, or fold, or more. In some embodiments, the second activity greater than the first activity by about fold, 0.5 fold, 2 fold, 5 fold, 10 fold, 50 fold, 100 fold, 500 fold, 1,000 fold, 5,000 fold, or 10,000 fold. In some embodiments, the second activity is greater than the first activity by 10,000 fold, 5,000 fold, 1,000 fold, 500 fold, 100 fold, 50 fold, 10 fold, 5 fold, 2 fold, 1 fold, 0.5 fold, 0.1 fold, or less.

In some embodiments, the percentage of antigen can be at least about 0.001%, 0.005%, 0.01%, 0.1%, 0.2%, 0.3%, 0.4%, 0.5%, 0.6%, 0.7%, 0.8%, 0.9%, 1.0%, 2%, 5%, 10%, 20%, 30%, 40%, 50%, 60%, 70%, 80%, 90%, or more. In some embodiments, the percentage of antigen can be at most about 90%, 80%, 70%, 60%, 40%, 30%, 20%, 10%, 5%, 2%, 1%, 0.9%, 0.8%, 0.7%, 0.6%, 0.5%, 0.3%, 0.2%, 0.1%, 0.05%, 0.01%, 0.005%, 0.001%, or less.

In some embodiments, the surface densities can be determined by percentage of antigen in the total volume during the conjugation process. In some cases, a total volume of the conjugation process may comprise (i) a first volume comprising one or more antigens to be conjugated to a substrate (e.g., a bead) and (ii) a second volume comprising the substrate. A volume fraction of the first volume to the total volume may be referred to a percentage of the antigen(s) in the total volume during the conjugation process. In some embodiments, a surface density of an antigen on a noncellular substrate can be measured with flow cytometry. In some cases, a surface density of an antigen on a noncellular substrate can be measured as antibody binding capacity (ABC) per area (e.g., micrometer (μM) squared) or as a total number of antigen per each unit of noncellular substrate. Without wishing to be bound by theory, in some cases, a higher ratio of the first volume to the second volume may yield a higher density of the antigen(s) bound to a surface of the substrate.

In some embodiments, a surface density may be defined by a ratio of an amount of an antigen to an amount of a conjugating moiety (e.g., a carbodiimide crosslinker) in a reaction volume during the conjugation process. In an example, a final product of an antigen-conjugated substrate may be referred to having a 100% antigen surface density when a 1:1 molar ratio of the antigen to its conjugating moiety is used. In another example, a final product of an antigen-conjugated substrate may be referred to having a 50% antigen surface density when a 5:10 molar ratio of the antigen to its conjugating moiety is used. In a different example, a final product of an antigen-conjugated substrate may be referred to having a 10% antigen surface density when a 1:10 molar ratio of the antigen to its conjugating moiety is used. Without wishing to be bound by theory, the ratio of the amount of the antigen to the amount of the conjugating moiety may determine the surface density of the antigen on a surface of the substrate.

In some embodiments, the surface densities can be determined by ratio between antigen and non-cellular substrate. In some embodiments, the ratio between the antigen and the non-cellular substrate can be 1:1,000,000, 1:500,000, 1:100,000, 1:50,000, 1:10,000, 1:5,000, 1:1,000, 1:500, 1:100, 1:50, 1:10, 1:5, 1:4, 1:3, 1:2, 1:1, 2:1, 3:1, 4:1, 5:1, 10:1, 50:1, 100:1, 500:1, 1,000:1, 5,000:1, 50,000:1, 100,000:1, 500,000:1, 1,000,000:1, or any range therebetween. In some embodiments, the ratio between the antigen and the non-cellular substrate can be 5:1,000,000, 5:100,000, 5:50,000, 5:10,000, 5:5,000, 5:1,000, 5:500, 5:100, 5:50, 5:10, 5:4, 5:3, 5:2, 5:1, or any range therebetween.

In some embodiments, the first surface density and the second surface density can have the same density. In some instances, the first surface density and the second surface density can be different densities. In some embodiments, the first surface density is greater than the second surface density by at least 0.1 fold, 0.5 fold, 2 fold, 5 fold, 10 fold, 50 fold, 100 fold, 500 fold, 1,000 fold, fold, 10,000 fold, or more. In some embodiments, the first surface density is greater than the second surface density by about 0.1 fold, 0.5 fold, 2 fold, 5 fold, 10 fold, 50 fold, 100 fold, 500 fold, 1,000 fold, 5,000 fold, or 10,000 fold. In some embodiments, the first surface density is greater than the second surface density by at most 10,000 fold, 5,000 fold, 1,000 fold, 500 fold, 200 fold, 100 fold, fold, 20 fold, 10 fold, 5 fold, 2 fold, 1 fold, 0.5 fold, 0.2 fold, 0.1 fold, or less.

In some embodiments, the second surface density is greater than the first surface density by at least 0.1 fold, 0.5 fold, 2 fold, 5 fold, 10 fold, 50 fold, 100 fold, 500 fold, 1,000 fold, 5,000 fold, fold, or more. In some embodiments, the second surface density is greater than the first surface density by about 0.1 fold, 0.5 fold, 2 fold, 5 fold, 10 fold, 50 fold, 100 fold, 500 fold, 1,000 fold, 5,000 fold, or 10,000 fold. In some embodiments, the second surface density is greater than the first surface density by at most 10,000 fold, 5,000 fold, 1,000 fold, 500 fold, 200 fold, 100 fold, 50 fold, 20 fold, 10 fold, 5 fold, 2 fold, 1 fold, 0.5 fold, 0.2 fold, 0.1 fold, or less.

In some embodiments, the first and second sub-population of the plurality of cells assayed by the antigen at different surface density can be immune cell, myeloid cell, a T cell such as alpha beta cytotoxic T cell, a gamma delta T cell, a regulatory T cell, a natural killer T cell, a B cell, a natural killer cell, macrophages, mast cells, endothelial cells, fibroblasts, or various stromal cells.

In some embodiments, the antigen conjugated to the non-cellular substrate at different surface density can be any antigen provided in the present disclosure, e.g., 707-AP, a biotinylated molecule, a-Actinin-4, abl-bcr alb-b3 (b2a2), abl-bcr alb-b4 (b3a2), adipophilin, AFP, AIM-2, Annexin II, ART-4, BAGE, b-Catenin, bcr-abl, bcr-abl p190 (e1a2), bcr-abl p210 (b2a2), bcr-abl p210 (b3a2), BING-4, CAG-3, CAIX, CAMEL, Caspase-8, CD171, CD19, CD20, CD22, CD23, CD24, CD30, CD33, CD38, CD44v7/8, CDC27, CDK-4, CEA, CLCA2, Cyp-B, DAM-10, DAM-6, DEK-CAN, EGFRvIII, EGP-2, EGP-40, ELF2, Ep-CAM, EphA2, EphA3, erb-B2, erb-B3, erb-B4, ES-ESO-1a, ETV6/AML, FBP, fetal acetylcholine receptor, FGF-5, FN, G250, GAGE-1, GAGE-2, GAGE-3, GAGE-4, GAGE-GAGE-6, GAGE-7B, GAGE-8, GD2, GD3, GnT-V, Gp100, gp75, Her-2, HLA-A*0201-R170I, HMW-MAA, HSP70-2 M, HST-2 (FGF6), HST-2/neu, hTERT, iCE, IL-11Rα, IL-13Rα2, KDR, KIAA0205, K-RAS, L1-cell adhesion molecule, LAGE-1, LDLR/FUT, Lewis Y, MAGE-1, MAGE-MAGE-12, MAGE-2, MAGE-3, MAGE-4, MAGE-6, MAGE-A1, MAGE-A2, MAGE-A3, MAGE-A6, MAGE-B1, MAGE-B2, Malic enzyme, Mammaglobin-A, MART-1/Melan-A, MART-2, MC1R, M-CSF, mesothelin, MUC1, MUC16, MUC2, MUM-1, MUM-2, MUM-3, Myosin, NA88-A, Neo-PAP, NKG2D, NPM/ALK, N-RAS, NY-ESO-1, OA1, OGT, oncofetal antigen (h5T4), OS-9, P polypeptide, P15, P53, PRAME, PSA, PSCA, PSMA, PTPRK, RAGE, ROR1, RU1, RU2, SART-1, SART-2, SART-3, SOX10, SSX-2, Survivin, Survivin-2B, SYT/SSX, TAG-72, TEL/AML1, TGFaRII, TGFbRII, TP1, TRAG-3, TRG, TRP-1, TRP-2, TRP-2/INT2, TRP-2-6b, Tyrosinase, VEGF-R2, WT1, α-folate receptor, κ-light chain, a tumor associated antigen, a neoantigen, or any combination or variation thereof. In some embodiments, the antigen is not CD3. In some embodiments, the antigen is not CD28.

In implementing any one of the subject methods of the present disclosure, or in any one of the subject systems of the present disclosure, non-limiting examples of materials for non-cellular substrates include polymers, glass, plastics, metal, or any other synthetic material. In some embodiments the non-cellular substrate can be a plate, a membrane, a matrix, a chip, a plurality of beads, or any surface for the conjugation of the antigen.

In some embodiments, the first non-cellular substrate and the second non-cellular substrate disclosed herein can be the same. In some embodiments, the first non-cellular substrate and the second non-cellular substrate can be the different. In some embodiments, the first non-cellular substrate and the second non-cellular substrate can share the same materials. In some embodiments, the first non-cellular substrate and the second non-cellular substrate can share at least 0.1%, 0.5%, 1%, 5%, 10%, 20%, 30%, 40%, 50%, 60%, 70%, 80%, 90%, 95%, or 99% of the same materials.

In another aspect, the present disclosure provides a system for assessing activity of a plurality cells expressing one or more chimeric receptors comprising a ligand binding domain specific for an antigen. The system may comprise a first non-cellular substrate comprising the antigen at a first surface density. The system may comprise a second non-cellular substrate comprising the antigen at a second surface density that is different than the first surface density. In some cases, the first non-cellular substrate and the second non-cellular substrate may be substantially the same in material(s), size, shape, density, and/or weight. In a different aspect, the present disclosure provides a kit comprising the system disclosed herein.

In an aspect, the present disclosure provides a method of assessing activity of plurality of cells by treating the cells expressing one or more chimeric receptors comprising a ligand binding domain specific for multiple antigens. The method further comprises assessing an activity of a first sub-population of the plurality of cells upon treatment with a non-cellular substrate comprising a first antigen and a second antigen. The method further comprises assessing an activity of a first sub-population of the plurality of cells expressing a first chimeric receptor comprising a first ligand binding domain specific for the first antigen. In some embodiments, the first chimeric receptor comprises a second ligand binding domain specific for the second antigen.

In some embodiments, the activity of the plurality of cells can be assayed by a first antigen conjugated to the non-cellular substrate. In some embodiments, the activity of the plurality of cells can be assayed by a first antigen and a second antigen conjugated to the same non-cellular substrate, wherein the first and second antigens are different. In some embodiments, the activity of the plurality of cells can be assayed by a first antigen, a second antigen, and a third antigen conjugated to the same non-cellular substrate, wherein the first, second, and third antigens are different. In some embodiments, the activity of the plurality of cells can be assayed by a first antigen, a second antigen, a third antigen, and a fourth antigen conjugated to the same non-cellular substrate, wherein the first, second, third, and fourth antigens are different. In some embodiments, the activity of the plurality of cells can be assayed by a first antigen, a second antigen, a third antigen, a fourth, and a fifth antigen conjugated to the same non-cellular substrate, wherein the first, second, third, fourth, and fifth antigens are different. In some embodiments, the activity of the plurality of cells can be assayed by a first antigen, a second antigen, a third antigen, a fourth antigen, a fifth antigen, and any additional antigens all conjugated to the same non-cellular substrate, wherein the first, second, third, fourth, fifth, and the additional antigens are different. In some embodiments, the activity of the plurality of cells can be assayed by a plurality of different antigens conjugated to the same non-cellular substrate. In some embodiments, the plurality of different antigens can be conjugated to the non-cellular substrates at the same surface density. In some embodiments, the plurality of different antigens can be conjugated to the non-cellular substrates at the different surface densities. Surface densities can be any ratio or percentage described in the instant disclosure.

In some embodiments, the activity assayed with the first and second antigen can be any one of the threshold measurements described in this instant disclosure. In some embodiments, the activity assayed with the first antigen can be the same as the activity assayed with the second antigen. In some embodiments, the activity assayed with the first antigen can be different from the activity assayed with the second antigen.

In some embodiments, the first and/or the second antigen may be any antigen as disclosed in the present disclosure. In some embodiments, the cell may be any type of immune cell as disclosed herein.

In some embodiments, the plurality of cells assayed with a first and a second antigen express a second chimeric receptor comprising a second ligand binding domain specific for the second antigen. In some instances, the plurality of cells expresses an endogenous receptor configured to bind the second antigen. In some embodiments, the second antigen may be an immune checkpoint inhibitor. In some embodiments, the second antigen can include PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GALS, adenosine, TGF (e.g., TGFbeta), variations thereof, and combinations thereof.

In one aspect, the present disclosure describes a method of assessing activity of a plurality cells expressing one or more chimeric receptors comprising a ligand binding domain specific for an antigen, comprising: assessing an activity of a first sub-population of the plurality of cells upon treatment with a first non-cellular substrate comprising the antigen at a first surface density; assessing the activity of a second sub-population of the plurality of cells upon treatment with a second non-cellular substrate comprising the antigen at a second surface density that is lower than the first surface density. In some embodiments of any of the methods disclosed herein the first surface density is at least 1.1-fold, 1.5-fold, 2-fold, 3-fold, 4-fold, 5-fold, 10-fold, 15-fold, 20-fold or 50-fold greater than the second surface density.

In some embodiments of any of the methods disclosed herein, the method further comprises comparing the activity of the first sub-population and the activity of the second sub-population. In some embodiments of any of the methods disclosed herein, the method further comprises assessing a suitability of the plurality of cells for use in a cell therapy for a subject in need thereof, based at least in part on the comparing.

In some embodiments of any of the methods disclosed herein, the method further comprises determining that the plurality of cells is suitable for the use when the activity of the first sub-population and the activity of the second sub-population are about the same.

In some embodiments of any of the methods disclosed herein, the method further comprises determining that the plurality of cells is suitable for the use when the activity of the first sub-population is greater than the activity of the second sub-population by at least 10%. In some embodiments, the plurality of cells is suitable for the use when the activity of the first sub-population is greater than the activity of the second sub-population by at least 20%. In some embodiments, the plurality of cells is suitable for the use when the activity of the first sub-population is greater than the activity of the second sub-population by at least 30%. In some embodiments, the plurality of cells is suitable for the use when the activity of the first sub-population is greater than the activity of the second sub-population by at least 40%. In some embodiments, the plurality of cells is suitable for the use when the activity of the first sub-population is greater than the activity of the second sub-population by at least 50%. In some embodiments, the plurality of cells is suitable for the use when the activity of the first sub-population is greater than the activity of the second sub-population by at least 60%. In some embodiments, the plurality of cells is suitable for the use when the activity of the first sub-population is greater than the activity of the second sub-population by at least 70%. In some embodiments, the plurality of cells is suitable for the use when the activity of the first sub-population is greater than the activity of the second sub-population by at least 80%. In some embodiments, the plurality of cells is suitable for the use when the activity of the first sub-population is greater than the activity of the second sub-population by at least 90%. In some embodiments, the plurality of cells is suitable for the use when the activity of the first sub-population is greater than the activity of the second sub-population by at least 100%. In some embodiments, the plurality of cells is suitable for the use when the activity of the first sub-population is greater than the activity of the second sub-population by at least 120%. In some embodiments, the plurality of cells is suitable for the use when the activity of the first sub-population is greater than the activity of the second sub-population by at least about 150%. In some embodiments, the plurality of cells is suitable for the use when the activity of the first sub-population is greater than the activity of the second sub-population by at least 200%.

In some embodiments, the plurality of cells is suitable for the use when the activity of the second sub-population is greater than the activity of the first sub-population by at least 10%. In some embodiments, the plurality of cells is suitable for the use when the activity of the second sub-population is greater than the activity of the first sub-population by at least 20%. In some embodiments, the plurality of cells is suitable for the use when the activity of the second sub-population is greater than the activity of the first sub-population by at least 30%. In some embodiments, the plurality of cells is suitable for the use when the activity of the second sub-population is greater than the activity of the first sub-population by at least 40%. In some embodiments, the plurality of cells is suitable for the use when the activity of the second sub-population is greater than the activity of the first sub-population by at least 50%. In some embodiments, the plurality of cells is suitable for the use when the activity of the second sub-population is greater than the activity of the first sub-population by at least 60%. In some embodiments, the plurality of cells is suitable for the use when the activity of the second sub-population is greater than the activity of the first sub-population by at least 70%. In some embodiments, the plurality of cells is suitable for the use when the activity of the second sub-population is greater than the activity of the first sub-population by at least 80%. In some embodiments, the plurality of cells is suitable for the use when the activity of the second sub-population is greater than the activity of the first sub-population by at least 90%. In some embodiments, the plurality of cells is suitable for the use when the activity of the first sub-population is greater than the activity of the second sub-population by about 92%, 95% or about 100%.

In another aspect, the present disclosure provides a system for assessing activity of a plurality of cells expressing one or more chimeric receptors comprising a ligand binding domain specific for an antigen. The system may comprise a non-cellular substrate comprising a first antigen and a second antigen, wherein a cell of the first sub-population expresses a first chimeric receptor comprising a first ligand binding domain specific for the first antigen. In some examples, the first chimeric receptor comprises a second ligand binding domain specific for the second antigen. In additional examples, the cell expresses a second chimeric receptor comprising a second ligand binding domain specific for the second antigen. In other examples, the cell expresses an endogenous receptor configured to bind the second antigen.

III. Non-Cellular Substrates

In implementing any one of the methods and systems provided in the present disclosure, treating a plurality of cells expressing one or more chimeric receptors comprising a ligand binding domain specific for an antigen with a non-cellular substrate (e.g., a non-cellular particle, e.g., a bead such as a magnetic bead) comprising the antigen may comprise subjecting the plurality of cells with a desired amount of the non-cellular substrate. In some cases, a desired amount of a non-cellular substrate comprising the antigen may be at least about 1×10² particles/milliner (particles/mL), 2×10² particles/mL, 4×10² particles/mL, 6×10² particles/mL, 8×10² particles/mL, 1×10³ particles/mL, 2×10³ particles/mL, 4×10³ particles/mL, 6×10³ particles/mL, 8×10³ particles/mL, 1×10⁴ particles/mL, 2×10⁴ particles/mL, 4×10⁴ particles/mL, 6×10⁴ particles/mL, 8×10⁴ particles/mL, 1×10⁵ particles/mL, 2×10⁵ particles/mL, 4×10⁵ particles/mL, 6×10⁵ particles/mL, 8×10⁵ particles/mL, 1×10⁶ particles/mL, 2×10⁶ particles/mL, 4×10⁶ particles/mL, 6×10⁶ particles/mL, 8×10⁶ particles/mL, 1×10⁷ particles/mL, 2×10⁷ particles/mL, 4×10⁷ particles/mL, 6×10⁷ particles/mL, 8×10⁷ particles/mL, 1×10⁸ particles/mL, 2×10⁸ particles/mL, 4×10⁸ particles/mL, 6×10⁸ particles/mL, 8×10⁸ particles/mL, 1×10⁹ particles/mL, 2×10⁹ particles/mL, 4×10⁹ particles/mL, 6×10⁹ particles/mL, 8×10⁹ particles/mL, 1×10¹⁰ particles/mL or more. In some cases, a desired amount of a non-cellular substrate comprising the antigen may be at most about 1×10¹⁰ particles/mL, 8×10⁹ particles/mL, 6×10⁹ particles/mL, 4×10⁹ particles/mL, 2×10⁹ particles/mL, 8×10⁸ particles/mL, 6×10⁸ particles/mL, 4×10⁸ particles/mL, 2×10⁸ particles/mL, 1×10⁸ particles/mL, 8×10⁷ particles/mL, 6×10⁷ particles/mL, 4×10⁷ particles/mL, 2×10⁷ particles/mL, 1×10⁷ particles/mL, 8×10⁶ particles/mL, 6×10⁶ particles/mL, 4×10⁶ particles/mL, 2×10⁶ particles/mL, 1×10⁶ particles/mL, 8×10⁵ particles/mL, 6×10⁵ particles/mL, 4×10⁵ particles/mL, 2×10⁵ particles/mL, 1×10⁵ particles/mL, 8×10⁴ particles/mL, 6×10⁴ particles/mL, 4×10⁴ particles/mL, 2×10⁴ particles/mL, 1×10⁴ particles/mL, 8×10³ particles/mL, 6×10³ particles/mL, 4×10³ particles/mL, 2×10³ particles/mL, 1×10³ particles/mL, 8×10² particles/mL, 6×10² particles/mL, 4×10² particles/mL, 2×10² particles/mL, 1×10² particles/mL, or less.

In some cases, a diameter of a non-cellular substrate comprising the antigen may be at least about 10 nanometers (nm), 20 nm, 40 nm, 60 nm, 80 nm, 100 nm, 200 nm, 400 nm, 600 nm, 800 nm, 1 micrometer (μm), 2 μm, 4 μm, 6 μm, 8 μm, 10 μm, 20 μm, 40 μm, 60 μm, 80 μm, 100 μm, 200 μm, 400 μm, 500 μm, or more. In some cases, a dimeter of a non-cellular substrate comprising the antigen may be at most about 500 μm, 400 μm, 200 μm, 100 μm, 80 μm, 60 μm, 40 μm, 20 μm, 10 μm, 8 μm, 6 μm, 4 μm, 2 μm, 1 μm, 800 nm, 600 nm, 400 nm, 200 nm, 100 nm, 80 nm, 60 nm, 40 nm, 20 nm, 10 nm, or less.

In some cases, a surface density of an antigen on a non-cellular substrate comprising the antigen may be at least about 1×10¹⁴ moles of the antigen per square centimeters of a surface of the non-cellular substrate (moles/cm 2), 2×10¹⁴ moles/cm², 5×10¹⁴ moles/cm 2 1×10¹³ moles/cm², 2×10¹³ moles/cm², 5×10¹³ moles/cm², 1×10¹² moles/cm², 2×10¹² moles/cm², 5×10¹² moles/cm², 1×10¹¹ moles/cm², 2×10¹¹ moles/cm², 5×10¹¹ moles/cm², 1×10¹⁰ moles/cm², 2×10¹⁰ moles/cm², 5×10¹⁰ moles/cm², 1×10⁻⁹ moles/cm², 2×10⁻⁹ moles/cm², 5×10⁻⁹ moles/cm², 1×10⁻⁸ moles/cm², 2×10⁻⁸ moles/cm², 5×10⁻⁸ moles/cm², 1×10⁻⁷ moles/cm², 2×10⁻⁷ moles/cm², 5×10⁻⁷ moles/cm², 1×10⁻⁶ moles/cm 2, or more. In some cases, a surface density of an antigen on a non-cellular substrate comprising the antigen may be at most about 1×10⁻⁶ moles/cm², 2×10⁻⁶ moles/cm², 5×10⁻⁶ moles/cm², 1×10⁻⁷ moles/cm², 2×10⁻⁷ moles/cm², 5×10⁻⁷ moles/cm², 1×10⁻⁸ moles/cm², 2×10⁻⁸ moles/cm², 5×10⁻⁸ moles/cm², 1×10⁻⁹ moles/cm², 2×10⁻⁹ moles/cm², 5×10⁻⁹ moles/cm², 1×10⁻¹¹ moles/cm², 2×10⁻¹¹ moles/cm², 5×10⁻¹¹ moles/cm², 1×10⁻¹² moles/cm², 2×10⁻¹² moles/cm², 5×10⁻¹² moles/cm², 1×10⁻¹³ moles/cm², 2×10⁻¹³ moles/cm², 5×10⁻¹³ moles/cm², 1×10⁻¹⁴ moles/cm², or less.

In some cases, a ligand binding domain configured to bind an antigen of a non-cellular substrate may be an antibody, a fragment, or a derivative thereof, and a surface density of the antigen on the non-cellular substrate may be defined in terms of an antibody binding capacity (ABC). The term “antibody binding capacity” or “ABC” as used herein generally refers to a number of antibodies (or a fragment or a derivative thereof) that a sample (or an amount of a sample) will bind. An ABC value can correlate to a number of antigens presented by one or more of the substrates comprising the antigen. Thus, a substrate comprising an antigen can be assigned an ABC value based on, e.g., a quantitative indirect immunofluorescence (QIIF) assessments comprising a plurality of beads with well-defined quantifies of the antibody (or a fragment or a derivative thereof) against the antigen of the non-cellular substrate. In some examples, a surface density of an antigen on a non-cellular substrate comprising the antigen may be characterized by an ABC value of at least about 1, 2, 4, 6, 8, 20, 40, 60, 80, 100, 200, 400, 600, 800, 1,000, 2,000, 4,000, 6,000, 8,000, 10,000, 20,000, or more. In some samples, a surface density of an antigen on a non-cellular substrate comprising the antigen may be characterized by an ABC value of at least about 1 to about 20,000. In some samples, a surface density of an antigen on a non-cellular substrate comprising the antigen may be characterized by an ABC value of at least about 1 to about 5, about 1 to about 10, about 1 to about 50, about 1 to about 100, about 1 to about 200, about 1 to about 500, about 1 to about 1,000, about 1 to about 2,000, about 1 to about 5,000, about 1 to about 10,000, about 1 to about 20,000, about 5 to about 10, about 5 to about 50, about 5 to about 100, about 5 to about 200, about 5 to about 500, about 5 to about 1,000, about 5 to about 2,000, about 5 to about 5,000, about 5 to about 10,000, about 5 to about 20,000, about to about 50, about 10 to about 100, about 10 to about 200, about 10 to about 500, about 10 to about 1,000, about 10 to about 2,000, about 10 to about 5,000, about 10 to about 10,000, about 10 to about about 50 to about 100, about 50 to about 200, about 50 to about 500, about 50 to about 1,000, about 50 to about 2,000, about 50 to about 5,000, about 50 to about 10,000, about 50 to about 20,000, about 100 to about 200, about 100 to about 500, about 100 to about 1,000, about 100 to about 2,000, about 100 to about 5,000, about 100 to about 10,000, about 100 to about 20,000, about 200 to about 500, about 200 to about 1,000, about 200 to about 2,000, about 200 to about 5,000, about 200 to about about 200 to about 20,000, about 500 to about 1,000, about 500 to about 2,000, about 500 to about 5,000, about 500 to about 10,000, about 500 to about 20,000, about 1,000 to about 2,000, about 1,000 to about 5,000, about 1,000 to about 10,000, about 1,000 to about 20,000, about 2,000 to about about 2,000 to about 10,000, about 2,000 to about 20,000, about 5,000 to about 10,000, about to about 20,000, or about 10,000 to about 20,000. In some samples, a surface density of an antigen on a non-cellular substrate comprising the antigen may be characterized by an ABC value of at least about 1, about 5, about 10, about 50, about 100, about 200, about 500, about 1,000, about 2,000, about 5,000, about 10,000, or about 20,000. In some samples, a surface density of an antigen on a non-cellular substrate comprising the antigen may be characterized by an ABC value of at least at least about 1, about 5, about 10, about 50, about 100, about 200, about 500, about 1,000, about 2,000, about or about 10,000. In some samples, a surface density of an antigen on a non-cellular substrate comprising the antigen may be characterized by an ABC value of at least at most about 5, about 10, about 50, about 100, about 200, about 500, about 1,000, about 2,000, about 5,000, about 10,000, or about 20,000.

In some examples, a surface density of an antigen on a non-cellular substrate comprising the antigen may be characterized by an ABC value of at most about 20,000, 10,000, 8,000, 6,000, 4,000, 2,000, 1,000, 800, 600, 400, 200, 100, 80, 60, 40, 20, 10, or less. In some samples, a surface density of an antigen on a non-cellular substrate comprising the antigen may be characterized by an ABC value of at most about 1 to about 20,000. In some samples, a surface density of an antigen on a non-cellular substrate comprising the antigen may be characterized by an ABC value of at most about 20,000 to about 10,000, about 20,000 to about 5,000, about 20,000 to about 2,000, about 20,000 to about 1,000, about 20,000 to about 500, about 20,000 to about 200, about 20,000 to about 100, about 20,000 to about 50, about 20,000 to about 10, about 20,000 to about 5, about 20,000 to about 1, about 10,000 to about 5,000, about 10,000 to about 2,000, about 10,000 to about 1,000, about 10,000 to about 500, about 10,000 to about 200, about 10,000 to about 100, about 10,000 to about 50, about 10,000 to about about 10,000 to about 5, about 10,000 to about 1, about 5,000 to about 2,000, about 5,000 to about 1,000, about 5,000 to about 500, about 5,000 to about 200, about 5,000 to about 100, about 5,000 to about 50, about 5,000 to about 10, about 5,000 to about 5, about 5,000 to about 1, about 2,000 to about 1,000, about 2,000 to about 500, about 2,000 to about 200, about 2,000 to about 100, about 2,000 to about 50, about 2,000 to about 10, about 2,000 to about 5, about 2,000 to about 1, about 1,000 to about 500, about 1,000 to about 200, about 1,000 to about 100, about 1,000 to about 50, about 1,000 to about about 1,000 to about 5, about 1,000 to about 1, about 500 to about 200, about 500 to about 100, about 500 to about 50, about 500 to about 10, about 500 to about 5, about 500 to about 1, about 200 to about 100, about 200 to about 50, about 200 to about 10, about 200 to about 5, about 200 to about 1, about 100 to about 50, about 100 to about 10, about 100 to about 5, about 100 to about 1, about 50 to about 10, about 50 to about 5, about 50 to about 1, about 10 to about 5, about 10 to about 1, or about 5 to about 1. In some samples, a surface density of an antigen on a non-cellular substrate comprising the antigen may be characterized by an ABC value of at most about 20,000, about 10,000, about 5,000, about 2,000, about 1,000, about 500, about 200, about 100, about 50, about 10, about 5, or about 1. In some samples, a surface density of an antigen on a non-cellular substrate comprising the antigen may be characterized by an ABC value of at most at most about 20,000, about 10,000, about 5,000, about 2,000, about 1,000, about 500, about 200, about 100, about 50, about 10, or about 5. In some samples, a surface density of an antigen on a non-cellular substrate comprising the antigen may be characterized by an ABC value of at most at most about 10,000, about 5,000, about 2,000, about 1,000, about 500, about 200, about 100, about 50, about 10, about 5, or about 1.

In some cases, a total number of an antigen per a single non-cellular substrate may be at least about 100, 200, 400, 600, 800, 1,000, 2,000, 4,000, 6,000, 8,000, 10,000, 20,000, 40,000, 60,000, 100,000, 200,000, 400,000, 500,000, or more. In some cases, a total number of an antigen per a single non-cellular substrate may be at least about 100 to about 5,000,000. In some cases, a total number of an antigen per a single non-cellular substrate may be at least about 100 to about 500, about 100 to about 1,000, about 100 to about 5,000, about 100 to about 10,000, about 100 to about 50,000, about 100 to about 100,000, about 100 to about 500,000, about 100 to about 1,000,000, about 100 to about 5,000,000, about 500 to about 1,000, about 500 to about 5,000, about 500 to about 10,000, about 500 to about 50,000, about 500 to about 100,000, about 500 to about 500,000, about 500 to about 1,000,000, about 500 to about 5,000,000, about 1,000 to about 5,000, about 1,000 to about 10,000, about 1,000 to about 50,000, about 1,000 to about 100,000, about 1,000 to about 500,000, about 1,000 to about 1,000,000, about 1,000 to about 5,000,000, about 5,000 to about 10,000, about 5,000 to about about 5,000 to about 100,000, about 5,000 to about 500,000, about 5,000 to about 1,000,000, about 5,000 to about 5,000,000, about 10,000 to about 50,000, about 10,000 to about 100,000, about to about 500,000, about 10,000 to about 1,000,000, about 10,000 to about 5,000,000, about to about 100,000, about 50,000 to about 500,000, about 50,000 to about 1,000,000, about to about 5,000,000, about 100,000 to about 500,000, about 100,000 to about 1,000,000, about 100,000 to about 5,000,000, about 500,000 to about 1,000,000, about 500,000 to about 5,000,000, or about 1,000,000 to about 5,000,000. In some cases, a total number of an antigen per a single non-cellular substrate may be at least about 100, about 500, about 1,000, about 5,000, about 10,000, about about 100,000, about 500,000, about 1,000,000, or about 5,000,000. In some cases, a total number of an antigen per a single non-cellular substrate may be at least at least about 100, about 500, about 1,000, about 5,000, about 10,000, about 50,000, about 100,000, about 500,000, or about 1,000,000. In some cases, a total number of an antigen per a single non-cellular substrate may be at least at most about 500, about 1,000, about 5,000, about 10,000, about 50,000, about 100,000, about 500,000, about 1,000,000, or about 5,000,000.

In some cases, a total number of an antigen per a single non-cellular substrate may be at most about 500,000, 400,000, 200,000, 100,000, 80,000, 60,000, 40,000, 20,000, 10,000, 8,000, 6,000, 4,000, 2,000, 1,000, 800, 600, 400, 200, 100, or less. In some cases, a total number of an antigen per a single non-cellular substrate may be at least about 1,000 to about 50,000,000. In some cases, a total number of an antigen per a single non-cellular substrate may be at least about 50,000,000 to about about 50,000,000 to about 5,000,000, about 50,000,000 to about 1,000,000, about to about 500,000, about 50,000,000 to about 100,000, about 50,000,000 to about 50,000, about 50,000,000 to about 10,000, about 50,000,000 to about 5,000, about 50,000,000 to about 1,000, about 10,000,000 to about 5,000,000, about 10,000,000 to about 1,000,000, about 10,000,000 to about 500,000, about 10,000,000 to about 100,000, about 10,000,000 to about 50,000, about 10,000,000 to about 10,000, about 10,000,000 to about 5,000, about 10,000,000 to about 1,000, about 5,000,000 to about 1,000,000, about 5,000,000 to about 500,000, about 5,000,000 to about 100,000, about to about 50,000, about 5,000,000 to about 10,000, about 5,000,000 to about 5,000, about to about 1,000, about 1,000,000 to about 500,000, about 1,000,000 to about 100,000, about 1,000,000 to about 50,000, about 1,000,000 to about 10,000, about 1,000,000 to about 5,000, about 1,000,000 to about 1,000, about 500,000 to about 100,000, about 500,000 to about 50,000, about 500,000 to about 10,000, about 500,000 to about 5,000, about 500,000 to about 1,000, about 100,000 to about 50,000, about 100,000 to about 10,000, about 100,000 to about 5,000, about 100,000 to about 1,000, about 50,000 to about 10,000, about 50,000 to about 5,000, about 50,000 to about 1,000, about to about 5,000, about 10,000 to about 1,000, or about 5,000 to about 1,000. In some cases, a total number of an antigen per a single non-cellular substrate may be at least about 50,000,000, about about 5,000,000, about 1,000,000, about 500,000, about 100,000, about 50,000, about about 5,000, or about 1,000. In some cases, a total number of an antigen per a single non-cellular substrate may be at least at least about 50,000,000, about 10,000,000, about 5,000,000, about 1,000,000, about 500,000, about 100,000, about 50,000, about 10,000, or about 5,000. In some cases, a total number of an antigen per a single non-cellular substrate may be at least at most about 10,000,000, about 5,000,000, about 1,000,000, about 500,000, about 100,000, about 50,000, about 10,000, about or about 1,000.

In implementing any one of the methods and systems provided in the present disclosure, an antigen can be coupled to a non-cellular substrate (e.g., a particle, such as a gold nanoparticle, silver nanoparticle, diamond nanoparticle, graphene nanoparticle, carbon nanotube, ceramic nanoparticle, etc.). A coupling may be covalent, e.g., an antigen may be covalently coupled to a non-cellular substrate. Alternatively, an antigen may not and need not be covalently coupled to a non-cellular substrate. In such cases, an antigen may be non-covalently (e.g., via ionic boning and/or hydrogen bonding) coupled to a non-cellular substrate. In some cases, an antigen may be coupled to a non-cellular substrate via a linker. A linker can be of any length, can include carbon atoms and/or heteroatoms, can include linear and/or cyclic moieties, can be branched or unbranched, and can be substituted or unsubstituted. In some examples, a linker may be a polymeric material (e.g., a peptidic linker or polyethylene glycol (PEG)). Alternatively or in addition to, a linker may be a conjugating moiety, such as, for example, a moiety derived from a click chemistry reaction (e.g., triazole, diazole, diazine, sulfide bond, maleimide ring, succinimide ring, ester, amide). Examples of a coupling between a molecule and a substrate (e.g., an antibody polypeptide coupled to a nanoparticle) and use of a linker and/or conjugating moieties can be found in U.S. Patent Application No. 2018/0133343, which is incorporated herein by reference in its entirety.

IV. Chimeric receptor In some cases, the chimeric receptor or chimeric receptor polypeptide (receptor) as disclosed herein can be operatively coupled to a chimeric adaptor polypeptide (adaptor). In some cases, the receptor and the adaptor can be configured to form a complex (e.g., a signaling complex) upon binding of the ligand to the receptor (e.g., upon contacting the cell comprising the receptor with the ligand) and/or upon the receptor modification. The adaptor can be a transmembrane protein. Alternatively, the adaptor can be an intracellular protein. In some cases, the adaptor can be signaling protein of the receptor signaling pathway that is recruited towards the receptor upon the receptor modification.

In some cases, the complexation of the receptor and the adaptor can be direct and/or indirect. In a direct complexation, one of the receptor and the adaptor can be configured to directly bind (e.g., via covalent and/or non-covalent interactions) to the other of the receptor and the adaptor. In some examples, one of the receptor and the adaptor can comprise a binding domain (e.g., a polypeptide sequence) configured to bind to at least a portion (e.g., an intracellular portion) of the other of the receptor and the adaptor. In an indirect complexation, the receptor and the adaptor can be configured to be brought closer to each other (e.g., one is recruited towards the other) without any direct binding upon the receptor modification, relative to without the receptor modification. In some examples, the receptor can comprise a chimeric antigen receptor (CAR) or a modified immune cell receptor (e.g., a modified T cell receptor or “TCR”), and the adaptor can comprise at least a portion of Linker for activation of T cells (LAT) that is recruited as part of a signaling cascade of the receptor upon the receptor modification.

In some cases, one of the receptor and the adaptor can comprise a gene modulating polypeptide comprising the actuator moiety linked to a cleavage recognition site, and the other of the receptor and the adaptor can comprise a cleavage moiety configured to cleave the cleavage recognition site to release the actuator moiety from the GMP. In some examples, the cleaving of the cleavage recognition site by the cleavage moiety can occur upon a direct complexation between the receptor and the adaptor. In some examples, the cleaving of the cleavage recognition site by the cleavage moiety can occur upon an indirect complexation between the receptor and the adaptor. Upon the receptor confirmation, the receptor and the adaptor can be recruited towards each other, such that the cleavage moiety can cleave the actuator moiety from the GMP, thereby to activate the actuator moiety to regulate expression or activity of the endogenous protein (e.g., cytokine, immune checkpoint inhibitor, etc.), as disclosed herein.

In some cases, the chimeric receptor polypeptide (receptor) as disclosed herein can be operatively coupled to a first chimeric adaptor polypeptide (a first adaptor) and a second chimeric adaptor polypeptide (a second adaptor). In some cases, the first adaptor and the second adaptor can be signaling proteins of the receptor signaling pathway that are recruited towards the receptor or towards another signaling protein of the receptor signaling pathway upon the receptor modification. In some examples, the first adaptor and the second adaptor can be recruited towards each other upon the receptor modification. As disclosed herein, the first adaptor and the second adaptor can form a complex via a direct binding. Alternatively, the first adaptor and the second adaptor can form a complex via an indirect binding (e.g., in the vicinity of each other). In some cases, a first adaptor can comprise the GMP (comprising the actuator moiety linked to the cleavage recognition site) and a second adaptor can comprise the cleavage moiety, as disclosed herein. Upon the receptor confirmation, the first adaptor and the second adaptor can be recruited towards each other, such that the cleavage moiety can cleave the actuator moiety from the GMP, thereby to activate the actuator moiety to regulate expression or activity of the endogenous protein (e.g., cytokine, immune checkpoint inhibitor, etc.).

In some cases, one of the first and second adaptors can comprise a gene modulating polypeptide comprising the actuator moiety linked to a cleavage recognition site, and the other of the first and second adaptors can comprise a cleavage moiety configured to cleave the cleavage recognition site to release the actuator moiety from the GMP. In some examples, the cleaving of the cleavage recognition site by the cleavage moiety can occur upon a direct complexation between the first and second adaptors. In some examples, the cleaving of the cleavage recognition site by the cleavage moiety can occur upon an indirect complexation between the first and second adaptors.

In some cases, the receptor as disclosed herein can undergo a receptor modification including a conformational change or chemical modification (e.g., phosphorylation or dephosphorylation) upon binding to the ligand.

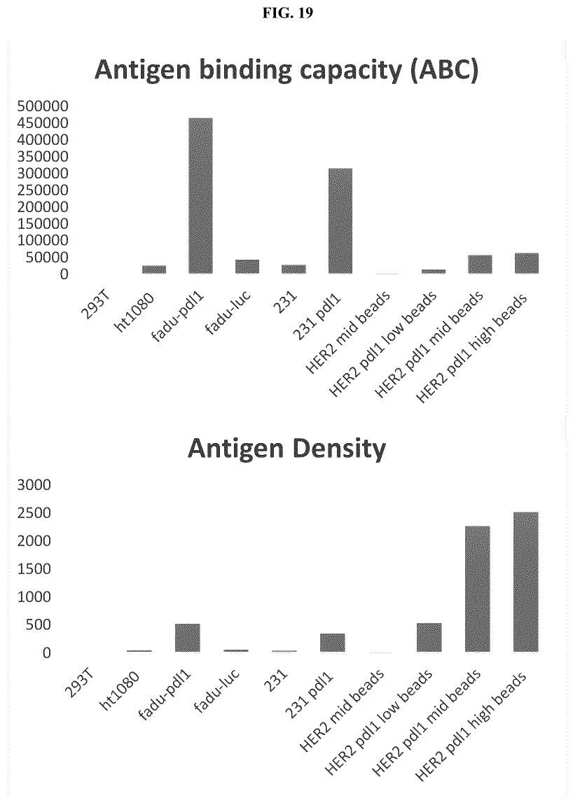

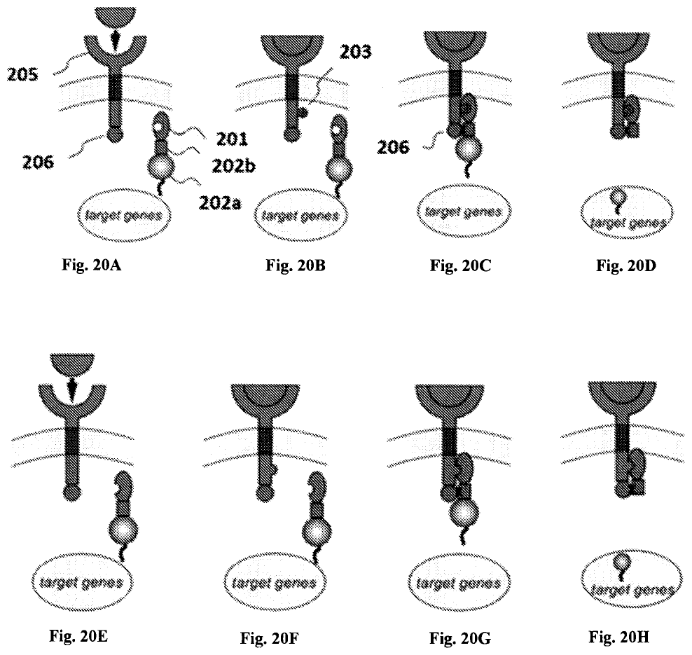

FIGS. 20A-20D illustrate schematically the release of an actuator moiety from a GMP. FIG. 20A shows the binding of an antigen to a transmembrane chimeric receptor polypeptide. The transmembrane chimeric receptor polypeptide comprises an extracellular region having an antigen interacting domain 205 and an intracellular region comprising a cleavage moiety 206. The cleavage moiety can be complexed with the receptor or linked, for example by a peptide bond and/or peptide linker, to the receptor. The GMP forms a portion of the chimeric adaptor polypeptide. The GMP, linked to a receptor binding moiety 201, includes an actuator moiety 202a linked to a cleavage recognition site 202b. In response to antigen binding, the receptor is modified by phosphorylation 203 in the intracellular region of the receptor (FIG. 20B). Following receptor modification (e.g., phosphorylation), the chimeric adaptor polypeptide is recruited to the receptor as shown in FIG. 20C. The receptor comprises a cleavage moiety 206. When in proximity to the cleavage recognition site, the cleavage moiety can cleave the recognition site to release the actuator moiety from the GMP as shown in FIG. 20D. Upon release, the actuator moiety can enter the nucleus to regulate the expression and/or activity of a target gene or edit a nucleic acid sequence. FIGS. 20E-20H show an analogous system wherein receptor modification comprises a conformational change. In some embodiments, the chimeric adaptor protein is tethered to the membrane (e.g., as a membrane bound protein).

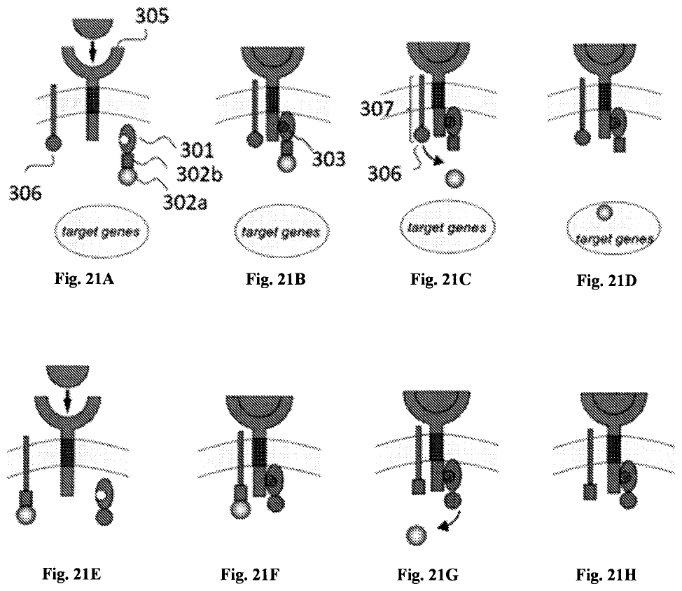

FIGS. 21A-21D illustrate schematically the release of an actuator moiety from a GMP. FIG. 21A shows the binding of an antigen to a transmembrane chimeric receptor polypeptide. The transmembrane chimeric receptor polypeptide comprises an extracellular region having an antigen interacting domain 305 and an intracellular region. The GMP, comprising an actuator moiety linked to a cleavage recognition site, forms a portion of a chimeric adaptor polypeptide. The cleavage recognition site 302b is flanked by the receptor binding moiety 301 and the actuator moiety 302a. In response to antigen binding, the receptor is modified by phosphorylation 303 in the intracellular region (FIG. 21B). Following receptor modification (e.g., phosphorylation), the chimeric adaptor polypeptide is recruited to the receptor as shown in FIG. 21B. A second adaptor polypeptide 307 comprising a cleavage moiety 306 is also recruited to the modified receptor (FIG. 21C). The cleavage moiety may be complexed with the second adaptor polypeptide or linked, for example by a peptide bond and/or peptide linker, to the adaptor. When in proximity to the cleavage recognition site, the cleavage moiety can cleave the recognition site to release the actuator moiety from the GMP as shown in FIG. 21D. Upon release, the actuator moiety can enter the nucleus to regulate the expression and/or activity of a target gene or edit a nucleic acid sequence. FIGS. 21E-21H show an analogous system wherein receptor modification comprises a conformational change. In some embodiments, the chimeric adaptor polypeptide is tethered to the membrane (e.g., as a membrane bound protein). In some embodiments, the second adaptor polypeptide is tethered to the membrane (e.g., as a membrane bound protein).

In some cases, the chimeric receptor polypeptide (receptor) can comprise a ligand binding domain, a transmembrane domain, and a signaling domain. The signaling domain may activate a signaling pathway of the cell upon binding of a ligand to the ligand binding domain. The cell can further comprise an expression cassette comprising a polynucleotide sequence encoding an actuator moiety as disclosed herein (e.g., a GMP comprising the actuator moiety) placed under control of a promoter. The actuator moiety can comprise a heterologous endonuclease. The promoter can be activated to drive expression of the actuator moiety upon binding of the ligand to the ligand binding domain. The expressed actuator moiety can complex with a target gene encoding the endogenous protein as disclosed herein to regulate expression or activity of the endogenous protein. The promoter can comprise an endogenous promoter of the cell. The endogenous promoter can be activated upon binding of the ligand to the ligand binding domain of the receptor.

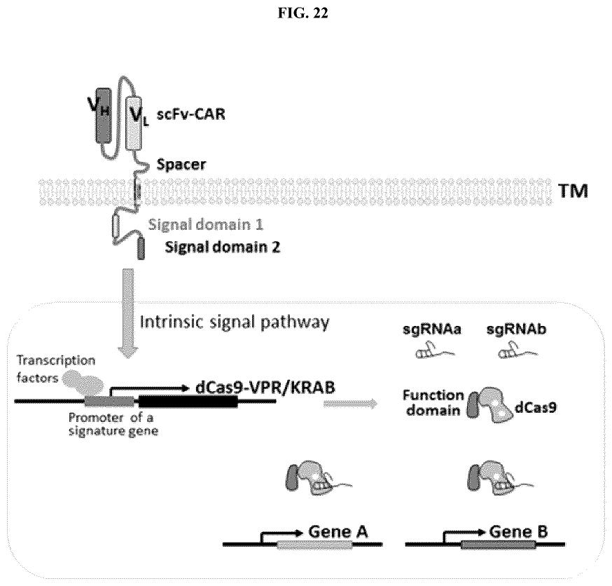

FIG. 22 illustrates an illustrative system comprising a transmembrane receptor useful for regulating expression of at least one target gene. Upon binding of a ligand with a chimeric receptor polypeptide (e.g., scFv-CAR), an intrinsic signal transduction pathway is activated, resulting in the recruitment of at least one cellular transcription factor (e.g., endogenous transcription factor) to the promoter region of an endogenous gene (a signature gene) at its natural locus. An actuator moiety coding sequence (e.g., a GMP coding sequence comprising an actuator moiety coding sequence) is integrated into the genome and is placed under the control of the promoter of the signature gene. Transcriptional activation of the promoter results in expression of the actuator moiety (e.g., comprising a dCas linked to a transcriptional activator (e.g., VPR) or a transcription repressor (e.g., KRAB). The expressed actuator moiety, upon complexing with a guide RNA (e.g., sgRNAa, sgRNAb) (e.g., constitutively or conditionally expressed), can regulate (activate or suppress) the expression of a target gene as disclosed herein.

In some cases, the chimeric receptor or chimeric receptor polypeptide (receptor) as disclosed herein can be a chimeric antigen receptor (CAR) and/or a modified T cell receptor (TCR).

In some cases, a CAR as disclosed herein can be a first-, second-, third-, or fourth-generation CAR system, a functional variant thereof, or any combination thereof. First-generation CARs (e.g., CD19R or CD19CAR) include an antigen binding domain with specificity for a particular antigen (e.g., an antibody or antigen-binding fragment thereof such as an scFv, a Fab fragment, a VHH domain, or a VH domain of a heavy-chain only antibody), a transmembrane domain derived from an adaptive immune receptor (e.g., the transmembrane domain from the CD28 receptor), and a signaling domain derived from an adaptive immune receptor (e.g., one or more (e.g., three) ITAM domains derived from the intracellular region of the CD3 receptor or FcεRIγ). Second-generation CARs modify the first-generation CAR by addition of a co-stimulatory domain to the intracellular signaling domain portion of the CAR (e.g., derived from co-stimulatory receptors that act alongside T-cell receptors such as CD28, CD137/4-1BB, and CD134/OX40), which abrogates the need for administration of a co-factor (e.g., IL-2) alongside a first-generation CAR. Third-generation CARs add multiple co-stimulatory domains to the intracellular signaling domain portion of the CAR (e.g., CD3-CD28-OX40, or CD3-CD28-41BB). Fourth-generation CARs modify second- or third-generation CARs by the addition of an activating cytokine (e.g., IL-23, or IL-27) to the intracellular signaling portion of the CAR (e.g., between one or more of the costimulatory domains and the CD3t ITAM domain) or under the control of a CAR-induced promoter (e.g., the NFAT/IL-2 minimal promoter).

In some cases, the plurality of cells as disclosed herein may comprise the chimeric receptor in absence of the adaptor as disclosed herein. For example, the plurality of cells can be a plurality of conventional CAR immune cells (e.g., CAR-T or CAR-NK cells) or a plurality of immune cells expressing a modified TCR.

In various embodiments of the methods and systems herein, a chimeric receptor may be exogenous to a subject cell of the present disclosure. A gene encoding the chimeric receptor may be chromosomal (e.g., integrated into a nuclear chromosome and/or mitochondrial chromosome) or epichromosomal.

In some embodiments, a receptor of the systems and methods in the present disclosure may be a chimeric receptor. The chimeric receptor may be a transmembrane receptor. In some cases, the ligand binding domain of the chimeric receptor may be heterologous to the cell. In some cases, the chimeric receptor may comprise a CAR. The CAR may comprise at least a portion of a Notch receptor, a G-protein coupled receptor (GPCR), an integrin receptor, a cadherin receptor, a receptor tyrosine kinase, a death receptor, an immune receptor. The immune receptor may comprise a T cell receptor (TCR). The TCR may comprise TCRA, TCRB, TCRG, and/or TCRD. The TCR may comprise a co-receptor of TCR, such as, CD3, CD4, and/or CD8. CD3 may comprise CD3E, CD3D, CD3G, and/or CD3Z. The CAR may comprise at least a portion of an intracellular portion of a TCR complex. As an alternative, the CAR may not comprise any portion of an intracellular portion of the TCR complex. The CAR may comprise one or more signaling capabilities of the TCR complex. As an alternative, the CAR may not comprise any signaling capability of the TCR complex.

In some cases, the chimeric receptor of a subject system can comprise at least a portion of an endogenous receptor, or any derivative, variant or fragment thereof. The chimeric receptor can bind specifically to at least one antigen (e.g., at least one ligand), for example via an antigen interacting domain (also referred to herein as an “extracellular sensor domain”). The chimeric receptor can, in response to ligand binding, undergo a modification such as a conformational change and/or chemical modification. Such modification(s) can recruit to the chimeric receptor binding partners (e.g., partners such as proteins) including, but not limited to, signaling proteins involved in signaling events and various cellular processes. Signaling proteins, for example, can be involved in regulating (e.g., activating and/or de-activating) a cellular response such as programmed changes in gene expression via translational regulation; transcriptional regulation; and epigenetic modification including the regulation of methylation, acetylation, phosphorylation, ubiquitylation, sumoylation, ribosylation, and citrullination. Conformational changes of the chimeric receptor can expose one or more regions of the chimeric receptor which was previously not exposed, and the exposed region can recruit and/or bind signaling protein(s). Chemical modifications on a receptor, for example phosphorylation and/or dephosphorylation (e.g., at tyrosine, serine, threonine, and/or any other suitable amino acid residue), can also recruit signaling proteins involved in regulating intracellular processes. Signaling proteins can bind directly to a receptor or indirectly to a receptor, for example as part of a larger complex.

In some cases, the chimeric receptor can comprise at least a portion of a transmembrane receptor. The transmembrane receptor may detect at least one signal (i.e., ligand), such as a small molecule, ion, or protein, from the surrounding environment (e.g., extracellular and/or intracellular environment) and can initiate a cellular response via at least one signaling cascade involving additional proteins and signaling molecules. The transmembrane receptor may translocate from one region of a cell to another, for example from the plasma membrane or cytoplasm to the nucleus and vice versa. Such translocation can be conditional upon ligand binding to the transmembrane receptor. Examples of the transmembrane receptor may include, but are not limited to, Notch receptors; G-protein coupled receptors (GPCRs); integrin receptors; cadherin receptors; catalytic receptors including receptors possessing enzymatic activity and receptors, which, rather than possessing intrinsic enzymatic activity, act by stimulating non-covalently associated enzymes (e.g., kinases); death receptors such as members of the tumor necrosis factor receptor (TNFR) superfamily; and immune receptors.

In some cases, the chimeric receptor may comprise a Notch, or any derivative, variant or fragment thereof, selected from Notch1, Notch2, Notch3, and Notch4 or any homolog thereof.

In some cases, the chimeric receptor may comprise a GPCR, or any derivative, variant or fragment thereof, selected from Class A Orphans; Class B Orphans; Class C Orphans; taste receptors, type 1; taste receptors, type 2; 5-hydroxytryptamine receptors; acetylcholine receptors (muscarinic); adenosine receptors; adhesion class GPCRs; adrenoceptors; angiotensin receptors; apelin receptor; bile acid receptor; bombesin receptors; bradykinin receptors; calcitonin receptors; calcium-sensing receptors; cannabinoid receptors; chemerin receptor; chemokine receptors; cholecystokinin receptors; class Frizzled GPCRs (e.g., Wnt receptors); complement peptide receptors; corticotropin-releasing factor receptors; dopamine receptors; endothelin receptors; G protein-coupled estrogen receptor; formylpeptide receptors; free fatty acid receptors; GABAB receptors; galanin receptors; ghrelin receptor; glucagon receptor family; glycoprotein hormone receptors; gonadotrophin-releasing hormone receptors; GPR18, GPR55 and GPR119; histamine receptors; hydroxycarboxylic acid receptors; kisspeptin receptor; leukotriene receptors; lysophospholipid (LPA) receptors; lysophospholipid (SIP) receptors; melanin-concentrating hormone receptors; melanocortin receptors; melatonin receptors; metabotropic glutamate receptors; motilin receptor; neuromedin U receptors; neuropeptide FF/neuropeptide AF receptors; neuropeptide S receptor; neuropeptide W/neuropeptide B receptors; neuropeptide Y receptors; neurotensin receptors; opioid receptors; orexin receptors; oxoglutarate receptor; P2Y receptors; parathyroid hormone receptors; platelet-activating factor receptor; prokineticin receptors; prolactin-releasing peptide receptor; prostanoid receptors; proteinase-activated receptors; QRFP receptor; relaxin family peptide receptors; somatostatin receptors; succinate receptor; tachykinin receptors; thyrotropin-releasing hormone receptors; trace amine receptor; urotensin receptor; vasopressin and oxytocin receptors; VIP and PACAP receptors.

In some cases, the chimeric receptor may comprise a GPCR selected from the group consisting of: 5-hydroxytryptamine (serotonin) receptor 1A (HTR1A), 5-hydroxytryptamine (serotonin) receptor 1B (HTR1B), 5-hydroxytryptamine (serotonin) receptor 1D (HTR1D), 5-hydroxytryptamine (serotonin) receptor 1E (HTR1E), 5-hydroxytryptamine (serotonin) receptor 1F (HTR1F), 5-hydroxytryptamine (serotonin) receptor 2A (HTR2A), 5-hydroxytryptamine (serotonin) receptor 2B (HTR2B), 5-hydroxytryptamine (serotonin) receptor 2C (HTR2C), 5-hydroxytryptamine (serotonin) receptor 4 (HTR4), 5-hydroxytryptamine (serotonin) receptor 5A (HTR5A), 5-hydroxytryptamine (serotonin) receptor 5B (HTR5BP), 5-hydroxytryptamine (serotonin) receptor 6 (HTR6), 5-hydroxytryptamine (serotonin) receptor 7, adenylate cyclase-coupled (HTR7), cholinergic receptor, muscarinic 1 (CHRM1), cholinergic receptor, muscarinic 2 (CHRM2), cholinergic receptor, muscarinic 3 (CHRM3), cholinergic receptor, muscarinic 4 (CHRM4), cholinergic receptor, muscarinic 5 (CHRM5), adenosine A1 receptor (ADORA1), adenosine A2a receptor (ADORA2A), adenosine A2b receptor (ADORA2B), adenosine A3 receptor (ADORA3), adhesion G protein-coupled receptor A1 (ADGRA1), adhesion G protein-coupled receptor A2 (ADGRA2), adhesion G protein-coupled receptor A3 (ADGRA3), adhesion G protein-coupled receptor B1 (ADGRB1), adhesion G protein-coupled receptor B2 (ADGRB2), adhesion G protein-coupled receptor B3 (ADGRB3), cadherin EGF LAG seven-pass G-type receptor 1 (CELSR1), cadherin EGF LAG seven-pass G-type receptor 2 (CELSR2), cadherin EGF LAG seven-pass G-type receptor 3 (CELSR3), adhesion G protein-coupled receptor D1 (ADGRD1), adhesion G protein-coupled receptor D2 (ADGRD2), adhesion G protein-coupled receptor E1 (ADGRE1), adhesion G protein-coupled receptor E2 (ADGRE2), adhesion G protein-coupled receptor E3 (ADGRE3), adhesion G protein-coupled receptor E4 (ADGRE4P), adhesion G protein-coupled receptor E5 (ADGRE5), adhesion G protein-coupled receptor F1 (ADGRF1), adhesion G protein-coupled receptor F2 (ADGRF2), adhesion G protein-coupled receptor F3 (ADGRF3), adhesion G protein-coupled receptor F4 (ADGRF4), adhesion G protein-coupled receptor F5 (ADGRF5), adhesion G protein-coupled receptor G1 (ADGRG1), adhesion G protein-coupled receptor G2 (ADGRG2), adhesion G protein-coupled receptor G3 (ADGRG3), adhesion G protein-coupled receptor G4 (ADGRG4), adhesion G protein-coupled receptor G5 (ADGRG5), adhesion G protein-coupled receptor G6 (ADGRG6), adhesion G protein-coupled receptor G7 (ADGRG7), adhesion G protein-coupled receptor L1 (ADGRL1), adhesion G protein-coupled receptor L2 (ADGRL2), adhesion G protein-coupled receptor L3 (ADGRL3), adhesion G protein-coupled receptor L4 (ADGRL4), adhesion G protein-coupled receptor V1 (ADGRV1), adrenoceptor alpha 1A (ADRA1A), adrenoceptor alpha 1B (ADRA1B), adrenoceptor alpha 1D (ADRA1D), adrenoceptor alpha 2A (ADRA2A), adrenoceptor alpha 2B (ADRA2B), adrenoceptor alpha 2C (ADRA2C), adrenoceptor beta 1 (ADRB1), adrenoceptor beta 2 (ADRB2), adrenoceptor beta 3 (ADRB3), angiotensin II receptor type 1 (AGTR1), angiotensin II receptor type 2 (AGTR2), apelin receptor (APLNR), G protein-coupled bile acid receptor 1 (GPBAR1), neuromedin B receptor (NMBR), gastrin releasing peptide receptor (GRPR), bombesin like receptor 3 (BRS3), bradykinin receptor B1 (BDKRB1), bradykinin receptor B2 (BDKRB2), calcitonin receptor (CALCR), calcitonin receptor like receptor (CALCRL), calcium sensing receptor (CASR), G protein-coupled receptor, class C (GPRC6A), cannabinoid receptor 1 (brain) (CNR1), cannabinoid receptor 2 (CNR2), chemerin chemokine-like receptor 1 (CMKLR1), chemokine (C-C motif) receptor 1 (CCR1), chemokine (C-C motif) receptor 2 (CCR2), chemokine (C-C motif) receptor 3 (CCR3), chemokine (C-C motif) receptor 4 (CCR4), chemokine (C-C motif) receptor 5 (gene/pseudogene) (CCR5), chemokine (C-C motif) receptor 6 (CCR6), chemokine (C-C motif) receptor 7 (CCR7), chemokine (C-C motif) receptor 8 (CCR8), chemokine (C-C motif) receptor 9 (CCR9), chemokine (C-C motif) receptor 10 (CCR10), chemokine (C-X-C motif) receptor 1 (CXCR1), chemokine (C-X-C motif) receptor 2 (CXCR2), chemokine (C-X-C motif) receptor 3 (CXCR3), chemokine (C-X-C motif) receptor 4 (CXCR4), chemokine (C-X-C motif) receptor 5 (CXCR5), chemokine (C-X-C motif) receptor 6 (CXCR6), chemokine (C-X3-C motif) receptor 1 (CX3CR1), chemokine (C motif) receptor 1 (XCR1), atypical chemokine receptor 1 (Duffy blood group) (ACKR1), atypical chemokine receptor 2 (ACKR2), atypical chemokine receptor 3 (ACKR3), atypical chemokine receptor 4 (ACKR4), chemokine (C-C motif) receptor-like 2 (CCRL2), cholecystokinin A receptor (CCKAR), cholecystokinin B receptor (CCKBR), G protein-coupled receptor 1 (GPR1), bombesin like receptor 3 (BRS3), G protein-coupled receptor 3 (GPR3), G protein-coupled receptor 4 (GPR4), G protein-coupled receptor 6 (GPR6), G protein-coupled receptor 12 (GPR12), G protein-coupled receptor 15 (GPR15), G protein-coupled receptor 17 (GPR17), G protein-coupled receptor 18 (GPR18), G protein-coupled receptor 19 (GPR19), G protein-coupled receptor 20 (GPR20), G protein-coupled receptor 21 (GPR21), G protein-coupled receptor 22 (GPR22), G protein-coupled receptor 25 (GPR25), G protein-coupled receptor 26 (GPR26), G protein-coupled receptor 27 (GPR27), G protein-coupled receptor 31 (GPR31), G protein-coupled receptor 32 (GPR32), G protein-coupled receptor 33 (gene/pseudogene) (GPR33), G protein-coupled receptor 34 (GPR34), G protein-coupled receptor 35 (GPR35), G protein-coupled receptor 37 (endothelin receptor type B-like) (GPR37), G protein-coupled receptor 37 like 1 (GPR37L1), G protein-coupled receptor 39 (GPR39), G protein-coupled receptor 42 (gene/pseudogene) (GPR42), G protein-coupled receptor 45 (GPR45), G protein-coupled receptor 50 (GPR50), G protein-coupled receptor 52 (GPR52), G protein-coupled receptor 55 (GPR55), G protein-coupled receptor 61 (GPR61), G protein-coupled receptor 62 (GPR62), G protein-coupled receptor 63 (GPR63), G protein-coupled receptor 65 (GPR65), G protein-coupled receptor 68 (GPR68), G protein-coupled receptor 75 (GPR75), G protein-coupled receptor 78 (GPR78), G protein-coupled receptor 79 (GPR79), G protein-coupled receptor 82 (GPR82), G protein-coupled receptor 83 (GPR83), G protein-coupled receptor 84 (GPR84), G protein-coupled receptor 85 (GPR85), G protein-coupled receptor 87 (GPR87), G protein-coupled receptor 88 (GPR88), G protein-coupled receptor 101 (GPR101), G protein-coupled receptor 119 (GPR119), G protein-coupled receptor 132 (GPR132), G protein-coupled receptor 135 (GPR135), G protein-coupled receptor 139 (GPR139), G protein-coupled receptor 141 (GPR141), G protein-coupled receptor 142 (GPR142), G protein-coupled receptor 146 (GPR146), G protein-coupled receptor 148 (GPR148), G protein-coupled receptor 149 (GPR149), G protein-coupled receptor 150 (GPR150), G protein-coupled receptor 151 (GPR151), G protein-coupled receptor 152 (GPR152), G protein-coupled receptor 153 (GPR153), G protein-coupled receptor 160 (GPR160), G protein-coupled receptor 161 (GPR161), G protein-coupled receptor 162 (GPR162), G protein-coupled receptor 171 (GPR171), G protein-coupled receptor 173 (GPR173), G protein-coupled receptor 174 (GPR174), G protein-coupled receptor 176 (GPR176), G protein-coupled receptor 182 (GPR182), G protein-coupled receptor 183 (GPR183), leucine-rich repeat containing G protein-coupled receptor 4 (LGR4), leucine-rich repeat containing G protein-coupled receptor 5 (LGR5), leucine-rich repeat containing G protein-coupled receptor 6 (LGR6), MASI proto-oncogene (MAS 1), MASI proto-oncogene like (MAS1L), MAS related GPR family member D (MRGPRD), MAS related GPR family member E (MRGPRE), MAS related GPR family member F (MRGPRF), MAS related GPR family member G (MRGPRG), MAS related GPR family member X1 (MRGPRX1), MAS related GPR family member X2 (MRGPRX2), MAS related GPR family member X3 (MRGPRX3), MAS related GPR family member X4 (MRGPRX4), opsin 3 (OPN3), opsin 4 (OPN4), opsin 5 (OPNS), purinergic receptor P2Y (P2RY8), purinergic receptor P2Y (P2RY10), trace amine associated receptor 2 (TAAR2), trace amine associated receptor 3 (gene/pseudogene) (TAAR3), trace amine associated receptor 4 (TAAR4P), trace amine associated receptor 5 (TAAR5), trace amine associated receptor 6 (TAAR6), trace amine associated receptor 8 (TAAR8), trace amine associated receptor 9 (gene/pseudogene) (TAAR9), G protein-coupled receptor 156 (GPR156), G protein-coupled receptor 158 (GPR158), G protein-coupled receptor 179 (GPR179), G protein-coupled receptor, class C (GPRCSA), G protein-coupled receptor, class C (GPRCSB), G protein-coupled receptor, class C (GPRCSC), G protein-coupled receptor, class C (GPRCSD), frizzled class receptor 1 (FZD1), frizzled class receptor 2 (FZD2), frizzled class receptor 3 (FZD3), frizzled class receptor 4 (FZD4), frizzled class receptor 5 (FZD5), frizzled class receptor 6 (FZD6), frizzled class receptor 7 (FZD7), frizzled class receptor 8 (FZD8), frizzled class receptor 9 (FZD9), frizzled class receptor 10 (FZD10), smoothened, frizzled class receptor (SMO), complement component 3a receptor 1 (C3AR1), complement component 5a receptor 1 (C5AR1), complement component 5a receptor 2 (C5AR2), corticotropin releasing hormone receptor 1 (CRHR1), corticotropin releasing hormone receptor 2 (CRHR2), dopamine receptor D1 (DRD1), dopamine receptor D2 (DRD2), dopamine receptor D3 (DRD3), dopamine receptor D4 (DRD4), dopamine receptor D5 (DRD5), endothelin receptor type A (EDNRA), endothelin receptor type B (EDNRB), formyl peptide receptor 1 (FPR1), formyl peptide receptor 2 (FPR2), formyl peptide receptor 3 (FPR3), free fatty acid receptor 1 (FFAR1), free fatty acid receptor 2 (FFAR2), free fatty acid receptor 3 (FFAR3), free fatty acid receptor 4 (FFAR4), G protein-coupled receptor 42 (gene/pseudogene) (GPR42), gamma-aminobutyric acid (GABA) B receptor, 1 (GABBR1), gamma-aminobutyric acid (GABA) B receptor, 2 (GABBR2), galanin receptor 1 (GALR1), galanin receptor 2 (GALR2), galanin receptor 3 (GALR3), growth hormone secretagogue receptor (GHSR), growth hormone releasing hormone receptor (GHRHR), gastric inhibitory polypeptide receptor (GIPR), glucagon like peptide 1 receptor (GLP1R), glucagon-like peptide 2 receptor (GLP2R), glucagon receptor (GCGR), secretin receptor (SCTR), follicle stimulating hormone receptor (FSHR), luteinizing hormone/choriogonadotropin receptor (LHCGR), thyroid stimulating hormone receptor (TSHR), gonadotropin releasing hormone receptor (GNRHR), gonadotropin releasing hormone receptor 2 (pseudogene) (GNRHR2), G protein-coupled receptor 18 (GPR18), G protein-coupled receptor 55 (GPR55), G protein-coupled receptor 119 (GPR119), G protein-coupled estrogen receptor 1 (GPER1), histamine receptor H1 (HRH1), histamine receptor H2 (HRH2), histamine receptor H3 (HRH3), histamine receptor H4 (HRH4), hydroxycarboxylic acid receptor 1 (HCAR1), hydroxycarboxylic acid receptor 2 (HCAR2), hydroxycarboxylic acid receptor 3 (HCAR3), KISS1 receptor (KISS1R), leukotriene B4 receptor (LTB4R), leukotriene B4 receptor 2 (LTB4R2), cysteinyl leukotriene receptor 1 (CYSLTR1), cysteinyl leukotriene receptor 2 (CYSLTR2), oxoeicosanoid (OXE) receptor 1 (OXER1), formyl peptide receptor 2 (FPR2), lysophosphatidic acid receptor 1 (LPAR1), lysophosphatidic acid receptor 2 (LPAR2), lysophosphatidic acid receptor 3 (LPAR3), lysophosphatidic acid receptor 4 (LPAR4), lysophosphatidic acid receptor 5 (LPAR5), lysophosphatidic acid receptor 6 (LPAR6), sphingosine-1-phosphate receptor 1 (S1PR1), sphingosine-1-phosphate receptor 2 (S1PR2), sphingosine-1-phosphate receptor 3 (S1PR3), sphingosine-1-phosphate receptor 4 (S1PR4), sphingosine-1-phosphate receptor 5 (S1PR5), melanin concentrating hormone receptor 1 (MCHR1), melanin concentrating hormone receptor 2 (MCHR2), melanocortin 1 receptor (alpha melanocyte stimulating hormone receptor) (MC1R), melanocortin 2 receptor (adrenocorticotropic hormone) (MC2R), melanocortin 3 receptor (MC3R), melanocortin 4 receptor (MC4R), melanocortin 5 receptor (MC5R), melatonin receptor 1A (MTNR1A), melatonin receptor 1B (MTNR1B), glutamate receptor, metabotropic 1 (GRM1), glutamate receptor, metabotropic 2 (GRM2), glutamate receptor, metabotropic 3 (GRM3), glutamate receptor, metabotropic 4 (GRM4), glutamate receptor, metabotropic 5 (GRM5), glutamate receptor, metabotropic 6 (GRM6), glutamate receptor, metabotropic 7 (GRM7), glutamate receptor, metabotropic 8 (GRM8), motilin receptor (MLNR), neuromedin U receptor 1 (NMUR1), neuromedin U receptor 2 (NMUR2), neuropeptide FF receptor 1 (NPFFR1), neuropeptide FF receptor 2 (NPFFR2), neuropeptide S receptor 1 (NPSR1), neuropeptides B/W receptor 1 (NPBWR1), neuropeptides B/W receptor 2 (NPBWR2), neuropeptide Y receptor Y1 (NPY1R), neuropeptide Y receptor Y2 (NPY2R), neuropeptide Y receptor Y4 (NPY4R), neuropeptide Y receptor Y5 (NPY5R), neuropeptide Y receptor Y6 (pseudogene) (NPY6R), neurotensin receptor 1 (high affinity) (NTSR1), neurotensin receptor 2 (NTSR2), opioid receptor, delta 1 (OPRD1), opioid receptor, kappa 1 (OPRK1), opioid receptor, mu 1 (OPRM1), opiate receptor-like 1 (OPRL1), hypocretin (orexin) receptor 1 (HCRTR1), hypocretin (orexin) receptor 2 (HCRTR2), G protein-coupled receptor 107 (GPR107), G protein-coupled receptor 137 (GPR137), olfactory receptor family 51 subfamily E member 1 (OR51E1), transmembrane protein, adipocyte associated 1 (TPRA1), G protein-coupled receptor 143 (GPR143), G protein-coupled receptor 157 (GPR157), oxoglutarate (alpha-ketoglutarate) receptor 1 (OXGR1), purinergic receptor P2Y (P2RY1), purinergic receptor P2Y (P2RY2), pyrimidinergic receptor P2Y (P2RY4), pyrimidinergic receptor P2Y (P2RY6), purinergic receptor P2Y (P2RY11), purinergic receptor P2Y (P2RY12), purinergic receptor P2Y (P2RY13), purinergic receptor P2Y (P2RY14), parathyroid hormone 1 receptor (PTH1R), parathyroid hormone 2 receptor (PTH2R), platelet-activating factor receptor (PTAFR), prokineticin receptor 1 (PROKR1), prokineticin receptor 2 (PROKR2), prolactin releasing hormone receptor (PRLHR), prostaglandin D2 receptor (DP) (PTGDR), prostaglandin D2 receptor 2 (PTGDR2), prostaglandin E receptor 1 (PTGER1), prostaglandin E receptor 2 (PTGER2), prostaglandin E receptor 3 (PTGER3), prostaglandin E receptor 4 (PTGER4), prostaglandin F receptor (PTGFR), prostaglandin 12 (prostacyclin) receptor (IP) (PTGIR), thromboxane A2 receptor (TBXA2R), coagulation factor II thrombin receptor (F2R), F2R like trypsin receptor 1 (F2RL1), coagulation factor II thrombin receptor like 2 (F2RL2), F2R like thrombin/trypsin receptor 3 (F2RL3), pyroglutamylated RFamide peptide receptor (QRFPR), relaxin/insulin-like family peptide receptor 1 (RXFP1), relaxin/insulin-like family peptide receptor 2 (RXFP2), relaxin/insulin-like family peptide receptor 3 (RXFP3), relaxin/insulin-like family peptide receptor 4 (RXFP4), somatostatin receptor 1 (SSTR1), somatostatin receptor 2 (SSTR2), somatostatin receptor 3 (SSTR3), somatostatin receptor 4 (SSTR4), somatostatin receptor 5 (SSTR5), succinate receptor 1 (SUCNR1), tachykinin receptor 1 (TACR1), tachykinin receptor 2 (TACR2), tachykinin receptor 3 (TACR3), taste 1 receptor member 1 (TAS1R1), taste 1 receptor member 2 (TAS1R2), taste 1 receptor member 3 (TAS1R3), taste 2 receptor member 1 (TAS2R1), taste 2 receptor member 3 (TAS2R3), taste 2 receptor member 4 (TAS2R4), taste 2 receptor member 5 (TAS2R5), taste 2 receptor member 7 (TAS2R7), taste 2 receptor member 8 (TAS2R8), taste 2 receptor member 9 (TAS2R9), taste 2 receptor member 10 (TAS2R10), taste 2 receptor member 13 (TAS2R13), taste 2 receptor member 14 (TAS2R14), taste 2 receptor member 16 (TAS2R16), taste 2 receptor member 19 (TAS2R19), taste 2 receptor member 20 (TAS2R20), taste 2 receptor member 30 (TAS2R30), taste 2 receptor member 31 (TAS2R31), taste 2 receptor member 38 (TAS2R38), taste 2 receptor member 39 (TAS2R39), taste 2 receptor member 40 (TAS2R40), taste 2 receptor member 41 (TAS2R41), taste 2 receptor member 42 (TAS2R42), taste 2 receptor member 43 (TAS2R43), taste 2 receptor member 45 (TAS2R45), taste 2 receptor member 46 (TAS2R46), taste 2 receptor member 50 (TAS2R50), taste 2 receptor member 60 (TAS2R60), thyrotropin-releasing hormone receptor (TRHR), trace amine associated receptor 1 (TAAR1), urotensin 2 receptor (UTS2R), arginine vasopressin receptor 1A (AVPR1A), arginine vasopressin receptor 1B (AVPR1B), arginine vasopressin receptor 2 (AVPR2), oxytocin receptor (OXTR), adenylate cyclase activating polypeptide 1 (pituitary) receptor type I (ADCYAP1R1), vasoactive intestinal peptide receptor 1 (VIPR1), vasoactive intestinal peptide receptor 2 (VIPR2), any derivative thereof, any variant thereof, and any fragment thereof.

The chimeric receptor comprising a GPCR, or any derivative, variant or fragment thereof, may bind an antigen comprising any suitable GPCR ligand, or any derivative, variant or fragment thereof. Non-limiting examples of ligands which can be bound by a GPCR include (-)-adrenaline, (-)-noradrenaline, (lyso)phospholipid mediators, [des-Arg10]kallidin, [des-Arg9]bradykinin, [des-Gln14]ghrelin, [Hyp3]bradykinin, [Leu] enkephalin, [Met]enkephalin, 12-hydroxyheptadecatrienoic acid, 12R-HETE, 12S-HETE, 12S-HPETE, 15S-HETE, 17β-estradiol, 20-hydroxy-LTB4, 2-arachidonoylglycerol, 2-oleoyl-LPA, 3-hydroxyoctanoic acid, 5-hydroxytryptamine, 5-oxo-15-HETE, 5-oxo-ETE, 5-oxo-ETrE, 5-oxo-ODE, 5S-HETE, 5S-HPETE, 7α,25-dihydroxycholesterol, acetylcholine, ACTH, adenosine diphosphate, adenosine, adrenomedullin 2/intermedin, adrenomedullin, amylin, anandamide, angiotensin II, angiotensin III, annexin I, apelin receptor early endogenous ligand, apelin-13, apelin-17, apelin-36, aspirin triggered lipoxin A4, aspirin-triggered resolvin D1, ATP, beta-defensin 4A, big dynorphin, bovine adrenal medulla peptide 8-22, bradykinin, C3a, C5a, Ca2+, calcitonin gene related peptide, calcitonin, cathepsin G, CCK-33, CCK-4, CCK-8, CCL1, CCL11, CCL13, CCL14, CCL15, CCL16, CCL17, CCL19, CCL2, CCL20, CCL21, CCL22, CCL23, CCL24, CCL25, CCL26, CCL27, CCL28, CCL3, CCL4, CCL5, CCL7, CCL8, chemerin, chenodeoxycholic acid, cholic acid, corticotrophin-releasing hormone, CST-17, CX3CL1, CXCL1, CXCL10, CXCL11, CXCL12a, CXCL12β, CXCL13, CXCL16, CXCL2, CXCL3, CXCL5, CXCL6, CXCL7, CXCL8, CXCL9, cysteinyl-leukotrienes (CysLTs), uracil nucleotides, deoxycholic acid, dihydrosphingosine-1-phosphate, dioleoylphosphatidic acid, dopamine, dynorphin A, dynorphin A-(1-13), dynorphin A-(1-8), dynorphin B, endomorphin-1, endothelin-1, endothelin-2, endothelin-3, F2L, Free fatty acids, FSH, GABA, galanin, galanin-like peptide, gastric inhibitory polypeptide, gastrin-17, gastrin-releasing peptide, ghrelin, GHRH, glucagon, glucagon-like peptide 1-(7-36) amide, glucagon-like peptide 1-(7-37), glucagon-like peptide 2, glucagon-like peptide 2-(3-33), GnRH I, GnRH II, GRP-(18-27), hCG, histamine, humanin, INSL3, INSL5, kallidin, kisspeptin-10, kisspeptin-13, kisspeptin-14, kisspeptin-54, kynurenic acid, large neuromedin N, large neurotensin, L-glutamic acid, LH, lithocholic acid, L-lactic acid, long chain carboxylic acids, LPA, LTB4, LTC4, LTD4, LTE4, LXA4, Lys-[Hyp3]-bradykinin, lysophosphatidylinositol, lysophosphatidylserine, Medium-chain-length fatty acids, melanin-concentrating hormone, melatonin, methylcarbamyl PAF, Mg2+, motilin, N-arachidonoylglycine, neurokinin A, neurokinin B, neuromedin B, neuromedin N, neuromedin S-33, neuromedin U-25, neuronostatin, neuropeptide AF, neuropeptide B-23, neuropeptide B-29, neuropeptide FF, neuropeptide S, neuropeptide SF, neuropeptide W-23, neuropeptide W-30, neuropeptide Y, neuropeptide Y-(3-36), neurotensin, nociceptin/orphanin FQ, N-oleoylethanolamide, obestatin, octopamine, orexin-A, orexin-B, Oxysterols, oxytocin, PACAP-27, PACAP-38, PAF, pancreatic polypeptide, peptide YY, PGD2, PGE2, PGF2a, PGI2, PGJ2, PHM, phosphatidylserine, PHV, prokineticin-1, prokineticin-2, prokineticin-23, prosaposin, PrRP-20, PrRP-31, PTH, PTHrP, PTHrP-(1-36), QRFP43, relaxin, relaxin-1, relaxin-3, resolvin D1, resolvin E1, RFRP-1, RFRP-3, R-spondins, secretin, serine proteases, sphingosine 1-phosphate, sphingosylphosphorylcholine, SRIF-14, SRIF-28, substance P, succinic acid, thrombin, thromboxane A2, T1P39, T-kinin, TRH, TSH, tyramine, UDP-glucose, uridine diphosphate, urocortin 1, urocortin 2, urocortin 3, urotensin II-related peptide, urotensin-II, vasopressin, VIP, Wnt, Wnt-1, Wnt-10a, Wnt-10b, Wnt-11, Wnt-16, Wnt-2, Wnt-2b, Wnt-3, Wnt-3a, Wnt-4, Wnt-5a, Wnt-5b, Wnt-6, Wnt-7a, Wnt-7b, Wnt-8a, Wnt-8b, Wnt-9a, Wnt-9b, XCL1, XCL2, Zn2+, α-CGRP, α-ketoglutaric acid, α-MSH, α-neoendorphin, β-alanine, β-CGRP, β-D-hydroxybutyric acid, β-endorphin, β-MSH, β-neoendorphin, β-phenylethylamine, and γ-MSH.

In some cases, the chimeric receptor may comprise an integrin receptor a subunit, or any derivative, variant or fragment thereof, selected from the group consisting of: α1, α2, α3, α4, α5, α6, α7, α8, α9, α10, α11, αV, αL, αM, αX, αD, αE, and αIIb. In some embodiments, a chimeric receptor polypeptide comprises an integrin receptor β subunit, or any derivative, variant or fragment thereof, selected from the group consisting of: β1, β2, β3, β4, β5, β6, β7, and β8. Chimeric receptor polypeptides comprising an a subunit, a β subunit, or any derivative, variant or fragment thereof, can heterodimerize (e.g., a subunit dimerizing with a β subunit) to form an integrin receptor, or any derivative, variant or fragment thereof. Non-limiting examples of integrin receptors include an α1β1, α2β1, α3β1, α4β1, α5β1, α6β1, α7β1, α8β1, α9β1, α10β1, αVβ1, αLβ1, αMβ1, αXβ1, αDβ1, αIIbβ1, αEβ1, α1β2, α2β2, α3β2, α4β2, α5β2, α6β2, α7β2, α8β2, α9β2, α10β2, αVβ2, αLβ2, αMβ2, αXβ2, αDβ2, αIIbβ2, αEβ2, α1β3, α2β3, α3β3, α4β3, α5β3, α6β3, α7β3, α8β3, α9β3, α10β3, αVβ3, αLβ3, αMβ3, αXβ3, αDβ3, αIIbβ3, αEβ3, α1β4, α2β4, α3β4, α4β4, α5β4, α6β4, α7β4, α8β4, α9β4, α10β4, αVβ4, αLβ4, αMβ4, αXβ4, αDβ4, αIIBβ4, αEβ4, α1β5, α2β5, α3β5, α4β5, α5β5, α6β5, α7β5, α8β5, α9β5, α10β5, αVβ5, αLβ5, αMβ5, αXβ5, αDβ5, αIIbβ5, αEβ5, α1β6, α2β6, α3β6, α4β6, α5β6, α6β6, α7β6, α8β6, α9β6, α10β6, αVβ6, αLβ6, αMβ6, αXβ6, αDβ6, αIIbβ6, αEβ6, α1β7, α2β7, α3β7, α4β7, α5β7, α6β7, α7β7, α8β7, α9β7, α10β7, αVβ7, αLβ7, αMβ7, αXβ7, αDβ7, αIIbβ7, αEβ7, α1β8, α2β8, α3β8, α4β8, α5β8, α6β8, α7β8, α8β8, α9β8, α10β8, αVβ8, αLβ8, αMβ8, αXβ8, αDβ8, αIIbβ8, and αEβ8 receptor. The chimeric receptor comprising an integrin subunit, or any derivative, variant or fragment thereof, may dimerize with an endogenous integrin subunit (e.g., wild-type integrin subunit).

In some cases, the chimeric receptor may comprise an integrin subunit, or any derivative, variant or fragment thereof, can bind an antigen comprising any suitable integrin ligand, or any derivative, variant or fragment thereof. Non-limiting examples of ligands which can be bound by an integrin receptor may include adenovirus penton base protein, beta-glucan, bone sialoprotein (BSP), Borrelia burgdorferi, Candida albicans, collagens (CN, e.g., CNI-IV), cytotactin/tenascin-C, decorsin, denatured collagen, disintegrins, E-cadherin, echovirus 1 receptor, epiligrin, Factor X, Fc epsilon RII (CD23), fibrin (Fb), fibrinogen (Fg), fibronectin (Fn), heparin, HIV Tat protein, iC3b, intercellular adhesion molecule (e.g., ICAM-1,2,3,4,5), invasin, L1 cell adhesion molecule (L1-CAM), laminin, lipopolysaccharide (LPS), MAdCAM-1, matrix metalloproteinase-2 (MMPe), neutrophil inhibitory factor (NIF), osteopontin (OP or OPN), plasminogen, prothrombin, sperm fertilin, thrombospondin (TSP), vascular cell adhesion molecule 1 (VCAM-1), vitronectin (VN or VTN), and von Willebrand factor (vWF).

In some cases, the chimeric receptor can comprise a cadherin, or any derivative, variant or fragment thereof, selected from a classical cadherin, a desmosoma cadherin, a protocadherin, and an unconventional cadherin. In some embodiments, a chimeric receptor polypeptide comprises a classical cadherin, or any derivative, variant or fragment thereof, selected from CDH1 (E-cadherin, epithelial), CDH2 (N-cadherin, neural), CDH12 (cadherin 12, type 2, N-cadherin 2), and CDH3 (P-cadherin, placental). In some embodiments, a chimeric receptor polypeptide comprises a desmosoma cadherin, or any derivative, variant or fragment thereof, selected from desmoglein (DSG1, DSG2, DSG3, DSG4) and desmocollin (DSC1, DSC2, DSC3). In some embodiments, a chimeric receptor polypeptide comprises a protocadherin, or any derivative, variant or fragment thereof, selected from PCDH1, PCDH10, PCDH11X, PCDH11Y, PCDH12, PCDH15, PCDH17, PCDH18, PCDH19, PCDH20, PCDH7, PCDH8, PCDH9, PCDHA1, PCDHA10, PCDHA11, PCDHA12, PCDHA13, PCDHA2, PCDHA3, PCDHA4, PCDHA5, PCDHA6, PCDHA7, PCDHA8, PCDHA9, PCDHAC1, PCDHAC2, PCDHB1, PCDHB10, PCDHB11, PCDHB12, PCDHB13, PCDHB14, PCDHB15, PCDHB16, PCDHB17, PCDHB18, PCDHB2, PCDHB3, PCDHB4, PCDHB5, PCDHB6, PCDHB7, PCDHB8, PCDHB9, PCDHGA1, PCDHGA10, PCDHGA11, PCDHGA12, PCDHGA2, PCDHGA3, PCDHGA4, PCDHGA5, PCDHGA6, PCDHGA7, PCDHGA8, PCDHGA9, PCDHGB1, PCDHGB2, PCDHGB3, PCDHGB4, PCDHGB5, PCDHGB6, PCDHGB7, PCDHGC3, PCDHGC4, PCDHGC5, FAT, FAT2, and FAT). In some embodiments, a chimeric receptor polypeptide comprises an unconventional cadherin selected from CDH4 (R-cadherin, retinal), CDH5 (VE-cadherin, vascular endothelial), CDH6 (K-cadherin, kidney), CDH7 (cadherin 7, type 2), CDH8 (cadherin 8, type 2), CDH9 (cadherin 9, type 2, T1-cadherin), CDH10 (cadherin 10, type 2, T2-cadherin), CDH11 (OB-cadherin, osteoblast), CDH13 (T-cadherin, H-cadherin, heart), CDH15 (M-cadherin, myotubule), CDH16 (KSP-cadherin), CDH17 (LI cadherin, liver-intestine), CDH18 (cadherin 18, type 2), CDH19 (cadherin 19, type 2), CDH20 (cadherin 20, type 2), CDH23 (cadherin 23, neurosensory epithelium), CDH24, CDH26, CDH28, CELSR1, CELSR2, CELSR3, CLSTN1, CLSTN2, CLSTN3, DCHS1, DCHS2, LOC389118, PCLKC, RESDA1, and RET.

In some cases, the chimeric receptor may comprise at least an extracellular region (e.g., ligand binding domain) of a catalytic receptor such as a RTK, or any derivative, variant or fragment thereof. The chimeric receptor may comprise at least a membrane spanning region of a catalytic receptor such as a RTK, or any derivative, variant or fragment thereof. The chimeric receptor may comprise at least an intracellular region (e.g., cytosolic domain) of a catalytic receptor such as a RTK, or any derivative, variant or fragment thereof. The chimeric receptor may comprise an RTK, or any derivative, variant or fragment thereof, can recruit a binding partner. In some cases, ligand binding to a chimeric receptor comprising an RTK, or any derivative, variant or fragment thereof, results in a conformational change, chemical modification, or combination thereof, which recruits a binding partner to the chimeric receptor.

In some cases, the chimeric receptor may comprise a class I RTK (e.g., the epidermal growth factor (EGF) receptor family including EGFR; the ErbB family including ErbB-2, ErbB-3, and ErbB-4), a class II RTK (e.g., the insulin receptor family including INSR, IGF-1R, and IRR), a class III

RTK (e.g., the platelet-derived growth factor (PDGF) receptor family including PDGFR-α, PDGFR-β, CSF-1R, KIT/SCFR, and FLK2/FLT3), a class IV RTK (e.g., the fibroblast growth factor (FGF) receptor family including FGFR-1, FGFR-2, FGFR-3, and FGFR-4), a class V RTK (e.g., the vascular endothelial growth factor (VEGF) receptor family including VEGFR1, VEGFR2, and VEGFR3), a class VI RTK (e.g., the hepatocyte growth factor (HGF) receptor family including hepatocyte growth factor receptor (HGFR/MET) and RON), a class VII RTK (e.g., the tropomyosin receptor kinase (Trk) receptor family including TRKA, TRKB, and TRKC), a class VIII RTK (e.g., the ephrin (Eph) receptor family including EPHA1, EPHA2, EPHA3, EPHA4, EPHA5, EPHA6, EPHA7, EPHA8, EPHB1, EPHB2, EPHB3, EPHB4, EPHB5, and EPHB6), a class IX RTK (e.g., AXL receptor family such as AXL, MER, and TRYO3), a class X RTK (e.g., LTK receptor family such as LTK and ALK), a class XI RTK (e.g., TIE receptor family such as TIE and TEK), a class XII RTK (e.g., ROR receptor family ROR1 and ROR2), a class XIII RTK (e.g., the discoidin domain receptor (DDR) family such as DDR1 and DDR2), a class XIV RTK (e.g., RET receptor family such as RET), a class XV RTK (e.g., KLG receptor family including PTK7), a class XVI RTK (e.g., RYK receptor family including Ryk), a class XVII RTK (e.g., MuSK receptor family such as MuSK), or any derivative, variant or fragment thereof.

The chimeric receptor comprising a RTK, or any derivative, variant or fragment thereof, may bind an antigen comprising any suitable RTK ligand, or any derivative, variant or fragment thereof. Non limiting examples of RTK ligands include growth factors, cytokines, and hormones. Growth factors include, for example, members of the epidermal growth factor family (e.g., epidermal growth factor or EGF, heparin-binding EGF-like growth factor or HB-EGF, transforming growth factor-α or TGF-a, amphiregulin or AR, epiregulin or EPR, epigen, betacellulin or BTC, neuregulin-1 or NRG1, neuregulin-2 or NRG2, neuregulin-3 or NRG3, and neuregulin-4 or NRG4), the fibroblast growth factor family (e.g., FGF1, FGF2, FGF3, FGF4, FGF5, FGF6, FGF7, FGF8, FGF9, FGF10, FGF11, FGF12, FGF13, FGF14, FGF15/19, FGF16, FGF17, FGF18, FGF20, FGF21, and FGF23), the vascular endothelial growth factor family (e.g., VEGF-A, VEGF-B, VEGF-C, VEGF-D, and PIGF), and the platelet-derived growth factor family (e.g., PDGFA, PDGFB, PDGFC, and PDGFD). Hormones include, for example, members of the insulin/IGF/relaxin family (e.g., insulin, insulin-like growth factors, relaxin family peptides including relaxin1, relaxin2, relaxin3, Leydig cell-specific insulin-like peptide (gene INSL3), early placenta insulin-like peptide (ELIP) (gene INSL4), insulin-like peptide 5 (gene INSL5), and insulin-like peptide 6).

In some cases, the chimeric receptor may comprise at least an extracellular region (e.g., ligand binding domain) of a catalytic receptor such as an RTSK, or any derivative, variant or fragment thereof. The chimeric receptor may comprise at least a membrane spanning region of a catalytic receptor such as an RTSK, or any derivative, variant or fragment thereof. The chimeric receptor may comprise at least an intracellular region (e.g., cytosolic domain) of a catalytic receptor such as an RTSK, or any derivative, variant or fragment thereof. The chimeric receptor polypeptide comprising an RTSK, or any derivative, variant or fragment thereof, may recruit a binding partner. In some cases, ligand binding to the chimeric receptor comprising an RTSK, or any derivative, variant or fragment thereof, may result in a conformational change, chemical modification, or combination thereof, which recruits a binding partner to the chimeric receptor.

The chimeric receptor comprising an RTSK, or any derivative, variant or fragment thereof, may phosphorylate a substrate at serine and/or threonine residues, and may select specific residues based on a consensus sequence. The chimeric receptor may comprise a type I RTSK, type II RTSK, or any derivative, variant or fragment thereof. The chimeric receptor comprising a type I receptor serine/threonine kinase may be inactive unless complexed with a type II receptor. In some cases, the chimeric receptor comprising a type II receptor serine/threonine may comprise a constitutively active kinase domain that can phosphorylate and activate a type I receptor when complexed with the type I receptor. A type II receptor serine/threonine kinase can phosphorylate the kinase domain of the type I partner, causing displacement of protein partners. Displacement of protein partners can allow binding and phosphorylation of other proteins, for example certain members of the SMAD family. The chimeric receptor can comprise a type I receptor, or any derivative, variant or fragment thereof, selected from the group consisting of: ALK1 (ACVRL1), ALK2 (ACVR1A), ALK3 (BMPR1A), ALK4 (ACVR1B), ALK5 (TGFβR1), ALK6 (BMPR1B), and ALK7 (ACVR1C). The chimeric receptor can comprise a type II receptor, or any derivative, variant or fragment thereof, selected from the group consisting of: TGFOR2, BMPR2, ACVR2A, ACVR2B, and AMHR2 (AMHR). The chimeric receptor can comprise a TGF-β receptor, or any derivative, variant or fragment thereof.

In some cases, the chimeric receptor can comprise a receptor which stimulates non-covalently associated intracellular kinases, such as a Src kinase (e.g., c-Src, Yes, Fyn, Fgr, Lck, Hck, Blk, Lyn, and Frk) or a JAK kinase (e.g., JAK1, JAK2, JAK3, and TYK2) rather than possessing intrinsic enzymatic activity, or any derivative, variant or fragment thereof. These include the cytokine receptor superfamily such as receptors for cytokines and polypeptide hormones. Cytokine receptors generally contain an N-terminal extracellular ligand-binding domain, transmembrane a helices, and a C-terminal cytosolic domain. The cytosolic domains of cytokine receptors are generally devoid of any known catalytic activity. Cytokine receptors instead can function in association with non-receptor kinases (e.g., tyrosine kinases or threonine/serine kinases), which can be activated as a result of ligand binding to the receptor. The chimeric receptor can comprise at least an extracellular region (e.g., ligand binding domain) of a catalytic receptor that non-covalently associates with an intracellular kinase (e.g., a cytokine receptor), or any derivative, variant or fragment thereof. The chimeric receptor can comprise at least a membrane spanning region of a catalytic receptor that non-covalently associates with an intracellular kinase (e.g., a cytokine receptor), or any derivative, variant or fragment thereof. The chimeric receptor can comprise at least an intracellular region (e.g., cytosolic domain) of a catalytic receptor that non-covalently associates with an intracellular kinase (e.g., a cytokine receptor), or any derivative, variant or fragment thereof. The chimeric receptor comprising a catalytic receptor that non-covalently associates with an intracellular kinase, or any derivative, variant or fragment thereof, can recruit a binding partner. In some cases, ligand binding to the chimeric receptor comprising a catalytic receptor that non-covalently associates with an intracellular kinase, or any derivative, variant or fragment thereof, may result in a conformational change, chemical modification, or combination thereof, which recruits a binding partner to the receptor.

In some cases, the chimeric receptor can comprise a cytokine receptor, for example a type I cytokine receptor or a type II cytokine receptor, or any derivative, variant or fragment thereof. The chimeric receptor can comprise an interleukin receptor (e.g., IL-2R, IL-3R, IL-4R, IL-5R, IL-6R, IL-7R, IL-9R, IL-11R, IL-12R, IL-13R, IL-15R, IL-21R, IL-23R, IL-27R, and IL-31R), a colony stimulating factor receptor (e.g., erythropoietin receptor, CSF-1R, CSF-2R, GM-CSFR, and G-CSFR), a hormone receptor/neuropeptide receptor (e.g., growth hormone receptor, prolactin receptor, and leptin receptor), or any derivative, variant or fragment thereof. The chimeric receptor can comprise a type II cytokine receptor, or any derivative, variant or fragment thereof. The chimeric receptor can comprise an interferon receptor (e.g., IFNAR1, IFNAR2, and IFNGR), an interleukin receptor (e.g., IL-10R, IL-20R, IL-22R, and IL-28R), a tissue factor receptor (also called platelet tissue factor), or any derivative, variant or fragment thereof.

In some cases, the chimeric receptor comprising a cytokine receptor can bind an antigen comprising any suitable cytokine receptor ligand, or any derivative, variant or fragment thereof. Non-limiting examples of cytokine receptor ligands include interleukins (e.g., IL-2, IL-3, IL-4, IL-5, IL-6, IL-7, IL-9, IL-10, IL-11, IL-12, IL-13, IL-15, IL-20, IL-21, IL-22, IL-23, IL-27, IL-28, and IL-31), interferons (e.g., IFN-α, IFN-β, IFN-γ), colony stimulating factors (e.g., erythropoietin, macrophage colony-stimulating factor, granulocyte macrophage colony-stimulating factors or GM-CSFs, and granulocyte colony-stimulating factors or G-CSFs), and hormones (e.g., prolactin and leptin).

In some cases, the chimeric receptor can comprise a death receptor, a receptor containing a death domain, or any derivative, variant or fragment thereof. Death receptors are often involved in regulating apoptosis and inflammation. Death receptors include members of the TNF receptor family such as TNFR1, Fas receptor, DR4 (also known as TRAIL receptor 1 or TRAILR1) and DR5 (also known as TRAIL receptor 2 or TRAILR2). The chimeric receptor can comprise at least an extracellular region (e.g., ligand binding domain) of a death receptor, or any derivative, variant or fragment thereof. The chimeric receptor can comprise at least a membrane spanning region of a death receptor, or any derivative, variant or fragment thereof. The chimeric receptor can comprise at least an intracellular region (e.g., cytosolic) domain of a death receptor, or any derivative, variant or fragment thereof. The chimeric receptor polypeptide comprising a death receptor, or any derivative, variant or fragment thereof, can undergo receptor oligomerization in response to ligand binding, which in turn can result in the recruitment of specialized adaptor proteins and activation of signaling cascades, such as caspase cascades. The chimeric receptor can comprise a death receptor, or any derivative, variant or fragment thereof, results in a conformational change, chemical modification, or combination thereof, which recruits a binding partner to the chimeric receptor.

The chimeric receptor comprising a death receptor can bind an antigen comprising any suitable ligand of a death receptor, or any derivative, variant or fragment thereof. Non-limiting examples of ligands bound by death receptors include TNFα, Fas ligand, and TNF-related apoptosis-inducing ligand (TRAIL).

In some cases, the chimeric receptor can comprise an immune receptor, or any derivative, variant or fragment thereof. Immune receptors can include members of the immunoglobulin superfamily (IgSF) which share structural features with immunoglobulins, e.g., a domain known as an immunoglobulin domain or fold. IgSF members include, but are not limited to, cell surface antigen receptors, co-receptors and costimulatory molecules of the immune system, and molecules involved in antigen presentation to lymphocytes. The chimeric receptor can comprise at least an extracellular region (e.g., ligand binding domain) of an immune receptor, or any derivative, variant or fragment thereof. The chimeric receptor can comprise at least a region spanning a membrane of an immune receptor, or any derivative, variant or fragment thereof. The chimeric receptor can comprise at least an intracellular region (e.g., cytoplasmic domain) of an immune receptor, or any derivative, variant or fragment thereof. The chimeric receptor comprising an immune receptor, or any derivative, variant or fragment thereof, can recruit a binding partner. Ligand binding to a chimeric receptor comprising an immune receptor, or any derivative, variant or fragment thereof, can result in a conformational change, chemical modification, or combination thereof, which recruits a binding partner to the chimeric receptor.

In some cases, the chimeric receptor can comprise a cell surface antigen receptor such as a T cell receptor (TCR), a B cell receptor (BCR), or any derivative, variant or fragment thereof. T cell receptors generally comprise two chains, either the TCR-alpha and -beta chains or the TCR-delta and -gamma chains. A chimeric polypeptide comprising a TCR, or any derivative, variant or fragment thereof, can bind a major histocompatibility complex (WIC) protein. B cell receptors generally comprises a membrane bound immunoglobulin and a signal transduction moiety. A chimeric polypeptide comprising a BCR, or any derivative, variant or fragment thereof, can bind a cognate BCR antigen. A chimeric polypeptide comprising at least an immunoreceptor tyrosine-based activation motif (ITAM) found in the cytoplasmic domain of certain immune receptors. A chimeric polypeptide may comprise at least an immunoreceptor tyrosine-based inhibition motif (ITIM) found in the cytoplasmic domain of certain immune receptors. A chimeric polypeptide comprising ITAM and/or ITIM domains can be phosphorylated following ligand binding to an antigen interacting domain. The phosphorylated regions can serve as docking sites for other proteins involved in immune cell signaling.

The antigen interacting domain of a chimeric receptor can bind a membrane bound antigen, for example an antigen bound to the extracellular surface of a cell (e.g., a target cell). The antigen interacting domain may bind a non-membrane bound antigen, for example an extracellular antigen that is secreted by a cell (e.g., a target cell) or an antigen located in the cytoplasm of a cell. Antigens (e.g., membrane bound and non-membrane bound) can be associated with a disease such as a viral, bacterial, and/or parasitic infection; inflammatory and/or autoimmune disease; or neoplasm such as a cancer and/or tumor. Cancer antigens, for example, may be proteins produced by tumor cells that can elicit an immune response, particularly a T-cell mediated immune response. The selection of the antigen binding portions of a chimeric receptor can depend on the particular type of cancer antigen to be targeted. In some cases, the tumor antigen may comprise one or more antigenic cancer epitopes associated with a malignant tumor. Malignant tumors can express a number of proteins that can serve as target antigens for an immune attack. The antigen interaction domains can bind to cell surface signals, extracellular matrix (ECM), paracrine signals, juxtacrine signals, endocrine signals, autocrine signals, signals that can trigger or control genetic programs in cells, or any combination thereof. In some cases, interactions between the cell signals that bind to the chimeric receptor involve a cell-cell interaction, cell-soluble chemical interaction, and cell-matrix or microenvironment interaction.

### V. Actuator Moiety

The actuator moiety (e.g., an actuator moiety that is a part of a GMP) as disclosed herein can be capable of editing (e.g., via insertion and/or deletion (indel), homology directed repair (HDR), non-homologous end joining (NHEJ)) the target gene, to regulate expression or activity of the endogenous protein (e.g., cytokine, immune checkpoint inhibitor, etc.).

The actuator moiety (e.g., an actuator moiety that is a part of a GMP) as disclosed herein can be operatively coupled to at least one effector domain. The at least one effector domain can be configured to regulate the expression or activity of the endogenous protein (e.g., cytokine, immune checkpoint inhibitor, etc.), In some cases, the actuator moiety can be fused to at least one effector domain, to form a fusion moiety. In some cases, the actuator moiety can comprise a first coupling moiety (e.g., a polynucleotide) and the at least one effector domain can comprise a second coupling moiety (e.g., a second polynucleotide having complementarity to the first polynucleotide), such that the actuator moiety and the at least one effector domain can be coupled to one another. In some examples, the at least one effector domain can be a cleavage domain, an epigenetic modification domain, a transcriptional activation domain, or a transcriptional repressor domain, to regulate expression or activity of the endogenous protein.

Non-limiting examples of a function of the at least one effector domain as disclosed herein can include methyltransferase activity, demethylase activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, remodeling activity, protease activity, oxidoreductase activity, transferase activity, hydrolase activity, lyase activity, isomerase activity, synthase activity, synthetase activity, and demyristoylation activity.

Non-limiting examples of the at least one effector domain as disclosed herein can include methyltransferase, demethylase, dismutase, alkylation enzyme, depurination enzyme, oxidation enzyme, pyrimidine dimer forming enzyme, integrase, transposase, recombinase, polymerase, ligase, helicase, photolyase or glycosylase, acetyltransferase, deacetylase, kinase, phosphatase, ubiquitin ligase, deubiquitinating enzyme, adenylation enzyme, deadenylation enzyme, SUMOylating enzyme, deSUMOylating enzyme, ribosylation enzyme, deribosylation enzyme, myristoylation enzyme, remodeling enzyme, protease, oxidoreductase, transferase, hydrolase, lyase, isomerase, synthase, synthetase, and demyristoylation enzyme.

The actuator moiety as disclosed herein can comprise a nuclease, such as an endonuclease (e.g., Cas). The endonuclease can be heterologous to any of the cells disclosed herein.

The actuator moiety as disclosed herein can comprise a Cas endonuclease, zinc finger nuclease (ZFN), zinc finger associate gene regulation polypeptides, transcription activator-like effector nuclease (TALEN), transcription activator-like effector associated gene regulation polypeptides, meganuclease, natural master transcription factors, epigenetic modifying enzymes, recombinase, flippase, transposase, RNA-binding proteins (RBP), an Argonaute protein, any derivative thereof, any variant thereof, or any fragment thereof. In some embodiments, the actuator moiety comprises a Cas protein, and the system further comprises a guide RNA (gRNA) which complexes with the Cas protein. In some embodiments, the actuator moiety comprises an RBP complexed with a gRNA which is able to form a complex with a Cas protein. In some embodiments, the gRNA comprises a targeting segment which exhibits at least 80% sequence identity to a target polynucleotide. In some embodiments, the Cas protein substantially lacks DNA cleavage activity (i.e., dead Cas, deactivated Cas, or dCas). For example, the Cas protein is mutated and/or modified to yield a nuclease deficient protein or a protein with decreased nuclease activity relative to a wild-type Cas protein. A nuclease deficient protein can retain the ability to bind DNA, but may lack or have reduced nucleic acid cleavage activity.

In some cases, a suitable actuator moiety comprises CRISPR-associated (Cas) proteins or Cas nucleases including type I CRISPR-associated (Cas) polypeptides, type II CRISPR-associated (Cas) polypeptides, type III CRISPR-associated (Cas) polypeptides, type IV CRISPR-associated (Cas) polypeptides, type V CRISPR-associated (Cas) polypeptides, and type VI CRISPR-associated (Cas) polypeptides; zinc finger nucleases (ZFN); transcription activator-like effector nucleases (TALEN); meganucleases; RNA-binding proteins (RBP); CRISPR-associated RNA binding proteins; recombinases; flippases; transposases; Argonaute (Ago) proteins (e.g., prokaryotic Argonaute (pAgo), archaeal Argonaute (aAgo), and eukaryotic Argonaute (eAgo)); any derivative thereof, any variant thereof and any fragment thereof.

A Cas protein referred to herein can be a type of protein or polypeptide. A Cas protein can refer to a nuclease. A Cas protein can refer to an endoribonuclease. A Cas protein can refer to any modified (e.g., shortened, mutated, lengthened) polypeptide sequence or homologue of the Cas protein. A Cas protein can be codon optimized. A Cas protein can be a codon-optimized homologue of a Cas protein. A Cas protein can be enzymatically inactive, partially active, constitutively active, fully active, inducible active and/or more active, (e.g. more than the wild type homologue of the protein or polypeptide.). A Cas protein can be Cas9. A Cas protein can be Cpf1. A Cas protein can be C2c2. A Cas protein (e.g., variant, mutated, enzymatically inactive and/or conditionally enzymatically inactive site-directed polypeptide) can bind to a target nucleic acid. A Cas protein (e.g., variant, mutated, enzymatically inactive and/or conditionally enzymatically inactive endoribonuclease) can bind to a target RNA or DNA.

Non-limiting examples of Cas proteins include c2c1, C2c2, c2c3, Cas1, Cas1B, Cas2, Cas3, Cas4, Cas5, Cas5e (CasD), Cash, Cas6e, Cas6f, Cas7, Cas8a, Cas8a1, Cas8a2, Cas8b, Cas8c, Cas9 (Csn1 or Csx12), Cas10, Cas10d, Cas10, Cas10d, CasF, CasG, CasH, Cpf1, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx1O, Csx16, CsaX, Csx3, Csx1, Csx15, Csf1, Csf2, Csf3, Csf4, and Cul966, and homologs or modified versions thereof.

In some cases, a nuclease disclosed herein (e.g., Cas) can be a nucleic acid-guided nuclease (e.g., an RNA guided endonuclease). The term “guide nucleic acid” generally refers to a nucleic acid that can hybridize to another nucleic acid. A guide nucleic acid can be RNA. A guide nucleic acid can be DNA. The guide nucleic acid can be programmed to bind to a sequence of nucleic acid site-specifically. The nucleic acid to be targeted, or the target nucleic acid, can comprise nucleotides. The guide nucleic acid can comprise nucleotides. A portion of the target nucleic acid can be complementary to a portion of the guide nucleic acid. The strand of a double-stranded target polynucleotide that is complementary to and hybridizes with the guide nucleic acid can be called the complementary strand. The strand of the double-stranded target polynucleotide that is complementary to the complementary strand, and therefore may not be complementary to the guide nucleic acid can be called noncomplementary strand. A guide nucleic acid can comprise a polynucleotide chain and can be called a “single guide nucleic acid.” A guide nucleic acid can comprise two polynucleotide chains and can be called a “double guide nucleic acid.” If not otherwise specified, the term “guide nucleic acid” can be inclusive, referring to both single guide nucleic acids and double guide nucleic acids.

A guide nucleic acid can comprise a segment that can be referred to as a “nucleic acid-targeting segment” or a “nucleic acid-targeting sequence.” A nucleic acid-targeting segment can comprise a sub-segment that can be referred to as a “protein binding segment” or “protein binding sequence” or “Cas protein binding segment.”

A guide nucleic acid can comprise two separate nucleic acid molecules, which can be referred to as a double guide nucleic acid. A guide nucleic acid can comprise a single nucleic acid molecule, which can be referred to as a single guide nucleic acid (e.g., sgRNA). In some cases, the guide nucleic acid is a single guide nucleic acid comprising a fused CRISPR RNA (crRNA) and a transactivating crRNA (tracrRNA). In some cases, the guide nucleic acid is a single guide nucleic acid comprising a crRNA. In some cases, the guide nucleic acid is a single guide nucleic acid comprising a crRNA but lacking a tracrRNA. In some cases, the guide nucleic acid is a double guide nucleic acid comprising non-fused crRNA and tracrRNA. An exemplary double guide nucleic acid can comprise a crRNA-like molecule and a tracrRNA-like molecule. An exemplary single guide nucleic acid can comprise a crRNA-like molecule. An exemplary single guide nucleic acid can comprise a fused crRNA-like and tracrRNA-like molecules.

The term “crRNA,” as used herein, generally refers to a nucleic acid with at least about 5%, 10%, 20%, 30%, 40%, 50%, 60%, 70%, 80%, 90%, or 100% sequence identity and/or sequence similarity to a wild type exemplary crRNA (e.g., a crRNA from S. pyogenes). crRNA can generally refer to a nucleic acid with at most about 5%, 10%, 20%, 30%, 40%, 50%, 60%, 70%, 80%, 90%, or 100% sequence identity and/or sequence similarity to a wild type exemplary crRNA (e.g., a crRNA from S. pyogenes). crRNA can refer to a modified form of a crRNA that can comprise a nucleotide change such as a deletion, insertion, or substitution, variant, mutation, or chimera. A crRNA can be a nucleic acid having at least about 60% sequence identity to a wild type exemplary crRNA (e.g., a crRNA from S. pyogenes) sequence over a stretch of at least 6 contiguous nucleotides. For example, a crRNA sequence can be at least about 60% identical, at least about 65% identical, at least about 70% identical, at least about 75% identical, at least about 80% identical, at least about 85% identical, at least about 90% identical, at least about 95% identical, at least about 98% identical, at least about 99% identical, or 100% identical to a wild type exemplary crRNA sequence (e.g., a crRNA from S. pyogenes) over a stretch of at least 6 contiguous nucleotides.

The term “tracrRNA,” as used herein, generally refers to a nucleic acid with at least about 5%, 10%, 20%, 30%, 40%, 50%, 60%, 70%, 80%, 90%, or 100% sequence identity and/or sequence similarity to a wild type exemplary tracrRNA sequence (e.g., a tracrRNA from S. pyogenes). tracrRNA can refer to a nucleic acid with at most about 5%, 10%, 20%, 30%, 40%, 50%, 60%, 70%, 80%, 90%, or 100% sequence identity and/or sequence similarity to a wild type exemplary tracrRNA sequence (e.g., a tracrRNA from S. pyogenes). tracrRNA can refer to a modified form of a tracrRNA that can comprise a nucleotide change such as a deletion, insertion, or substitution, variant, mutation, or chimera. A tracrRNA can refer to a nucleic acid that can be at least about 60% identical to a wild type exemplary tracrRNA (e.g., a tracrRNA from S. pyogenes) sequence over a stretch of at least 6 contiguous nucleotides. For example, a tracrRNA sequence can be at least about 60% identical, at least about 65% identical, at least about 70% identical, at least about 75% identical, at least about 80% identical, at least about 85% identical, at least about 90% identical, at least about 95% identical, at least about 98% identical, at least about 99% identical, or 100% identical to a wild type exemplary tracrRNA (e.g., a tracrRNA from S. pyogenes) sequence over a stretch of at least 6 contiguous nucleotides.

A crRNA can comprise the nucleic acid-targeting segment (e.g., spacer region) of the guide nucleic acid and a stretch of nucleotides that can form one half of a double-stranded duplex of the Cas protein-binding segment of the guide nucleic acid.

A tracrRNA can comprise a stretch of nucleotides that forms the other half of the double-stranded duplex of the Cas protein-binding segment of the gRNA. A stretch of nucleotides of a crRNA can be complementary to and hybridize with a stretch of nucleotides of a tracrRNA to form the double-stranded duplex of the Cas protein-binding domain of the guide nucleic acid.

The crRNA and tracrRNA can hybridize to form a guide nucleic acid. The crRNA can also provide a single-stranded nucleic acid targeting segment (e.g., a spacer region) that hybridizes to a target nucleic acid recognition sequence (e.g., protospacer). The sequence of a crRNA, including spacer region, or tracrRNA molecule can be designed to be specific to the species in which the guide nucleic acid is to be used.

In some cases, the effector domain can be a transcriptional activation domain selected from the group consisting of GAL4, VP16, VP64, p65, Rta, VPR, and variants thereof (e.g., mini-VPR). In some examples, the actuator moiety can be a Cas protein (e.g., dCas such as dCas9) fused to the transcriptional activation domain, as disclosed herein.

In some cases, the effector domain can be a transcriptional repressor domain selected from the group consisting of KRAB, SID, ERD, and variants thereof. In some examples, the actuator moiety can be a Cas protein (e.g., dCas such as dCas9) fused to the transcriptional repressor domain as disclosed herein.

VI. Therapeutic Applications

The plurality of cells (e.g., a plurality of engineered immune cells) can comprise an actuator moiety as disclosed herein to regulate expression (e.g., increase or decrease expression) of one or more target genes encoding one or more endogenous proteins. Example genes encoding the endogenous protein as disclosed herein are provided in Tables 1, 2, and 3. Exemplary genes associated with certain diseases and disorders are provided in Tables 1 and 2. Examples of signaling biochemical pathway-associated genes and polynucleotides are listed in Table 3.

TABLE 1

| DISEASE/DISORDERS | GENE(S) |
|---|---|
| Neoplasia | PTEN; ATM; ATR; EGFR; ERBB2; ERBB3; ERBB4; |
| | Notch1; Notch2; Notch3; Notch4; AKT; AKT2; AKT3; HIF; |
| | HIF1a; HIF3a; Met; HRG; Bcl2; PPAR alpha; PPAR |
| | gamma; WT1 (Wilms Tumor); FGF Receptor Family |
| | members (5 members: 1, 2, 3, 4, 5); CDKN2a; APC; RB |
| | (retinoblastoma); MEN1; VHL; BRCA1; BRCA2; AR |
| | (Androgen Receptor); TSG101; IGF; IGF Receptor; Igf1 (4 |
| | variants); Igf2 (3 variants); Igf 1 Receptor; Igf 2 Receptor; |
| | Bax; Bcl2; caspases family (9 members: |
| | 1, 2, 3, 4, 6, 7, 8, 9, 12); Kras; Apc |
| Age-related Macular | Abcr; Ccl2; Cc2; cp (ceruloplasmin); Timp3; cathepsinD; |
| Degeneration | Vldlr; Ccr2 |
| Schizophrenia | Neuregulin1 (Nrg1); Erb4 (receptor for Neuregulin); |
| | Complexin1 (Cplx1); Tph1 Tryptophan hydroxylase; Tph2 |
| | Tryptophan hydroxylase 2; Neurexin 1; GSK3; GSK3a; |
| | GSK3b |
| Disorders | 5-HTT (Slc6a4); COMT; DRD (Drd1a); SLC6A3; DAOA; |
| | DTNBP1; Dao (Dao1) |
| Trinucleotide Repeat | HTT (Huntington's Dx); SBMA/SMAX1/AR (Kennedy's |
| Disorders | Dx); FXN/X25 (Friedrich's Ataxia); ATX3 (Machado- |
| | Joseph's Dx); ATXN1 and ATXN2 (spinocerebellar |
| | ataxias); DMPK (myotonic dystrophy); Atrophin-1 and Atn1 |
| | (DRPLA Dx); CBP (Creb-BP - global instability); VLDLR |
| | (Alzheimer's); Atxn7; Atxn10 |
| Fragile X Syndrome | FMR2; FXR1; FXR2; mGLUR5 |
| Secretase Related | APH-1 (alpha and beta); Presenilin (Psen1); nicastrin |
| Disorders | (Ncstn); PEN-2 |
| Others | Nos1; Parp1; Nat1; Nat2 |
| Prion - related disorders | Prp |
| ALS | SOD1; ALS2; STEX; FUS; TARDBP; VEGF (VEGF-a; |
| | VEGF-b; VEGF-c) |
| Drug addiction | Prkce (alcohol); Drd2; Drd4; ABAT (alcohol); GRIA2; |
| | Grm5; Grin1; Htr1b; Grin2a; Drd3; Pdyn; Gria1 (alcohol) |
| Autism | Mecp2; BZRAP1; MDGA2; Sema5A; Neurexin 1; Fragile X |
| | (FMR2 (AFF2); FXR1; FXR2; Mglur5) |
| Alzheimer's Disease | E1; CHIP; UCH; UBB; Tau; LRP; PICALM; Clusterin; PS1; |
| | SORL1; CR1; Vldlr; Uba1; Uba3; CHIP28 (Aqp1, |
| | Aquaporin 1); Uchl1; Uchl3; APP |
| Inflammation | IL-10; IL-1 (IL-1a; IL-1b); IL-13; IL-17 (IL-17a (CTLA8); IL- |
| | 17b; IL-17c; IL-17d; IL-17f); II-23; Cx3cr1; ptpn22; TNFa; |
| | NOD2/CARD15 for IBD; IL-6; IL-12 (IL-12a; IL-12b); |
| | CTLA4; Cx3cl1 |
| Parkinson's Disease | x-Synuclein; DJ-1; LRRK2; Parkin; PINK1 |

TABLE 2

| | |
|---|---|
| Blood and | Anemia (CDAN1, CDA1, RPS19, DBA, PKLR, PK1, NT5C3, UMPH1, |
| coagulation | PSN1, RHAG, RH50A, NRAMP2, SPTB, ALAS2, ANH1, ASB, |
| diseases and | ABCB7, ABC7, ASAT); Bare lymphocyte syndrome (TAPBP, TPSN, |
| disorders | TAP2, ABCB3, PSF2, RING11, MHC2TA, C2TA, RFX5, RFXAP, |
| | RFX5), Bleeding disorders (TBXA2R, P2RX1, P2X1); Factor H and |
| | factor H-like 1 (HF1, CFH, HUS); Factor V and factor VIII (MCFD2); |
| | Factor VII deficiency (F7); Factor X deficiency (F10); Factor XI |
| | deficiency (F11); Factor XII deficiency (F12, HAF); Factor XIIIA |
| | deficiency (F13A1, F13A); Factor XIIIB deficiency (F13B); Fanconi |
| | anemia (FANCA, FACA, FA1, FA, FAA, FAAP95, FAAP90, FLJ34064, |
| | FANCB, FANCC, FACC, BRCA2, FANCD1, FANCD2, FANCD, |
| | FACD, FAD, FANCE, FACE, FANCF, XRCC9, FANCG, BRIP1, |
| | BACH1, FANCJ, PHF9, FANCL, FANCM, KIAA1596); |
| | Hemophagocytic lymphohistiocytosis disorders (PRF1, HPLH2, |
| | UNC13D, MUNC13-4, HPLH3, HLH3, FHL3); Hemophilia A (F8, F8C, |
| | HEMA); Hemophilia B (F9, HEMB), Hemorrhagic disorders (PI, ATT, |
| | F5); Leukocyde deficiencies and disorders (ITGB2, CD18, LCAMB, |
| | LAD, EIF2B1, EIF2BA, EIF2B2, EIF2B3, EIF2B5, LVWM, CACH, |
| | CLE, EIF2B4); Sickle cell anemia (HBB); Thalassemia (HBA2, HBB, |
| | HBD, LCRB, HBA1). |
| Cell dysregulation | B-cell non-Hodgkin lymphoma (BCL7A, BCL7); Leukemia (TAL1, |
| and oncology | TCL5, SCL, TAL2, FLT3, NBS1, NBS, ZNFN1A1, IK1, LYF1, |
| diseases and | HOXD4, HOX4B, BCR, CML, PHL, ALL, ARNT, KRAS2, RASK2, |
| disorders | GMPS, AF10, ARHGEF12, LARG, KIAA0382, CALM, CLTH, |
| | CEBPA, CEBP, CHIC2, BTL, FLT3, KIT, PBT, LPP, NPM1, NUP214, |
| | D9546E, CAN, CAIN, RUNX1, CBFA2, AML1, WHSC1L1, NSD3, |
| | FLT3, AF1Q, NPM1, NUMA1, ZNF145, PLZF, PML, MYL, STAT5B, |
| | AF10, CALM, CLTH, ARL11, ARLTS1, P2RX7, P2X7, BCR, CML, |
| | PHL, ALL, GRAF, NF1, VRNF, WSS, NFNS, PTPN11, PTP2C, SHP2, |
| | NS1, BCL2, CCND1, PRAD1, BCL1, TCRA, GATA1, GF1, ERYF1, |
| | NFE1, ABL1, NQO1, DIA4, NMOR1, NUP214, D9S46E, CAN, CAIN). |
| Inflammation and | AIDS (KIR3DL1, NKAT3, NKB1, AMB11, KIR3DS1, IFNG, CXCL12, |
| immune related | SDF1); Autoimmune lymphoproliferative syndrome (TNFRSF6, APT1, |
| diseases and | FAS, CD95, ALPS1A); Combined immunodeficiency, (IL2RG, |
| disorders | SCIDX1, SCIDX, IMD4); HIV-1 (CCL5, SCYA5, D17S136E, TCP228), |
| | HIV susceptibility or infection (IL10, CSIF, CMKBR2, CCR2, |
| | CMKBR5, CCCKR5 (CCR5)); Immunodeficiencies (CD3E, CD3G, |
| | AICDA, AID, HIGM2, TNFRSF5, CD40, UNG, DGU, HIGM4, |
| | TNFSF5, CD40LG, HIGM1, IGM, FOXP3, IPEX, AIID, XPID, PIDX, |
| | TNFRSF14B, TACI); Inflammation (IL-10, IL-1 (IL-1a, IL-1b), IL-13, |
| | IL-17 (IL-17a (CTLA8), IL-17b, IL-17c, IL-17d, IL-17f), II-23, Cx3cr1, |
| | ptpn22, TNFa, NOD2/CARD15 for IBD, IL-6, IL-12 (IL-12a, IL-12b), |
| | CTLA4, Cx3cl1); Severe combined immunodeficiencies (SCIDs)(JAK3, |
| | JAKL, DCLRE1C, ARTEMIS, SCIDA, RAG1, RAG2, ADA, PTPRC, |
| | CD45, LCA, IL7R, CD3D, T3D, IL2RG, SCIDX1, SCIDX, IMD4). |
| Metabolic, liver, | Amyloid neuropathy (TTR, PALB); Amyloidosis (APOA1, APP, AAA, |
| kidney and | CVAP, AD1, GSN, FGA, LYZ, TTR, PALB); Cirrhosis (KRT18, KRT8, |
| protein diseases | CIRH1A, NAIC, TEX292, KIAA1988); Cystic fibrosis (CFTR, ABCC7, |
| and disorders | CF, MRP7); Glycogen storage diseases (SLC2A2, GLUT2, G6PC, |
| | G6PT, G6PT1, GAA, LAMP2, LAMPB, AGL, GDE, GBE1, GYS2, |
| | PYGL, PFKM); Hepatic adenoma, 142330 (TCF1, HNF1A, MODY3), |
| | Hepatic failure, early onset, and neurologic disorder (SCOD1, SCO1), |
| | Hepatic lipase deficiency (LIPC), Hepatoblastoma, cancer and |
| | carcinomas (CTNNB1, PDGFRL, PDGRL, PRLTS, AXIN1, AXIN, |
| | CTNNB1, TP53, P53, LFS1, IGF2R, MPRI, MET, CASP8, MCH5; |
| | Medullary cystic kidney disease (UMOD, HNFJ, FJHN, MCKD2, |
| | ADMCKD2); Phenylketonuria (PAH, PKU1, QDPR, DHPR, PTS); |
| | Polycystic kidney and hepatic disease (FCYT, PKHD1, ARPKD, PKD1, |
| | PKD2, PKD4, PKDTS, PRKCSH, G19P1, PCLD, SEC63). |
| Muscular/Skeletal | Becker muscular dystrophy (DMD, BMD, MYF6), Duchenne Muscular |
| diseases and | Dystrophy (DMD, BMD); Emery-Dreifuss muscular dystrophy (LMNA, |
| disorders | LMN1, EMD2, FPLD, CMD1A, HGPS, LGMD1B, LMNA, LMN1, |
| | EMD2, FPLD, CMD1A); Facioscapulohumeral muscular dystrophy |
| | (FSHMD1A, FSHD1A); Muscular dystrophy (FKRP, MDC1C, |
| | LGMD2I, LAMA2, LAMM, LARGE, KIAA0609, MDC1D, FCMD, |
| | TTID, MYOT, CAPN3, CANP3, DYSF, LGMD2B, SGCG, LGMD2C, |
| | DMDA1, SCG3, SGCA, ADL, DAG2, LGMD2D, DMDA2, SGCB, |
| | LGMD2E, SGCD, SGD, LGMD2F, CMD1L, TCAP, LGMD2G, |
| | CMD1N, TRIM32, HT2A, LGMD2H, FKRP, MDC1C, LGMD2I, TTN, |
| | CMD1G, TMD, LGMD2J, POMT1, CAV3, LGMD1C, SEPN1, SELN, |
| | RSMD1, PLEC1, PLTN, EBS1); Osteopetrosis (LRP5, BMND1, LRP7, |
| | LR3, OPPG, VBCH2, CLCN7, CLC7, OPTA2, OSTM1, GL, TCIRG1, |
| | TIRC7, OC116, OPTB1); Muscular atrophy (VAPB, VAPC, ALS8, |
| | SMN1, SMA1, SMA2, SMA3, SMA4, BSCL2, SPG17, GARS, SMAD1, |
| | CMT2D, HEXB, IGHMBP2, SMUBP2, CATF1, SMARD1). |
| Neurological and | ALS (SOD1, ALS2, STEX, FUS, TARDBP, VEGF (VEGF-a, VEGF-b, |
| neuronal diseases | VEGF-c); Alzheimer disease (APP, AAA, CVAP, AD1, APOE, AD2, |
| and disorders | PSEN2, AD4, STM2, APBB2, FE65L1, NOS3, PLAU, URK, ACE, |
| | DCP1, ACE1, MPO, PACIP1, PAXIP1L, PTIP, A2M, BLMH, BMH, |
| | PSEN1, AD3); Autism (Mecp2, BZRAP1, MDGA2, Sema5A, Neurexin |
| | 1, GLO1, MECP2, RTT, PPMX, MRX16, MRX79, NLGN3, NLGN4, |
| | KIAA1260, AUTSX2); Fragile X Syndrome (FMR2, FXR1, FXR2, |
| | mGLUR5); Huntington's disease and disease like disorders (HD, IT15, |
| | PRNP, PRIP, JPH3, JP3, HDL2, TBP, SCA17); Parkinson disease |
| | (NR4A2, NURR1, NOT, TINUR, SNCAIP, TBP, SCA17, SNCA, |
| | NACP, PARK1, PARK4, DJ1, PARK7, LRRK2, PARK8, PINK1, |
| | PARK6, UCHL1, PARK5, SNCA, NACP, PARK1, PARK4, PRKN, |
| | PARK2, PDJ, DBH, NDUFV2); Rett syndrome (MECP2, RTT, PPMX, |
| | MRX16, MRX79, CDKL5, STK9, MECP2, RTT, PPMX, MRX16, |
| | MRX79, x-Synuclein, DJ-1); Schizophrenia (Neuregulin1 (Nrg1), Erb4 |
| | (receptor for Neuregulin), Complexin1 (Cplx1), Tph1 Tryptophan |
| | hydroxylase, Tph2, Tryptophan hydroxylase 2, Neurexin 1, GSK3, |
| | GSK3a, GSK3b, 5-HTT (Slc6a4), COMT, DRD (Drd1a), SLC6A3, |
| | DAOA, DTNBP1, Dao (Dao1)); Secretase Related Disorders (APH-1 |
| | (alpha and beta), Presenilin (Psen1), nicastrin, (Ncstn), PEN-2, Nos1, |
| | Parp1, Nat1, Nat2); Trinucleotide Repeat Disorders (HTT (Huntington's |
| | Dx), SBMA/SMAX1/AR (Kennedy's Dx), FXN/X25 (Friedrich's |
| | Ataxia), ATX3 (Machado-Joseph's Dx), ATXN1 and ATXN2 |
| | (spinocerebellar ataxias), DMPK (myotonic dystrophy), Atrophin-1 and |
| | Atn1 (DRPLA Dx), CBP (Creb-BP - global instability), VLDLR |
| | (Alzheimer's), Atxn7, Atxn10). |
| Occular diseases | Age-related macular degeneration (Abcr, Ccl2, Cc2, cp (ceruloplasmin), |
| and disorders | Timp3, cathepsinD, Vldlr, Ccr2); Cataract (CRYAA, CRYA1, CRYBB2, |
| | CRYB2, PITX3, BFSP2, CP49, CP47, CRYAA, CRYA1, PAX6, AN2, |
| | MGDA, CRYBA1, CRYB1, CRYGC, CRYG3, CCL, LIM2, MP19, |
| | CRYGD, CRYG4, BFSP2, CP49, CP47, HSF4, CTM, HSF4, CTM, |
| | MIP, AQP0, CRYAB, CRYA2, CTPP2, CRYBB1, CRYGD, CRYG4, |
| | CRYBB2, CRYB2, CRYGC, CRYG3, CCL, CRYAA, CRYA1, GJA8, |
| | CX50, CAE1, GJA3, CX46, CZP3, CAE3, CCM1, CAM, KRIT1); |
| | Corneal clouding and dystrophy (APOA1, TGFBI, CSD2, CDGG1, |
| | CSD, BIGH3, CDG2, TACSTD2, TROP2, M1S1, VSX1, RINX, PPCD, |
| | PPD, KTCN, COL8A2, FECD, PPCD2, PIP5K3, CFD); Cornea plana |
| | congenital (KERA, CNA2); Glaucoma (MYOC, TIGR, GLC1A, JOAG, |
| | GPOA, OPTN, GLC1E, FIP2, HYPL, NRP, CYP1B1, GLC3A, OPA1, |
| | NTG, NPG, CYP1B1, GLC3A); Leber congenital amaurosis (CRB1, |
| | RP12, CRX, CORD2, CRD, RPGRIP1, LCA6, CORD9, RPE65, RP20, |
| | AIPL1, LCA4, GUCY2D, GUC2D, LCA1, CORD6, RDH12, LCA3); |
| | Macular dystrophy (ELOVL4, ADMD, STGD2, STGD3, RDS, RP7, |
| | PRPH2, PRPH, AVMD, AOFMD, VMD2). |

TABLE 3

| CELLULAR FUNCTION | GENES |
|---|---|
| PI3K/AKT Signaling | PRKCE; ITGAM; ITGA5; IRAK1; PRKAA2; EIF2AK2; |
| | PTEN; EIF4E; PRKCZ; GRK6; MAPK1; TSC1; PLK1; |
| | AKT2; IKBKB; PIK3CA; CDK8; CDKN1B; NFKB2; BCL2; |
| | PIK3CB; PPP2R1A; MAPK8; BCL2L1; MAPK3; TSC2; |
| | ITGA1; KRAS; EIF4EBP1; RELA; PRKCD; NOS3; |
| | PRKAA1; MAPK9; CDK2; PPP2CA; PIM1; ITGB7; |
| | YWHAZ; ILK; TP53; RAF1; IKBKG; RELB; DYRK1A; |
| | CDKN1A; ITGB1; MAP2K2; JAK1; AKT1; JAK2; PIK3R1; |
| | CHUK; PDPK1; PPP2R5C; CTNNB1; MAP2K1; NFKB1; |
| | PAK3; ITGB3; CCND1; GSK3A; FRAP1; SFN; ITGA2; |
| | TTK; CSNK1A1; BRAF; GSK3B; AKT3; FOXO1; SGK; |
| | HSP90AA1; RPS6KB1 |
| ERK/MAPK Signaling | PRKCE; ITGAM; ITGA5; HSPB1; IRAK1; PRKAA2; |
| | EIF2AK2; RAC1; RAP1A; TLN1; EIF4E; ELK1; GRK6; |
| | MAPK1; RAC2; PLK1; AKT2; PIK3CA; CDK8; CREB1; |
| | PRKCI; PTK2; FOS; RPS6KA4; PIK3CB; PPP2R1A; |
| | PIK3C3; MAPK8; MAPK3; ITGA1; ETS1; KRAS; MYCN; |
| | EIF4EBP1; PPARG; PRKCD; PRKAA1; MAPK9; SRC; |
| | CDK2; PPP2CA; PIM1; PIK3C2A; ITGB7; YWHAZ; |
| | PPP1CC; KSR1; PXN; RAF1; FYN; DYRK1A; ITGB1; |
| | MAP2K2; PAK4; PIK3R1; STAT3; PPP2R5C; MAP2K1; |
| | PAK3; ITGB3; ESR1; ITGA2; MYC; TTK; CSNK1A1; |
| | CRKL; BRAF; ATF4; PRKCA; SRF; STAT1; SGK |
| Glucocorticoid Receptor | RAC1; TAF4B; EP300; SMAD2; TRAF6; PCAF; ELK1; |
| Signaling | MAPK1; SMAD3; AKT2; IKBKB; NCOR2; UBE2I; |
| | PIK3CA; CREB1; FOS; HSPA5; NFKB2; BCL2; |
| | MAP3K14; STAT5B; PIK3CB; PIK3C3; MAPK8; BCL2L1; |
| | MAPK3; TSC22D3; MAPK10; NRIP1; KRAS; MAPK13; |
| | RELA; STAT5A; MAPK9; NOS2A; PBX1; NR3C1; |
| | PIK3C2A; CDKN1C; TRAF2; SERPINE1; NCOA3; |
| | MAPK14; TNF; RAF1; IKBKG; MAP3K7; CREBBP; |
| | CDKN1A; MAP2K2; JAK1; IL8; NCOA2; AKT1; JAK2; |
| | PIK3R1; CHUK; STAT3; MAP2K1; NFKB1; TGFBR1; |
| | ESR1; SMAD4; CEBPB; JUN; AR; AKT3; CCL2; MMP1; |
| | STAT1; IL6; HSP90AA1 |
| Axonal Guidance Signaling | PRKCE; ITGAM; ROCK1; ITGA5; CXCR4; ADAM12; |
| | IGF1; RAC1; RAP1A; E1F4E; PRKCZ; NRP1; NTRK2; |
| | ARHGEF7; SMO; ROCK2; MAPK1; PGF; RAC2; |
| | PTPN11; GNAS; AKT2; PIK3CA; ERBB2; PRKCI; PTK2; |
| | CFL1; GNAQ; PIK3CB; CXCL12; PIK3C3; WNT11; |
| | PRKD1; GNB2L1; ABL1; MAPK3; ITGA1; KRAS; RHOA; |
| | PRKCD; PIK3C2A; ITGB7; GLI2; PXN; VASP; RAF1; |
| | FYN; ITGB1; MAP2K2; PAK4; ADAM17; AKT1; PIK3R1; |
| | GLI1; WNT5A; ADAM10; MAP2K1; PAK3; ITGB3; |
| | CDC42; VEGFA; ITGA2; EPHA8; CRKL; RND1; GSK3B; |
| | AKT3; PRKCA |
| Ephrin Receptor Signaling | PRKCE; ITGAM; ROCK1; ITGA5; CXCR4; IRAK1; |
| | PRKAA2; EIF2AK2; RAC1; RAP1A; GRK6; ROCK2; |
| | MAPK1; PGF; RAC2; PTPN11; GNAS; PLK1; AKT2; |
| | DOK1; CDK8; CREB1; PTK2; CFL1; GNAQ; MAP3K14; |
| | CXCL12; MAPK8; GNB2L1; ABL1; MAPK3; ITGA1; |
| | KRAS; RHOA; PRKCD; PRKAA1; MAPK9; SRC; CDK2; |
| | PIM1; ITGB7; PXN; RAF1; FYN; DYRK1A; ITGB1; |
| | MAP2K2; PAK4; AKT1; JAK2; STAT3; ADAM10; |
| | MAP2K1; PAK3; ITGB3; CDC42; VEGFA; ITGA2; |
| | EPHA8; TTK; CSNK1A1; CRKL; BRAF; PTPN13; ATF4; |
| | AKT3; SGK |
| Actin Cytoskeleton | ACTN4; PRKCE; ITGAM; ROCK1; ITGA5; IRAK1; |
| Signaling | PRKAA2; EIF2AK2; RAC1; INS; ARHGEF7; GRK6; |
| | ROCK2; MAPK1; RAC2; PLK1; AKT2; PIK3CA; CDK8; |
| | PTK2; CFL1; PIK3CB; MYH9; DIAPH1; PIK3C3; MAPK8; |
| | F2R; MAPK3; SLC9A1; ITGA1; KRAS; RHOA; PRKCD; |
| | PRKAA1; MAPK9; CDK2; PIM1; PIK3C2A; ITGB7; |
| | PPP1CC; PXN; VIL2; RAF1; GSN; DYRK1A; ITGB1; |
| | MAP2K2; PAK4; PIP5K1A; PIK3R1; MAP2K1; PAK3; |
| | ITGB3; CDC42; APC; ITGA2; TTK; CSNK1A1; CRKL; |
| | BRAF; VAV3; SGK |
| Huntington's Disease | PRKCE; IGF1; EP300; RCOR1; PRKCZ; HDAC4; TGM2; |
| Signaling | MAPK1; CAPNS1; AKT2; EGFR; NCOR2; SP1; CAPN2; |
| | PIK3CA; HDAC5; CREB1; PRKC1; HSPA5; REST; |
| | GNAQ; PIK3CB; PIK3C3; MAPK8; IGF1R; PRKD1; |
| | GNB2L1; BCL2L1; CAPN1; MAPK3; CASP8; HDAC2; |
| | HDAC7A; PRKCD; HDAC11; MAPK9; HDAC9; PIK3C2A; |
| | HDAC3; TP53; CASP9; CREBBP; AKT1; PIK3R1; |
| | PDPK1; CASP1; APAF1; FRAP1; CASP2; JUN; BAX; |
| | ATF4; AKT3; PRKCA; CLTC; SGK; HDAC6; CASP3 |
| Apoptosis Signaling | PRKCE; ROCK1; BID; IRAK1; PRKAA2; EIF2AK2; BAK1; |
| | BIRC4; GRK6; MAPK1; CAPNS1; PLK1; AKT2; IKBKB; |
| | CAPN2; CDK8; FAS; NFKB2; BCL2; MAP3K14; MAPK8; |
| | BCL2L1; CAPN1; MAPK3; CASP8; KRAS; RELA; |
| | PRKCD; PRKAA1; MAPK9; CDK2; PIM1; TP53; TNF; |
| | RAF1; IKBKG; RELB; CASP9; DYRK1A; MAP2K2; |
| | CHUK; APAF1; MAP2K1; NFKB1; PAK3; LMNA; CASP2; |
| | BIRC2; TTK; CSNK1A1; BRAF; BAX; PRKCA; SGK; |
| | CASP3; BIRC3; PARP1 |
| B Cell Receptor Signaling | RAC1; PTEN; LYN; ELK1; MAPK1; RAC2; PTPN11; |
| | AKT2; IKBKB; PIK3CA; CREB1; SYK; NFKB2; CAMK2A; |
| | MAP3K14; PIK3CB; PIK3C3; MAPK8; BCL2L1; ABL1; |
| | MAPK3; ETS1; KRAS; MAPK13; RELA; PTPN6; MAPK9; |
| | EGR1; PIK3C2A; BTK; MAPK14; RAF1; IKBKG; RELB; |
| | MAP3K7; MAP2K2; AKT1; PIK3R1; CHUK; MAP2K1; |
| | NFKB1; CDC42; GSK3A; FRAP1; BCL6; BCLI0; JUN; |
| | GSK3B; ATF4; AKT3; VAV3; RPS6KB1 |
| Leukocyte Extravasation | ACTN4; CD44; PRKCE; ITGAM; ROCK1; CXCR4; CYBA; |
| Signaling | RAC1; RAP1A; PRKCZ; ROCK2; RAC2; PTPN11; |
| | MMP14; PIK3CA; PRKCI; PTK2; PIK3CB; CXCL12; |
| | PIK3C3; MAPK8; PRKD1; ABL1; MAPK10; CYBB; |
| | MAPK13; RHOA; PRKCD; MAPK9; SRC; PIK3C2A; BTK; |
| | MAPK14; NOX1; PXN; VIL2; VASP; ITGB1; MAP2K2; |
| | CTNND1; PIK3R1; CTNNB1; CLDN1; CDC42; F11R; ITK; |
| | CRKL; VAV3; CTTN; PRKCA; MMP1; MMP9 |
| Integrin Signaling | ACTN4; ITGAM; ROCK1; ITGA5; RAC1; PTEN; RAP1A; |
| | TLN1; ARHGEF7; MAPK1; RAC2; CAPNS1; AKT2; |
| | CAPN2; PIK3CA; PTK2; PIK3CB; PIK3C3; MAPK8; |
| | CAV1; CAPN1; ABL1; MAPK3; ITGAl; KRAS; RHOA; |
| | SRC; PIK3C2A; ITGB7; PPP1CC; ILK; PXN; VASP; |
| | RAF1; FYN; ITGB1; MAP2K2; PAK4; AKT1; PIK3R1; |
| | TNK2; MAP2K1; PAK3; ITGB3; CDC42; RND3; ITGA2; |
| | CRKL; BRAF; GSK3B; AKT3 |
| Acute Phase Response | IRAK1; SOD2; MYD88; TRAF6; ELK1; MAPK1; PTPN11; |
| Signaling | AKT2; IKBKB; PIK3CA; FOS; NFKB2; MAP3K14; |
| | PIK3CB; MAPK8; RIPK1; MAPK3; IL6ST; KRAS; |
| | MAPK13; IL6R; RELA; SOCS1; MAPK9; FTL; NR3C1; |
| | TRAF2; SERPINE1; MAPK14; TNF; RAF1; PDK1; |
| | IKBKG; RELB; MAP3K7; MAP2K2; AKT1; JAK2; PIK3R1; |
| | CHUK; STAT3; MAP2K1; NFKB1; FRAP1; CEBPB; JUN; |
| | AKT3; IL1R1; IL6 |
| PTEN Signaling | ITGAM; ITGA5; RAC1; PTEN; PRKCZ; BCL2L11; |
| | MAPK1; RAC2; AKT2; EGFR; IKBKB; CBL; PIK3CA; |
| | CDKN1B; PTK2; NFKB2; BCL2; PIK3CB; BCL2L1; |
| | MAPK3; ITGA1; KRAS; ITGB7; ILK; PDGFRB; INSR; |
| | RAF1; IKBKG; CASP9; CDKN1A; ITGB1; MAP2K2; |
| | AKT1; PIK3R1; CHUK; PDGFRA; PDPK1; MAP2K1; |
| | NFKB1; ITGB3; CDC42; CCND1; GSK3A; ITGA2; |
| | GSK3B; AKT3; FOXO1; CASP3; RPS6KB1 |
| p53 Signaling | PTEN; EP300; BBC3; PCAF; FASN; BRCA1: GADD45A; |
| | BIRC5; AKT2; PIK3CA; CHEK1; TP53INP1; BCL2; |
| | PIK3CB; PIK3C3; MAPK8; THBS1; ATR; BCL2L1; E2F1; |
| | PMAIP1; CHEK2; TNFRSF10B; TP73; RB1; HDAC9; |
| | CDK2; PIK3C2A; MAPK14; TP53; LRDD; CDKN1A; |
| | HIPK2; AKT1; PIK3R1; RRM2B; APAF1; CTNNB1; |
| | SIRT1; CCND1; PRKDC; ATM; SFN; CDKN2A; JUN; |
| | SNAI2; GSK3B; BAX; AKT3 |
| Aryl Hydrocarbon Receptor | HSPB1; EP300; FASN; TGM2; RXRA; MAPK1; NQO1; |
| Signaling | NCOR2; SP1; ARNT; CDKN1B; FOS; CHEK1; |
| | SMARCA4; NFKB2; MAPK8; ALDH1A1; ATR; E2F1; |
| | MAPK3; NRIP1; CHEK2; RELA; TP73; GSTP1; RB1; |
| | SRC; CDK2; AHR; NFE2L2; NCOA3; TP53; TNF; |
| | CDKN1A; NCOA2; APAF1; NFKB1; CCND1; ATM; ESR1; |
| | CDKN2A; MYC; JUN; ESR2; BAX; IL6; CYP1B1; |
| | HSP90AA1 |
| Xenobiotic Metabolism | PRKCE; EP300; PRKCZ; RXRA; MAPK1; NQO1; |
| Signaling | NCOR2; PIK3CA; ARNT; PRKCI; NFKB2; CAMK2A; |
| | PIK3CB; PPP2R1A; PIK3C3; MAPK8; PRKD1; |
| | ALDH1A1; MAPK3; NRIP1; KRAS; MAPK13; PRKCD; |
| | GSTP1; MAPK9; NOS2A; ABCB1; AHR; PPP2CA; FTL; |
| | NFE2L2; PIK3C2A; PPARGC1A; MAPK14; TNF; RAF1; |
| | CREBBP; MAP2K2; PIK3R1; PPP2R5C; MAP2K1; |
| | KFKB1; KEAP1; PRKCA; EIF2AK3; IL6; CYP1B1; |
| | HSP90AA1 |
| SAPK/JNK Signaling | PRKCE; IRAK1; PRKAA2; EIF2AK2; RAC1; ELK1; |
| | GRK6; MAPK1; GADD45A; RAC2; PLK1; AKT2; PIK3CA; |
| | FADD; CDK8; PIK3CB; PIK3C3; MAPK8; RIPK1; |
| | GNB2L1; IRS1; MAPK3; MAPK10; DAXX; KRAS; |
| | PRKCD; PRKAA1; MAPK9; CDK2; PIM1; PIK3C2A; |
| | TRAF2; TP53; LCK; MAP3K7; DYRK1A; MAP2K2; |
| | PIK3R1; MAP2K1; PAK3; CDC42; JUN; TTK; CSNK1A1; |
| | CRKL; BRAF; SGK |
| PPAr/RXR Signaling | PRKAA2; EP300; INS; SMAD2; TRAF6; PPARA; FASN; |
| | RXRA; MAPK1; SMAD3; GNAS; IKBKB; NCOR2; |
| | ABCA1; GNAQ; NFKB2; MAP3K14; STAT5B; MAPK8; |
| | IRS1; MAPK3; KRAS; RELA; PRKAA1; PPARGC1A; |
| | NCOA3; MAPK14; INSR; RAF1; IKBKG; RELB; MAP3K7; |
| | CREBBP; MAP2K2; JAK2; CHUK; MAP2K1; NFKB1; |
| | TGFBR1; SMAD4; JUN; IL1R1; PRKCA; IL6; HSP9AA1; |
| | ADIPOQ |
| NF-KB Signaling | IRAK1; EIF2AK2; EP300; INS; MYD88; PRKCZ; TRAF6; |
| | TBK1; AKT2; EGFR; IKBKB; PIK3CA; BTRC; NFKB2; |
| | MAP3K14; PIK3CB; PIK3C3; MAPK8; RIPK1; HDAC2; |
| | KRAS; RELA; PIK3C2A; TRAF2; TLR4; PDGFRB; TNF; |
| | INSR; LCK; IKBKG; RELB; MAP3K7; CREBBP; AKT1; |
| | PIK3R1; CHUK; PDGFRA; NFKB1; TLR2; BCL10; |
| | GSK3B; AKT3; TNFAIP3; IL1R1 |
| Neuregulin Signaling | ERBB4; PRKCE; ITGAM; ITGA5; PTEN; PRKCZ; ELK1; |
| | MAPK1; PTPN11; AKT2; EGFR; ERBB2; PRKCI; |
| | CDKN1B; STAT5B; PRKD1; MAPK3; ITGA1; KRAS; |
| | PRKCD; STAT5A; SRC; ITGB7; RAF1; ITGB1; MAP2K2; |
| | ADAM17; AKT1; PIK3R1; PDPK1; MAP2K1; ITGB3; |
| | EREG; FRAP1; PSEN1; ITGA2; MYC; NRG1; CRKL; |
| | AKT3; PRKCA; HSP90AA1; RP S6KB1 |
| Wnt & Beta catenin | CD44; EP300; LRP6; DVL3; CSNK1E; GJA1; SMO; |
| Signaling | AKT2; PIN1; CDH1; BTRC; GNAQ; MARK2; PPP2R1A; |
| | WNT11; SRC; DKK1; PPP2CA; SOX6; SFRP2; ILK; |
| | LEF1; SOX9; TP53; MAP3K7; CREBBP; TCF7L2; AKT1; |
| | PPP2R5C; WNT5A; LRP5; CTNNB1; TGFBR1; CCND1; |
| | GSK3A; DVL1; APC; CDKN2A; MYC; CSNK1A1; GSK3B; |
| | AKT3; SOX2 |
| Insulin Receptor | PTEN; INS; EIF4E; PTPN1; PRKCZ; MAPK1; TSC1; |
| | PTPN11; AKT2; CBL; PIK3CA; PRKCI; PIK3CB; PIK3C3; |
| | MAPK8; IRS1; MAPK3; TSC2; KRAS; EIF4EBP1; |
| | SLC2A4; PIK3C2A; PPP1CC; INSR; RAF1; FYN; |
| | MAP2K2; JAK1; AKT1; JAK2; PIK3R1; PDPK1; MAP2K1; |
| | GSK3A; FRAP1; CRKL; GSK3B; AKT3; FOXO1; SGK; |
| | RPS6KB1 |
| IL-6 Signaling | HSPB1; TRAF6; MAPKAPK2; ELK1; MAPK1; PTPN11; |
| | IKBKB; FOS; NFKB2; MAP3K14; MAPK8; MAPK3; |
| | MAPK10; IL6ST; KRAS; MAPK13; IL6R; RELA; SOCS1; |
| | MAPK9; ABCB1; TRAF2; MAPK14; TNF; RAF1; IKBKG; |
| | RELB; MAP3K7; MAP2K2; IL8; JAK2; CHUK; STAT3; |
| | MAP2K1; NFKB1; CEBPB; JUN; IL1R1; SRF; IL6 |
| Hepatic Cholestasis | PRKCE; IRAK1; INS; MYD88; PRKCZ; TRAF6; PPARA; |
| | RXRA; IKBKB; PRKCI; NFKB2; MAP3K14; MAPK8; |
| | PRKD1; MAPK10; RELA; PRKCD; MAPK9; ABCB1; |
| | TRAF2; TLR4; TNF; INSR; IKBKG; RELB; MAP3K7; IL8; |
| | CHUK; NR1H2; TJP2; NFKB1; ESR1; SREBF1; FGFR4; |
| | JUN; IL1R1; PRKCA; IL6 |
| IGF-1 Signaling | IGF1; PRKCZ; ELK1; MAPK1; PTPN11; NEDD4; AKT2; |
| | PIK3CA; PRKCI; PTK2; FOS; PIK3CB; PIK3C3; MAPK8; |
| | IGF1R; IRS1; MAPK3; IGFBP7; KRAS; PIK3C2A; |
| | YWHAZ; PXN; RAF1; CASP9; MAP2K2; AKT1; PIK3R1; |
| | PDPK1; MAP2K1; IGFBP2; SFN; JUN; CYR61; AKT3; |
| | FOXO1; SRF; CTGF; RPS6KB1 |
| NRF2-mediated Oxidative | PRKCE; EP300; SOD2; PRKCZ; MAPK1; SQSTM1; |
| Stress Response | NQO1; PIK3CA; PRKCI; FOS; PIK3CB; PIK3C3; MAPK8; |
| | PRKD1; MAPK3; KRAS; PRKCD; GSTP1; MAPK9; FTL; |
| | NFE2L2; PIK3C2A; MAPK14; RAF1; MAP3K7; CREBBP; |
| | MAP2K2; AKT1; PIK3R1; MAP2K1; PPIB; JUN; KEAP1; |
| | GSK3B; ATF4; PRKCA; EIF2AK3; HSP90AA1 |
| Hepatic Fibrosis/Hepatic | EDN1; IGF1; KDR; FLT1; SMAD2; FGFR1; MET; PGF; |
| Stellate Cell Activation | SMAD3; EGFR; FAS; CSF1; NFKB2; BCL2; MYH9; |
| | IGF1R; IL6R; RELA; TLR4; PDGFRB; TNF; RELB; IL8; |
| | PDGFRA; NFKB1; TGFBR1; SMAD4; VEGFA; BAX; |
| | IL1R1; CCL2; HGF; MMP1; STAT1; IL6; CTGF; MMP9 |
| PPAR Signaling | EP300; INS; TRAF6; PPARA; RXRA; MAPK1; IKBKB; |
| | NCOR2; FOS; NFKB2; MAP3K14; STAT5B; MAPK3; |
| | NRIP1; KRAS; PPARG; RELA; STAT5A; TRAF2; |
| | PPARGC1A; PDGFRB; TNF; INSR; RAF1; IKBKG; |
| | RELB; MAP3K7; CREBBP; MAP2K2; CHUK; PDGFRA; |
| | MAP2K1; NFKB1; JUN; IL1R1; HSP90AA1 |
| Fc Epsilon RI Signaling | PRKCE; RAC1; PRKCZ; LYN; MAPK1; RAC2; PTPN11; |
| | AKT2; PIK3CA; SYK; PRKC; PIK3CB; PIK3C3; MAPK8; |
| | PRKD1; MAPK3; MAPK10; KRAS; MAPK13; PRKCD; |
| | MAPK9; PIK3C2A; BTK; MAPK14; TNF; RAF1; FYN; |
| | MAP2K2; AKT1; PIK3R1; PDPK1; MAP2K1; AKT3; |
| | VAV3; PRKCA |
| G-Protein Coupled | PRKCE; RAP1A; RGS16; MAPK1; GNAS; AKT2; IKBKB; |
| Receptor Signaling | PIK3CA; CREB1; GNAQ; NFKB2; CAMK2A; PIK3CB; |
| | PIK3C3; MAPK3; KRAS; RELA; SRC; PIK3C2A; RAF1; |
| | IKBKG; RELB; FYN; MAP2K2; AKT1; PIK3R1; CHUK; |
| | PDPK1; STAT3; MAP2K1; NFKB1; BRAF; ATF4; AKT3; |
| | PRKCA |
| Inositol Phosphate | PRKCE; IRAK1; PRKAA2; EIF2AK2; PTEN; GRK6; |
| Metabolism | MAPK1; PLK1; AKT2; PIK3CA; CDK8; PIK3CB; PIK3C3; |
| | MAPK8; MAPK3; PRKCD; PRKAA1; MAPK9; CDK2; |
| | PIM1; PIK3C2A; DYRK1A; MAP2K2; PIP5K1A; PIK3R1; |
| | MAP2K1; PAK3; ATM; TTK; CSNK1A1; BRAF; SGK |
| PDGF Signaling | EIF2AK2; ELK1; ABL2; MAPK1; PIK3CA; FOS; PIK3CB; |
| | PIK3C3; MAPK8; CAV1; ABL1; MAPK3; KRAS; SRC; |
| | PIK3C2A; PDGFRB; RAF1; MAP2K2; JAK1; JAK2; |
| | PIK3R1; PDGFRA; STAT3; SPHK1; MAP2K1; MYC; |
| | JUN; CRKL; PRKCA; SRF; STAT1; SPHK2 |
| VEGF Signaling | ACTN4; ROCK1; KDR; FLT1; ROCK2; MAPK1; PGF; |
| | AKT2; PIK3CA; ARNT; PTK2; BCL2; PIK3CB; PIK3C3; |
| | BCL2L1; MAPK3; KRAS; HIF1A; NOS3; PIK3C2A; PXN; |
| | RAF1; MAP2K2; ELAVL1; AKT1; PIK3R1; MAP2K1; SFN; |
| | VEGFA; AKT3; FOXO1; PRKCA |
| Natural Killer Cell Signaling | PRKCE; RAC1; PRKCZ; MAPK1; RAC2; PTPN11; |
| | KIR2DL3; AKT2; PIK3CA; SYK; PRKCI; PIK3CB; |
| | PIK3C3; PRKD1; MAPK3; KRAS; PRKCD; PTPN6; |
| | PIK3C2A; LCK; RAF1; FYN; MAP2K2; PAK4; AKT1; |
| | PIK3R1; MAP2K1; PAK3; AKT3; VAV3; PRKCA |
| Cell Cycle: G1/S | HDAC4; SMAD3; SUV39H1; HDAC5; CDKN1B; BTRC; |
| Checkpoint Regulation | ATR; ABL1; E2F1; HDAC2; HDAC7A; RB1; HDAC11; |
| | HDAC9; CDK2; E2F2; HDAC3; TP53; CDKN1A; CCND1; |
| | E2F4; ATM; RBL2; SMAD4; CDKN2A; MYC; NRG1; |
| | GSK3B; RBL1; HDAC6 |
| T Cell Receptor Signaling | RAC1; ELK1; MAPK1; IKBKB; CBL; PIK3CA; FOS; |
| | NFKB2; PIK3CB; PIK3C3; MAPK8; MAPK3; KRAS; |
| | RELA; PIK3C2A; BTK; LCK; RAF1; IKBKG; RELB; FYN; |
| | MAP2K2; PIK3R1; CHUK; MAP2K1; NFKB1; ITK; BCL10; |
| | JUN; VAV3 |
| Death Receptor Signaling | CRADD; HSPB1; BID; BIRC4; TBK1; IKBKB; FADD; |
| | FAS; NFKB2; BCL2; MAP3K14; MAPK8; RIPK1; CASP8; |
| | DAXX; TNFRSF10B; RELA; TRAF2; TNF; IKBKG; RELB; |
| | CASP9; CHUK; APAF1; NFKB1; CASP2; BIRC2; CASP3; |
| | BIRC3 |
| FGF Signaling | RAC1; FGFR1; MET; MAPKAPK2; MAPK1; PTPN11; |
| | AKT2; PIK3CA; CREB1; PIK3CB; PIK3C3; MAPK8; |
| | MAPK3; MAPK13; PTPN6; PIK3C2A; MAPK14; RAF1; |
| | AKT1; PIK3R1; STAT3; MAP2K1; FGFR4; CRKL; ATF4; |
| | AKT3; PRKCA; HGF |
| GM-CSF Signaling | LYN; ELK1; MAPK1; PTPN11; AKT2; PIK3CA; CAMK2A; |
| | STAT5B; PIK3CB; PIK3C3; GNB2L1; BCL2L1; MAPK3; |
| | ETS1; KRAS; RUNX1; PIM1; PIK3C2A; RAF1; MAP2K2; |
| | AKT1; JAK2; PIK3R1; STAT3; MAP2K1; CCND1; AKT3; |
| | STAT1 |
| Amyotrophic Lateral | BID; IGF1; RAC1; BIRC4; PGF; CAPNS1; CAPN2; |
| Sclerosis Signaling | PIK3CA; BCL2; PIK3CB; PIK3C3; BCL2L1; CAPN1; |
| | PIK3C2A; TP53; CASP9; PIK3R1; RAB5A; CASP1; |
| | APAF1; VEGFA; BIRC2; BAX; AKT3; CASP3; BIRC3 |
| JAK/Stat Signaling | PTPN1; MAPK1; PTPN11; AKT2; PIK3CA; STAT5B; |
| | PIK3CB; PIK3C3; MAPK3; KRAS; SOCS1; STAT5A; |
| | PTPN6; PIK3C2A; RAF1; CDKN1A; MAP2K2; JAK1; |
| | AKT1; JAK2; PIK3R1; STAT3; MAP2K1; FRAP1; AKT3; |
| | STAT1 |
| Nicotinate and Nicotinamide | PRKCE; IRAK1; PRKAA2; EIF2AK2; GRK6; MAPK1; |
| Metabolism | PLK1; AKT2; CDK8; MAPK8; MAPK3; PRKCD; PRKAA1; |
| | PBEF1; MAPK9; CDK2; PIM1; DYRK1A; MAP2K2; |
| | MAP2K1; PAK3; NT5E; TTK; CSNK1A1; BRAF; SGK |
| Chemokine Signaling | CXCR4; ROCK2; MAPK1; PTK2; FOS; CFL1; GNAQ; |
| | CAMK2A; CXCL12; MAPK8; MAPK3; KRAS; MAPK13; |
| | RHOA; CCR3; SRC; PPP1CC; MAPK14; NOX1; RAF1; |
| | MAP2K2; MAP2K1; JUN; CCL2; PRKCA |
| IL-2 Signaling | ELK1; MAPK1; PTPN11; AKT2; PIK3CA; SYK; FOS; |
| | STAT5B; PIK3CB; PIK3C3; MAPK8; MAPK3; KRAS; |
| | SOCS1; STAT5A; PIK3C2A; LCK; RAF1; MAP2K2; |
| | JAK1; AKT1; PIK3R1; MAP2K1; JUN; AKT3 |
| Synaptic Long Term | PRKCE; IGF1; PRKCZ; PRDX6; LYN; MAPK1; GNAS; |
| Depression | PRKCI; GNAQ; PPP2R1A; IGF1R; PRKD1; MAPK3; |
| | KRAS; GRN; PRKCD; NO53; NOS2A; PPP2CA; |
| | YWHAZ; RAF1; MAP2K2; PPP2R5C; MAP2K1; PRKCA |
| Estrogen Receptor | TAF4B; EP300; CARM1; PCAF; MAPK1; NCOR2; |
| Signaling | SMARCA4; MAPK3; NRIP1; KRAS; SRC; NR3C1; |
| | HDAC3; PPARGC1A; RBM9; NCOA3; RAF1; CREBBP; |
| | MAP2K2; NCOA2; MAP2K1; PRKDC; ESR1; ESR2 |
| Protein Ubiquitination | TRAF6; SMURF1; BIRC4; BRCA1; UCHL1; NEDD4; |
| Pathway | CBL; UBE2I; BTRC; HSPA5; USP7; USP10; FBXW7; |
| | USP9X; STUB1; USP22; B2M; BIRC2; PARK2; USP8; |
| | USP1; VHL; HSP90AA1; BIRC3 |
| IL-10 Signaling | TRAF6; CCR1; ELK1; IKBKB; SP1; FOS; NFKB2; |
| | MAP3K14; MAPK8; MAPK13; RELA; MAPK14; TNF; |
| | IKBKG; RELB; MAP3K7; JAK1; CHUK; STAT3; NFKB1; |
| | JUN; IL1R1; IL6 |
| VDR/RXR Activation | PRKCE; EP300; PRKCZ; RXRA; GADD45A; HES1; |
| | NCOR2; SP1; PRKC1; CDKN1B; PRKD1; PRKCD; |
| | RUNX2; KLF4; YY1; NCOA3; CDKN1A; NCOA2; SPP1; |
| | LRP5; CEBPB; FOXO1; PRKCA |
| TGF-beta Signaling | EP300; SMAD2; SMURF1; MAPK1; SMAD3; SMAD1; |
| | FOS; MAPK8; MAPK3; KRAS; MAPK9; RUNX2; |
| | SERPINE1; RAF1; MAP3K7; CREBBP; MAP2K2; |
| | MAP2K1; TGFBR1; SMAD4; JUN; SMAD5 |
| Toll-like Receptor Signaling | IRAK1; EIF2AK2; MYD88; TRAF6; PPARA; ELK1; |
| | IKBKB; FOS; NFKB2; MAP3K14; MAPK8; MAPK13; |
| | RELA; TLR4; MAPK14; IKBKG; RELB; MAP3K7; CHUK; |
| | NFKB1; TLR2; JUN |
| p38 MAPK Signaling | HSPB1; IRAK1; TRAF6; MAPKAPK2; ELK1; FADD; FAS; |
| | CREB1; DDIT3; RPS6KA4; DAXX; MAPK13; TRAF2; |
| | MAPK14; TNF; MAP3K7; TGFBR1; MYC; ATF4; IL1R1; |
| | SRF; STAT1 |
| Neurotrophin/TRK Signaling | NTRK2; MAPK1; PTPN11; PIK3CA; CREB1; FOS; |
| | PIK3CB; PIK3C3; MAPK8; MAPK3; KRAS; PIK3C2A; |
| | RAF1; MAP2K2; AKT1; PIK3R1; PDPK1; MAP2K1; |
| | CDC42; JUN; ATF4 |
| FXR/RXR Activation | INS; PPARA; FASN; RXRA; AKT2; SDC1; MAPK8; |
| | APOB; MAPK10; PPARG; MTTP; MAPK9; PPARGC1A; |
| | TNF; CREBBP; AKT1; SREBF1; FGFR4; AKT3; FOXO1 |
| Synaptic Long Term | PRKCE; RAP1A; EP300; PRKCZ; MAPK1; CREB1; |
| Potentiation | PRKCI; GNAQ; CAMK2A; PRKD1; MAPK3; KRAS; |
| | PRKCD; PPP1CC; RAF1; CREBBP; MAP2K2; MAP2K1; |
| | ATF4; PRKCA |
| Calcium Signaling | RAP1A; EP300; HDAC4; MAPK1; HDAC5; CREB1; |
| | CAMK2A; MYH9; MAPK3; HDAC2; HDAC7A; HDAC11; |
| | HDAC9; HDAC3; CREBBP; CALR; CAMKK2; ATF4; |
| | HDAC6 |
| EGF Signaling | ELK1; MAPK1; EGFR; PIK3CA; FOS; PIK3CB; PIK3C3; |
| | MAPK8; MAPK3; PIK3C2A; RAF1; JAK1; PIK3R1; |
| | STAT3; MAP2K1; JUN; PRKCA; SRF; STAT1 |
| Hypoxia Signaling in the | EDN1; PTEN; EP300; NQO1; UBE2I; CREB1; ARNT; |
| Cardiovascular System | HIF1A; SLC2A4; NOS3; TP53; LDHA; AKT1; ATM; |
| | VEGFA; JUN; ATF4; VHL; HSP90AA1 |
| LPS/IL-1 Mediated Inhibition | IRAK1; MYD88; TRAF6; PPARA; RXRA; ABCA1; |
| of RXR Function | MAPK8; ALDH1A1; GSTP1; MAPK9; ABCB1; TRAF2; |
| | TLR4; TNF; MAP3K7; NR1H2; SREBF1; JUN; IL1R1 |
| LXR/RXR Activation | FASN; RXRA; NCOR2; ABCA1; NFKB2; IRF3; RELA; |
| | NOS2A; TLR4; TNF; RELB; LDLR; NR1H2; NFKB1; |
| | SREBF1; IL1R1; CCL2; IL6; MMP9 |
| Amyloid Processing | PRKCE; CSNK1E; MAPK1; CAPNS1; AKT2; CAPN2; |
| | CAPN1; MAPK3; MAPK13; MAPT; MAPK14; AKT1; |
| | PSEN1; CSNK1A1; GSK3B; AKT3; APP |
| IL-4 Signaling | AKT2; PIK3CA; PIK3CB; PIK3C3; IRS1; KRAS; SOCS1; |
| | PTPN6; NR3C1; PIK3C2A; JAK1; AKT1; JAK2; PIK3R1; |
| | FRAP1; AKT3; RPS6KB1 |
| Cell Cycle: G2/M DNA | EP300; PCAF; BRCA1; GADD45A; PLK1; BTRC; |
| Damage Checkpoint | CHEK1; ATR; CHEK2; YWHAZ; TP53; CDKN1A; |
| Regulation | PRKDC; ATM; SFN; CDKN2A |
| Nitric Oxide Signaling in the | KDR; FLT1; PGF; AKT2; PIK3CA; PIK3CB; PIK3C3; |
| Cardiovascular System | CAV1; PRKCD; NOS3; PIK3C2A; AKT1; PIK3R1; |
| | VEGFA; AKT3; HSP90AA1 |
| Purine Metabolism | NME2; SMARCA4; MYH9; RRM2; ADAR; EIF2AK4; |
| | PKM2; ENTPD1; RAD51; RRM2B; TJP2; RAD51C; |
| | NT5E; POLD1; NME1 |
| cAMP-mediated Signaling | RAP1A; MAPK1; GNAS; CREB1; CAMK2A; MAPK3; |
| | SRC; RAF1; MAP2K2; STAT3; MAP2K1; BRAF; ATF4 |
| Mitochondrial Dysfunction | SOD2; MAPK8; CASP8; MAPK10; MAPK9; CASP9; |
| | PARK7; PSEN1; PARK2; APP; CASP3 |
| Notch Signaling | HES1; JAG1; NUMB; NOTCH4; ADAM17; NOTCH2; |
| | PSEN1; NOTCH3; NOTCH1; DLL4 |
| Endoplasmic Reticulum | HSPA5; MAPK8; XBPI; TRAF2; ATF6; CASP9; ATF4; |
| Stress Pathway | EIF2AK3; CASP3 |
| Pyrimidine Metabolism | NME2; AICDA; RRM2; EIF2AK4; ENTPD1; RRM2B; |
| | NT5E; POLD1; NME1 |
| Parkinson's Signaling | UCHL1; MAPK8; MAPK13; MAPK14; CASP9; PARK7; |
| | PARK2; CASP3 |
| Cardiac & Beta Adrenergic | GNAS; GNAQ; PPP2R1A; GNB2L1; PPP2CA; PPP1CC; |
| Signaling | PPP2R5C |
| Glycolysis/Gluconeogenesis | HK2; GCK; GPI; ALDH1A1; PKM2; LDHA; HK1 |
| Interferon Signaling | IRF1; SOCS1; JAK1; JAK2; IFITM1; STAT1; IFIT3 |
| Sonic Hedgehog Signaling | ARRB2; SMO; GLI2; DYRK1A; GLI1; GSK3B; DYRKIB |
| Glycerophospholipid | PLD1; GRN; GPAM; YWHAZ; SPHK1; SPHK2 |
| Metabolism | |
| Phospholipid Degradation | PRDX6; PLD1; GRN; YWHAZ; SPHK1; SPHK2 |
| Tryptophan Metabolism | SIAH2; PRMT5; NEDD4; ALDH1A1; CYP1B1; SIAH1 |
| Lysine Degradation | SUV39H1; EHMT2; NSD1; SETD7; PPP2R5C |
| Nucleotide Excision Repair | ERCC5; ERCC4; XPA; XPC; ERCC1 |
| Pathway | |
| Starch and Sucrose | UCHL1; HK2; GCK; GPI; HK1 |
| Metabolism | |
| Amnosugars Metabolism | NQO1; HK2; GCK; HK1 |
| Arachidonic Acid | PRDX6; GRN; YWHAZ; CYP1B1 |
| Metabolism | |
| Circadian Rhythm Signaling | CSNK1E; CREB1; ATF4; NR1D1 |
| Coagulation System | BDKRB1; F2R; SERPINE1; F3 |
| Dopamine Receptor | PPP2R1A; PPP2CA; PPP1CC; PPP2R5C |
| Signaling | |
| Glutathione Metabolism | IDH2; GSTP1; ANPEP; IDH1 |
| Glycerolipid Metabolism | ALDH1A1; GPAM; SPHK1; SPHK2 |
| Linoleic Acid Metabolism | PRDX6; GRN; YWHAZ; CYP1B1 |
| Methionine Metabolism | DNMT1; DNMT3B; AHCY; DNMT3A |
| Pyruvate Metabolism | GLO1; ALDH1A1; PKM2; LDHA |
| Arginine and Proline | ALDH1A1; NOS3; NOS2A |
| Metabolism | |
| Eicosanoid Signaling | PRDX6; GRN; YWHAZ |
| Fructose and Mannose | HK2; GCK; HK1 |
| Metabolism | |
| Galactose Metabolism | HK2; GCK; HK1 |
| Stilbene, Coumarine and | PRDX6; PRDX1; TYR |
| Lignin Biosynthesis | |
| Antigen Presentation | CALR; B2M |
| Pathway | |
| Biosynthesis of Steroids | NQO1; DHCR7 |
| Butanoate Metabolism | ALDH1A1; NLGN1 |
| Citrate Cycle | IDH2; IDH1 |
| Fatty Acid Metabolism | ALDH1A1; CYP1B1 |
| Glycerophospholipid | PRDX6; CHKA |
| Metabolism | |
| Histidine Metabolism | PRMT5; ALDH1A1 |
| Inositol Metabolism | ERO1L; APEX1 |
| Metabolism of Xenobiotics | GSTP1; CYP1B1 |
| by Cytochrome p450 | |
| Methane Metabolism | PRDX6; PRDX1 |
| Phenylalanine Metabolism | PRDX6; PRDX1 |
| Propanoate Metabolism | ALDH1A1; LDHA |
| Selenoamino Acid | PRMT5; AHCY |
| Metabolism | |
| Sphingolipid Metabolism | SPHK1; SPHK2 |
| Amnophosphonate | PRMT5 |
| Metabolism | |
| Androgen and Estrogen | PRMT5 |
| Metabolism | |
| Ascorbate and Aldarate | ALDH1A1 |
| Metabolism | |
| Bile Acid Biosynthesis | ALDH1A1 |
| Cysteine Metabolism | LDHA |
| Fatty Acid Biosynthesis | FASN |
| Glutamate Receptor | GNB2L1 |
| Signaling | |
| NRF2-mediated Oxidative | PRDX1 |
| Stress Response | |
| Pentose Phosphate | GPI |
| Pathway | |
| Pentose and Glucuronate | UCHL1 |
| Interconversions | |
| Retinol Metabolism | ALDH1A1 |
| Riboflavin Metabolism | TYR |
| Tyrosine Metabolism | PRMT5, TYR |
| Ubiquinone Biosynthesis | PRMT5 |
| Valine, Leucine and | ALDH1A1 |
| Isoleucine Degradation | |
| Glycine, Serine and | CHKA |
| Threonine Metabolism | |
| Lysine Degradation | ALDH1A1 |
| Pain/Taste | TRPM5; TRPA1 |
| Pain | TRPM7; TRPC5; TRPC6; TRPC1; Cnr1; cnr2; Grk2; |
| | Trpa1; Pomc; Cgrp; Crf; Pka; Era; Nr2b; TRPM5; Prkaca; |
| | Prkacb; Prkar1a; Prkar2a |
| Mitochondrial Function | AIF; CytC; SMAC (Diablo); Aifm-1; Aifm-2 |
| Developmental Neurology | BMP-4; Chordin (Chrd); Noggin (Nog); WNT (Wnt2; |
| | Wnt2b; Wnt3a; Wnt4; Wnt5a; Wnt6; Wnt7b; Wnt8b; |
| | Wnt9a; Wnt9b; Wnt10a; Wnt10b; Wnt16); beta-catenin; |
| | Dkk-1; Frizzled related proteins; Otx-2; Gbx2; FGF-8; |
| | Reelin; Dab1; unc-86 (Pou4fl or Brn3a); Numb; Reln |

The plurality of cells (e.g., a plurality of engineered immune cells) as disclosed herein can be administered to a subject in need thereof to treat a target cell, a target tissue, a target condition, or a target disease of a subject.

A target disease can be a viral, bacterial, and/or parasitic infection; inflammatory and/or autoimmune disease; or neoplasm such as a cancer and/or tumor.

A target cell can be a diseased cell. A diseased cell can have altered metabolic, gene expression, and/or morphologic features. A diseased cell can be a cancer cell, a diabetic cell, and an apoptotic cell. A diseased cell can be a cell from a diseased subject. Exemplary diseases can include blood disorders, cancers, metabolic disorders, eye disorders, organ disorders, musculoskeletal disorders, cardiac disease, and the like.

A variety of target cells can be killed using any one of the methods or compositions disclosed herein. A target cell can include a wide variety of cell types. A target cell can be in vitro. A target cell can be in vivo. A target cell can be ex vivo. A target cell can be an isolated cell. A target cell can be a cell inside of an organism. A target cell can be an organism. A target cell can be a cell in a cell culture.

A target cell can be one of a collection of cells. A target cell can be a mammalian cell or derived from a mammalian cell. A target cell can be a rodent cell or derived from a rodent cell. A target cell can be a human cell or derived from a human cell. A target cell can be a prokaryotic cell or derived from a prokaryotic cell. A target cell can be a bacterial cell or can be derived from a bacterial cell. A target cell can be an archaeal cell or derived from an archaeal cell. A target cell can be a eukaryotic cell or derived from a eukaryotic cell. A target cell can be a pluripotent stem cell. A target cell can be a plant cell or derived from a plant cell. A target cell can be an animal cell or derived from an animal cell. A target cell can be an invertebrate cell or derived from an invertebrate cell. A target cell can be a vertebrate cell or derived from a vertebrate cell. A target cell can be a microbe cell or derived from a microbe cell. A target cell can be a fungi cell or derived from a fungi cell. A target cell can be from a specific organ or tissue.

A target cell can be a stem cell or progenitor cell. Target cells can include stem cells (e.g., adult stem cells, embryonic stem cells, induced pluripotent stem (iPS) cells) and progenitor cells (e.g., cardiac progenitor cells, neural progenitor cells, etc.). Target cells can include mammalian stem cells and progenitor cells, including rodent stem cells, rodent progenitor cells, human stem cells, human progenitor cells, etc. Clonal cells can comprise the progeny of a cell. A target cell can comprise a target nucleic acid. A target cell can be in a living organism. A target cell can be a genetically modified cell. A target cell can be a host cell.

A target cell can be a totipotent stem cell, however, in some embodiments of this disclosure, the term “cell” may be used but may not refer to a totipotent stem cell. A target cell can be a plant cell, but in some embodiments of this disclosure, the term “cell” may be used but may not refer to a plant cell. A target cell can be a pluripotent cell. For example, a target cell can be a pluripotent hematopoietic cell that can differentiate into other cells in the hematopoietic cell lineage but may not be able to differentiate into any other non-hematopoietic cell. A target cell may be able to develop into a whole organism. A target cell may or may not be able to develop into a whole organism. A target cell may be a whole organism.

A target cell can be a primary cell. For example, cultures of primary cells can be passaged 0 times, 1 time, 2 times, 4 times, 5 times, 10 times, 15 times or more. Cells can be unicellular organisms. Cells can be grown in culture.

A target cell can be a diseased cell. A diseased cell can have altered metabolic, gene expression, and/or morphologic features. A diseased cell can be a cancer cell, a diabetic cell, and a apoptotic cell. A diseased cell can be a cell from a diseased subject. Exemplary diseases can include blood disorders, cancers, metabolic disorders, eye disorders, organ disorders, musculoskeletal disorders, cardiac disease, and the like.

If the target cells are primary cells, they may be harvested from an individual by any method. For example, leukocytes may be harvested by apheresis, leukocytapheresis, density gradient separation, etc. Cells from tissues such as skin, muscle, bone marrow, spleen, liver, pancreas, lung, intestine, stomach, etc. can be harvested by biopsy. An appropriate solution may be used for dispersion or suspension of the harvested cells. Such solution can generally be a balanced salt solution, (e.g. normal saline, phosphate-buffered saline (PBS), Hank's balanced salt solution, etc.), conveniently supplemented with fetal calf serum or other naturally occurring factors, in conjunction with an acceptable buffer at low concentration. Buffers can include HEPES, phosphate buffers, lactate buffers, etc. Cells may be used immediately, or they may be stored (e.g., by freezing). Frozen cells can be thawed and can be capable of being reused. Cells can be frozen in a DMSO, serum, medium buffer (e.g., 10% DMSO, 50% serum, 40% buffered medium), and/or some other such common solution used to preserve cells at freezing temperatures.

Non-limiting examples of cells which can be target cells include, but are not limited to, lymphoid cells, such as B cell, T cell (Cytotoxic T cell, Natural Killer T cell, Regulatory T cell, T helper cell), Natural killer cell, cytokine induced killer (CIK) cells (see e.g. US20080241194); myeloid cells, such as granulocytes (Basophil granulocyte, Eosinophil granulocyte, Neutrophil granulocyte/Hypersegmented neutrophil), Monocyte/Macrophage, Red blood cell (Reticulocyte), Mast cell, Thrombocyte/Megakaryocyte, Dendritic cell; cells from the endocrine system, including thyroid (Thyroid epithelial cell, Parafollicular cell), parathyroid (Parathyroid chief cell, Oxyphil cell), adrenal (Chromaffin cell), pineal (Pinealocyte) cells; cells of the nervous system, including glial cells (Astrocyte, Microglia), Magnocellular neurosecretory cell, Stellate cell, Boettcher cell, and pituitary (Gonadotrope, Corticotrope, Thyrotrope, Somatotrope, Lactotroph); cells of the Respiratory system, including Pneumocyte (Type I pneumocyte, Type II pneumocyte), Clara cell, Goblet cell, Dust cell; cells of the circulatory system, including Myocardiocyte, Pericyte; cells of the digestive system, including stomach (Gastric chief cell, Parietal cell), Goblet cell, Paneth cell, G cells, D cells, ECL cells, I cells, K cells, S cells; enteroendocrine cells, including enterochromaffm cell, APUD cell, liver (Hepatocyte, Kupffer cell), Cartilage/bone/muscle; bone cells, including Osteoblast, Osteocyte, Osteoclast, teeth (Cementoblast, Ameloblast); cartilage cells, including Chondroblast, Chondrocyte; skin cells, including Trichocyte, Keratinocyte, Melanocyte (Nevus cell); muscle cells, including Myocyte; urinary system cells, including Podocyte, Juxtaglomerular cell, Intraglomerular mesangial cell/Extraglomerular mesangial cell, Kidney proximal tubule brush border cell, Macula densa cell; reproductive system cells, including Spermatozoon, Sertoli cell, Leydig cell, Ovum; and other cells, including Adipocyte, Fibroblast, Tendon cell, Epidermal keratinocyte (differentiating epidermal cell), Epidermal basal cell (stem cell), Keratinocyte of fingernails and toenails, Nail bed basal cell (stem cell), Medullary hair shaft cell, Cortical hair shaft cell, Cuticular hair shaft cell, Cuticular hair root sheath cell, Hair root sheath cell of Huxley's layer, Hair root sheath cell of Henle's layer, External hair root sheath cell, Hair matrix cell (stem cell), Wet stratified barrier epithelial cells, Surface epithelial cell of stratified squamous epithelium of cornea, tongue, oral cavity, esophagus, anal canal, distal urethra and vagina, basal cell (stem cell) of epithelia of cornea, tongue, oral cavity, esophagus, anal canal, distal urethra and vagina, Urinary epithelium cell (lining urinary bladder and urinary ducts), Exocrine secretory epithelial cells, Salivary gland mucous cell (polysaccharide-rich secretion), Salivary gland serous cell (glycoprotein enzyme-rich secretion), Von Ebner's gland cell in tongue (washes taste buds), Mammary gland cell (milk secretion), Lacrimal gland cell (tear secretion), Ceruminous gland cell in ear (wax secretion), Eccrine sweat gland dark cell (glycoprotein secretion), Eccrine sweat gland clear cell (small molecule secretion). Apocrine sweat gland cell (odoriferous secretion, sex-hormone sensitive), Gland of Moll cell in eyelid (specialized sweat gland), Sebaceous gland cell (lipid-rich sebum secretion), Bowman's gland cell in nose (washes olfactory epithelium), Brunner's gland cell in duodenum (enzymes and alkaline mucus), Seminal vesicle cell (secretes seminal fluid components, including fructose for swimming sperm), Prostate gland cell (secretes seminal fluid components), Bulbourethral gland cell (mucus secretion), Bartholin's gland cell (vaginal lubricant secretion), Gland of Littre cell (mucus secretion), Uterus endometrium cell (carbohydrate secretion), Isolated goblet cell of respiratory and digestive tracts (mucus secretion), Stomach lining mucous cell (mucus secretion), Gastric gland zymogenic cell (pepsinogen secretion), Gastric gland oxyntic cell (hydrochloric acid secretion), Pancreatic acinar cell (bicarbonate and digestive enzyme secretion), Paneth cell of small intestine (lysozyme secretion), Type II pneumocyte of lung (surfactant secretion), Clara cell of lung, Hormone secreting cells, Anterior pituitary cells, Somatotropes, Lactotropes, Thyrotropes, Gonadotropes, Corticotropes, Intermediate pituitary cell, Magnocellular neurosecretory cells, Gut and respiratory tract cells, Thyroid gland cells, thyroid epithelial cell, parafollicular cell, Parathyroid gland cells, Parathyroid chief cell, Oxyphil cell, Adrenal gland cells, chromaffin cells, Ley dig cell of testes, Theca interna cell of ovarian follicle, Corpus luteum cell of ruptured ovarian follicle, Granulosa lutein cells, Theca lutein cells, Juxtaglomerular cell (renin secretion), Macula densa cell of kidney, Metabolism and storage cells, Barrier function cells (Lung, Gut, Exocrine Glands and Urogenital Tract), Kidney, Type I pneumocyte (lining air space of lung), Pancreatic duct cell (centroacinar cell), Nonstriated duct cell (of sweat gland, salivary gland, mammary gland, etc.), Duct cell (of seminal vesicle, prostate gland, etc.), Epithelial cells lining closed internal body cavities, Ciliated cells with propulsive function, Extracellular matrix secretion cells, Contractile cells; Skeletal muscle cells, stem cell, Heart muscle cells, Blood and immune system cells, Erythrocyte (red blood cell), Megakaryocyte (platelet precursor), Monocyte, Connective tissue macrophage (various types), Epidermal Langerhans cell, Osteoclast (in bone), Dendritic cell (in lymphoid tissues), Microglial cell (in central nervous system), Neutrophil granulocyte, Eosinophil granulocyte, Basophil granulocyte, Mast cell, Helper T cell, Suppressor T cell, Cytotoxic T cell, Natural Killer T cell, B cell, Natural killer cell, Reticulocyte, Stem cells and committed progenitors for the blood and immune system (various types), Pluripotent stem cells, Totipotent stem cells, Induced pluripotent stem cells, adult stem cells, Sensory transducer cells, Autonomic neuron cells, Sense organ and peripheral neuron supporting cells, Central nervous system neurons and glial cells, Lens cells, Pigment cells, Melanocyte, Retinal pigmented epithelial cell, Germ cells, Oogonium/Oocyte, Spermatid, Spermatocyte, Spermatogonium cell (stem cell for spermatocyte), Spermatozoon, Nurse cells, Ovarian follicle cell, Sertoli cell (in testis), Thymus epithelial cell, Interstitial cells, and Interstitial kidney cells.

Of particular interest are cancer cells. In some embodiments, the target cell is a cancer cell. Non-limiting examples of cancer cells include cells of cancers including Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloblastoma, Medulloepithelioma, Melanoma, Melanoma, Meningioma, Merkel Cell Carcinoma, Mesothelioma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic Cancer, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, Wilms' tumor, and combinations thereof. In some embodiments, the targeted cancer cell represents a subpopulation within a cancer cell population, such as a cancer stem cell. In some embodiments, the cancer is of a hematopoietic lineage, such as a lymphoma. The antigen can be a tumor associated antigen.

In some cases, the subject can have or can be suspected of having an autoimmune disease. Non-limiting examples of an autoimmune disease can include acute disseminated encephalomyelitis (ADEM), acute necrotizing hemorrhagic leukoencephalitis, Addison's disease, agammaglobulinemia, allergic asthma, allergic rhinitis, alopecia areata, amyloidosis, ankylosing spondylitis, antibody-mediated transplantation rejection, anti-GBM/Anti-TBM nephritis, antiphospholipid syndrome (APS), autoimmune angioedema, autoimmune aplastic anemia, autoimmune dysautonomia, autoimmune hepatitis, autoimmune hyperlipidemia, autoimmune immunodeficiency, autoimmune inner ear disease (AIED), autoimmune myocarditis, autoimmune pancreatitis, autoimmune retinopathy, autoimmune thrombocytopenic purpura (ATP), autoimmune thyroid disease, autoimmune urticaria, axonal & neuronal neuropathies, Balo disease, Behcet's disease, bullous pemphigoid, cardiomyopathy, Castleman disease, celiac disease, Chagas disease, chronic fatigue syndrome, chronic inflammatory demyelinating polyneuropathy (CIDP), chronic recurrent multifocal ostomyelitis (CRMO), Churg-Strauss syndrome, cicatricial pemphigoid/benign mucosal pemphigoid, Crohn's disease, Cogans syndrome, cold agglutinin disease, congenital heart block, coxsackie myocarditis, CREST disease, essential mixed cryoglobulinemia, demyelinating neuropathies, dermatitis herpetiformis, dermatomyositis, Devic's disease (neuromyelitis optica), discoid lupus, Dressler's syndrome, endometriosis, eosinophilic fasciitis, erythema nodosum, experimental allergic encephalomyelitis, Evans syndrome, fibromyalgia, fibrosing alveolitis, giant cell arteritis (temporal arteritis), glomerulonephritis, goodpasture's syndrome, granulomatosis with polyangiitis (GPA), Graves' disease, Guillain-Barre syndrome, Hashimoto's encephalitis, Hashimoto's thyroiditis, hemolytic anemia, Henoch-Schonlein purpura, herpes gestationis, hypogammaglobulinemia, hypergammaglobulinemia, idiopathic thrombocytopenic purpura (ITP), IgA nephropathy, IgG4-related sclerosing disease, immunoregulatory lipoproteins, inclusion body myositis, inflammatory bowel disease, insulin-dependent diabetes (type 1), interstitial cystitis, juvenile arthritis, juvenile diabetes, Kawasaki syndrome, Lambert-Eaton syndrome, leukocytoclastic vasculitis, lichen planus, lichen sclerosus, ligneous conjunctivitis, linear IgA disease (LAD), lupus (SLE), lyme disease, Meniere's disease, microscopic polyangiitis, mixed connective tissue disease (MCTD), monoclonal gammopathy of undetermined significance (MGUS), Mooren's ulcer, Mucha-Habermann disease, multiple sclerosis, myasthenia gravis, myositis, narcolepsy, neuromyelitis optica (Devic's), neutropenia, ocular cicatricial pemphigoid, optic neuritis, palindromic rheumatism, PANDAS (Pediatric Autoimmune Neuropsychiatric Disorders Associated with Streptococcus), paraneoplastic cerebellar degeneration, paroxysmal nocturnal hemoglobinuria (PNH), Parry Romberg syndrome, Parsonnage-Turner syndrome, pars planitis (peripheral uveitis), pemphigus, peripheral neuropathy, perivenous encephalomyelitis, pernicious anemia, POEMS syndrome, polyarteritis nodosa, type I, II, & III autoimmune polyglandular syndromes, polymyalgia rheumatic, polymyositis, postmyocardial infarction syndrome, postpericardiotomy syndrome, progesterone dermatitis, primary biliary cirrhosis, primary sclerosing cholangitis, psoriasis, psoriatic arthritis, idiopathic pulmonary fibrosis, pyoderma gangrenosum, pure red cell aplasia, Raynauds phenomenon, reflex sympathetic dystrophy, Reiter's syndrome, relapsing polychondritis, restless legs syndrome, retroperitoneal fibrosis, rheumatic fever, rheumatoid arthritis, sarcoidosis, Schmidt syndrome, scleritis, scleroderma, Sjogren's syndrome, sperm & testicular autoimmunity, stiff person syndrome, subacute bacterial endocarditis (SBE), Susac's syndrome, sympathetic ophthalmia, Takayasu's arteritis, temporal arteritis/Giant cell arteritis, thrombocytopenic purpura (TTP), Tolosa-Hunt syndrome, transverse myelitis, ulcerative colitis, undifferentiated connective tissue disease (UCTD), uveitis, vasculitis, vesiculobullous dermatosis, vitiligo, Waldenstrom's macroglobulinemia (WM), and Wegener's granulomatosis (Granulomatosis with Polyangiitis (GPA)).

In some cases, the autoimmune disease comprises one or more members selected from the group comprising rheumatoid arthritis, type 1 diabetes, systemic lupus erythematosus (lupus or SLE), myasthenia gravis, multiple sclerosis, scleroderma, Addison's Disease, bullous pemphigoid, pemphigus vulgaris, Guillain-Barré syndrome, Sjogren syndrome, dermatomyositis, thrombotic thrombocytopenic purpura, hypergammaglobulinemia, monoclonal gammopathy of undetermined significance (MGUS), Waldenstrom's macroglobulinemia (WM), chronic inflammatory demyelinating polyradiculoneuropathy (CIDP), Hashimoto's Encephalopathy (HE), Hashimoto's Thyroiditis, Graves' Disease, Wegener's Granulomatosis, and antibody-mediated transplantation rejection (e.g., for tissue transplants such as renal transplant). In examples, the autoimmune disease can be type 1 diabetes, lupus, or rheumatoid arthritis.

In some cases, the target cells form a tumor (i.e., a solid tumor). A tumor treated with the methods herein can result in stabilized tumor growth (e.g., one or more tumors do not increase more than 1%, 5%, 10%, 15%, or 20% in size, and/or do not metastasize). In some cases, a tumor is stabilized for at least about 1, 2, 3, 4, 5, 6, 7, 8, 9, 10, 11, 12, or more weeks. In some cases, a tumor is stabilized for at least about 1, 2, 3, 4, 5, 6, 7, 8, 9, 10, 11, 12, or more months. In some cases, a tumor is stabilized for at least about 1, 2, 3, 4, 5, 6, 7, 8, 9, 10, or more years. In some cases, the size of a tumor or the number of tumor cells is reduced by at least about 5%, 10%, 15%, 20%, 25, 30%, 35%, 40%, 45%, 50%, 55%, 60%, 65%, 70%, 75%, 80%, 85%, 90%, 95% or more. In some cases, the tumor is completely eliminated, or reduced below a level of detection. In some cases, a subject remains tumor free (e.g. in remission) for at least about 1, 2, 3, 4, 5, 6, 7, 8, 9, 10, 11, 12, or more weeks following treatment. In some cases, a subject remains tumor free for at least about 1, 2, 3, 4, 5, 6, 7, 8, 9, 10, 11, 12, or more months following treatment. In some cases, a subject remains tumor free for at least about 1, 2, 3, 4, 5, 6, 7, 8, 9, 10, or more years after treatment.

In various embodiments, the subject methods of the disclosure can be performed in a subject. A subject can be a human. A subject can be a mammal (e.g., rat, mouse, cow, dog, pig, sheep, horse). A subject can be a vertebrate or an invertebrate. A subject can be a laboratory animal. A subject can be a patient. A subject can be suffering from a disease. A subject can display symptoms of a disease. A subject may not display symptoms of a disease, but still have a disease. A subject can be under medical care of a caregiver (e.g., the subject is hospitalized and is treated by a physician).

Systems and methods of the present disclosure may be combined with or modified by other methods and systems, such as, for example, those described in International Patent Application No. PCT/US2017/012885, International Patent Application No. PCT/US2017/012881, U.S. Patent Application No. 2018/0346543, and International Patent Application No. PCT/US2018/041704, each of which is entirely incorporated herein by reference.

### EXAMPLES

Various aspects of the disclosure are further illustrated by the following non-limiting examples.

### Example 1. Titration of T Cells with a Single Antigen

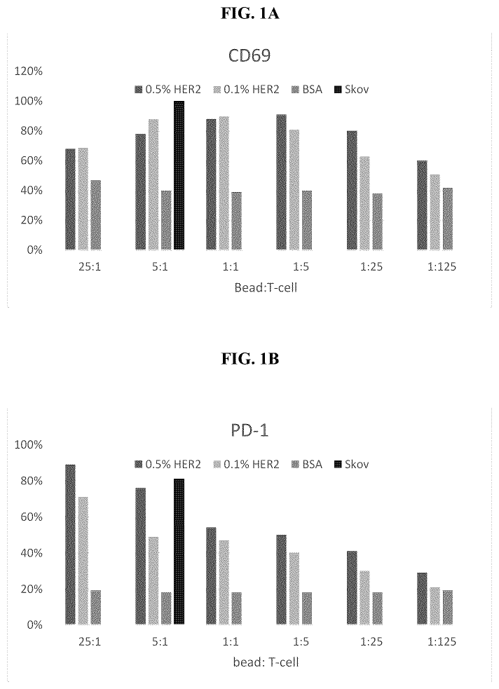

CAR-T or TCR-T cells can be titrated with a single antigen conjugated to non-cellular substrates (e.g. beads). HER2 is conjugated with the beads at two densities, 0.5% and 0.1%. The HER2 beads are co-cultured with HER2-CAR T cells or HER2-TCR-T cells at different beads to cells (E:T) ratios for 1-3 days. T-cell activation can then be measured by flow cytometry using staining for relevant biomarkers such as CD69, CD25, PD1, etc. Using different densities of HER2 conjugated to the beads and different ratio of HER2 beads to T cells, the minimal amount of HER2 needed for effective CAR-T cell activation can be quantified. FIG. 1A illustrates CAR-T cell activation by different combinations of HER2 densities and beads to T cell ratios as determined by CD69 fluorescent intensity. FIG. 1B illustrates CAR-T cell activation by different combinations of HER2 densities and beads to T cell ratios as determined by PD-1 fluorescent intensity.

### Example 2. Titration of T Cells with Multi-Antigen

CAR-T or TCR-T cells can be titrated with multiple antigens conjugated to non-cellular substrates (e.g. beads). Various densities of HER2 and PDL1 can be conjugated to the same non-cellular substrates (i.e. HER2 and PDL1 can be conjugated to the same beads) and co-cultured with HER2-CAR-T cells or HER2-TCR-T cells. The HER2-CAR-T cells or HER2-TCR-T cells can be genetically modified to downregulate PD1. The HER2-CAR-T cells or HER2-TCR-T cells can be co-cultured with the conjugated beads at different beads to cell rations (E:T) for 1-3 days. T-cell activation can then be measured by flow cytometry using staining for relevant biomarkers such as CD69, CD25, PD1, etc. Using different densities and different numbers and ratio of HER2-PDL1 beads, the minimal amount of HER2 needed for effective CAR-T or TCR-T cell activation in combination with the additional signaling coming from PDL1 can be quantified. The multiple antigen bead titration method can be applied to dual and tri CAR-T or TCR-T cells by using beads conjugated to both scFv antigens.

### Example 3. A System for Quantifying T Cell Potency Using Single Antigen Conjugated Beads

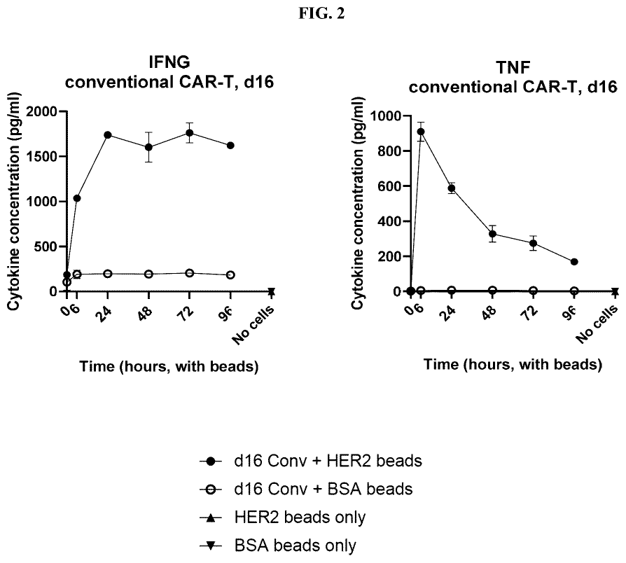

HER2 conjugated beads are co-cultured with HER2-CAR T cells or TCR-T cells at different E:T ratios for up to 6 days. CAR-T cell potency is measured using ELISA for relevant secreted cytokines such as IL-2, TNFα, IFNg, etc. T-cell proliferation is also measured by cell counting or flow cytometry using specific staining. Many other T-cell parameters such as cell biomarkers, cell signaling, cell viability, and RNA expression profiling can be measured using the system described herein. FIG. 2 illustrates an exemplary measurement of cytokine secretion by CAR-T cells. Conventional HER2-CAR-T cells were cocultured with HER2-beads at 1:1 cell:bead ratio. BSA beads were use as negative control. Co-culturing of HER2 beads with CAR-T cells led to prolonged IFNG secretion and acute secretion of TNF.

### Example 4. A System for Quantifying T Cell Potency Using Multi-Antigen Conjugated Beads

HER2-PDL1 conjugated beads are co-cultured with HER2-CAR-T cells or TCR-T cells that are genetically modified to down regulate PD1 at different E:T ratios for up to 6 days. T cell potency is measured using ELISA for relevant secreted cytokines such as IL-2, TNFα, IFNg, etc. The efficacy of PD1 down-regulation is measured by flow cytometry using PD1 staining. T-cell proliferation is also measured by cell counting or flow cytometry using CF SE staining. Other T-cell parameters that can be measured using this system include cell biomarkers, cell signaling, cell viability, RNA expression profiling, etc.

### Example 5. A System for Evaluating T Cell Exhaustion Using Multi-Antigen Conjugated Beads

HER2-PDL1 conjugated beads are co-cultured with HER2-CAR-T cells or TCR-T cells that are genetically modified to down regulate PD1 at different E:T ratios for 7-14 days. T-cell exhaustion is then measured by flow cytometry using staining for relevant biomarkers such as PD1, Tim3, Lag3, etc. Exhaustion is also measured using ELISA for relevant secreted cytokines such as IL-2, TNFα, IFNg, etc. The efficacy of PD1 down-regulation is measured by flow cytometry using PD1 staining.

### Example 6. T Cell Activation Assay

Modified autologous or allogenic T cells are assayed for functionally, efficacy, and safety. If the modified T cells meet the thresholds as determined from the assays, the modified T cells can be administered to patients. The modified T cells are first expanded. A first sub-population of the modified T cells are then activated by treatment with antigen conjugated to the non-cellular substrate. The cytokine secretion and mRNA expression encoding the cytokine secreted are measured from the activated first sub-population of modified T cells. Additionally, the modified T cells are co-cultured with target cells, where the target cells are examined for cytotoxicity exerted by the activated T cells. Upon meeting the thresholds, the modified T cells can be safely administered to the subjects. A second sub-population of modified T cells is administered to subjects for therapeutic purposes. Alternately, the modified T cells that do not meet the thresholds cannot be administered to the subjects.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### Example 7. Antigen Covered Beads as Platform Substrate for Assessing CAR T Cell Potency

In this example, multiple examples demonstrating optimization of antigen coated beads as platform for assessing T cell potency is described.

### Methods:

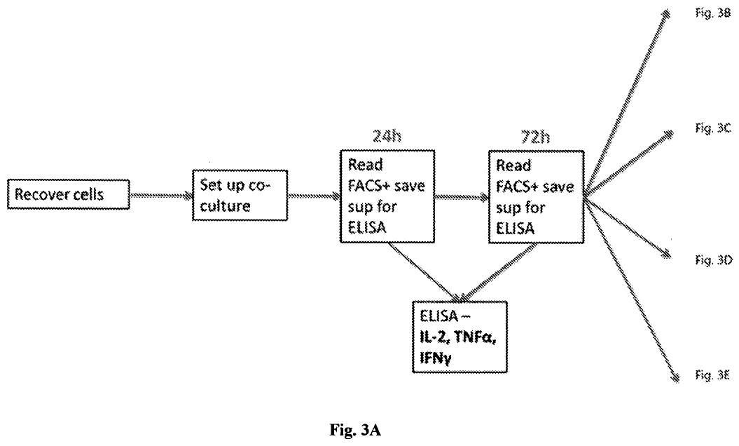

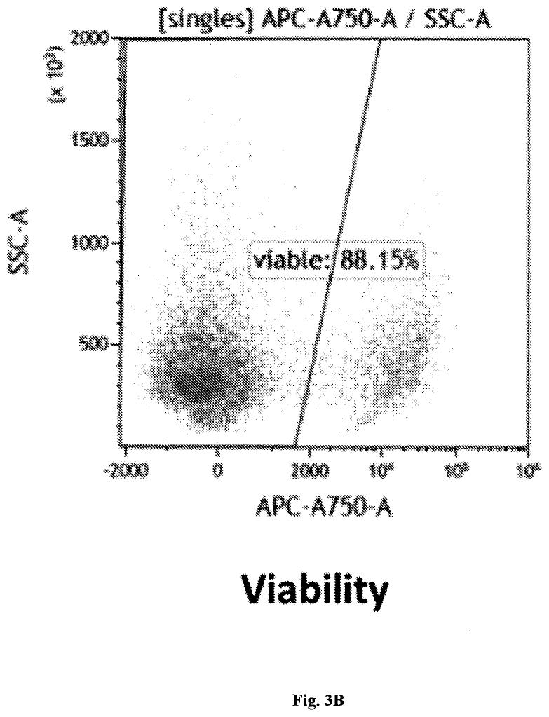

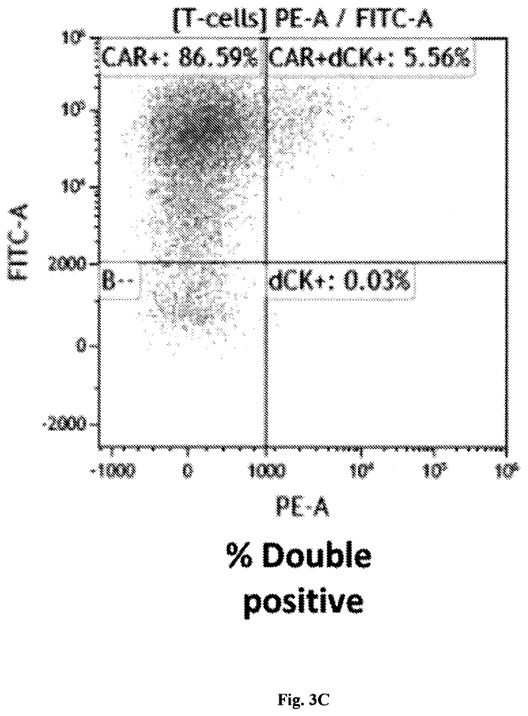

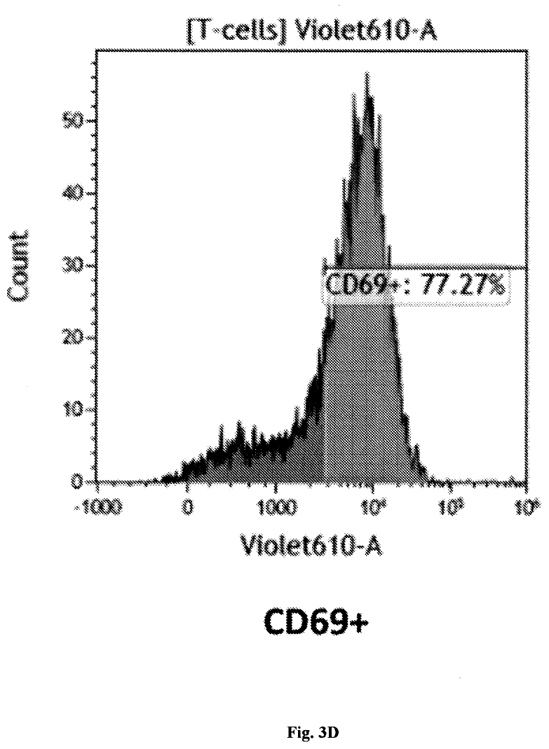

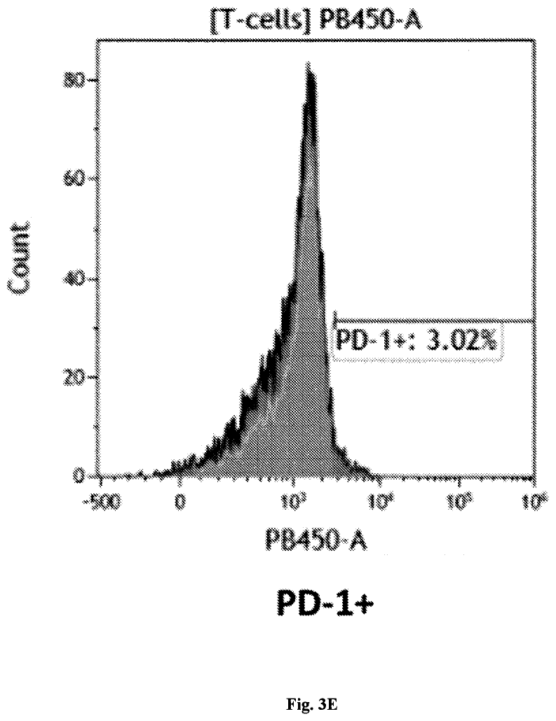

Co-culture of HER2-conjugated bead with CAR-T cells—FIG. 3A-FIG. 3E show a schematic flow diagram of an assay set up for assessing effect of antigen coated beads on CAR T cell potentiation.

Engineered HER-2 CAR-T cells were collected by centrifugation, counted and plated in 96-well plate at 50K cells/well in T-cell growth media without cytokines (RPMI, 10% human serum, 1% Glutamax, 1% Pen/Strep mix). HER2-conjugated beads with different densities (low-mid-high, BSA as negative control, HER2 positive tumor cells as positive control) were washed and added to the cells at different bead: cell ratios (5:1-1:125). Cells are supernatants were harvested at different time points (6 hrs-96 hrs).

Flow cytometry—To measure the presence and level of various surface markers, Cells were collected by centrifugation, washed with PBS, then stained with a panel of fluorophore-conjugated-antibodies for 30 minutes. Cells were washed again with Flow cytometry buffer (PBS+2% serum/BSA), resuspended with same buffer and read by flow cytometer (Beckman coulter cytoflex). Analysis was performed using Kaluza and Excel software.

Proliferation analysis by flow cytometry—Celltrace Violet dye was added to the cells together with HER-2 conjugated beads. At the desired time point cells were collected by centrifugation, washed with PBS and read by flow cytometer (with or without staining for additional surface markers). Analysis was performed using Kaluza and Excel software.

ELISA—To measure secreted cytokines, supernatants were collected and ELISA was performed using Biolegend ELISA kits, according to the manufacturer's protocol. For inflammatory cytokine panel, CBA (cytokine bead assay) from BD bioscience was used according to the manufacturer's protocol.

mRNA expression analysis—co culture was performed as described above, at the desired time point, cells were harvested, separated from beads using magnet and washed. RNA was extracted with Qiagen RNAeasy kit according to the manufacturer's protocol. cDNA was synthesized by reverse transcription (Applied biosciences) and used as template for qPCR using Quantstudio3/5. Analysis was performed using Quantstudio and Excel software.

T-cell exhaustion assay—Engineered T-cells were stimulated using tumor cells for 1-2 weeks. Then T-cells were harvested and incubated with HER-2 conjugated beads for 24-72 hrs, as described above. Exhaustion markers were measured by flow cytometry.

Quantification of conjugated antigens—Beads (and cells) were washed and stained using unconjugated primary antibody for 60 minutes. Cells were then washed and stained with secondary fluorophore-conjugated-antibody for 30 minutes. Agilent Qi-Fi calibration beads were used to create a standard curve. Data was read by flow-cytometer and analyzed using Kaluza and Excel software.

### Results and Discussion

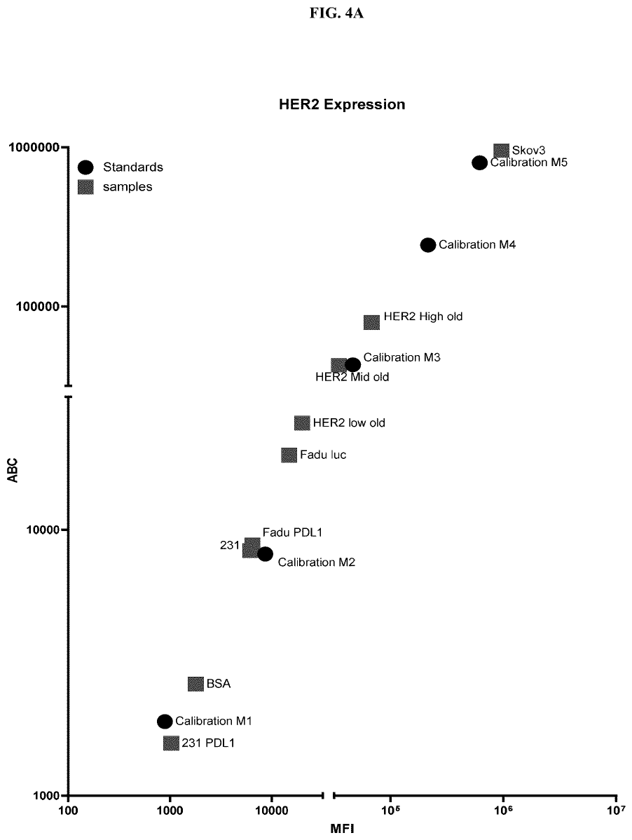

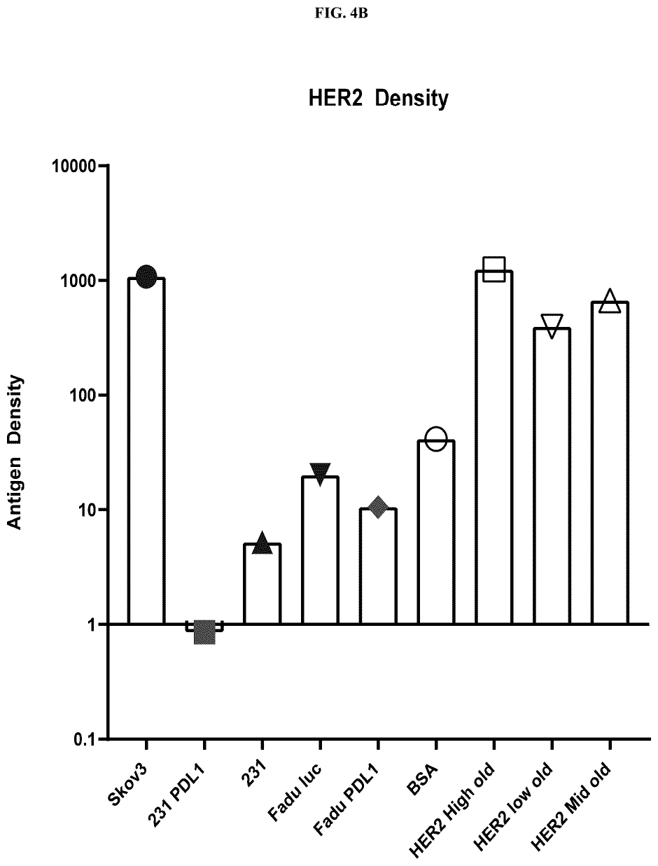

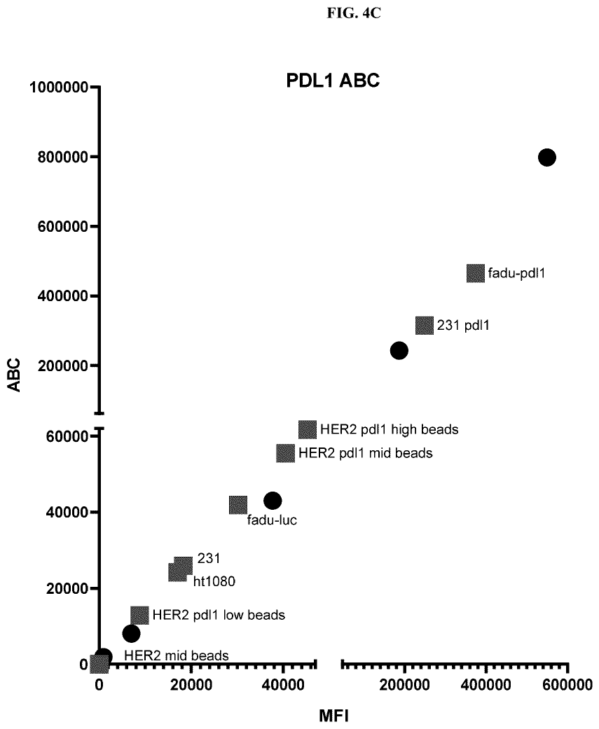

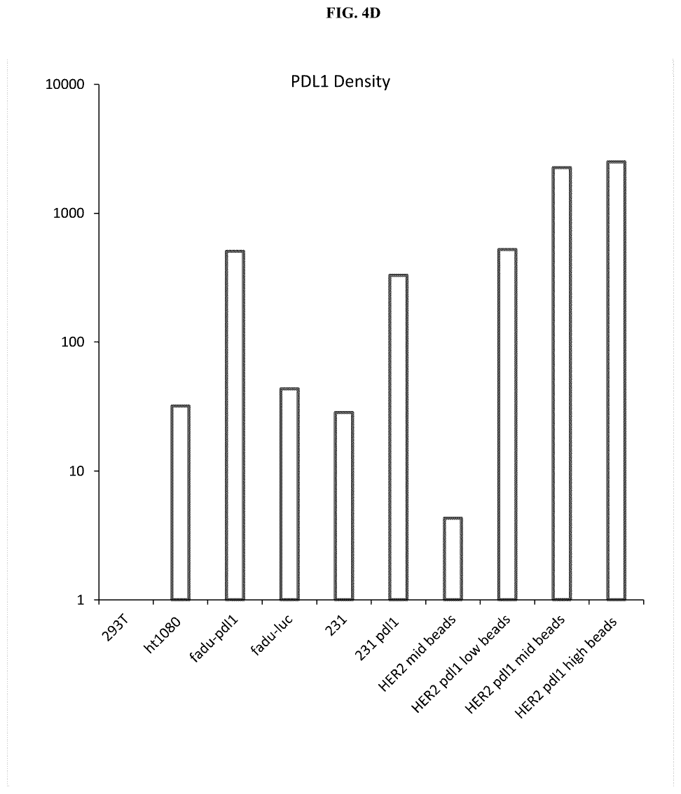

I. Quantification of antigen on beads and comparison with tumor model cells. HER2 conjugated beads and HER2-PDL1 conjugated beads with various densities of either HER2 or PDL1 were compared to various tumor cells lines. Antigen binding capacity (ABC, representing the amount of antigen on the beads) was measured for each of the antigens using Agilent Qi-Fi system. Bead/cell size was also measured and antigen density was calculated for each antigen. For HER2 (FIGS. 4A and 4B) results demonstrate that all 3 HER2 beads have significant amounts of HER2 on them, higher than most cells tested. When observing densities this is even more pronounced, with HER2-high beads having a density as high as Skov3 cells, the highest expressing HER2 cell line. When looking at PD-L1 (FIGS. 4C and 4D) beads show lower ABC than most PDL1 positive cells, however, the calculated PDL1 density reveals that even HER2-PDL1-low beads have higher PDL1 density than most PDL1 positive cells, and PDL1-mid and -high bead have even higher densities that might not be biologically relevant.

II. Analysis of Potency—Activation Markers and Activation of Engineered T-Cells

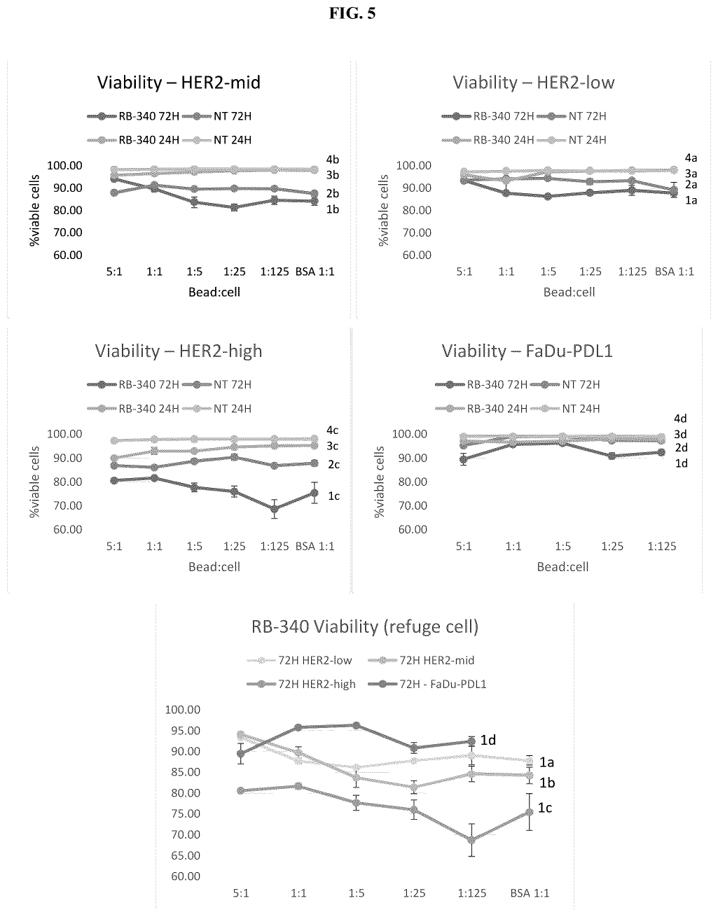

A. Cell Viability. Engineered cells (RB-340—HER2-CAR-PD1sgRNA+dCAS9-KRAB), and non-transduced control cells (NT) were incubated with HER-2 conjugated beads or control (BSA) beads, and assessed for activation marker CD69. Co-culture with FaDU-PDL1 cells was used as positive control. Activation was assessed by flow cytometry. As an initial study, cell viability was determined. No significant differences in viability observed, though slightly higher cell death was observed after 72h compared to 24h, in both RB-340 and NT samples (FIG. 5).

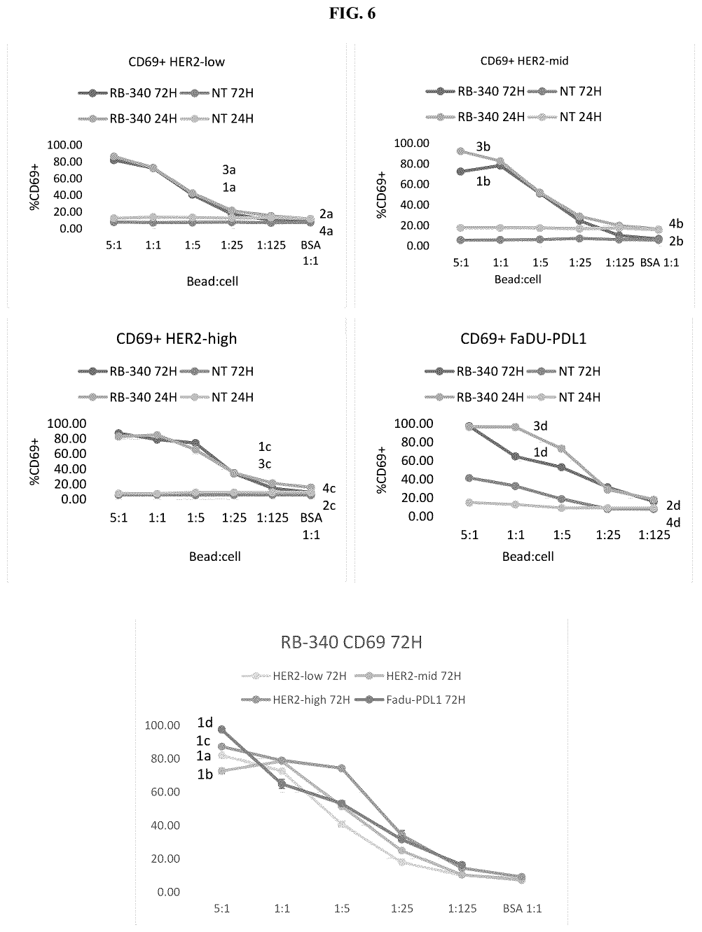

B. CD69 activation. CD69, an early T-cell activation marker showed specific up-regulation in RB-340 cells in response to HER2 stimulation by both beads and cells (FIG. 6). This upregulation correlated with bead: cell ratio. All bead densities were able to stimulate CD69 to similar levels compared to Fadu-PDL1 cells. Interestingly, using bead stimulation, CD69 levels were maintained at 24h and 72 hrs. However, upon stimulation with FaDu-PDL1 cells, CD69 level was lower at 72h compared to 24h. This could be because during the co-culture, FaDu-PDL1 cells are killed by the T-cells, thus reducing the level of antigen stimulation.

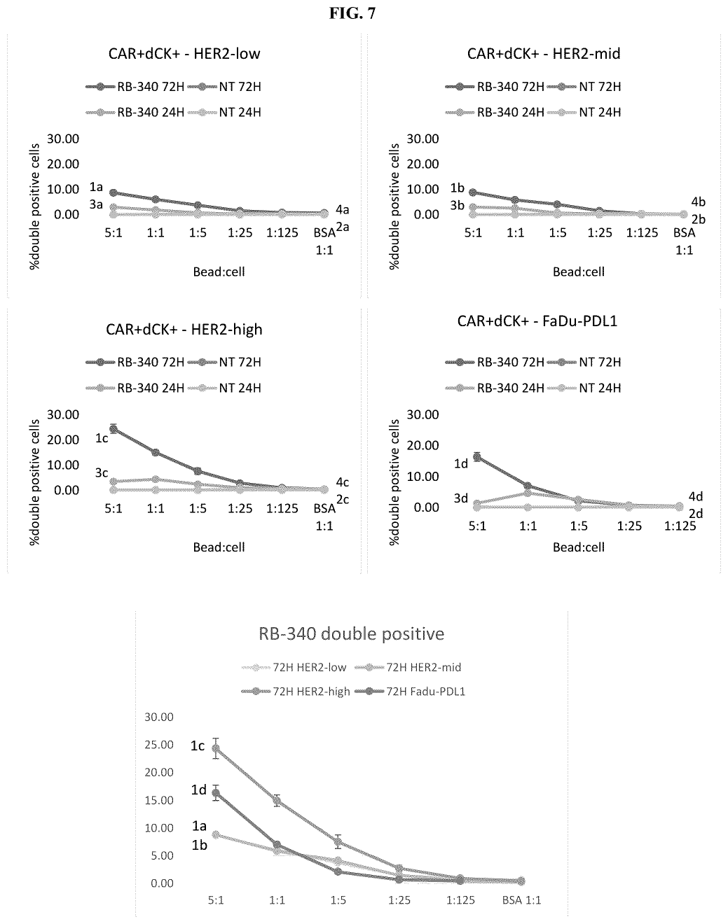

C. Recovery of engineered cells transduced with CAR and CAS actuator moiety (double positive cells, CAR+dCK) requires stimulation. RB-340 cells are engineered with 2 major elements— HER2-CAR (along with a sgRNA) and dCAS9-KRAB (dCK). While the level of CAR was mostly stable, the expression of dCK depends on the activation status of the cells. Without stimulation dCK expression is minimal, and only upon antigen stimulation its expression is restored. Thus, one marker of T-cell activation is the percentage of double positive (CAR+/dCK+) cells in the culture. In this experiment, it was observed that this parameter correlated with both HER2 density and bead: cell ratio. The highest percentage of double positive cells was observed using HER2-high beads for 72h (FIG. 7). These results showed that the best recovery of double positives requires strong and longer stimulation.

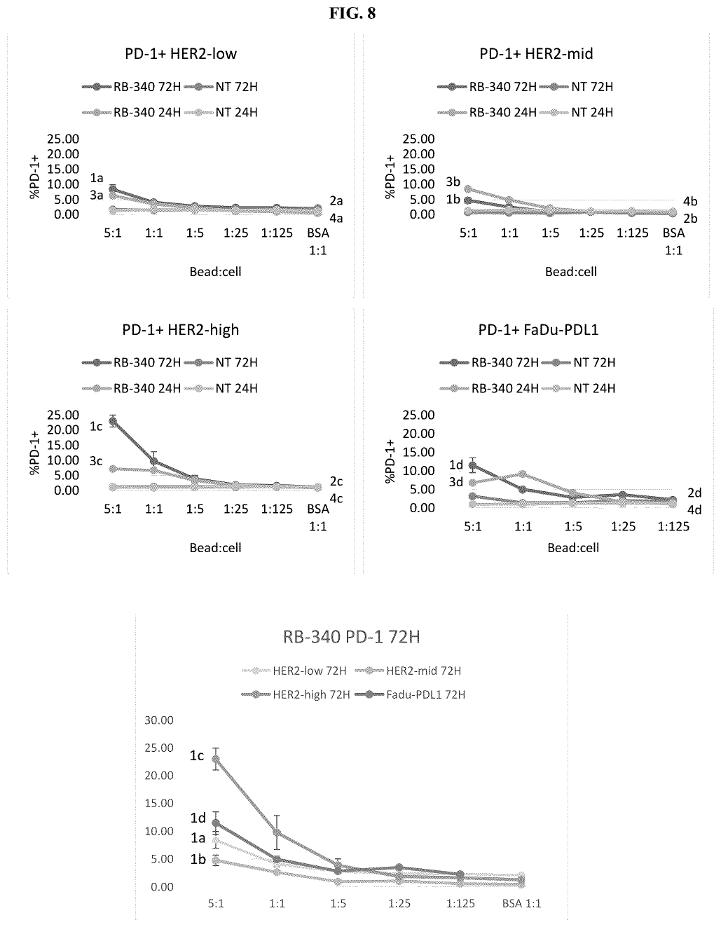

D. PD-1 activation is correlated to HER2 density and ratio (in RB-340 cells). PD-1 is a marker of activation which at later stages acts as a T-cell check point, facilitating exhaustion. In the tested cells, RB-340, the combination of dCAS9 and the PD-1 specific sgRNA inhibits the expression of PD-1 in response to stimulation. PD-1 is not completely shut-off, but its levels are markedly reduced in these cells. Despite this, it is still evident that stimulation using HER2-beads increases the level of PD-1 in the cells (FIG. 8). This up regulation is correlated to both HER2 density on the bead as well as bead: cell ratio. Compared to FaDu-PDL1 cells, HER2-high beads resulted in stronger stimulation and higher PD-1 levels.

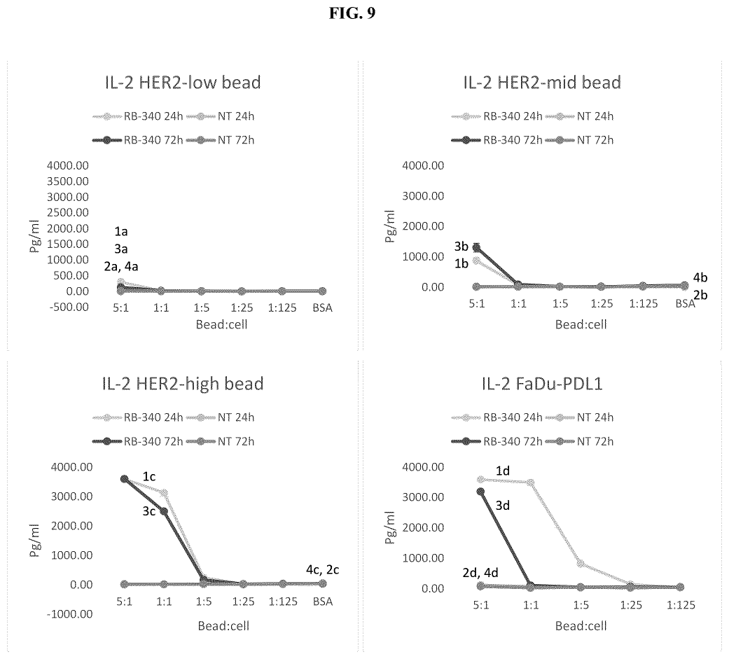

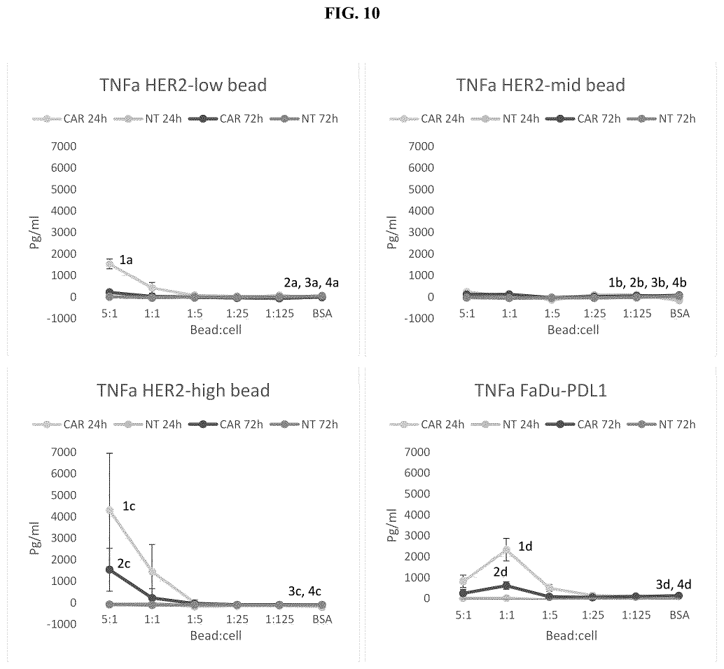

III. Analysis of Potency-cytokine secretion. Engineered cells transduced with (1) RB-340—HER2-CAR-PD1sgRNA+dCAS9-KRAB (“RB-340”) or (2) conventional anti-HER2 CAR (“CAR”)), as well as non-transduced control cells (NT) were incubated with HER-2 conjugated beads or control (BSA) beads. Co-culture with FaDU-PDL1 cells was used as positive control. Culture supernatant were collected and secreted cytokine levels were determines by ELISA. Three cytokines were measured in these assays—IL-2 (FIG. 9), TNFα (FIG. 10) and IFNg (FIG. 11). For all three cytokines, a similar result was observed; stimulation of the engineered cells with HER2 beads was able to promote cytokine secretion and this secretion correlated with both HER2 density and bead: cell ratio. To achieve cytokine levels comparable to tumor cell stimulation, HER2-high density bead was required. It also demonstrated that the beads show more consistent stimulation over time than live tumor cells. For IFNg secretion, Fadu stimulation at 1:1 ratio was comparable to high HER2 at 5:1 ratio.

In another experiment, using conventional HER2-CAR-T cells (without dCAS9 or sgRNA) (Cony) that were incubated with HER-2-high beads additional inflammatory cytokines were also explored. Secretion of most inflammatory (TH1) cytokines was induced by stimulation with HER2 beads (FIG. 12). In this assay, the kinetics of cytokine secretion was observed and for the induced cytokines, the kinetics were similar to those described in literature in response to antigen stimulation. IL10 and IL17a were not significantly induced by the beads in this assay.

IV. Comparison of different CAR-T products in response to stimulation. RB-340—HER2-CAR-PD1 sgRNA+dCAS9-KRAB (CAR), and control cells (HER2-CAR-Ctrl sgRNA+dCAS9-KRAB) (Cony) were incubated with HER-2 conjugated beads or control (BSA) beads. PD-1 level was measured by flow cytometry at various time point. FIG. 13A shows data indicating a percentage of PD1 positive cells in an experiment comparing conventional HER2-CAR-T cells (“d16 Cony”. without dCas9 or sgRNA), treated with either HER2 beads or control BSA beads. The results depicted in FIG. 13B demonstrate that bead were able to induce PD-1 in both cell types. However, in the presence of the sgRNA targeting PD-1, its level was significantly lower, even upon activation. Notably, the kinetics of PD-1 expression were very similar between the two cell types, and only the level of PD-1 was different. In addition, the basal level of PD-1 was lower in the presence of PD1-sgRNA. This could be the result of dCK activity during the cell engineering process itself, or due to some leakiness of the system, resulting in some PD-1 down-regulation even without antigen stimulation. These results demonstrate that activation markers (and even potency) of different CAR-T cell products can be directly compared by using bead stimulation of these products.

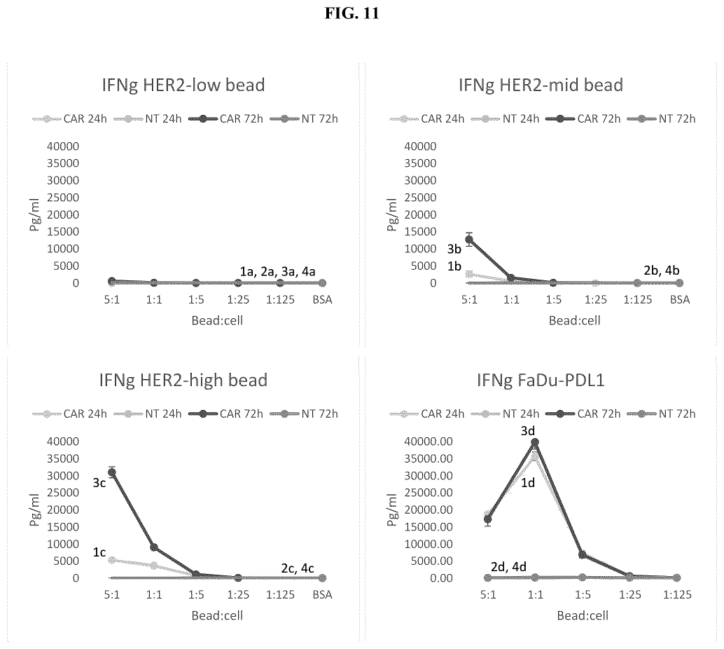

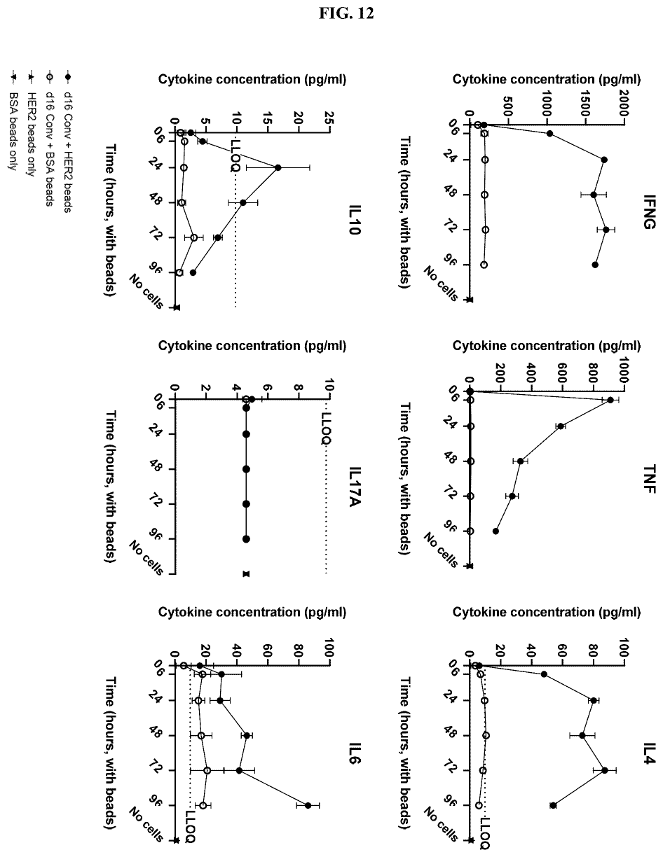

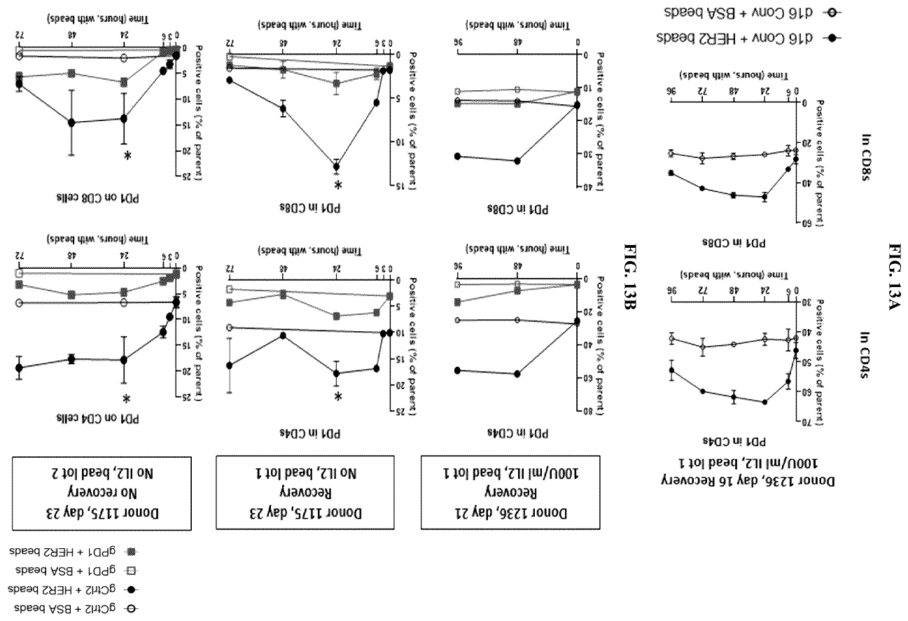

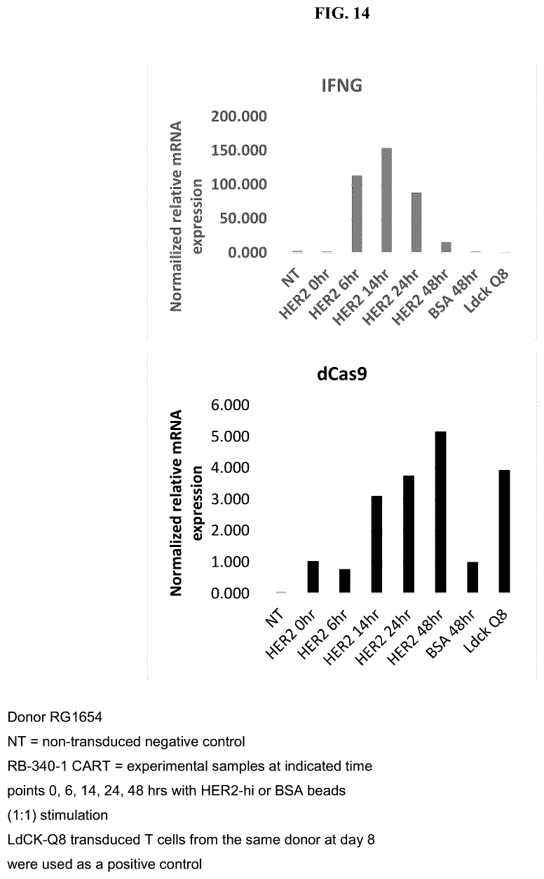

V. Gene Expression Analysis. Expression of dCAS9-KRAB is dependent on the activation status of the cells as indicated above. To directly measure dCAS9 expression, engineered cells (RB-340—HER2-CAR-PD1sgRNA+dCAS9-KRAB), (CAR), and non-transduced control cells (NT) were incubated with HER2-high beads or control (BSA) beads. RNA was extracted at various time points and qPCR was performed to assess IFNg and dCAS9-KRAB mRNA expression. T-cells newly transduced with dCAS9-KRAB were used as positive control. IFNg is a hallmark gene for T-cell activation. Upon stimulation with HER2 beads, IFNg expression was rapidly increased, peaking around 14h of stimulation (FIG. 14). In agreement with flow cytometry data in FIG. 11, dCK expression is also induced and reach its highest level at 48h, which was the latest tested time point (FIG. 14). It is possible that dCK expression continues to increase at later time points which were not explored in this assay.

Messenger RNA expression assays cannot be performed on experimental CAR T cells with tumor cells because presence of two different cell types in the culture would create a mixture of RNA unless cells are sorted/separated before extraction. Using antigen coated beads, it was possible to avoid this issue and extract T-cell RNA only, without compromising the ability to activate the cells.

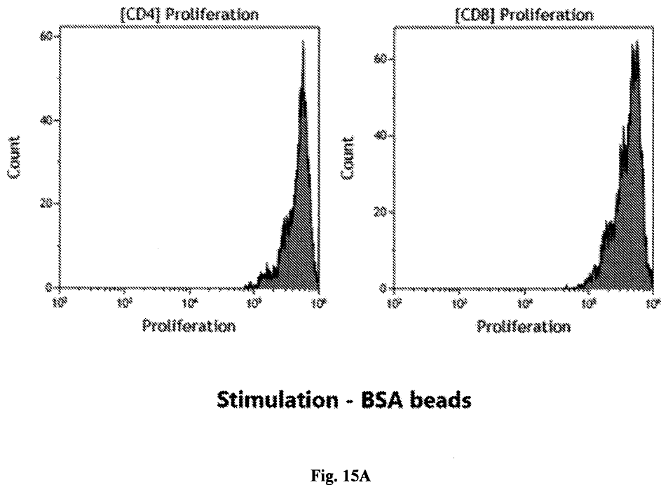

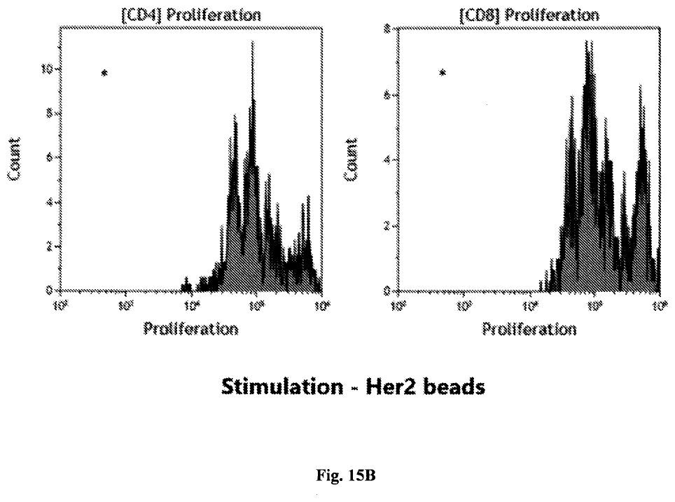

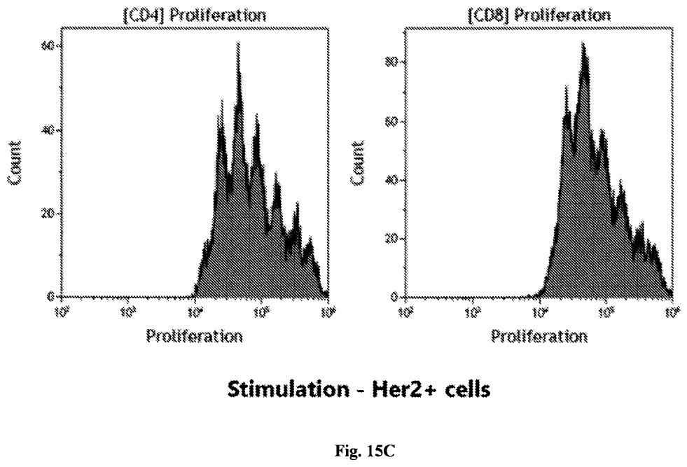

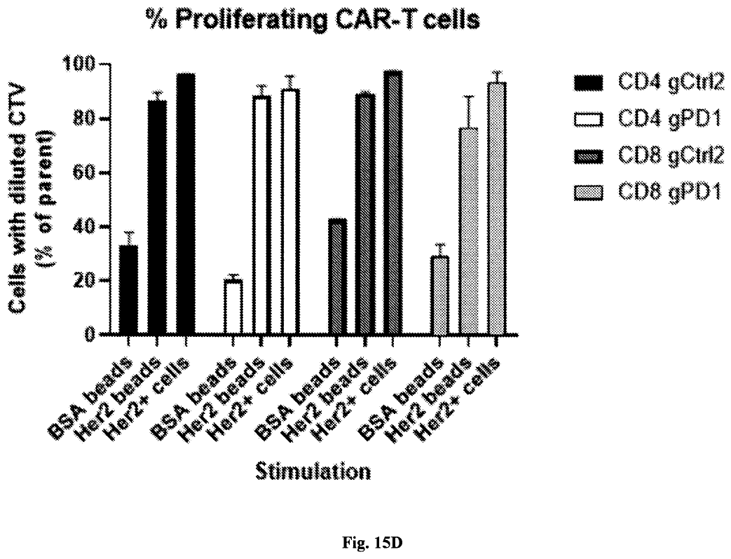

VI. Analysis of proliferation of CAR T cells. One of the important responses of T-cells to stimulation is their ability to rapidly proliferate. To explore this response, conventional HER2-CAR-T cells were incubated with either HER2 beads or FaDu-PDL1 cells (HER2+ tumor cells). Celltrace Violet fluorescent dye (CVT) was added to the culture to follow cell proliferation. The results depicted in FIG. 15A-FIG. 15D show that proliferation occurred in response to both types of stimulation at comparable levels.

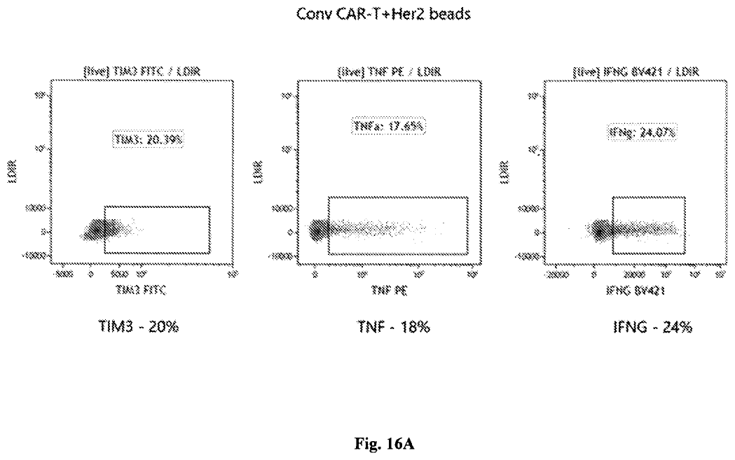

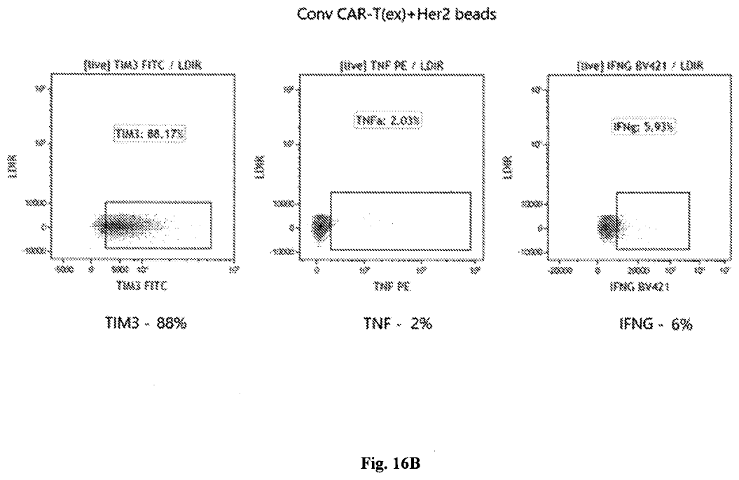

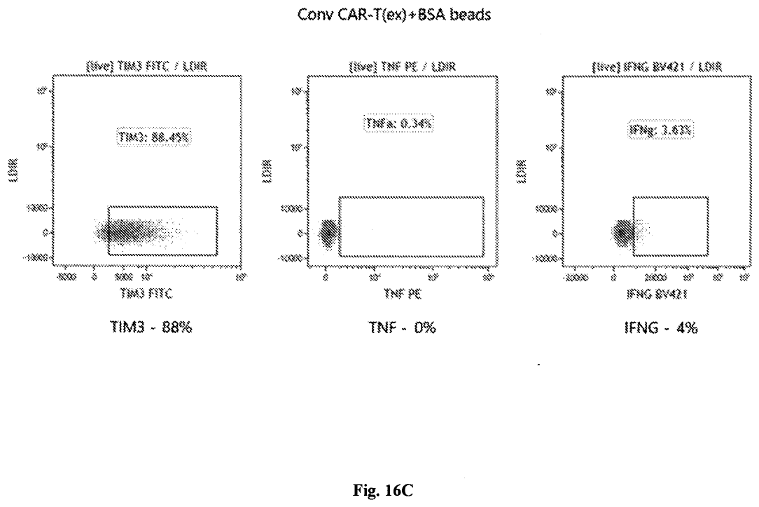

Analysis of T cell exhaustion. T-cells become exhausted and cease functioning following prolonged activation. Exhausted T-cells express specific markers such as PD-1, Tim2 and LAG3 and are unable to produce sufficient levels of cytokines. To investigate this, CAR T cells were stimulated with HER2+ tumor cells for 1-2 weeks followed by stimulation with HER2 or BSA beads. Flow cytometry was used to measure Tim3 as well as intracellular cytokine production of TNFα and IFNg. As shown in FIG. 16A-FIG. 16C, antigen coated bead assay successfully demonstrated T cell exhaustion. Compared to fresh (non-exhausted) cells, exhausted T-cells expressed higher level of Tim3 and lower levels of both cytokine. As expected, Tim3 expression was stable and was not dependent on antigen stimulation, as can be observed in the BSA bead sample.

Analysis of multiple antigen coated beads. To study the effect of co-signaling molecules conjugated together with the targeted antigen, beads conjugated to both HER2 (antigen) and PD-L1 (co-signal) were used. PDL1 is a repressor of T-cell activation and upon binding to the PD1 receptor on T-cells, suppresses T-cell function and promotes exhaustion. HER2-PDL1 beads with three PDL1 densities were used (low-mid-high) and BSA beads were used as control. Beads were incubated with conventional HER2-CAR-T cells for 24 or 72 hours at a cell: bead ratio of 1:1 (50,000 cells/well). Flow cytometry was used to evaluate CD69 and PD1 expression. As shown in FIG. 17, CD69 level was not changed by the presence of PDL1 on the beads. One explanation could be that CD69 is an early marker of activation, and T-cell activation precedes T-cell exhaustion. PD1 was slightly lower in the presence on PDL1, but not significantly.

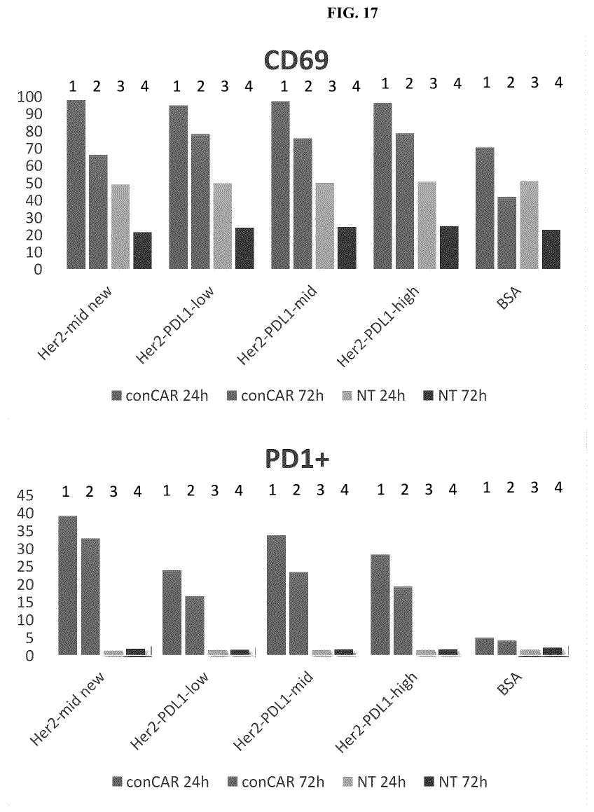

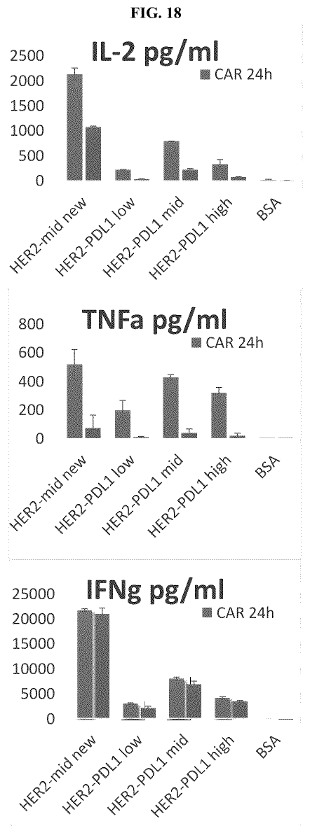

ELISA was used to measure cytokine secretion of IL2, TNFa and IFNg. All three cytokines are significantly reduced in the presence of PDL1 on the beads (FIG. 18), with PDL1 low exhibiting the strongest effect. This could be explained by the previous observation described in section I, that PDL1 density on PDL1-low beads is already very high compared to cells and PDL1-mid or -high beads likely exceed biologically relevant densities.

FIG. 19 further demonstrates the bead and antigen parameters used in the study. It was observed that while antigen amount differs significantly between beads and PDL1 positive cells, antigen density was similar, and therefore supports the functional results. HER2-PDL1-low beads are equivalent to PDL1 positive cells and higher densities do not provide additional functional effect.

Thus, by using two antigens conjugated together on one bead, it was possible to mimic not only activation, but also T-cell repression and potentially exhaustion, without the addition of a second cell type to the culture.

1. A method of assessing a plurality of cells for use in a cell therapy for a subject in need thereof, comprising:
providing the plurality of cells capable of expressing one or more chimeric receptors comprising a ligand binding domain specific for an antigen;
assessing an activity of a first sub-population of the plurality of cells upon treatment with a non-cellular substrate comprising the antigen; and
assessing a suitability of the plurality of cells for the cell therapy based upon a threshold of the activity, wherein the threshold of the activity comprises one or more members selected from the group consisting of:
(i) at least about 50% viability over at least about 24 hours;
(ii) at least about 10% increase in a percentage of the plurality of cells expressing a cell activation marker over at least about 24 hours, as compared to control cells;
(iii) at least about 5% increase in a percentage of the plurality of cells expressing the one or more chimeric receptors over 48 hours, as compared to control cells;
(iv) at least about 5% increase in a percentage of the plurality of cells expressing an immune checkpoint inhibitor over at least about 24 hours, as compared to control cells;
(v) at least about 10% increase in expression of a cytokine over at least about 24 hours, as compared to control cells;
(vi) at least about 20% increase in a percentage of the plurality of cells in a proliferative state over at least about 48 hours, as compared to control cells; or
(vii) at least about 10% change in a percentage of the plurality of cells expressing a cell exhaustion marker over at least about 24 hours, as compared to control cells.

2. The method of claim 1, wherein the threshold of the activity comprises two, three, four, five, or all members of (i)-(vi).

3. The method of claim 1, wherein the threshold of the activity comprises (i) at least about 50%, 60%, 70%, 80%, or 90% viability over at least about 24 hours.

4. The method of claim 1, wherein the threshold of the activity comprises (ii) at least about 10%, 20%, 40%, or 60% increase in the percentage of the plurality of cells expressing the cell activation marker over at least about 24 hours, as compared to control cells.

5. The method of claim 1, wherein the threshold of the activity comprises (iii) at least about 5%, 10%, 15%, or 20% increase in the percentage of the plurality of cells expressing the one or more chimeric receptors over 48 hours, as compared to control cells.

6. The method of claim 5, wherein the plurality of cells is capable of expressing an additional heterologous polypeptide, and wherein the threshold of the activity comprises at least about 5%, 10%, 15%, or 20% increase in the percentage of the plurality of cells expressing both (1) the one or more chimeric receptors and (2) the additional heterologous polypeptide over 48 hours, as compared to control cells.

7. The method of claim 6, wherein the additional heterologous polypeptide is an actuator moiety capable of regulating expression of a target gene in the plurality of cells.

8. The method of claim 7, wherein the actuator moiety comprises a Cas endonuclease or a modification thereof.

9. The method of claim 1, wherein the threshold of the activity comprises (iv) at least about 5%, 10%, 20%, 40%, or 60% increase in the percentage of the plurality of cells expressing the immune checkpoint inhibitor over at least about 24 hours, as compared to control cells.

10. The method of claim 1, wherein the threshold of the activity comprises (v) at least about 10%, 20%, 50%, 100%, 200%, 500%, or 1000% increase in expression of the cytokine over at least about 24 hours, as compared to control cells.

11. The method of claim 1, wherein the threshold of the activity comprises (vi) at least about 20%, 40%, 60%, 80%, or 100% increase in the percentage of the plurality of cells in the proliferative state over at least about 48 hours, as compared to control cells.

12. The method of claim 1, wherein the threshold of the activity comprises (vii) at least about 10%, 20%, 30%, 40%, or 50% change in the percentage of the plurality of cells expressing the cell exhaustion marker over at least about 24 hours, as compared to control cells.

13. The method of claim 12, wherein the change is an increase in the percentage of the plurality of cells.

14. The method of claim 12, wherein the change is a decrease in the percentage of the plurality of cells.

15-28. (canceled)

29. The method of claim 1, wherein the activity comprises one or more members selected from the group consisting of viability, expression of a cell activation marker, expression of one or more chimeric receptors, expression of an immune checkpoint inhibitor, expression of a cytokine, proliferation, expression of a cell exhaustion marker, chemotaxis, and metabolism.

30. The method of claim 1, wherein the cytokine comprises one or more members selected from the group consisting of IFN gamma, TNF alpha, IL-2, IL-4, and IL-10.

31. (canceled)

32. The method of claim 1, wherein the immune checkpoint inhibitor comprises one or more members selected from the group consisting of PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GALS, adenosine, and TGF.

33. The method of claim 1, wherein the cell activation marker comprises one or more members selected from the group consisting of CD69, CD25, and HLA-DR.

34. The method of claim 1, wherein the cell exhaustion marker comprise one or more members selected from the group consisting of PD1, Tim3, Lag3, IL-2, TNF alpha, and IFN gamma.

35-43. (canceled)

44. The method of claim 1, wherein the antigen, the first antigen, or the second antigen is selected from the group consisting of 707-AP, a biotinylated molecule, a-Actinin-4, abl-bcr alb-b3 (b2a2), abl-bcr alb-b4 (b3a2), adipophilin, AFP, AIM-2, Annexin II, ART-4, BAGE, b-Catenin, bcr-abl, bcr-abl p190 (e1a2), bcr-abl p210 (b2a2), bcr-abl p210 (b3a2), BING-4, CAG-3, CAIX, CAMEL, Caspase-8, CD171, CD19, CD20, CD22, CD23, CD24, CD30, CD33, CD38, CD44v7/8, CDC27, CDK-4, CEA, CLCA2, Cyp-B, DAM-10, DAM-6, DEK-CAN, EGFRvIII, EGP-2, EGP-40, ELF2, Ep-CAM, EphA2, EphA3, erb-B2, erb-B3, erb-B4, ES-ESO-1a, ETV6/AML, FBP, fetal acetylcholine receptor, FGF-5, FN, G250, GAGE-1, GAGE-2, GAGE-3, GAGE-4, GAGE-5, GAGE-6, GAGE-7B, GAGE-8, GD2, GD3, GnT-V, Gp100, gp75, Her-2, HLA-A*0201-R170I, HMW-MAA, HSP70-2 M, HST-2 (FGF6), HST-2/neu, hTERT, iCE, IL-11Rα, IL-13Rα2, KDR, KIAA0205, K-RAS, L1-cell adhesion molecule, LAGE-1, LDLR/FUT, Lewis Y, MAGE-1, MAGE-10, MAGE-12, MAGE-2, MAGE-3, MAGE-4, MAGE-6, MAGE-A1, MAGE-A2, MAGE-A3, MAGE-A6, MAGE-B1, MAGE-B2, Malic enzyme, Mammaglobin-A, MART-1/Melan-A, MART-2, MC1R, M-CSF, mesothelin, MUC1, MUC16, MUC2, MUM-1, MUM-2, MUM-3, Myosin, NA88-A, Neo-PAP, NKG2D, NPM/ALK, N-RAS, NY-ESO-1, OA1, OGT, oncofetal antigen (h5T4), OS-9, P polypeptide, P15, P53, PRAME, PSA, PSCA, PSMA, PTPRK, RAGE, ROR1, RU1, RU2, SART-1, SART-2, SART-3, SOX10, SSX-2, Survivin, Survivin-2B, SYT/SSX, TAG-72, TEL/AML1, TGFaRII, TGFbRII, TP1, TRAG-3, TRG, TRP-1, TRP-2, TRP-2/INT2, TRP-2-6b, Tyrosinase, VEGF-R2, WT1, GPC3 α-folate receptor, κ-light chain, a tumor associated antigen, and a neoantigen.

45-54. (canceled)
